SEQUENCE LISTING (108 sequences)

SEQ ID NO: 1 - DNA, 2969 bp, homo sapiens
gggtttacgctgccgccggcatccgctcggacgcggccacgttgtcttgcgcgctttgcccgcctggccctgggactctgaccctcggctaccctttcctgccccactagcgtggccgcgagcctcggtgagccggccgtattcccgctctcgcttagggggcacaggcgcaggcatcggcccggccactccaagccttcggtgcgcgggcgcgtctgggatacgggcccgggaggcgccgccctccgtccgcccggtgcctctcaggaacagcgaaccggagagagcgccggagagttgggctcagtgcagagctcggcgccggggcccatgcccgtgcgcccccgcaggccggcgccatggcctccgggagtgtggccgagtgcctgcagcaggagaccacctgccccgtgtgcctgcagtacttcgcagagcccatgatgctcgactgcggccataacatctgttgcgcgtgcctcgcccgctgctggggcacggcagagactaacgtgtcgtgcccgcagtgccgggagaccttcccgcagaggcacatgcggcccaaccggcacctggccaacgtgacccaactggtaaagcagctgcgcaccgagcggccgtcggggcccggcggcgagatgggcgtgtgcgagaagcaccgcgagcccctgaagctgtactgcgaggaggaccagatgcccatctgcgtggtgtgcgaccgctcccgcgagcaccgcggccacagcgtgctgccgctcgaggaggcggtggagggcttcaaggagcaaatccagaaccagctcgaccatttaaaaagagtgaaagatttaaagaagagacgtcgggcccagggggaacaggcacgagctgaactcttgagcctaacccagatggagagggagaagattgtttgggagtttgagcagctgtatcactccttaaaggagcatgagtatcgcctcctggcccgccttgaggagctagacttggccatctacaatagcatcaatggtgccatcacccagttctcttgcaacatctcccacctcagcagcctgatcgctcagctagaagagaagcagcagcagcccaccagggagctcctgcaggacattggggacacattgagcagggctgaaagaatcaggattcctgaaccttggatcacacctccagatttgcaagagaaaatccacatttttgcccaaaaatgtctattcttgacggagagtctaaagcagttcacagaaaaaatgcagtcagatatggagaaaatccaagaattaagagaggctcagttatactcagtggacgtgactctggacccagacacggcctaccccagcctgatcctctctgataatctgcggcaagtgcggtacagttacctccaacaggacctgcctgacaaccccgagaggttcaatctgtttccctgtgtcttgggctctccatgcttcatcgccgggagacattattgggaggtagaggtgggagataaagccaagtggaccataggtgtctgtgaagactcagtgtgcagaaaaggtggagtaacctcagccccccagaatggattctgggcagtgtctttgtggtatgggaaagaatattgggctcttacctccccaatgactgccctacccctgcggaccccgctccagcgggtggggattttcttggactatgatgctggtgaggtctccttctacaacgtgacagagaggtgtcacaccttcactttctctcatgctaccttttgtgggcctgtccggccctacttcagtctgagttactcgggagggaaaagtgcagctcctctgatcatctgccccatgagtgggatagatgggttttctggccatgttgggaatcatggtcattccatggagacctccccttgaggaggtgaattcaggccaaaagggctgttggctgtaatcctacgccaggcacaaggcatcttgttgccttgccacgtcctgtcacagctgggtatccttaccatgttccacgcccttgcagtgggagacaggatgtccatgttctctaccatccttttccttcccatgcagattgtgaaatgtaatgagatgtatcaagatatcctagaaataaaaaccagatgtccacctccagtgtttcatactttctggttttacacatcgctggagggataaagagtatggataatctttggatttggagagccgttcaagatacttccagcttcttggctcagcctggcttcctctggttcagccccacataatgattatggctatttgctgtcatttctgggctagggctcctttctaacaacctagactggaataaggccctgtcagcatggctccctttatcccagttttccgtctgggaacagtacctctgcccctgattcccaatgtgccatagttttattaactccattaaagaagcctgtatgtgttttggttagttacagttattttacaataatggtgggtaatggccccacctctgttatgagataatgttctaatcaatgtctctgcctttgtatcttttctgagggctttgtctgttctcttcattctaatgaaaggtgtattctagtgctgggtgcatatcatccaggataatattctgcccaactccatcctctgttactagatcccttaccagtcacatttgtggactggtggccagtcgtataccatccctggaaggattctgggacaatattccagggattcattgacttcttggctccttttctccatttcctttgggggaagggggaattgaccatgcttaagtgcatcctatcaaggggcagctccgtccccatggccattggatcatgagacactctgaagtcagaaggctggggcagatcacttcaagcaagcccccatgatggttctcagtcctgcttctctgtgggtacgtgcccctctgtttaaaaataaactgaatatggatgttta

SEQ ID NO: 2 - protein, 513 aa, homo sapiens
MASGSVAECLQQETTCPVCLQYFAEPMMLDCGHNICCACLARCWGTAETNVSCPQCRETFPQRHMRPNRHLANVTQLVKQLRTERPSGPGGEMGVCEKHREPLKLYCEEDQMPICVVCDRSREHRGHSVLPLEEAVEGFKEQIQNQLDHLKRVKDLKKRRRAQGEQARAELLSLTQMEREKIVWEFEQLYHSLKEHEYRLLARLEELDLAIYNSINGAITQFSCNISHLSSLIAQLEEKQQQPTRELLQDIGDTLSRAERIRIPEPWITPPDLQEKIHIFAQKCLFLTESLKQFTEKMQSDMEKIQELREAQLYSVDVTLDPDTAYPSLILSDNLRQVRYSYLQQDLPDNPERFNLFPCVLGSPCFIAGRHYWEVEVGDKAKWTIGVCEDSVCRKGGVTSAPQNGFWAVSLWYGKEYWALTSPMTALPLRTPLQRVGIFLDYDAGEVSFYNVTERCHTFTFSHATFCGPVRPYFSLSYSGGKSAAPLIICPMSGIDGFSGHVGNHGHSMETSP

SEQ ID NO: 3 - DNA, 1820 bp, homo sapiens
ggcgctgggcggctgcggcgcgcggtgcgcggtgcgtagtctggagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggtacaatgca

SEQ ID NO: 4 - protein, 261 aa, homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

SEQ ID NO: 5 - DNA, 1810 bp, homo sapiens
ggcgctgggcggctgcggcgcgcggtgcgcggtgcgtagtctggagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggtacaatgcaaaaaaaaaaaaaaaaa

SEQ ID NO: 6 - protein, 376 aa, homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 7 - DNA, 4912 bp, homo sapiens
attggggtctgctctaagctgcagcaagagaaactgtgtgtgaggggaagaggcctgtttcgctgtcgggtctctagttcttgcacgctctttaagagtctgcactggaggaactcctgccattaccagctcccttcttgcagaagggagggggaaacatacatttattcatgccagtctgttgcatgcaggctttttggcttcctaccttgcaacaaaataattgcaccaactccttagtgccgattccgcccacagagagtcctggagccacagtcttttttgctttgcattgtaggagagggactaagtgctagagactatgtcgctttcctgagctaccgagagcgctcgtgaactggaatcaactgcttcagggaaaaagaaaaaaaaaaaaaaaagacttgcctgggaggccgcgagaaacttgcattggaagcttcagcaaccagcattcgagaaactcctctctactttagcacggtctccagactcagccgagagacagcaaactgcagcgcggtgagagagcgagagagagggagagagagactctccagcctgggaactataactcctctgcgagaggcggagaactccttccccaaatcttttggggacttttctctctttacccacctccgcccctgcgaggagttgaggggccagttcggccgccgcgcgcgtcttcccgttcggcgtgtgcttggcccggggaaccgggagggcccggcgatcgcgcggcggccgccgcgagggtgtgagcgcgcgtgggcgcccgccgagccgaggccatggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctactgaagggcgcgcaggcagggagaagggccggggggggtaggagaggagaaaaaaaaagtgaaaaaaagaaacgaaaaggacagacgaagagtttaaagagaaaagggaaaaaagaaagaaaaagtaagcagggctggcttcgcccgcgttctcgtcgtcggatcaaggagcgcggcggcgttttggacccgcgctcccatcccccaccttcccgggccggggacccactctgcccagccggagggacgcggaggaggaagagggtagacaggggcgacctgtgattgttgttattgatgttgttgttgatggcaaaaaaaaaaaagcgacttcgagtttgctcccctttgcttgaagagaccccctcccccttccaacgagcttccggacttgtctgcacccccagcaagaaggcgagttagttttctagagacttgaaggagtctcccccttcctgcatcaccaccttggttttgttttattttgcttcttggtcaagaaaggaggggagaacccagcgcacccctccccccctttttttaaacgcgtgatgaagacagaaggctccggggtgacgaatttggccgatggcagatgttttgggggaacgccgggactgagagactccacgcaggcgaattcccgtttggggcttttttttcctccctcttttccccttgccccctctgcagccggaggaggagatgttgaggggaggaggccagccagtgtgaccggcgctaggaaatgacccgagaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttcccagaccccggaggcgtggaggagaggagactgtttgatgtggtacaggggcagtcagtggagggcgagtggtttcggaaaaaaaaaaagaaaaaaagaaaaaaaaagaaaaaaaaaagatttttttcttctcttaatcggaatcgtgatggtgttggattatttcaatggtggggttaatatagcatgttatcctgtctatcttttaaagatttctgtataagactgttgagcagtttttaaaatagtgtaggataatataaaaagcagatagatggcgctatgtttgattcctacaacgaaattatcaccagctttttttcattcttaactctttaaaggattcaaacgcaactcaaatctgtgctggactttaaaaaaacaattcaggaccaaattttttctcagtgtgtgtgtttattccttataggtgtaaatgagaagacgtgtttttttccttcaccgatgctccatcctcgtatttctttttccttgtaaatgtaatcagatgccattttatatgtggacgtatttatactggccaaacatattttttcttttgtccctttttttctttcctttctttttacttcctttatttctttattccttccttttcctttttttcttttttttttctttttttttttttttttttggtagttgttgttacccacgccattttacgtctccttcactgaagggctagagttttaacttttaattttttatatttaaatgtagacttttgacacttttaaaaaacaaaaaaagacaagagagatgaaaacgtttgattattttctcagtgtatttttgtaaaaaatatataaagggggtgttaatcggtgtaaatcgctgtttggatttcctgattttataacagggcggctggttaatatctcacacagtttaaaaaatcagcccctaatttctccatgtttacacttcaatctgcaggcttcttaaagtgacagtatcccttaacctgccaccagtgtccaccctccggcccccgtcttgtaaaaaggggaggagaattagccaaacactgtaagcttttaagaaaaacaaagttttaaacgaaatactgctctgtccagaggctttaaaactggtgcaattacagcaaaaagggattctgtagctttaacttgtaaaccacatcttttttgcactttttttataagcaaaaacgtgccgtttaaaccactggatctatctaaatgccgatttgagttcgcgacactatgtactgcgtttttcattcttgtatttgactatttaatcctttctacttgtcgctaaatataattgttttagtcttatggcatgatgatagcatatgtgttcaggtttatagctgttgtgtttaaaaattgaaaaaagtggaaaacatctttgtacatttaagtctgtattataataagcaaaaagattgtgtgtatgtatgtttaatataacatgacaggcactaggacgtctgcctttttaaggcagttccgttaagggtttttgtttttaaacttttttttgccatccatcctgtgcaatatgccgtgtagaatatttgtcttaaaattcaaggccacaaaaacaatgtttgggggaaaaaaaagaaaaaatcatgccagctaatcatgtcaagttcactgcctgtcagattgttgatatataccttctgtaaataactttttttgagaaggaaataaaatcagctggaactgaaccctaaaaaaaaaaaaaaaaaaaaaaaaaaaaaaaaaaaa

SEQ ID NO: 8 - protein, 474 aa, homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 9 - DNA, 5780 bp, homo sapiens
ctgggcggggccgggaagcggcagtggcggctacgcgcgcgggggtgcgcgcgggaacgaccgggaaacaccgcgagggccggggtgggccaggctgtggggacgacgggctgcgacgatggccgcagcggcgggcggcggcgggccggggacagcggtaggcgccacgggctcggggattgcggcggcagccgcaggcctagctgtttatcgacggaaggatgggggcccggccaccaagttttgggagagcccggagacggtgtcccagctggattcggtgcgggtctggctgggcaagcactacaagaagtatgttcatgcggatgctcctaccaataaaacactggctgggctggtggtgcagcttcttcagttccaggaagatgcctttgggaagcatgtcaccaacccggccttcaccaaactccctgcaaagtgtttcatggatttcaaagctggaggcgccttatgtcacattcttggggctgcttacaagtataaaaatgaacagggatggcggaggtttgacctacagaacccatctcgaatggatcgtaatgtggaaatgtttatgaacattgaaaaaacattggtgcagaacaattgtttgaccagacccaacatctacctcattccagacattgatctgaagttggctaacaaattgaaagatatcatcaaacgacatcagggaacatttacggatgagaagtcaaaagcttcccaccacatttacccatattcttcctcacaagacgatgaagaatggttgagaccggtgatgagaaaagagaagcaagtgttagtgcattggggcttttacccagacagctatgatacttgggtccatagtaatgatgttgatgctgaaattgaagatccaccaattccagaaaaaccatggaaggttcatgtgaaatggattttggacactgatattttcaatgaatggatgaatgaggaggattatgaggtggatgaaaataggaagcctgtgagttttcgtcagcggatttcaaccaagaatgaagagccagtcagaagtccagaaagaagagatagaaaagcatcagctaatgctcgaaagaggaaacattcgccttcgcctccccctccgacaccaacagaatcacggaagaagagtgggaagaaaggccaagctagcctttatgggaagcgcagaagtcagaaagaggaagatgagcaagaagatctaaccaaggatatggaagacccaacacctgtacccaatatagaagaagtagtacttcccaaaaatgtgaacctaaagaaagatagtgaaaatacacctgttaaaggaggaactgtagcggatctagatgagcaggatgaagaaacagtcacagcaggaggaaaggaagatgaagatcctgccaaaggtgatcagagtcgatcagttgaccttggggaagataatgtgacagagcagaccaatcacattattattcctagttatgcatcatggtttgattataactgtattcatgtgattgaacggcgtgctcttcctgagttcttcaatggaaaaaacaaatccaagactccagaaatatacttggcatatcgaaattttatgattgacacgtatcgtctaaacccccaagagtatttaactagcactgcttgtcggaggaacttgactggagatgtgtgtgctgtgatgagggtccatgcctttttagagcagtggggactcgttaattaccaagttgacccggaaagtagacccatggcaatgggacctcctcctactcctcattttaatgtattagctgataccccctctgggcttgtgcctctgcatcttcgatcacctcaggttcctgctgctcaacagatgctaaattttcctgagaaaaacaaggaaaaaccagttgatttgcagaactttggtctccgtactgacatttactccaagaaaacattagcaaagagtaaaggtgctagtgctggaagagaatggactgaacaggagacccttctactcctggaggccctggagatgtacaaggatgattggaacaaagtgtcggaacatgttggaagtcgtactcaggatgaatgcatcctccactttttgagacttcccattgaggacccataccttgagaattcagatgcttcccttgggcctttggcctaccagcctgtccccttcagtcagtcaggaaatccagttatgagtactgttgcttttttggcatctgtggtggaccctcgcgtggcatctgctgcagcaaaagcggctttggaggagttttctcgggtccgggaggaggtaccactggaattggttgaagctcatgtcaagaaagtacaagaagcagcacgagcctctgggaaagtggatcccacctacggtctggagagcagctgcattgcaggcacagggcccgatgagccagagaagcttgaaggagctgaagaggaaaaaatggaagccgaccctgatggtcagcagcctgaaaaggcagaaaataaagtggaaaatgaaacggatgaaggtgataaagcacaagatggagaaaatgaaaaaaatagtgaaaaggaacaggatagtgaagtgagtgaggataccaaatcagaagaaaaggagactgaagagaacaaagaactcactgatacatgtaaagaaagagaaagtgatactgggaagaagaaagtagaacatgaaatttccgaaggaaatgttgccacagccgcagcagctgctcttgcctcagcggctaccaaagccaagcacctggctgcagtggaagaaagaaagatcaagtccctggtagctctcttggttgagacacaaatgaagaaactagagatcaaacttcgacattttgaagagctggaaactatcatggacagagagaaagaagctctagaacaacagaggcagcagttgcttactgaacgccaaaacttccacatggaacagctgaagtatgctgaattacgagcacgacagcaaatggaacagcagcagcatggccagaaccctcaacaggcacaccagcactcaggaggacctggcctggccccacttggagcagcagggcaccctggcatgatgcctcatcaacagccccctccctaccctctgatgcaccaccagatgccaccacctcatccaccccagccaggtcagataccaggcccaggttccatgatgcccgggcagcacatgccaggccgcatgattcccactgttgcagccaacatccacccctctgggagtggccctacccctcctggcatgccaccaatgccaggaaacatcttaggaccccgggtacccctgacagcacctaacggcatgtatccccctccaccacagcagcagccaccgccaccaccacctgcagatggggtccctccgcctcctgctcctggcccgccagcctcagctgctccttagcctggaagatgcagggaacctccacgcccaccaccatgagctggagtggggatgacaagacttgtgttcctcaactttcttgggtttctttcaggatttttcttctcacagctccaagcacgtgtcccgtgcctccccactcctcttaccacccctctctctgacactttttgtgttgggtcctcagccaacactcaaggggaaacctgtagtgacagtgtgccctggtcatccttaaaataacctgcatctcccctgtcctggtgtgggagtaagctgacagtttctctgcaggtcctgtcaactttagcatgctatgtctttaccatttttgctctcttgcagttttttgctttgtcttatgcttctatggataatgctatataatcattatctttttatctttctgttattattgttttaaaggagagcatcctaagttaataggaaccaaaaaataatgatgggcagaagggggggaatagccacaggggacaaaccttaaggcattataagtgaccttatttctgcttttctgagctaagaatggtgctgatggtaaagtttgagacttttgccacacacaaatttgtgaaaattaaacgagatgtggaaggagaacctcagtgattttattccctagtgaggcctctgagggcctccacactgcctggcagaacataccactgaactagtatgtgctagaggagggcacaaacatccgctccttccctaggcctgctggctctggttttctatgcagatgattcattggattgggggtgagtgttttgtttttctgggggcagtgtgagctttgagggttggaatattgggaggcattccttagtttcctcaactagcctggaaagttaggagtctagggtaattacccccaatgagtctagcctactattcactgctttgtgtgcatttttttctccctctttaaaaaaccctttaaaagaaaaaaaaaagtagatagtgctaaatattttagctcatgaaacttggttaggatggctgggggtacaagtccccaaactacctcttgttacagtagccagggagtggaatttcgtcaaccggtacttttaaggttaggatgggacgggaaaagtgaagcaggatattagctccttataccttctcccttccatttctgagatctcacattccatctatcacagggttttcaaagagatgctgagggtaacaaggaactcacttggcagtcagagcatcatgctttgaggtttggggtgctcaggctgggagggtagaatgccattccagaggacaagccacaaaaatgccttaatttgagctcgtatttacccctgctgataagtgacttgagagttcccggttttttcctcttgtccttccctcccttctgtccttccatgtgtggggaaagggtgtttttggtagagcttggtttccaaagcgcctggctttctcacttcacattctcaagtggcagtttcattatttagaatgcaaggtggacatcttttggatatctttttctatatattttctaaagctttacatatgagagggtatagggaggtgtttataaaacacttgagaacttttttccttaatatcagaaagcaaaaaaataaaaccacaattgagatttgcctttcaaaccctcaggtttgcctctaaccaggtgtccctggtcaccatcagagtactggaatacgggaaccgaggagaccttggtccttttgtttttgttctggactcttgggagtggaaatgagaatgagtttattcctactggagcttagttccaatgcatttggctccagaaagaccccagtgccttttgacaatggccagggttttacctacttcctgccagtctttcccaaaggaaactcattccaaatacttcttttttcccctggagtccgagaaggaaaatggaattctggttcatactgtggtcccttgtaacctcaggtctttaatgtgatcactttcaaatttaaaagatccaggtggaaatatttttactatagtaataattctacaaaatacctgaattcttaacactgttatatttcagtataagtggtggctttttcttttcatgtctttgatctggttttattcctgtaattcagccacctgattttgtgaggggggggaataatatgtggtttttgtacaaacatgtttctcagtgtgttgttattttggaaaaaatgaggggagggagtttggcaagaatggagaaaatgaatgaagaaggcctaatctctctctttttcagtgaataaatggaacaccatttctggattctaaaaaaaaaaaaaaaaaaaaaaaa

SEQ ID NO: 10 - protein, 1105 aa, homo sapiens
MAAAAGGGGPGTAVGATGSGIAAAAAGLAVYRRKDGGPATKFWESPETVSQLDSVRVWLGKHYKKYVHADAPTNKTLAGLVVQLLQFQEDAFGKHVTNPAFTKLPAKCFMDFKAGGALCHILGAAYKYKNEQGWRRFDLQNPSRMDRNVEMFMNIEKTLVQNNCLTRPNIYLIPDIDLKLANKLKDIIKRHQGTFTDEKSKASHHIYPYSSSQDDEEWLRPVMRKEKQVLVHWGFYPDSYDTWVHSNDVDAEIEDPPIPEKPWKVHVKWILDTDIFNEWMNEEDYEVDENRKPVSFRQRISTKNEEPVRSPERRDRKASANARKRKHSPSPPPPTPTESRKKSGKKGQASLYGKRRSQKEEDEQEDLTKDMEDPTPVPNIEEVVLPKNVNLKKDSENTPVKGGTVADLDEQDEETVTAGGKEDEDPAKGDQSRSVDLGEDNVTEQTNHIIIPSYASWFDYNCIHVIERRALPEFFNGKNKSKTPEIYLAYRNFMIDTYRLNPQEYLTSTACRRNLTGDVCAVMRVHAFLEQWGLVNYQVDPESRPMAMGPPPTPHFNVLADTPSGLVPLHLRSPQVPAAQQMLNFPEKNKEKPVDLQNFGLRTDIYSKKTLAKSKGASAGREWTEQETLLLLEALEMYKDDWNKVSEHVGSRTQDECILHFLRLPIEDPYLENSDASLGPLAYQPVPFSQSGNPVMSTVAFLASVVDPRVASAAAKAALEEFSRVREEVPLELVEAHVKKVQEAARASGKVDPTYGLESSCIAGTGPDEPEKLEGAEEEKMEADPDGQQPEKAENKVENETDEGDKAQDGENEKNSEKEQDSEVSEDTKSEEKETEENKELTDTCKERESDTGKKKVEHEISEGNVATAAAAALASAATKAKHLAAVEERKIKSLVALLVETQMKKLEIKLRHFEELETIMDREKEALEQQRQQLLTERQNFHMEQLKYAELRARQQMEQQQHGQNPQQAHQHSGGPGLAPLGAAGHPGMMPHQQPPPYPLMHHQMPPPHPPQPGQIPGPGSMMPGQHMPGRMIPTVAANIHPSGSGPTPPGMPPMPGNILGPRVPLTAPNGMYPPPPQQQPPPPPPADGVPPPPAPGPPASAAP

SEQ ID NO: 11 - DNA, 1426 bp, homo sapiens
gactatctcccagcggcaggcccttcgataaaatcaggaacttgtgctggccctgcaatgtcaagggagggggctcacccagggctcctgtagctcagggggcaggcctgagccctgcacccgccccacgaccgtccagcccctgacggggcaccccatcctgaggggctctgcattggcccccaccgaggcaggggatctgaccgactcggagcccggctggatgttacaggcgtgcaaaatggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactgagcccgcagcccccgccgggccccgccaggcctccccgctggccatagcattaagccctcgcccggcccggacacagggaggacgctcccggggcccagaggcaggactgtggcgggccgggcctcgcctcacccgccccctccccccactccaggccccctccacatcccgcttcgcctccctccaggactccaccccggctcccggacgccagctgggcgtcagaccccaccggggcaaccttgcagaggacgacccggggtactgccttgggagtctcaagtccgtatgtaaatcagatctcccctctcacccctcccacccattaacctcctcccaaaaaacaagtaaagttattctcaatccatcaaaaaaaaaaaaaaaaaa

SEQ ID NO: 12 - protein, 271 aa, homo sapiens
MLQACKMEGFPLVPPQPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 13 - DNA, 1423 bp, homo sapiens
gactatctcccagcggcaggcccttcgataaaatcaggaacttgtgctggccctgcaatgtcaagggagggggctcacccagggctcctgtagctcagggggcaggcctgagccctgcacccgccccacgaccgtccagcccctgacggggcaccccatcctgaggggctctgcattggcccccaccgaggcaggggatctgaccgactcggagcccggctggatgttacaggcgtgcaaaatggaagggtttcccctcgtcccccctccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactgagcccgcagcccccgccgggccccgccaggcctccccgctggccatagcattaagccctcgcccggcccggacacagggaggacgctcccggggcccagaggcaggactgtggcgggccgggcctcgcctcacccgccccctccccccactccaggccccctccacatcccgcttcgcctccctccaggactccaccccggctcccggacgccagctgggcgtcagaccccaccggggcaaccttgcagaggacgacccggggtactgccttgggagtctcaagtccgtatgtaaatcagatctcccctctcacccctcccacccattaacctcctcccaaaaaacaagtaaagttattctcaatccatcaaaaaaaaaaaaaaaaaa

SEQ ID NO: 14 - protein, 270 aa, homo sapiens
MLQACKMEGFPLVPPPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 15 - DNA, 2084 bp, homo sapiens
aaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctctgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaa

SEQ ID NO: 16 - protein, 380 aa, homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 17 - DNA, 3428 bp, homo sapiens
tggagagtaatgttacagagcggagagagtgaggaggctgcgtctggctcccgctctcacagccattgcagtacattgagctccatagagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaa

SEQ ID NO: 18 - protein, 215 aa, homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 19 - DNA, 3510 bp, homo sapiens
gaggccgcgcggggcccgggcttcggccgatcagcccgggaggccccgccgcgcccccttggcccgcgcgcccgtggtcacagtggaagaggcgcccgcgctgcgctgcccggaggagccgtcgcgcgcccgcttcctgttcggctggttcctgccagctcgaggacaaaacacgcgtgcgcgcggcgggcgagcgcgctcgccgcctcagtcgccagcgccgggcgcagtccgcctttttccggagcagactggccgcggtgctagtcggtagcagcggccgccgcagcggctccgcactggcgaaccgagggcagaaaaaggcggggttgacggctttttggtaggagtgggctggaccggacgccagagacaaaggctcccaaggcaagagggactgtggccctgcgtcggctctgctcgggactgctgaccccaggaatttacgccccttcgtttttctcttctgattcttctcttctcccaagcccgcgtcccctcacgcgtggcctctctccttgccgggagggccgcgatggaggtcccgcccaggctttcccatgtgccgccgccattgttcccctccgctcccgctactttagcctcccgcagcctctcccattggcggccgcggccgccgcggcagctagccccgctcctcccttcgctcgctcccagctccgcccggcagggggcgcgccgggcccagcgccacgtcaccgcccagcagccctcccgattggcgggcggggcggctataaagggagggcgcaggcggcgcccggatctcttccgccgccattttaaatccagctccatacaacgctccgccgccgctgctgccgcgacccggactgcgcgccagcacccccctgccgacagctccgtcactatggaggatatgaacgagtacagcaatatagaggaattcgcagagggatccaagatcaacgcgagcaagaatcagcaggatgacggtaaaatgtttattggaggcttgagctgggatacaagcaaaaaagatctgacagagtacttgtctcgatttggggaagttgtagactgcacaattaaaacagatccagtcactgggagatcaagaggatttggatttgtgcttttcaaagatgctgctagtgttgataaggttttggaactgaaagaacacaaactggatggcaaattgatagatcccaaaagggccaaagctttaaaagggaaagaacctcccaaaaaggtttttgtgggtggattgagcccggatacttctgaagaacaaattaaagaatattttggagcctttggagagattgaaaatattgaacttcccatggatacaaaaacaaatgaaagaagaggattttgttttatcacatatactgatgaagagccagtaaaaaaattgttagaaagcagataccatcaaattggttctgggaagtgtgaaatcaaagttgcacaacccaaagaggtatataggcagcaacagcaacaacaaaaaggtggaagaggtgctgcagctggtggacgaggtggtacgaggggtcgtggccgaggtcagggccaaaactggaaccaaggatttaataactattatgatcaaggatatggaaattacaatagtgcctatggtggtgatcaaaactatagtggctatggcggatatgattatactgggtataactatgggaactatggatatggacagggatatgcagactacagtggccaacagagcacttatggcaaggcatctcgagggggtggcaatcaccaaaacaattaccagccatactaaaggagaacattggagaaaacaggaggagatgttaaagtaacccatcttgcaggacgacattgaagattggtcttctgttgatctaagatgattattttgtaaaagactttctagtgtacaagacaccattgtgtccaactgtatatagctgccaattagttttctttgtttttactttgtcctttgctatctgtgttatgactcaatgtggatttgtttatacacattttatttgtatcatttcatgttaaacctcaaataaatgcttccttatgtgattgcttttctgcgtcaggtactacatagctctgtaaaaaatgtaatttaaaataagcaataattaaggcacagttgattttgtagagtattggtccatacagagaaactgtggtcctttataaatagccagccagcgtcaccctcttctccaatttgtaggtgtattttatgctcttaaggcttcatcttctccctgtaactgagatttctaccacacctttgaacaatgttctttcccttctggttatctgaagactgtcctgaaaggaagacataagtgttgtgattagtagaagctttctagtagaccatatttcttctggattgtaataaaattgttagtagctccttttactttgttcctgtctctggaaagccatttttgaattgctgattactttggctttaatcagtggtcacctagaaaaagctttgtaatcataacacaatgagtaattcttgataaaagttcagatacaaaaggagcactgtaaaactggtaggagctatggtttaagagcattggaagtagttacaactcaaggattttggtagaaaggtatgagtttggtcgaaaaattaaaatagtggcaaaataagatttagttgtgttttctcagagccgccacaagattgaacaaaatgttttctgtttgggcatcctgaggaagttgtattagctgttaatgctctgtgagtttagaaaaagtcttgatagtaaatctagtttttgacacagtgcatgaactaagtagttaaatatttacatattcagaaaggaatagtggaaaaggtatcttggttatgacaaagtcattacaaatgtgactaagtcattacaaatgtgactgagtcattacagtggaccctctgggtgcattgaaaagaatccgttttatatccaggtttcagaggacctggaataataaaaagctttggattttgcattcagtgtagttggattttgggaccttggcctcagtgttatttactgggattggcatacgtgttcacaggcagagtagttgatctcacacaacgggtgatctcacaaaactggtaagtttcttatgctcatgagccctccctttttttttttaatttggtgcctgcaactttcttaacaatgattctacttcctgggctatcacattataatgctcttggcctcttttttgctgctgttttgctattcttaaacttaggccaagtaccaatgttggctgttagaagggattctgttcattcaacatgcaactttagggaatggaagtaagttcatttttaagttgtgttgtcagtaggtgcggtgtctagggtagtgaatcctgtaagttcaaatttatgattaggtgacgagttgacattgagattgtccttttccctgatcaaaaaatgaataaagcctttttaaacaaaatccaaacttttaa

SEQ ID NO: 20 - protein, 420 aa, homo sapiens
MEVPPRLSHVPPPLFPSAPATLASRSLSHWRPRPPRQLAPLLPSLAPSSARQGARRAQRHVTAQQPSRLAGGAAIKGGRRRRPDLFRRHFKSSSIQRSAAAAAATRTARQHPPADSSVTMEDMNEYSNIEEFAEGSKINASKNQQDDGKMFIGGLSWDTSKKDLTEYLSRFGEVVDCTIKTDPVTGRSRGFGFVLFKDAASVDKVLELKEHKLDGKLIDPKRAKALKGKEPPKKVFVGGLSPDTSEEQIKEYFGAFGEIENIELPMDTKTNERRGFCFITYTDEEPVKKLLESRYHQIGSGKCEIKVAQPKEVYRQQQQQQKGGRGAAAGGRGGTRGRGRGQGQNWNQGFNNYYDQGYGNYNSAYGGDQNYSGYGGYDYTGYNYGNYGYGQGYADYSGQQSTYGKASRGGGNHQNNYQPY

SEQ ID NO: 21 - DNA, 1324 bp, homo sapiens
ctggtgaggggctgcaggtggcggcgcagtctcggtaggcggtatgagtttggctgggggccgggcaccccggaagaccgctgggaaccggctttctgggcttttggaggcagaggaggaagatgagttctaccagacgacttatgggggtttcacagaggaatccggagatgatgagtatcaaggggaccagtcagacacagaggacgaagtggactctgactttgacattgatgaaggggatgaaccatccagtgatggagaagcagaagagccaagaaggaagcgccgagtagtcaccaaggcctataaggaacctctcaagagcttaaggcctcgaaaggtcaacaccccggctggtagctctcagaaggcgcgagaagagaaggcactactgccattagaactacaagatgacggctctgacagtcggaagtctatgcgtcagtctacagctgagcatacacgacaaacgttccttcgggtacaggagaggcagggccagtcaagacggcgaaaggggccccactgtgagcggccactaacccaggaggaactgctccgggaggccaagatcacagaagagcttaatttacggtcactggagacatatgagcggctcgaggctgataaaaagaagcaggttcataagaagcggaagtgccccgggcccataatcacctatcattcagtgacagtgccacttgttggggagccaggccccaaggaagagaacgttgacatagaaggacttgatcctgctccctcggtgtctgcattgactcctcatgctgggactggacccgtcaacccccctgctcgctgctcacgtaccttcatcacttttagtgatgatgcaactttcgaggaatggttcccccaagggcggcccccaaaagtccctgttcgtgaggtctgtccagtgacccatcgtccagccctataccgggaccctgttacagacataccctatgccactgctcgagccttcaagatcattcgtgaggcttacaagaagtacattactgcccatggactgccgcccactgcctcagccctgggccccggcccgccacctcctgagcccctccctggctctgggccccgagccttgcgccagaaaattgtcattaaatgaagagatgtctagtcctcagaaacttctttcctgccctgattggggctcttgctgttccgtttcttctccctgcttctcccctttgtcatctctgatctttgcctaatctgtttctttttccttttcccctagttcttacaggtttcgttgtgttttttaatctaataaaatagaaagatccctttt

SEQ ID NO: 22 - protein, 364 aa, homo sapiens
MSLAGGRAPRKTAGNRLSGLLEAEEEDEFYQTTYGGFTEESGDDEYQGDQSDTEDEVDSDFDIDEGDEPSSDGEAEEPRRKRRVVTKAYKEPLKSLRPRKVNTPAGSSQKAREEKALLPLELQDDGSDSRKSMRQSTAEHTRQTFLRVQERQGQSRRRKGPHCERPLTQEELLREAKITEELNLRSLETYERLEADKKKQVHKKRKCPGPIITYHSVTVPLVGEPGPKEENVDIEGLDPAPSVSALTPHAGTGPVNPPARCSRTFITFSDDATFEEWFPQGRPPKVPVREVCPVTHRPALYRDPVTDIPYATARAFKIIREAYKKYITAHGLPPTASALGPGPPPPEPLPGSGPRALRQKIVIK

SEQ ID NO: 23 - DNA, 1540 bp, homo sapiens
caaaggccaggcctgacggaagagcccagccttcgccgagggccgcagggagccagagactcgagcctgcggttccagccccgccgcggttccggccccgccgcgatcccgaggcctgcgcatgctcggtgctgcgcggggctccctccttaccgcaggagctcctcatcgagaaacccaggttggtgtcttttccacgtggtagaagctggctaacctccacttccacattccagcccaagggaaagtccaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 24 - protein, 252 aa, homo sapiens
MDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 25 - DNA, 1369 bp, homo sapiens
cacaacatttccttgtatgtggatagcgtgaaagaagcttgtgatcctgcccccaccctccccaccgccacattttttaaagtgtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 26 - protein, 252 aa, homo sapiens; identical to SEQ ID NO: 24

SEQ ID NO: 27 - DNA, 1382 bp, homo sapiens
gtttctggacccggccaccccccgcgtccgcaccgccgcccccgagggacgagtgacagcggccgccgcgctcggtccagtcttcccggctggaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 28 - protein, 252 aa, homo sapiens; identical to SEQ ID NO: 24

SEQ ID NO: 29 - DNA, 1508 bp, homo sapiens
gcggccgccgcgctcggtccagtcttcccggctggaaggagcgggcgcctgggatcagagcgtcctgtttagcaataacggctggagcacgtcctacaagttacgggagagtcggctgtgaaggagacgttcgcttatcccctgtgtccccgctcctggcccctccagacccccgccttgcctcgcgctgggaggggagatccagaatgaaaggcaagaaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 30 - protein, 272 aa, homo sapiens
MKGKKGIVAASGSETEDEDSMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 31 - DNA, 2076 bp, homo sapiens
acattctttcagacccggcccgctgcggggcgttcctggggggtagcctcaaggccagcggggttccttcggctgcgtttctgtgggaggccctgaaacgcgcggagcttccctctgcctccaggctttcccagcgagagtgaaattaaacttgaaactcggatcaactggcagtcgttgttggtagaacgccctaaggacccctccccgcgggacggagggaggactcgggacagggaattggccctgggagaaaacgcgcggggggcgtccgagacgccccgtgaaagccgtgccgacccttgggaggactgacaggtctagagacacgcgctgtctgttgtggtgggcctcccgggaataagtgaggggctctgtgtttcgaggatggttctagcgcagagccgggtgtctgccggggtgggctccccgcattgttcgggctccggcgggggcggctctgattcctttccatggcccgcctcccaccccgggaatccccagtgctccttttccacggcttttctggcgtccccccgactctcccgcggcactttggcctaccttcccccagcgccgtggtcctccctggcgaccccctctgcgctcctggggtcctcctgcgccccccctcctccaccggcgcgctgcccacagccgcgtgccctctccccggagctggggaccaaggctgggccccgccggccgcatcggtgggaacttccgcggtccccatcccagggcgcacagggtccagctcctcggcgccgactcctggaaacaatgaaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 32 - protein, 401 aa, homo sapiens
MVLAQSRVSAGVGSPHCSGSGGGGSDSFPWPASHPGNPQCSFSTAFLASPRLSRGTLAYLPPAPWSSLATPSALLGSSCAPPPPPARCPQPRALSPELGTKAGPRRPHRWELPRSPSQGAQGPAPRRRLLETMKGIVAASGSETEDEDSMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 33 - DNA, 1618 bp, homo sapiens
tctgacagcgccgaggtgcgccgagcaggagcagggaacaaaggagcggagaggggaggggagagagttgggcgagggagagcccccggccggctgccagaagatcccggcgggaggaagcccaagtgtcacttgaattccacccaaggagcgggcgcctgggatcagagcgtcctgtttagcaataacggctggagcacgtcctacaagttacgggagagtcggctgtgaaggagacgttcgcttatcccctgtgtccccgctcctggcccctccagacccccgccttgcctcgcgctgggaggggagatccagaatgaaaggcaagaaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 34 - protein, 272 aa, homo sapiens; identical to SEQ ID NO: 30

SEQ ID NO: 35 - DNA, 1469 bp, homo sapiens
acattctttcagacccggcccgctgcggggcgttcctggggggtagcctcaaggccagcggggttccttcggctgcgtttctgtgggaggccctgaaacgcgcggagcttccctctgcctccaggctttcccagcgagagtgaaattaaacttgaaactcggatcaactggcagtcgttgttggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 36 - protein, 252 aa, homo sapiens; identical to SEQ ID NO: 24

SEQ ID NO: 37 - DNA, 1474 bp, homo sapiens
agcgttggctggggggaggcagtgggggtgcatctacgggagcggttgggctgtaagctttcgctacatcacagaattgtgccagtgtttctctttggagtgcctcgccagctttaacaaccgtttggatatgacttgctcgggcaaaagttgtgcattggcccgatcaagcctgacttctagccaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgattttttttttcctccccttccctttttttgttattttttcagactgtgcaatacttagagaacctatagcatcttctcattcccatgtggaacaggatgcccacatactgtctaattaataaattttccattttttttcaaacaagtatgaatctagttggttgatgccttttttttcatgacataataaagtattttctttaaaaaaaaaaaaaaaaa

SEQ ID NO: 38 - protein, 286 aa, homo sapiens
MTCSGKSCALARSSLTSSQGIVAASGSETEDEDSMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 39 - DNA, 2944 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 40 - protein, 781 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSCMEPMETAEPQSSPAHSSPAHSSPAHSSPVQSLLRAQGASSLPCGTYHPPAWPPHQPAEQAATPDAEPHSEPPDHQERPAVHRGIRYLLYRAQRAIRLRHALRLHPQLHRAPIRTWSPHVVQASTPAITGPLNHPANAQEHPAQLQRGISPPHRIRGAVRSRSRSLRGSSHLSQWLNNFFALPFSSMASQLDMSSVVGAGESRAQTLGAGVPPGDSVRGSMEASQVQVPLEASPITFPPPCAPERPPISPVPGARQAGL

SEQ ID NO: 41 - DNA, 3751 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcggtgagtggcccagaagttcagcccaggactcctgcctccccccatttcaggtcccagggggcacagccacagcaggtgactctcagacttgccttgcgcctggggaattttccagtgaggcattgagtcccaagctgtgctgaggacagtctccaaatgacagctgcatgcctggaccaccccagccctcccactacaccagggccaggagctcttctgaaatgctgatagcatgtgtccagagccctgtctcttttctggaatgtccaagacctttgtctggagcttccttatgcattttctgtcctctaagtcctcagtgaggagggctggacaagaatccattccttggtttattacagccagtgggggccagtgaaggggtgtcaggccacagggcagctatataggggccaaacaagtgaggtttgactccatccatgtagaaagatatataaatccattccacagtgaaacaggtggcctcgtgggtagtgacccttctgtccctagaggtttattactagaggctggactatcacctgtccaggggaaaaggggatctgaatagagtgaaaggttggactgggtggcccctgaggtctcttccagccctcagtctgaggttctgtattggaaagtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaaaaaaaaaaaaaaaaaaaaaaaa

SEQ ID NO: 42 - protein, 611 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSVSGPEVQPRTPASPHFRSQGAQPQQVTLRLALRLGNFPVRH

SEQ ID NO: 43 - DNA, 3088 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 44 - protein, 829 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSCMEPMETAEPQSSPAHSSPAHSSPAHSSPVQSLLRAQGASSLPCGTYHPPAWPPHQPAEQAATPDAEPHSEPPDHQERPAVHRGIRYLLYRAQRAIRLRHALRLHPQLHRAPIRTWSPHVVQASTPAITGPLNHPANAQEHPAQLQRGISPPHRIRGAVRSRSRSLRGSSHLSQWLNNFFALPFSSMASQLDMSSVVGAGESRAQTLGAGVPPGDSVRGSMEASQVQVPLEASPITFPPPCAPERPPISPVPGARQAGL

SEQ ID NO: 45 - DNA, 2254 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttagtctctgagtcccaaaaagaagtgcaggcagagccatctgccaggcccaggagagctctgagctctggccaacaactgcagccaggctgggcagagcactccggctcacctgggctcctggcgtgtcatttgctggcttgaataaagatgtccgccttatccagtgcctgagtgtgcgagagaggcagatgcctccaaaaaaaaaaaaaaaaa

SEQ ID NO: 46 - protein, 633 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNESGFSWGYPHPFLI

SEQ ID NO: 47 - DNA, 2110 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttagtctctgagtcccaaaaagaagtgcaggcagagccatctgccaggcccaggagagctctgagctctggccaacaactgcagccaggctgggcagagcactccggctcacctgggctcctggcgtgtcatttgctggcttgaataaagatgtccgccttatccagtgcctgagtgtgcgagagaggcagatgcctccaaaaaaaaaaaaaaaaa

SEQ ID NO: 48 - protein, 585 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNESGFSWGYPHPFLI

SEQ ID NO: 49 - DNA, 5600 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaacccagaagattagccagctggctgcggtgaaccgggaaagcaagttccgcgtggtcatccagcctgaagccttcttcagcatctactccaaggccgtgtccctggaggtggggctgcagcacttcctcagctttctgagctccatgcgccgccctatcttggcctgctacaagctgtgggggcctggcctcccaaacttcttccgggccctggaggacattaacaggctgtgggaattccaggaggccatctcgggcttcctggctgccctgcctctcatccgggagcgtgtgcccggggccagcagcttcaaactcaagaacctggcccagacctacctggcgagaaacatgagcgagcgcagcgccatggctgccgtgctggccatgcgtgacctgtgccgcctcctcgaggtctccccgggcccccagctggcccagcatgtctaccccttcagtagcctgcagtgctttgcctccctgcagcccctggtgcaggcagctgtgctgccccgggctgaggcccgcctcctggccctacacaacgtgagcttcatggagctgctgagtgcacaccgccgtgaccggcaggggggcctgaagaagtacagccgctatctaagcctgcagaccaccacgttgccccctgcccagcctgctttcaacctgcaggctctgggcacctactttgaaggcctgttggagggtccggcgctggcacgggcagaaggagtctccaccccacttgctggccgtggcttggcagagagggcctcccagcagagctgagaggagggggtgaccagcttggagtctctggtgggcagagagggatggggtccctgagccaggccccacccatcacagcattcccaggtcctggtacccagccctcagttgtcatttggttcagaatcagttccctttctctgggaccaaatttcccttctctaaacatcctacagagaaggttccaaagctgagcacccatcaccctaggtgtgcaccagactcctattagcccctccttccaggagctagaaccagggaggtcctgagtgaggaaggcatgacctctgggctctctaggtggccctggtcttgccccatccatcccacctgccactaccttgggtgtccttacagctctgccctgaccccagcccctgcccctggacacctgctgacagctcccacacagaacagtctgaggtgatgctggctacagccctggcagcccatggcaactcaaagtgccttccaaaccaaactgcccctcatgaggttagggtcccccaccctccacctgcccagaggccaactctgttcccttctcctttcatcccagaggggcctcatcagcaaagacagaaactgatgttccatgcatgtccctgcttccaaagcctgaccttccaaagcttgcttccttcctcctggctgcacagacacctctgcttggccacagctttgctctttggtcctcaggccaccagacccctcacagcatggcccttggctcttcctgcactgccctcaccctcagccgtcccaaggagtgcccagcactactcagcatgtagtccaggacctgcggcatcagcatcccctgggagcttcttagaaatgcagacctgtgggctctaccacaggcctctggaatcagaatcttcagggtggggcccagcagtctgtgttttaacaggttctcccggtgatgctggtgcatgctcaagtttgagaaccgctgctctcatagactgctcctccaaggggaagcagtgtggaacagcagagaaagtcccgtccctatctctgactgtgcagtgagtgtggcctaggaacaggtcccttcctaagctctagtgtccccatctgtaaaatgggctgattggcctccacggtcagagcggccatctggctgtgccatcctgcatttttaggaatggaaagcaggcctctgaggcagtggacaggaagacttctcatccttgaattctagctcccattccaaactgttagtccccaaccttgtggtcacatcagaggtcaacagcagaacagaggcaggtcagggttgtccgctctgattagcagaacgacagccccttcctcctcctccctccttcccatccctactcattagctccctgctcctgggatgcttcccaagcagcccaggcctctagcctccatggctgatgacctcagctttcacactggtgaagtctgtgtacccatactgcagccccatcccatggggcccctgaagacccactgaggaattccgtagggtcttgttcccacgaccggagtgctggctctcacagtgaattttgatgcatttaaaataagattctgatgccagactgttaaaacaggcgctggttctgaagcaccgatggaaacagattgatgcagatggaaggaggaagggagactgggcccactgatttccagccccaccatgtgtgctgttttcagatgtgattgaggggtgttctgccctgcctccactgtcacagcctttaataaagatgtgaatcttgaaagcctgatgctccaatcacagactctgctcagcatccccagaggaaccactgagaagccttttgcctggataggagggggctctcccaggcataggcttgtggccacttgcccagcacaactgtgttttgtggggcgtcttttaggacttagcagagctgagagccctttgttgctcaatgctgtgagccttaagcatgtgaatccccccacatgacaagtggggagacccagggtaggctttcctttggatcatctccaaatgggagtgccatgtggcaggaaagaagcaccgatttcaagaattacttcctagagaaagtcaggaactagagaccagagtctgcaggaactttgccactgcccttcactggctaatcctgtacttctctctgtgttccttgagtgacaggtggtaaaacccttaaaaagggaggtgtgggaggcccaggactttggtaacaggtgatgaatgtgttgtcttggccatcacaggtggagcttcagttagtacccgagggattcccctggcacaagcattataggaattagtccaccactggcgtgggtgagcagccaggtaatgggaattgatcagtgccaccacccttgaccccaactaccatcccagatgcccagattccagtcctggagttttattctctcagcttgatcctttgaccaagaagatccagtcccaatatgtcacctgtgcttcatctttggactatatctcaggtgtttacgtgtcaactaatgggagcttactgggttagaagtcaggacactgagtttcaatcctcacttagtagtctgtggccctctttggcacattaacctccctccttgatcctcaggctgcacctctggcaaatgggagaatgcggctccttttgacttcaagggatctttgtgggaaccaatgagaaaatgtgtgtgaagatagctttttggtatattttaaaagtgcacaaatttaagatcttactgctttataaagtgctttatgaattataggaaaataaacacattttagatcttagaaaaaaa

SEQ ID NO: 50 - protein, 882 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNETQKISQLAAVNRESKFRVVIQPEAFFSIYSKAVSLEVGLQHFLSFLSSMRRPILACYKLWGPGLPNFFRALEDINRLWEFQEAISGFLAALPLIRERVPGASSFKLKNLAQTYLARNMSERSAMAAVLAMRDLCRLLEVSPGPQLAQHVYPFSSLQCFASLQPLVQAAVLPRAEARLLALHNVSFMELLSAHRRDRQGGLKKYSRYLSLQTTTLPPAQPAFNLQALGTYFEGLLEGPALARAEGVSTPLAGRGLAERASQQS

SEQ ID NO: 51 - DNA, 3096 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcaggtagggagaggaacgcgttgtggtgatcagcagctcggaagactcagatgccgaaaactcgtgcatggagcccatggagaccgccgagccacagtcctcgccagcccactcctcgccagcccactcctcgccagcccactcctcgccagtccagtctctgctgagagcacaaggagcctccagcctgccctgtggcacataccaccccccagcttggcctccccaccagcccgctgagcaggctgccacccccgatgctgagcctcacagcgagcctcctgatcaccaggagcgccctgccgtccaccgtgggatccgctacctgttgtacagagcacagagagccatccgccttcgccatgccctccgcttgcaccctcaattgcatcgggcccctattcggacttggtctccccatgtggtccaagccagcactcctgccatcacagggcccctcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaagcctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 52 - protein, 560 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAGRERNALW

SEQ ID NO: 53 - DNA, 1797 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggctgagattcaagccaccattccaggcctctttgcccaagaaagaaacttctgtcacccttgcactctcctgtattctgagtccctggccaatagcacagccttccatgccccgacccccaccccaagcctctccactaggcctctgccaggatctaagcccatgagcacagggactggctatcccaagacctggcagatgtggctgctcaataaacacttgttgaaccatcaaaaaaaaaaaaaaaa

SEQ ID NO: 54 - protein, 435 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLLPPPAHALTGPAQSSTH

SEQ ID NO: 55 - DNA, 1851 bp, homo sapiens
ctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttagtctctgagtcccaaaaagaagtgcaggcagagccatctgccaggcccaggagagctctgagctctggccaacaactgcagccaggctgggcagagcactccggctcacctgggctcctggcgtgtcatttgctggcttgaataaagatgtccgccttatccagtgcctgagtgtgcgagagaggcagatgcctccaaaaaaaaaaaaaaaaa

SEQ ID NO: 56 - protein, 423 aa, homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLRNALW

SEQ ID NO: 57 - DNA, 6631 bp, homo sapiens
atcgtttactttggttgtcccttctggcatggtgcatatgttatgggaagagggattataatttggtgctgtttgtagagatgacaacactgataaaatccactcattgctggtcccagcacacctggaaagttctgcaaggcctcagctacagaaagcccagagacagaaagtaaactctttcatgacccttgatcatcagatcatcaatccaactcttaaatggtcacaacctgcagtgccaagtggtgggcctcttgtgcagcatgcacacacaactctggacagtgatgctggcctcacagaaaacccactcaccaagttactagctattgggaaagaagatgacaatgcacaatggcatatggaggacgttattgaggatataatcggtatggaatcaagttttaaagaggaaggagcagactctcctctgctaatgcaaagaacattatctggaagtattttggatgtgtatagcggtgaacaaggaatttcaccaattaacatggggcttacaagtgcttcttgtccaagtagtctaccaatgaaaagagaaattacagaaactgacactagagctttagcaaaagagagacaaaaaaaggacaaccacaacctcattgaaagaagaagaaggtataatattaattaccgaatcaaggagcttggcactcttattccaaagtctaatgatcctgatatgcgctggaacaaaggaaccattctaaaagcatcagtggagtacatcaagtggctacaaaaagaacaacagagagcccgagaattggaacacagacagaagaaattagagcaggctaacaggcgacttctacttcggattcaggaactagaaattcaggctcgtactcatggtctgccaaccctggcttcacttggcacggttgatttaggtgctcatgtcaccaaacagcagagccatcctgagcagaattcagtagactattgccaacaactgactgtgtctcaggggccaagccctgagctctgtgatcaagctatagccttttctgatcctttgtcatacttcacagatttatcatttagtgctgcattgaaagaggaacaaagattggatggcatgctattggatgacacaatctctccatttggaacagatcctctgctatctgccacttcccctgcagtttccaaagaaagcagtaggagaagtagctttagctcagatgatggtgatgaattataagaaataaacagacccaattcatcaactggaaagcaattctatgctggtgctatgcaattatgctctgtgtttcatatgttgctttggcttattttttttcttaaaggaatgtgttgttcatgaaaaactgatagaagcaacagaagaattcgcaggaagaaaaatcatagtgttaatgaattattgagggcgaaaaaaaggtgttttcttctttgactacggagtccaaatccacttaaattctgttttcctgaaaagaggtacagcataagaaatagctctttattgatgttttaaaagcagcaacttggtggtgtactactggaactaatgactgcaaagtgttaaacgactgaaatatacaaacagtctcttagttactcatttccatcttctcttcaactttcacatcagtcttccggaatcaagatcaacatatcaggtggtcattgcctttctccattgtctagtagacatgtctaaagttcaaactttataggataaataaatgtataatagattatctgtcacttgtggttgaaaggcaaatctacaataaatgtgagaattttccacaataaaatatggataacttataaaaacattggttactaaaattagatcctcattttattgtagttggttcaattacactaattctaaaagcatccatgcatatttatatctccagtctctgttcaggaaaaggaacatattgaatattttcctcaggaatatggaccagaattgtatcccttcacacaaacatacacatacacatatgcacatcattcaggtagtatatgttcttttgttttcttcatgcttctgactgcatcagaatcacattccaaattctcttttcttatgaagaagagatgtcagatcatcaattttagtaaataaaatataaaatgtccccctgcaaggacagttttcaggtacttaaaacttttcatcagtattggacagaaatcaattagttgttgatttggtttttctccaaatggataaaatattgaaaattgaattgccaattgacaaaataattattacaacaaactatttcttattattttcagttctgagaggaacgtaaggttctatttctataaacacttagagtgtcctatgatctttggttgcactgttagcatttattataagcacttataactatgatgcttcatttagatttttatctcttgcgcttgttttaggttaggaaattaagttaccaagcacgttgctctgtgctggacctccaagagtgatcatccgatcaatgagtattcatggaggcatactcagaactgcagtgagtcctgagaaaatgtagaagagttgaaaaagatatggtcttactcctaaggtagttaatgagaatgcatgaaaaacaaaaacaaaagatagaagacatatagaataaaaaatctaagcataaactgtaaatacagaaggtcagagaaaatgaaaatgactgacaaactttatgggaacagcagaatttgacttctctttgaaaggcattaattgagtgaaagtgagggcatataaaggaataataccaaccaaatgataaaagtgagaataataggggaagtatctttataggactttaattagaaagatctggatgccctagaagatgtctgttggggagttctgaagcataagactgacaacaggttgtgaaggcccttagaaaactatggtgaaataatatagtattaatataatagaaacaaatctttttagtagcagggaggatgctaaaaactaaaatatatattactaatgagtgagatgtaatctttcatgaatattaagctttgggataaattttgaaaggatcttttagttgcttctcacatggaaaatatgtcactatgaaatgtgagaccctaatgctgataaaagtagaaaaatagtaaatcattatggtataataattatctcaggcttagcttgtatcatttgctcagaatttggagaattaaaataattttattgtttgacatggaaaaataattactaacttctgggtacagtcattaaagcctgatagataaattcatctgaaatatgccatgtgaaacagctattagatacatcttctcaataattttcaagacgatattacatacttttaattttataacaaaattccacttcattatctgagttgataatttgattaaatgttaaaaattatagataaggaatatattttggagttcatagaaaacacaccactttattagatagagactggcttcagttctcattttatctgctcttctcatttcttcatattttgagtagacttgcactgatgatgtcattactcagtcattattttctgttctgttaaggtacaactactgggccttgaaatctatccttcacaatcttggctgagataaggacttggcacaggatgatagagcctggacacaagagatctggagagggaactgctatttgctgctacattcagattatgagatgtcagggaactgtaaaaagggtaatgaatttttaaagagagtaaagagttatgctgtgtcccggtcaactgaaatacattaaaaattaattagatggtagttcttaactttttaaggcagataccttgagaatctagtgagttatagaccctctccctagaaaagtatagaactgtttatatggacaataattcacatgcattcagagatcttagggatctcctgaatgttttcttggatcccaagtgaaaaattcctgaatcaggtctcttaggtgtgcggttaatgtcatgtataacaatggggggacccttacttatctaaatataattatcccaatcctaataatgatgaggttgcataggaaagtaatagtgtaccaaactttagattattcactaaaaaagttgttcatttatgaagtagtcgtttatcaaacatgtcctccccactcagcaaactatgccttctatttattatatatgggtcagtttcactataattactgagtgttataaacatctgaccataacattttgaaatgatgcaaataaatttccaaacaaaaatagtgtgaatttaaaagcaaattatttgagtatctaagaaacaagatagacttctagaaaaatttgactctctagaatatttcttgcagaaatgagatttttcataatagtaaaagaggcatatgtttatcaaacaatgctgtcacaaaaagcatcaactgtaatgggactattaatgcataattattgatttattcattcaattaatatacaattatcccttttcatttaaagatttaattcataattacaattataataaaacttcctttaaagtaagatacaataattttattgtttttcattcttttttcaacaaaatatcccatccaatcattttttatattattaaatattggctgctttttcttggattcacattaaacagccctttccaacttccaattgtcttaaaataatgatgacctcctgtgagtagatacagctctttacaatttttttctttagtgccttttcttcttgaatttttcctatatcaaatggagaatatatgtacagatggtattttctcagtttataggcatatcagtgaccatggctttctttatataggtttttaaaaaagccctaaataataaatagccagatgagctggggacattgagaaatagccttcctcttcctttttcaactcatttttttcccacctacatgactgtaaatcaaatatttaatagctcttacttaaaaaaacagatacaaagaatgtcttgatttggtgtgctcatttaccataatgtcatgaggggaattagatttcacaactttaaaaggaatatatttttattttattttgaaaaactgagtcatataggaattttcttatacttcaaggcatcatggaaacacttttttcctgtttggatattgtggaatttaaacgttcaaataaataaatggcataactaagtgttccaaatttttttacaatgtctttgaccctattcaaacactttaggtatttactgaccgtctgatgtgtaagatgtggaataaaactggaatcaattaattatttcactgtgttatcagcgcaagatcaaccatctgggtttcttaaagacacccgaaggattgaattttgtttcagtattgataatggcatagtctctatgtgctacatggaattacatcatttatccctccagtgccctatatgttgataagtatgtcagtttgacttagtatacatatatacagagatttcattacatttacctaataaatacaaaatacatttcgagtgttactgatctcctatgttacatgagcctcctgtaatcattgtgcattgatgttccaatgttttattgtttgtatgaattttaatttgaaaacaaggaaacaatccaaaagcagaaaaaatagcttttcttaaaattttcagtgctacattttccctctgaggtccatagagatttgaatgtataggagattatccgaaaacagctattttgattaaaaaatatatctcccaggattcaaccaacttaatgatgaagtactattgtctactgctttatacataaaagggaactttttatctgcttgtaaagggatttttatgtgtatttctgcataatcagatgacttctattgtgttttctactgatgaaattctctgtaaaatgtctttttcttacattatccaacaggcataaagaataacagtaaagacttttgtgtttgtaatactacctcttttatccctgcactactgttttattgcaaaaattctatattgtcactgtattttttccatagaatataaattttgttcttgtgctaaagctggtagtttatgtagcagacaaaatatacaaataaaagaagagactgattttgctgaaagaattatatataatcaagaagttacatattgttctttaaatatgatactgaattttaaaagcaaacgaaattcaagaatcttatctaacagcatagcagttgcttatggcatacaaggctaaaattaattcagctatttaatcttaataattattatgtagttaaaaatctttgactttaatagtgttttacatatacaaatagctgaagtaacattcctataattttaatctgacattggttagatcaagaaaacattgttaataagactgtagaatttgtaattattgctatttttcatttttaataacaaagtaatgtgtcttattttctaagaaaatggagaactttggtgtactttaatacatacaaaaatctttgtaaaaataccttaaaatgtaccaatattttctttgcatatattaaatgaaagactataattatgaaatgtt

SEQ ID NO: 58 - protein, 347 aa, homo sapiens
MTLDHQIINPTLKWSQPAVPSGGPLVQHAHTTLDSDAGLTENPLTKLLAIGKEDDNAQWHMEDVIEDIIGMESSFKEEGADSPLLMQRTLSGSILDVYSGEQGISPINMGLTSASCPSSLPMKREITETDTRALAKERQKKDNHNLIERRRRYNINYRIKELGTLIPKSNDPDMRWNKGTILKASVEYIKWLQKEQQRARELEHRQKKLEQANRRLLLRIQELEIQARTHGLPTLASLGTVDLGAHVTKQQSHPEQNSVDYCQQLTVSQGPSPELCDQAIAFSDPLSYFTDLSFSAALKEEQRLDGMLLDDTISPFGTDPLLSATSPAVSKESSRRSSFSSDDGDEL

SEQ ID NO: 59 - DNA, 6544 bp, homo sapiens
atcgtttactttggttgtcccttctggcatggtgcatatgttatgggaagagggattataatttggtgctgtttgtagagatgacaacactgataaaatccactcattgctggtcccagcacacctggaaagttctgcaaggcctcagctacagaaagcccagagacagaaagtaaactctttcatgacccttgatcatcagatcatcaatccaactcttaaatggtcacaacctgcagtgccaagtggtgggcctcttgtgcagcatgcacacacaactctggacagtgatgctggcctcacagaaaacccactcaccaagttactagctattgggaaagaagatgacaatgcacaatggcatttatctggaagtattttggatgtgtatagcggtgaacaaggaatttcaccaattaacatggggcttacaagtgcttcttgtccaagtagtctaccaatgaaaagagaaattacagaaactgacactagagctttagcaaaagagagacaaaaaaaggacaaccacaacctcattgaaagaagaagaaggtataatattaattaccgaatcaaggagcttggcactcttattccaaagtctaatgatcctgatatgcgctggaacaaaggaaccattctaaaagcatcagtggagtacatcaagtggctacaaaaagaacaacagagagcccgagaattggaacacagacagaagaaattagagcaggctaacaggcgacttctacttcggattcaggaactagaaattcaggctcgtactcatggtctgccaaccctggcttcacttggcacggttgatttaggtgctcatgtcaccaaacagcagagccatcctgagcagaattcagtagactattgccaacaactgactgtgtctcaggggccaagccctgagctctgtgatcaagctatagccttttctgatcctttgtcatacttcacagatttatcatttagtgctgcattgaaagaggaacaaagattggatggcatgctattggatgacacaatctctccatttggaacagatcctctgctatctgccacttcccctgcagtttccaaagaaagcagtaggagaagtagctttagctcagatgatggtgatgaattataagaaataaacagacccaattcatcaactggaaagcaattctatgctggtgctatgcaattatgctctgtgtttcatatgttgctttggcttattttttttcttaaaggaatgtgttgttcatgaaaaactgatagaagcaacagaagaattcgcaggaagaaaaatcatagtgttaatgaattattgagggcgaaaaaaaggtgttttcttctttgactacggagtccaaatccacttaaattctgttttcctgaaaagaggtacagcataagaaatagctctttattgatgttttaaaagcagcaacttggtggtgtactactggaactaatgactgcaaagtgttaaacgactgaaatatacaaacagtctcttagttactcatttccatcttctcttcaactttcacatcagtcttccggaatcaagatcaacatatcaggtggtcattgcctttctccattgtctagtagacatgtctaaagttcaaactttataggataaataaatgtataatagattatctgtcacttgtggttgaaaggcaaatctacaataaatgtgagaattttccacaataaaatatggataacttataaaaacattggttactaaaattagatcctcattttattgtagttggttcaattacactaattctaaaagcatccatgcatatttatatctccagtctctgttcaggaaaaggaacatattgaatattttcctcaggaatatggaccagaattgtatcccttcacacaaacatacacatacacatatgcacatcattcaggtagtatatgttcttttgttttcttcatgcttctgactgcatcagaatcacattccaaattctcttttcttatgaagaagagatgtcagatcatcaattttagtaaataaaatataaaatgtccccctgcaaggacagttttcaggtacttaaaacttttcatcagtattggacagaaatcaattagttgttgatttggtttttctccaaatggataaaatattgaaaattgaattgccaattgacaaaataattattacaacaaactatttcttattattttcagttctgagaggaacgtaaggttctatttctataaacacttagagtgtcctatgatctttggttgcactgttagcatttattataagcacttataactatgatgcttcatttagatttttatctcttgcgcttgttttaggttaggaaattaagttaccaagcacgttgctctgtgctggacctccaagagtgatcatccgatcaatgagtattcatggaggcatactcagaactgcagtgagtcctgagaaaatgtagaagagttgaaaaagatatggtcttactcctaaggtagttaatgagaatgcatgaaaaacaaaaacaaaagatagaagacatatagaataaaaaatctaagcataaactgtaaatacagaaggtcagagaaaatgaaaatgactgacaaactttatgggaacagcagaatttgacttctctttgaaaggcattaattgagtgaaagtgagggcatataaaggaataataccaaccaaatgataaaagtgagaataataggggaagtatctttataggactttaattagaaagatctggatgccctagaagatgtctgttggggagttctgaagcataagactgacaacaggttgtgaaggcccttagaaaactatggtgaaataatatagtattaatataatagaaacaaatctttttagtagcagggaggatgctaaaaactaaaatatatattactaatgagtgagatgtaatctttcatgaatattaagctttgggataaattttgaaaggatcttttagttgcttctcacatggaaaatatgtcactatgaaatgtgagaccctaatgctgataaaagtagaaaaatagtaaatcattatggtataataattatctcaggcttagcttgtatcatttgctcagaatttggagaattaaaataattttattgtttgacatggaaaaataattactaacttctgggtacagtcattaaagcctgatagataaattcatctgaaatatgccatgtgaaacagctattagatacatcttctcaataattttcaagacgatattacatacttttaattttataacaaaattccacttcattatctgagttgataatttgattaaatgttaaaaattatagataaggaatatattttggagttcatagaaaacacaccactttattagatagagactggcttcagttctcattttatctgctcttctcatttcttcatattttgagtagacttgcactgatgatgtcattactcagtcattattttctgttctgttaaggtacaactactgggccttgaaatctatccttcacaatcttggctgagataaggacttggcacaggatgatagagcctggacacaagagatctggagagggaactgctatttgctgctacattcagattatgagatgtcagggaactgtaaaaagggtaatgaatttttaaagagagtaaagagttatgctgtgtcccggtcaactgaaatacattaaaaattaattagatggtagttcttaactttttaaggcagataccttgagaatctagtgagttatagaccctctccctagaaaagtatagaactgtttatatggacaataattcacatgcattcagagatcttagggatctcctgaatgttttcttggatcccaagtgaaaaattcctgaatcaggtctcttaggtgtgcggttaatgtcatgtataacaatggggggacccttacttatctaaatataattatcccaatcctaataatgatgaggttgcataggaaagtaatagtgtaccaaactttagattattcactaaaaaagttgttcatttatgaagtagtcgtttatcaaacatgtcctccccactcagcaaactatgccttctatttattatatatgggtcagtttcactataattactgagtgttataaacatctgaccataacattttgaaatgatgcaaataaatttccaaacaaaaatagtgtgaatttaaaagcaaattatttgagtatctaagaaacaagatagacttctagaaaaatttgactctctagaatatttcttgcagaaatgagatttttcataatagtaaaagaggcatatgtttatcaaacaatgctgtcacaaaaagcatcaactgtaatgggactattaatgcataattattgatttattcattcaattaatatacaattatcccttttcatttaaagatttaattcataattacaattataataaaacttcctttaaagtaagatacaataattttattgtttttcattcttttttcaacaaaatatcccatccaatcattttttatattattaaatattggctgctttttcttggattcacattaaacagccctttccaacttccaattgtcttaaaataatgatgacctcctgtgagtagatacagctctttacaatttttttctttagtgccttttcttcttgaatttttcctatatcaaatggagaatatatgtacagatggtattttctcagtttataggcatatcagtgaccatggctttctttatataggtttttaaaaaagccctaaataataaatagccagatgagctggggacattgagaaatagccttcctcttcctttttcaactcatttttttcccacctacatgactgtaaatcaaatatttaatagctcttacttaaaaaaacagatacaaagaatgtcttgatttggtgtgctcatttaccataatgtcatgaggggaattagatttcacaactttaaaaggaatatatttttattttattttgaaaaactgagtcatataggaattttcttatacttcaaggcatcatggaaacacttttttcctgtttggatattgtggaatttaaacgttcaaataaataaatggcataactaagtgttccaaatttttttacaatgtctttgaccctattcaaacactttaggtatttactgaccgtctgatgtgtaagatgtggaataaaactggaatcaattaattatttcactgtgttatcagcgcaagatcaaccatctgggtttcttaaagacacccgaaggattgaattttgtttcagtattgataatggcatagtctctatgtgctacatggaattacatcatttatccctccagtgccctatatgttgataagtatgtcagtttgacttagtatacatatatacagagatttcattacatttacctaataaatacaaaatacatttcgagtgttactgatctcctatgttacatgagcctcctgtaatcattgtgcattgatgttccaatgttttattgtttgtatgaattttaatttgaaaacaaggaaacaatccaaaagcagaaaaaatagcttttcttaaaattttcagtgctacattttccctctgaggtccatagagatttgaatgtataggagattatccgaaaacagctattttgattaaaaaatatatctcccaggattcaaccaacttaatgatgaagtactattgtctactgctttatacataaaagggaactttttatctgcttgtaaagggatttttatgtgtatttctgcataatcagatgacttctattgtgttttctactgatgaaattctctgtaaaatgtctttttcttacattatccaacaggcataaagaataacagtaaagacttttgtgtttgtaatactacctcttttatccctgcactactgttttattgcaaaaattctatattgtcactgtattttttccatagaatataaattttgttcttgtgctaaagctggtagtttatgtagcagacaaaatatacaaataaaagaagagactgattttgctgaaagaattatatataatcaagaagttacatattgttctttaaatatgatactgaattttaaaagcaaacgaaattcaagaatcttatctaacagcatagcagttgcttatggcatacaaggctaaaattaattcagctatttaatcttaataattattatgtagttaaaaatctttgactttaatagtgttttacatatacaaatagctgaagtaacattcctataattttaatctgacattggttagatcaagaaaacattgttaataagactgtagaatttgtaattattgctatttttcatttttaataacaaagtaatgtgtcttattttctaagaaaatggagaactttggtgtactttaatacatacaaaaatctttgtaaaaataccttaaaatgtaccaatattttctttgcatatattaaatgaaagactataattatgaaatgtt

SEQ ID NO: 60 - protein, 318 aa, homo sapiens
MTLDHQIINPTLKWSQPAVPSGGPLVQHAHTTLDSDAGLTENPLTKLLAIGKEDDNAQWHLSGSILDVYSGEQGISPINMGLTSASCPSSLPMKREITETDTRALAKERQKKDNHNLIERRRRYNINYRIKELGTLIPKSNDPDMRWNKGTILKASVEYIKWLQKEQQRARELEHRQKKLEQANRRLLLRIQELEIQARTHGLPTLASLGTVDLGAHVTKQQSHPEQNSVDYCQQLTVSQGPSPELCDQAIAFSDPLSYFTDLSFSAALKEEQRLDGMLLDDTISPFGTDPLLSATSPAVSKESSRRSSFSSDDGDEL

SEQ ID NO: 61 - DNA, 3085 bp, homo sapiens
ctcacgctctctttccctgccgcctgcctttcttttttcctttttttgtattggcgtcttggggctgttacacacacgcgcgctgtccattgcagcttacataaaggcgggcgcgattatgcaattatattgttagcgatatttcaagagcaatggctcgttttcttaggatttcaacacgaaggcatcatgcatttttgaaaaactagtattgagaataataccttgcaacgtaaagaatgttttttggtatttttacacaatctctactttgaccaaacgagtctggacagttttcttttaatggaaaataggagaaatggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcattttgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacagtgacagaccggaaagtgaagagtcagaactaactttggtctcagcgggagccagtggtgatgtaaaaatgcttccactgcaagtctgtggccccacaacgtgggcttaaagcagaagccccacagcctggcacgaaggccccgtctgggttaggtgactaaaagggcttcggccacaggcaggtcacagaaaggcaggtttcatttcttatcacataagagagatgagaaagcttttattcctttgaatattttttgaaggtttcagatgaggtcaacacaggtagcacagattttgaatctgtgtgcatatttgttactttacttttgctgtttatacttgagaccaacttttcaatgtgattcttctaaagcactggtttcaagaatatggaggctggaaggaaataaacattacggtacagacatggagatgtaaaatgagtttgtattattacaaatattgtcatctttttctagagttatcttctttattattcctagtctttccagtcaacatcgtggatgtagtgattaaatatatctagaactatcatttttacactattgtgaatatttggaattgaacgactgtatattgctaagagggcccaaagaattggaatcctccttaatttaattgctttgaagcatagctacaatttgtttttgcatttttgttttgaaagtttaacaaatgactgtatctaggcatttcattatgctttgaactttagtttgcctgcagtttcttgtgtagatttgaaaattgtataccaatgtgttttctgtagactctaagatacactgcactttgtttagaaaaaaaactgaagatgaaatatatattgtaaagaagggatattaagaatcttagataacttcttgaaaaagatggcttatgtcatcagtaaagtacctttatgttatgaggatataatgtgtgctttattgaattagaaaattagtgaccattattcacaggtggacaaatgttgtcctgttaatttataggagttttttggggatgtggaggtagttgggtagaaaaattattagaacattcacttttgttaacagtatttctcttttattctgttatatagtggatgatatacacagtggcaaaacaaaagtacattgcttaaaatatatagtgaaaaatgtcactatatcttcccatttaacattgtttttgtatattgggtgtagatttctgacatcaaaacttggacccttggaaaacaaaagttttaattaaaaaaaatccttgtgacttacaatttgcacaatatttcttttgttgtactttatatcttgtttacaataaagaattccctttggtaaaaaaaaaaaaaaa

SEQ ID NO: 62 - protein, 469 aa, homo sapiens
MEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 63 - DNA, 2899 bp, homo sapiens
cagacggcgctgagcgcggcggcggcgggagcggcgtcgagtgtctccgtgcgcccgtctgtggccaagcagccagcagcctagcagccagtcagcttgccgccggcggccaagcagccaaccatgctcaacttcggtgcctctctccagcagactgcggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcattttgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacagtgacagaccggaaagtgaagagtcagaactaactttggtctcagcgggagccagtggtgatgtaaaaatgcttccactgcaagtctgtggccccacaacgtgggcttaaagcagaagccccacagcctggcacgaaggccccgcctgggttaggtgactaaaagggcttcggccacaggcaggtcacagaaaggcaggtttcatttcttatcacataagagagatgagaaagcttttattcctttgaatattttttgaaggtttcagatgaggtcaacacaggtagcacagattttgaatctgtgtgcatatttgttactttacttttgctgtttatacttgagaccaacttttcaatgtgattcttctaaagcactggtttcaagaatatggaggctggaaggaaataaacattacggtacagacatggagatgtaaaatgagtttgtattattacaaatattgtcatctttttctagagttatcttctttattattcctagtctttccagtcaacatcgtggatgtagtgattaaatatatctagaactatcatttttacactattgtgaatatttggaattgaacgactgtatattgctaagagggcccaaagaattggaatcctccttaatttaattgctttgaagcatagctacaatttgtttttgcatttttgttttgaaagtttaacaaatgactgtatctaggcatttcattatgctttgaactttagtttgcctgcagtttcttgtgtagatttgaaaattgtataccaatgtgttttctgtagactctaagatacactgcactttgtttagaaaaaaaactgaagatgaaatatatattgtaaagaagggatattaagaatcttagataacttcttgaaaaagatggcttatgtcatcagtaaagtacctttatgttatgaggatataatgtgtgctttattgaattagaaaattagtgaccattattcacaggtggacaaatgttgtcctgttaatttataggagttttttggggatgtggaggtagttgggtagaaaaattattagaacattcacttttgttaacagtatttctcttttattctgttatatagtggatgatatacacagtggcaaaacaaaagtacattgcttaaaatatatagtgaaaaatgtcactatatcttcccatttaacattgtttttgtatattgggtgtagatttctgacatcaaaacttggacccttggaaaacaaaagttttaattaaaaaaaatccttgtgacttacaatttgcacaatatttcttttgttgtactttatatcttgtttacaataaagaattc

SEQ ID NO: 64 - protein, 480 aa, homo sapiens
MLNFGASLQQTAEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 65 - DNA, 3150 bp, homo sapiens
gcggtgagcgctggggctgcgcgggcggcaggcaacggctgaggcggcggcggcggcggcggcggcgtgggttgggctcgagcgggcggcggcgcctcagactccccggaacgggagcccacgcgggcgggcgcctgaaacaaagggaagcgggcgtccgggcgccgcgggtgggcggtcagtcggtcagcgcggagccagccagcgggtgcccgcgcaagccccgagcgcggccggccggcgcggcctcagggcgggaagatgccgcgcgtcgtgcccgaccagagaagcaagttcgagaacgaggagttttttaggaagctgagccgcgagtgtgagattaagtacacgggcttcagggaccggccccacgaggaacgccaggcacgcttccagaacgcctgccgcgacggccgctcggaaatcgcttttgtggccacaggaaccaatctgtctctccagttttttccggccagctggcagggagaacagcgacaaacacctagccgagagtatgtcgacttagaaagagaagcaggcaaggtatatttgaaggctcccatgattctgaatggagtctgtgttatctggaaaggctggattgatctccaaagactggatggtatgggctgtctggagtttgatgaggagcgagcccagcaggaggatgcattagcacaacaggcctttgaagaggctcggagaaggacacgcgaatttgaagatagagacaggtctcatcgggaggaaatggaggcaagaagacaacaagaccctagtcctggttccaatttaggtggtggtgatgacctcaaacttcgttaattaatagcacagcagatgtgtgctgcccatctttacatacacattgcttctagttggcagaaataattgattaaaagaccagaaactgtgataactggaggtactacggtctatttctcaaccttaggcagtaatagacatcacaaactgccatggttttgcactatgattataatacctgcatttctaattttttaagcatgtagccagtaataatttgaagttttttttctatgcaagcttaccttgttggcattattttagggagttgaaactatcaactgtaaagctccttttcttccactttaatttaaaagttcatgtcatttaaaaacaagtcaagaaattaaaattgtatcagagggttttctctaatcattttttctatttttttttttgtacttctagatgttttggttatacagcttcattttagatgagcattcttattttttgttttgtttgccccatttccttttgtgtttttatagtctatagcattttaaaactgctgatgttgtttgcattatttacaggctaaaaacttagtagcatagagctgtctgccacagccttctgacaaagtttacagttattaaagttgcagtatccttttaaatgctagtaatcagcactctttcttttttttttttttaatagagacagggtctcgcagtgttgcccaggctggtctcgaactcctggcatcaagcgatcctcctgccttagcctcccagagtactgggattacaggctctttctttttaaacataaaagttttaaattggtattaactctgtactctgccctagattgttttagcttctgttctgtaatcatgagtttggttggagatattctccatagatgatcttctactgaaatgcctaaagaagtcacaggctggcttctgttttattcagggatttttttaaaaagtcaatcagaaaagggatactggagcttcttcatgtatgtaacagcatattaaactggagacagtgatgaatcagctacaaaggtaatattgtattaaaatcatgtttaagatagctgcttttatgtgtattttatattgcatgcttttgtaaaaacatgctgggtgatgaaagattagttttagagagaaaatgttcatctgtgcagaggatgcattttcttccattaattctggaaaaaacgttcacagttatatatatggtattttgcaaaaggactattaatagaaccttttgagatgaattaatgtaagaatattttttaaataggcttactgtcaaattgcaactttttttttagatacagagtggaaaacagtgctaagtcatttggcacctccttacaaatatttttcatggtcacatttattaaatgttactacatttctgaatttttgaaaaatgtattttatcattaaatggcattattttaaagggtgaaaaactgacacagtcaattcagaaaatggactgaagtctgaataaggtcattgcatttaaaaagcatataactgtacttgactgatgagggaggtgttactttcattgtatataggtcttatttcataaacagatatcctgtatcaaataaaagtatttgttatatatttgaagttatgcatggaaaggagtgtgtttaaattgttacaaacaataatgcgtcattaaaggccatgctgatcttgcataactataagtactatgaatgaatttggttggttttggtgttgtacagctcacatgtttacacactcagtgccctaatttcccctgagggaatcgctttttaagtgatccttacagtggtgttttatgttactttattacagagctccttggttttttacttctgcacttaaatttttttaaataacatgatgatggtacattttcctctattgtctagctaagggctttcggtccaccagtaaataagatcaaatgctcttaaatgttcctgttaccatcctaatgtaaatactggatttttctgtcatttagcaccatgctgcttctgtctgtcttaatgctggcattaagatcatgagccctttttctccagtagtacaggctttgaaaactacttctattaagttattgatgcaatttgatattttttcataatctatatttaaacaaaattacatcattgcatcatcttttctaaattcatctccattaaaacttgccttaagctaccagattgcttttgccaccattggccatactgtgtgtttgtttgtttaatttactttcacaataaacttctgtgtagtaaaaaaaaaaaaaaaaaaaaaaaaaaaaaaaaaaaaaaaaaaaaaaaaaa

SEQ ID NO: 66 - protein, 187 aa, homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEARRQQDPSPGSNLGGGDDLKLR

SEQ ID NO: 67 - DNA, 3181 bp, homo sapiens
gcggtgagcgctggggctgcgcgggcggcaggcaacggctgaggcggcggcggcggcggcggcggcgtgggttgggctcgagcgggcggcggcgcctcagactccccggaacgggagcccacgcgggcgggcgcctgaaacaaagggaagcgggcgtccgggcgccgcgggtgggcggtcagtcggtcagcgcggagccagccagcgggtgcccgcgcaagccccgagcgcggccggccggcgcggcctcagggcgggaagatgccgcgcgtcgtgcccgaccagagaagcaagttcgagaacgaggagttttttaggaagctgagccgcgagtgtgagattaagtacacgggcttcagggaccggccccacgaggaacgccaggcacgcttccagaacgcctgccgcgacggccgctcggaaatcgcttttgtggccacaggaaccaatctgtctctccagttttttccggccagctggcagggagaacagcgacaaacacctagccgagagtatgtcgacttagaaagagaagcaggcaaggtatatttgaaggctcccatgattctgaatggagtctgtgttatctggaaaggctggattgatctccaaagactggatggtatgggctgtctggagtttgatgaggagcgagcccagcaggaggatgcattagcacaacaggcctttgaagaggctcggagaaggacacgcgaatttgaagatagagacaggtctcatcgggaggaaatggaggtgagagtttcacagctgctggcagtaactggcaagaagacaacaagaccctagtcctggttccaatttaggtggtggtgatgacctcaaacttcgttaattaatagcacagcagatgtgtgctgcccatctttacatacacattgcttctagttggcagaaataattgattaaaagaccagaaactgtgataactggaggtactacggtctatttctcaaccttaggcagtaatagacatcacaaactgccatggttttgcactatgattataatacctgcatttctaattttttaagcatgtagccagtaataatttgaagttttttttctatgcaagcttaccttgttggcattattttagggagttgaaactatcaactgtaaagctccttttcttccactttaatttaaaagttcatgtcatttaaaaacaagtcaagaaattaaaattgtatcagagggttttctctaatcattttttctatttttttttttgtacttctagatgttttggttatacagcttcattttagatgagcattcttattttttgttttgtttgccccatttccttttgtgtttttatagtctatagcattttaaaactgctgatgttgtttgcattatttacaggctaaaaacttagtagcatagagctgtctgccacagccttctgacaaagtttacagttattaaagttgcagtatccttttaaatgctagtaatcagcactctttcttttttttttttttaatagagacagggtctcgcagtgttgcccaggctggtctcgaactcctggcatcaagcgatcctcctgccttagcctcccagagtactgggattacaggctctttctttttaaacataaaagttttaaattggtattaactctgtactctgccctagattgttttagcttctgttctgtaatcatgagtttggttggagatattctccatagatgatcttctactgaaatgcctaaagaagtcacaggctggcttctgttttattcagggatttttttaaaaagtcaatcagaaaagggatactggagcttcttcatgtatgtaacagcatattaaactggagacagtgatgaatcagctacaaaggtaatattgtattaaaatcatgtttaagatagctgcttttatgtgtattttatattgcatgcttttgtaaaaacatgctgggtgatgaaagattagttttagagagaaaatgttcatctgtgcagaggatgcattttcttccattaattctggaaaaaacgttcacagttatatatatggtattttgcaaaaggactattaatagaaccttttgagatgaattaatgtaagaatattttttaaataggcttactgtcaaattgcaactttttttttagatacagagtggaaaacagtgctaagtcatttggcacctccttacaaatatttttcatggtcacatttattaaatgttactacatttctgaatttttgaaaaatgtattttatcattaaatggcattattttaaagggtgaaaaactgacacagtcaattcagaaaatggactgaagtctgaataaggtcattgcatttaaaaagcatataactgtacttgactgatgagggaggtgttactttcattgtatataggtcttatttcataaacagatatcctgtatcaaataaaagtatttgttatatatttgaagttatgcatggaaaggagtgtgtttaaattgttacaaacaataatgcgtcattaaaggccatgctgatcttgcataactataagtactatgaatgaatttggttggttttggtgttgtacagctcacatgtttacacactcagtgccctaatttcccctgagggaatcgctttttaagtgatccttacagtggtgttttatgttactttattacagagctccttggttttttacttctgcacttaaatttttttaaataacatgatgatggtacattttcctctattgtctagctaagggctttcggtccaccagtaaataagatcaaatgctcttaaatgttcctgttaccatcctaatgtaaatactggatttttctgtcatttagcaccatgctgcttctgtctgtcttaatgctggcattaagatcatgagccctttttctccagtagtacaggctttgaaaactacttctattaagttattgatgcaatttgatattttttcataatctatatttaaacaaaattacatcattgcatcatcttttctaaattcatctccattaaaacttgccttaagctaccagattgcttttgccaccattggccatactgtgtgtttgtttgtttaatttactttcacaataaacttctgtgtagtaaaaaaaaaaaaaaaaaaaaaaaaaaaaaaaaaaaaaaaaaaaaaaaaaa

SEQ ID NO: 68 - protein, 182 aa, homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEVRVSQLLAVTGKKTTRP

SEQ ID NO: 69 - DNA, 2337 bp, homo sapiens
agctggagggaggagagttgtgcttgagcttcccggtgcctgttctgccgactgccgctcagtaggaagaacgcctggatgcatcagaagcaggaaatggagccagtgacctttgaggatgtggctgtgaacttcactctaggagagtggactatgttggattccagtcaaaagaagctctacagagatgtgatgaaggagacctttttgaacctgatctctatagggaaaacagaagaagaagatactgaagaagagtaccaaaatcctaagggaaacctgagaaccctgatggttgagagactctgtaaatatgattatggtaatcagtatggagaaactcaccaacagagtccagagcatgttgcgaatgaaaaaatgcctccagctataacagaatgtgaaagagacatctgtactcatttaccctcagacatgcacttcaaaggtcaaactggaaagaaatcatgccagtatcaagaacatacagagaaggctttgaaacataagaactgttggaaggacttcacttattctgagttgctttggatgcataagagccctcccatgagacagaagccctatgaagataaacaagggactgaagcctctaggagtttcactaccgatcatgattatgagggagcttacactgaagagaaatcttatatttgtaaacaatgtgggaaagcatggagtgattcctgtagccttctctggcatgaaagaagtcacattcaagagaaacgtcacacatgtaagcaatgtggaaaagcatttagtcgtccctcacagcttcacaaacatgaaaggattcacactggtgagaaaccttatgtgtgtacacattgtgggaaagccttcattgatcgcagaacctgtcacaaccatgaaaggactcacactggagtgaaaccctttgcctgtaagcaatgtgggaaagcatttcttcgttcctgccaacttctcatccatgaaagaatccatactggagagagaccttttgtgtgtaagcattgcggaaaagctttcacctattccagtgcctgctattatcacgaaagaattcacactggagagaagccctgtgtctgcaagcagtgtgggaaagcattcaaatgttctgcgtaccttcacatacatgaacgatctcacagtggagaaaaaccctatgtgtgtaagcattgtggcaaagcctttgcctatgccaccgggtgtcacaaacatgagaggattcacacaggagagaaaccgtatatgtgtgtgcagtgtgggaaactattcactttccccagatcccttcacatacacgagcgatctcatagtggagaaaaaccttatgtatgtaagcagtgtgggaaatccttcactcagggaacttctttgcgaagacatgaacgaattcacactggagaaaaaccttatgtatgtaagcagtgtgggaaagccttcatccaacgtgatgattgctacagtcatgaacgaactcacacaggagagaaaccattcatgtgtgttcaatgtgggaaaacattcaccttttccaaatctcttcagatacatgaaaaaaatcacactggagagaaaccctatatatgtaagcagtgtgggaaagcgttcacctgttccacataccttctcagacatgaacgaactcacagtgagaagaagctcagtggctaatatttctcgcactctaaatcatgggaatgtctgatgtcaacttttattttgaggtatgaaggaaaatggagagccactgtaagattatgagaagtgcaggaggataatttgggtctccatgctttctgtaagatggcaacctgtacaacaggttttgctgctgtgtgtcttttgttccagtaatgaaaaagtaaaagttatgcaattacaatattcaacgaagcctgtgctgtttactgggtttgaacacagcttgggaagtttatctaccaatttcaactaaatgaaattgtctgtaaatataaaaaagttgcatttttttaccaataagataaaatttatgacagtatctcatgtggttgagtcctatgaaagagatatgaagatactgaaggttaatttgtgttaaatatttctgaaaataaactgtgaaagtgatacggtaagttctgagtatttttaaaaaccttgattatttaaataaagtgtgttttattttgctgagtttataaggtcagtgtctcacaacagaacatgtctaccaaagtaactcaccaaaaaacctaaaacattagtctaattcacaagactgcctgtgctctcacagaaatttcctctattacatgaaattagtaataaacatttggaaacatt

SEQ ID NO: 70 - protein, 534 aa, homo sapiens
MHQKQEMEPVTFEDVAVNFTLGEWTMLDSSQKKLYRDVMKETFLNLISIGKTEEEDTEEEYQNPKGNLRTLMVERLCKYDYGNQYGETHQQSPEHVANEKMPPAITECERDICTHLPSDMHFKGQTGKKSCQYQEHTEKALKHKNCWKDFTYSELLWMHKSPPMRQKPYEDKQGTEASRSFTTDHDYEGAYTEEKSYICKQCGKAWSDSCSLLWHERSHIQEKRHTCKQCGKAFSRPSQLHKHERIHTGEKPYVCTHCGKAFIDRRTCHNHERTHTGVKPFACKQCGKAFLRSCQLLIHERIHTGERPFVCKHCGKAFTYSSACYYHERIHTGEKPCVCKQCGKAFKCSAYLHIHERSHSGEKPYVCKHCGKAFAYATGCHKHERIHTGEKPYMCVQCGKLFTFPRSLHIHERSHSGEKPYVCKQCGKSFTQGTSLRRHERIHTGEKPYVCKQCGKAFIQRDDCYSHERTHTGEKPFMCVQCGKTFTFSKSLQIHEKNHTGEKPYICKQCGKAFTCSTYLLRHERTHSEKKLSG

SEQ ID NO: 71 - DNA, 4616 bp, homo sapiens
gcgccgtacgtgcgcgcgcgcggcggcccagaaagcggcgctgggaccctgaggcggccgtggttaggcggctccccggcggctcctccgcggcggtgacggcgaccgcactccccgcttcccgctccccgcgctcctccgcccgggtccgccagccgaggccgctcccgagcgtcggaagatgccggccgtgtccaagggggacgggatgcggggcctggcggtcttcatctcggatatccgcaactgtaaaagtaaagaagcagaaataaaaaggataaacaaggaactggcaaatatcagatcaaaatttaaaggtgacaaggctcttgatggctatagtaaaaaaaagtacgtctgcaagttgctcttcatctttctccttggtcatgacattgactttggacacatggaggctgtgaacctgctgagttcaaacagatacacggaaaagcagatcggctaccttttcatctctgtgttggtgaactcaaacagtgagctgatccgcctgatcaacaacgccatcaagaatgacctggccagccgcaaccccaccttcatgggcctggccctgcactgcatcgccagcgtgggcagccgggagatggccgaggccttcgccggggagatccctaaggtcctcgtagccggagacactatggacagcgtgaagcagagcgcggccctgtgcttgctgcgcctgtacaggacgtcccccgatcttgtccccatgggcgactggacatcccgagtggtgcacctgctcaatgaccagcacttgggtgtggtaactgcagccacaagtctgatcaccactttagcacagaagaacccagaagagtttaaaacctccgtgtctctggctgtctctaggctaagcagaatcgtgacgtctgcatccacagatctccaggattacacttactattttgtcccggctccctggctgtctgtcaaactgctgagactgctgcagtgctacccacccccagaccctgcagtgcgaggccgcctgactgagtgcctggagaccatcctgaacaaagcccaagaaccgcccaagtcgaagaaggtccagcactccaacgcgaagaatgccgtgctcttcgaggccatcagcttaatcattcaccatgacagtgagccgaacctgctcgtccgtgcctgcaaccagttgggccagtttctgcagcaccgcgagaccaacctgcgctacctggccctggagagcatgtgcacgctggccagctctgagttctcccatgaggctgtcaagacgcacatcgagacggtcatcaacgccctgaagactgagcgggacgtgagcgtgcggcagcgggccgtggacctcctctacgccatgtgcgaccgcagcaacgccccacagatcgtggccgagatgctgagctatctggagacagctgactactccatccgagaagagattgtgctgaaggtcgccatcctggctgagaagtacgcggtggactacacctggtatgtggataccatcttgaacttgatccgaattgctggtgattacgtgagtgaagaggtgtggtaccgagtcattcagatcgtcatcaaccgggacgacgtgcagggctacgcggccaagactgtgttcgaggctcttcaggctcccgcgtgccacgagaacctggtcaaagtgggcggctacatcctgggggagtttggaaacttgatagctggagacccgagatccagcccgctgatccagttccacctgctgcactccaagttccacctgtgcagcgtccccacccgcgcgctgctcctgtccacctacatcaagttcgtgaacctcttcccggaggtgaagcccaccatccaggacgtgctgcgcagcgacagccagctcaggaacgcagacgtggagctgcagcagcgtgctgtggagtacctgcggctcagcaccgtggccagcaccgacattctggcgaccgtgctggaggagatgcccccattcccggagcgggagtcctccatcttggcaaagctcaagaagaagaagggccccagcacggtgacagacctggaggacaccaagcgggacaggagtgtggacgtgaacgggggtcctgagcctgccccagccagtaccagcgccgtgtctacgccttctccgtcggcagacctgctgggtctcggggctgccccccctgcccccgcgggccccccaccctcctccggcggcagcgggctgctcgtggacgtgttctcagactcggcctctgtggtcgcgcctctcgctcctggctccgaagacaactttgccaggtttgtttgtaaaaacaatggtgtgttgtttgaaaaccagctgcttcaaattggacttaagtctgaatttcggcagaatttaggtcggatgtttatcttttatggtaataagacctccacgcagttcctaaactttaccccaacactaatctgttcagacgaccttcagcctaacctgaacctgcagaccaagcccgtggacccgaccgtggaggggggcgcgcaggtgcagcaggtggtcaacatagagtgcgtgtccgacttcacggaggcgccagtcctcaacattcagttcaggtatgggggcaccttccagaacgtgtctgtgcagctgcccatcactctcaacaaattcttccagccgacagaaatggcttctcaggatttctttcaacgttggaagcagttgagcaatccacagcaggaagtgcagaacatcttcaaagcaaagcacccaatggacacagaagtcaccaaagccaagatcattggatttggttctgcacttcttgaagaagttgatcctaatcctgcgaatttcgtgggagctggaatcatccacacgaaaaccacccagattggatgcctgctgcgcttggagccgaacctgcaagcccagatgtaccggctcacgctgcgcacaagtaaggaagccgtttctcagagattatgtgaattgctctcagcgcagttttagtcctgaggatggaagaccaggctcgtgtgtcttgtgttgtcttcgtctgtgccgtttgtcttcgtggccatcctgcagatgagcaccgtgtccagtgccacagcacaaggcgcctccccgccccgccgccccacacctctcccctttgggctggacgggaacacacgtgtgtggctcaggaggaaaagctcagcctggactgtggcagccacggcagaaggtggatcttgggatcaatttttataaaaatcgagacagttctgtggttaaatctacaaattaaagggaaattagaagttggcgtgaacgtggcgtttgtgggagtgtcactgagatggcccgtgctgccgcccaccccgcctcggagcctctgggagcagcagtgccactgtgcatggcgtgggctgagccttggtgtgtggccgtcctggtggctgcacacctggcgtcgtcctgggcccttgggaggagcacagctgaccctggttttgctgcagtcccagctggactgttttcccaggcaggattttaatctagaatttagaaacatttgtatttgtaatgacttctggcaaaagcacgtgtcctggccggatgtaactgttctcctttcccagctcctgtttgtgaagggcgtctgttatgctcctgcagtcgccgaggccttggatgtgcagccaggggaggagcgtcctgccggccccgcagggcccccaggactccagggtaaagtgtgggccggtggcgcaagactcagaggtgtgctcgtctctttcctgtcagagtgggcgtccccaggccacggtgcaggcctgagtccttccaccggccccgtccagtcgtccctggaggggctgtggaggagggacgcctctgtgtggtcaggaagtgaaggggccattggccgcatgccatgtgccacctgcggcttgtgtctcacctgtcatctggactcagcacccaggctgcacgtctgacacctgagaggcgagagagtggggccggcctaggagccaaggctggggccttgcgctctgtccccaggatggtggccttgtttgtcctaaacacacccagcacaggttctggcttcctgacatgctgtggaggcagggagggtgggtggccacatgtgcttgagggttttcaccctggccctcagttgcctgctgtgcgggtccctggggcagctgcaggggctcatggacccatcagggtctccacagctcccctgcagtgtgtgcaccccacaatgtctgcggctcttcttccggcgtgtcgggctttgatcacagcatagccacgtcagtggcgtgcgcctctcgcacaggccattctgggtctggtggtgccaggtgccgtgacacgccgtgctgggcttgtgctgcagctgggtggtgtggccctcattctcatgttccagctgctgggcagtgctctgcctgtgtgctgcgcctgcaggctgcgtgtgctgccgtggatctcctgcatcccttgacccctcccgccatcagaggaaaggctgctccccgaggcaccgcttccctgtgcggcgctgcagaggggccctcagtgtggcactcctcgtcaaagaaaaataaaggctagaactgca

SEQ ID NO: 72 - protein, 939 aa, homo sapiens
MPAVSKGDGMRGLAVFISDIRNCKSKEAEIKRINKELANIRSKFKGDKALDGYSKKKYVCKLLFIFLLGHDIDFGHMEAVNLLSSNRYTEKQIGYLFISVLVNSNSELIRLINNAIKNDLASRNPTFMGLALHCIASVGSREMAEAFAGEIPKVLVAGDTMDSVKQSAALCLLRLYRTSPDLVPMGDWTSRVVHLLNDQHLGVVTAATSLITTLAQKNPEEFKTSVSLAVSRLSRIVTSASTDLQDYTYYFVPAPWLSVKLLRLLQCYPPPDPAVRGRLTECLETILNKAQEPPKSKKVQHSNAKNAVLFEAISLIIHHDSEPNLLVRACNQLGQFLQHRETNLRYLALESMCTLASSEFSHEAVKTHIETVINALKTERDVSVRQRAVDLLYAMCDRSNAPQIVAEMLSYLETADYSIREEIVLKVAILAEKYAVDYTWYVDTILNLIRIAGDYVSEEVWYRVIQIVINRDDVQGYAAKTVFEALQAPACHENLVKVGGYILGEFGNLIAGDPRSSPLIQFHLLHSKFHLCSVPTRALLLSTYIKFVNLFPEVKPTIQDVLRSDSQLRNADVELQQRAVEYLRLSTVASTDILATVLEEMPPFPERESSILAKLKKKKGPSTVTDLEDTKRDRSVDVNGGPEPAPASTSAVSTPSPSADLLGLGAAPPAPAGPPPSSGGSGLLVDVFSDSASVVAPLAPGSEDNFARFVCKNNGVLFENQLLQIGLKSEFRQNLGRMFIFYGNKTSTQFLNFTPTLICSDDLQPNLNLQTKPVDPTVEGGAQVQQVVNIECVSDFTEAPVLNIQFRYGGTFQNVSVQLPITLNKFFQPTEMASQDFFQRWKQLSNPQQEVQNIFKAKHPMDTEVTKAKIIGFGSALLEEVDPNPANFVGAGIIHTKTTQIGCLLRLEPNLQAQMYRLTLRTSKEAVSQRLCELLSAQF

SEQ ID NO: 73 - DNA, 3039 bp, homo sapiens
aggcgcagaggagggcggtgttgagaccggcggagcggcgggacccctaggtggcggagggacgctccgggaaagcgaggggcgctacgagctctggcccacgtgacctgccgggggcgggagcagggggcgcgccggcctcctgcggtgcccctgccttggggaggggccgtgaccacccgtctgtcgcccgaggcggccgccgctgcaccttcaccgcgtacccgggacccgcccgcccgcgggagaaatgttgctgaagtgctgctgaaagggccagagatgcaaggatttgggatacattttgaacctttaagctgtctgacattgacctcctttcattattaataaagaagaatcaggagcttaggatgtattaacaccaactcattaatatactaaccggacaatgttctacaaacaattctacattgtaaaggactggattggcacaaaataaaataattttattttattcagcttataatatgactcgatggaggaaaatttgataagcatgagagaagaccattcttttcatgttcgttacagaatggaagcttcttgcctagagctggccttggaaggggaacgtctatgtaaatcaggagactgccgcgctggcgtgtcattctttgaagctgcagttcaagttggaactgaagacctaaaaacacttagcgctatttacagccagttgggcaatgcttatttctatttgcatgattatgccaaagcattagaatatcaccatcatgatttaacccttgcaaggactattggagaccagctgggggaagcgaaagctagtggtaatctgggaaacaccttaaaagttcttgggaattttgacgaagccatagtttgttgtcagcgacacctagatatttccagagagcttaatgacaaggtgggagaagcaagagcactttacaatcttgggaatgtgtatcatgccaaagggaaaagttttggttgccctggtccccaggatgtaggagaatttccagaagaagtgagagatgctctgcaggcagccgtggatttttatgaggaaaacctatcattagtgactgctttgggtgaccgagcggcacaaggacgtgcctttggaaatcttggaaacacacattacctccttggcaacttcagggatgcagttatagctcatgagcagcgtctccttattgcaaaagaatttggagataaagcagctgaaagaagagcatatagcaaccttggaaatgcatatatatttcttggtgaatttgaaactgcctcggaatactacaagaagacactactgttggcccgacagcttaaagaccgagctgtagaagcacagtcttgttacagtcttggaaatacatatactttacttcaagactatgaaaaggccattgattatcatctgaagcacttagcaattgctcaagagctgaatgatagaattggtgaaggaagagcatgttggagcttaggaaatgcatacacagcactaggaaatcatgatcaagcaatgcattttgctgaaaagcacttggaaatttcaagagaggttggggataaaagtggtgaactaacagcacgacttaatctctcagaccttcaaatggttcttggtctgagctacagcacaaataactccataatgtctgaaaatactgaaattgatagcagtttgaatggtgtacgccccaagttgggacgccggcatagtatggaaaatatggaacttatgaagttaacaccagaaaaggtacagaactggaacagtgaaattcttgctaagcaaaaacctcttattgccaaaccttctgcaaagctactctttgtcaacagactgaaggggaaaaaatacaaaacgaattcctccactaaagttctccaagatgccagtaattctattgaccaccgaattccaaattctcagaggaaaatcagtgcagatactattggagatgaagggttctttgacttattaagccgatttcaaagcaataggatggatgatcagagatgttgcttacaagaaaagaactgccatacagcttcaacaacaacttcttccactccccctaaaatgatgctaaaaacatcatctgttcctgtggtatcccccaacacggatgagtttttagatcttcttgccagctcacagagtcgccgtctggatgaccagagggctagtttcagtaatttgccagggcttcgtctaacacaaaacagccagtcggtacttagccacctgatgactaatgacaacaaagaggctgatgaagatttctttgacatccttgtaaaatgtcaaggatccagattagatgatcaaagatgtgctccaccacctgctaccacaaagggtccgacagtaccagatgaagactttttcagccttattttacggtcccagggaaagagaatggatgaacagagagttcttttacaaagagatcaaaacagagacactgactttgggctaaaggactttttgcaaaataatgctttgttggagtttaaaaattcagggaaaaaatcggcagaccattagttactatggatttattttttttcctttcaaacacggtaaggaaacaatctattacttttttccttaaaaggagaatttatagcactgtaatacagcttaaaatatttttagaatgatgtaaatagttaaccttcagtagtctattaaggcattaatacttctctggacatgcgcgtttgagggtggaggggtcctgtaaggtgcttcatcgtctgtgattactgcttgggatgtgttctttggcagcttgtgagattactttacctagtgtttataaagtaggaagttaagtgaatcatagattagaatttaatactcttatggaaataattttttaacatcttaattgacaatggcgtttttttatacataaccatggatgtagtgggaaacaatgttgtttggtaaaaataatgtacttgatcaatgtaaaaaagtatataaaatagtcttactaaaaatctaggtttttttttcctccaaaaaaa

SEQ ID NO: 74 - protein, 684 aa, homo sapiens
MEENLISMREDHSFHVRYRMEASCLELALEGERLCKSGDCRAGVSFFEAAVQVGTEDLKTLSAIYSQLGNAYFYLHDYAKALEYHHHDLTLARTIGDQLGEAKASGNLGNTLKVLGNFDEAIVCCQRHLDISRELNDKVGEARALYNLGNVYHAKGKSFGCPGPQDVGEFPEEVRDALQAAVDFYEENLSLVTALGDRAAQGRAFGNLGNTHYLLGNFRDAVIAHEQRLLIAKEFGDKAAERRAYSNLGNAYIFLGEFETASEYYKKTLLLARQLKDRAVEAQSCYSLGNTYTLLQDYEKAIDYHLKHLAIAQELNDRIGEGRACWSLGNAYTALGNHDQAMHFAEKHLEISREVGDKSGELTARLNLSDLQMVLGLSYSTNNSIMSENTEIDSSLNGVRPKLGRRHSMENMELMKLTPEKVQNWNSEILAKQKPLIAKPSAKLLFVNRLKGKKYKTNSSTKVLQDASNSIDHRIPNSQRKISADTIGDEGFFDLLSRFQSNRMDDQRCCLQEKNCHTASTTTSSTPPKMMLKTSSVPVVSPNTDEFLDLLASSQSRRLDDQRASFSNLPGLRLTQNSQSVLSHLMTNDNKEADEDFFDILVKCQGSRLDDQRCAPPPATTKGPTVPDEDFFSLILRSQGKRMDEQRVLLQRDQNRDTDFGLKDFLQNNALLEFKNSGKKSADH

SEQ ID NO: 75 - DNA, 1779 bp, homo sapiens
gtcgccccgggacggggaggtggggagctgagggcaagtcgcgcccgcccctgaaatcccagccgcctagcgattggctgcaagggtctcggcttggccgcggattggtcacacccgagggcttgaaaggtggctgggagcgccggacacctcagacggacggtggccagggatcaggcagcggctcaggcgaccctgagtgtgcccccaccccgccatggcccggctgctgcaggcgtcctgcctgctttccctgctcctggccggcttcgtctcgcagagccggggacaagagaagtcgaagatggactgccatggtggcataagtggcaccatttacgagtacggagccctcaccattgatggggaggagtacatccccttcaagcagtatgctggcaaatacgtcctctttgtcaacgtggccagctactgaggcctgacgggccagtacattgaactgaatgcactacaggaagagcttgcaccattcggtctggtcattctgggctttccctgcaaccaatttggaaaacaggaaccaggagagaactcagagatccttcctaccctcaagtatgtccgaccaggtggaggctttgtccctaatttccagctctttgagaaaggggatgtcaatggagagaaagagcagaaattctacactttcctaaagaactcctgtcctcccacctcggagctcctgggtacatctgaccgcctcttctgggaacccatgaaggttcacgacatccgctggaactttgagaagttcctggtggggccagatggtatacccatcatgcgctggcaccaccggaccacggtcagcaacgtcaagatggacatcctgtcctacatgaggcggcaggcagccctgggggtcaagaggaagtaactgaaggccgtctcatcccatgtccaccatgtaggggagggactttgttcaggaagaaatccgtgtctccaaccacactatctacccatcacagacccctttcctatcactcaaggccccagcctggcacaaatggatgcatacagttctgtgtactgccaggcatgtgggtgtgggtgcatgtgggtgtttacacacatgcctacaggtatgcgtgattgtgtgtgtgtgcatgggtgtacagccacgtgtctacctatgtgtctttctgggaatgtgtaccatctgtgtgcctgcagctgtgtagtgctggacagtgacaaccctttctctccagttctccactccaatgataatagttcacttacacctaaacccaaaggaaaaaccagctctaggtccaattgttctgctctaactgatacctcaaccttggggccagcatctcccactgcctccaaatattagtaactatgactgacgtccccagaagtttctgggtctaccacactccccaaccccccactcctacttcctgaagggccctcccaaggctacatccccaccccacagttctccctgagagagatcaacctccctgagatcaaccaaggcagatgtgacagcaagggccacggaccccatggcaggggtggcgtcttcatgagggaggggcccaaagcccttgtgggcggacctcccctgagcctgtctgaggggccagcccttagtgcattcaggctaaggcccctgggcagggatgccacccctgctccttcggaggacgtgccctcacccctcactggtccactggcttgagactcaccccgtctgcccagtaaaagcctttctgcagcagctgaaaaaaaaaaaaaaaaaaa

SEQ ID NO: 76 - protein, 225 aa, homo sapiens
MARLLQASCLLSLLLAGFVSQSRGQEKSKMDCHGGISGTIYEYGALTIDGEEYIPFKQYAGKYVLFVNVASYGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 77 - DNA, 796 bp, homo sapiens
ttctcttttagccgcagctatggtttctgccctaattattcttgtccttatttgtaatttaattcttaatttaatttaatttataattttgttgtaagtttctctgtgggcgtgaatggaaagtctaacccgtgtttctctgttcagcgtccgccggtcacggccgccgcccccagcgacgtcacccacacgcgcagaagcggacgccgcggtcaagatgtctctgccatgcccacgggacgcacggacggacggacgcacggacggactccacaaggtaggaagcctgcgccgaccgcaccgctgcacccaccacagcacacaggacacacgcgggccccgcgcccgcccaggcacacgcggcacacacggcacacacggcaggcaggccaggcacacgcatccgcaggacccgccgcacccgccacgcagacacggacgagccgccgcggtcaagatgttcacccgccgcggtcaagatgtatgtgccaccgaccctcgccccgctggacggacggacggacgcacgcacgccgtcagcgtccaccggtcactgccgccgcccacagtgatgtcacccacgaaagcacacacgtagaagcggacgccgtggtcaagatgtctctgccatccccacaggacggacggacggactccacaaggtgcgcgtgtcgccgaggccgccaggacggagcgattctcacggaggaaggagcacgccaacagggcctgactgcgtacagaaatgccccccctcaataaaattgcagttgaaatggaaaaaaaaaaaaaaaaa

SEQ ID NO: 78 - protein, 177 aa, homo sapiens
MPTGRTDGRTHGRTPQGRKPAPTAPLHPPQHTGHTRAPRPPRHTRHTRHTRQAGQAHASAGPAAPATQTRTSRRGQDVHPPRSRCMCHRPSPRWTDGRTHARRQRPPVTAAAHSDVTHESTHVEADAVVKMSLPSPQDGRTDSTRCACRRGRQDGAILTEEGARQQGLTAYRNAPPQ

SEQ ID NO: 79 - DNA, 2664 bp, homo sapiens
agaaattcaggagacacagacaagttcttccacgatgtcgtacagacgagaactagagaaataccgtgacctggatgaagatgaaatccttggagccctaacagaggaagagctgaggaccctggaaaatgagctggatgagctggaccctgataatgcactgctgcctgcaggcctgaggcagaaggatcagaccaccaaggcgcccacgggcccctttaaaagagaggagctcttggatcacttggaaaagcaagcaaaggagtttaaggaccgagaagatctggtcccctacacaggggaaaaacgaggaaaggtctgggttcctaagcagaagccactggatcctgtgctggaaagtgtgacgctggaaccggagctggaggaagccttggcaaatgcttcagatgcagaactctgtgacattgcagcgatcctgggcatgcacacgctcatgagtaaccagcagtactaccaggccctgagcagcagctccatcatgaacaaggaggggctcaacagcgtgattaaacccacacaatacaagcctgtgcccgacgaagaaccaaattcaacagacgtagaggaaacgctggaacggataaagaacaacgacccaaaacttgaagaagttaacctcaataatatccggaatatccccatccccaccctcaaggcatatgcagaagccctgaaagaaaactcatatgtgaagaagttcagcatcgtggggacacggagtaatgaccccgtggcgtatgcccttgctgagatgctcaaggagaacaaggtgttgaagacactgaatgtggaatccaacttcatttctggagctgggattctgcgcctggtagaagccctcccatacaacacttctctggtggaaatgaaaattgacaaccagagccagcccctgggcaacaaagtggaaatggagattgtgagcatgttggaaaaaaacgcaacacttctcaaattcggctaccactttacccagcaaggaccccggcttcgggcatccaacgcaatgatgaacaacaatgaccttgtgaggaagaggaggcttgcggacctgactgggcccatcattcccaagtgccggagtggtgtctagtgtgtggcggtggagtccatgcctttgaactggatgtgttctattgatgacctgtgctctgcaggggaaaccagaaggcaaaatgctggcagcatgaaacccttttgtggttcagttctttatgcactaaggttttaggttgactagtggttgtagttgaaaattttataaaataccgttaatgtgaagtttttctttagtcacagaagttgaatctggttattatttaaaaactagaagcccccaaaccagcagatcttactgaagatgatgttccagcagcagcgacttagccccaggagcccagtttcaatggccttgctgtgtggtgtttcaagtgcatttaaaatgtgtgacacagaaacggcacactcttccacatgcttttgaagtattataaaacactttattacaaatttgtcttagctattagcaaataaaactgattatcattctttattaaccctccttggaattttgaaaacctcgattaaagttgccaaattgattactggatccagaacacaattttcccctcagaacagatagacagactgaagccactgaactctgccaggagtcaacatgagattccttttgctggatatgcagaaatgataggaaaaaaaccaatggtgaaatttcaagtttcaaaaaccaacctttcattaccaatcccaggcaacaaacatgtccctgagtgttctttaagaacatttgggatttatgtacaatttaatactggagttagaactttttccttattgaatgccaaccttatgatggatgtgaaaatctacggccaaatacttttgaaaacacctttctatattgcacagtgggcaaatggcttatgtgaggtaagacactagagggataaatttccagatcaacatggctatggtatttagtaatggcccagcttagagacttcagctactgatctcatcacttattagacaaattgctgctgaccttacgcctgtatattaagcctccgcaggatgccggacaatggtgaagaaactccagatatcaaggaattgggaaatcctggccaaaccaccccaagatgattacactgaaatgtagtattagtactgctgccagatctctttttaacatcatgtgcgtctcttgggatccagcaaaagtgttaagccacaatgcccttgtgccttttaatataccacagtgccagttaaactaatatttttgtttgttgcttttgggagttattttcattagtgatttcagcaaatctcatgataaaggacaaggtcaagaactccagagcactgagcagagaggctggtgatgaaaaggtgaaggcctgcgcactgaactgtaaggcagtgggcagtacagggtaactggaggcggggccagggcctcagcgctatggaagagtgtccactgaggctgcacatggcccaggagtggcaccatgttgcagggacaaccatccccatttggcttctccttaaaacacaattgcagctgcattctgcatcgctgaaaactgcaatataatattaaatctgttggtc

SEQ ID NO: 80 - protein, 359 aa, homo sapiens
MSYRRELEKYRDLDEDEILGALTEEELRTLENELDELDPDNALLPAGLRQKDQTTKAPTGPFKREELLDHLEKQAKEFKDREDLVPYTGEKRGKVWVPKQKPLDPVLESVTLEPELEEALANASDAELCDIAAILGMHTLMSNQQYYQALSSSSIMNKEGLNSVIKPTQYKPVPDEEPNSTDVEETLERIKNNDPKLEEVNLNNIRNIPIPTLKAYAEALKENSYVKKFSIVGTRSNDPVAYALAEMLKENKVLKTLNVESNFISGAGILRLVEALPYNTSLVEMKIDNQSQPLGNKVEMEIVSMLEKNATLLKFGYHFTQQGPRLRASNAMMNNNDLVRKRRLADLTGPIIPKCRSGV

SEQ ID NO: 81 - DNA, 3393 bp, homo sapiens
aaccccgcgcgctgattggccgcgtgggcgaggcggaggagagccgtgcgcagcggcgtatgtggggccgtgtgcagacccgcgtgtggcgcaggcaaggaccctcaaaataaacagcctctaccttgcgagccgtcttccccaggcctgcgtccgagtctccgccgctgcgggcccgctccgacgcggaagatctgactgcagccatgagcagcaatgagtgcttcaagtgtggacgatctggccactgggcccgggaatgtcctactggtggaggccgtggtcgtggaatgagaagccgtggcagaggtggttttacctcggatagaggtttccagtttgtttcctcgtctcttccagacatttgttatcgctgtggtgagtctggtcatcttgccaaggattgtgatcttcaggaggatgcctgctataactgcggtagaggtggccacattgccaaggactgcaaggagcccaagagagagcgagagcaatgctgctacaactgtggcaaaccaggccatctggctcgtgactgcgaccatgcagatgagcagaaatgctattcttgtggagaattcggacacattcaaaaagactgcaccaaagtgaagtgctataggtgtggtgaaactggtcatgtagccatcaactgcagcaagacaagtgaagtcaactgttaccgctgtggcgagtcagggcaccttgcacgggaatgcacaattgaggctacagcctaattattttcctttgtcgcccctcctttttctgattgatggttgtattattttctctgaatcctcttcactggccaaaggttggcagatagaggcaactcccaggccagtgagctttacttgccgtgtaaaaggaggaaaggggtggaaaaaaaccgactttctgcatttaactacaaaaaaagtttatgtttagtttggtagaggtgttatgtataatgctttgttaaagaaccccctttccgtgccactggtgaatagggattgatgaatgggaagagttgagtcagaccagtaagcccgtcctgggttccttgaacatgttcccatgtaggaggtaaaaccaattctggaagtgtctatgaacttccataaataactttaattttagtataatgatggtcttggattgtctgacctcagtagctattaaataacatcaagtaacatctgtatcaggccctacatagaacatacagttgagtgggagtaaacaaaaagataaacatgcgtgttaatggctgttcgagagaaatcggaataaaagcctaaacaggaacaacttcatcacagtgttgatgttggacacatagatggtgatggcaaaggtttagaacacattattttcaaagactaaatctaaaacccagagtaaacatcaatgctcagagttagcataatttggagctattcaggaattgcagagaaatgcattttcacagaaatcaagatgttatttttgtatactatatcacttagacaactgtgtttcatttgctgtaatcagtttttaaaagtcagatggaaagagcaactgaagtcctagaaaatagaaatgtaattttaaactattccaataaagctggaggaggaaggggagtttgactaaagttctttttgtttgtttcaaattttcattaatgtatatagtgcaaaataccatattaaagaggggaatgtggaggactgaaagctgacagtttggacttttctttttgtacttaagtcatgtcttcaataatgaaaattgctgttaaaaggatgtatgggatttagatacttttgcaaagctatagaaaattcactttgtaatctgttataataatgcccttgagttctgtgttcagtctgaacaggttttttggtggtggtggttttgttttgttttggagacggagtctcactcttgtcgcccaggctggagtgcaggcttggctcactgcaacctccacctcccgggttcaagcaattctcctgcctcagcctcctgagtagctgggattacaggcacccgccaccaccccccgctaattttttgtatttttatttttattttatttttttatttttttttgagacagagtgtcgctctgttgcccaggctggagtgtagtggtgcgatctcggctcactgcaagctccgcctcctgggttcgtgccattctcctgcctcagcctcctgagtagctggggctacaggtacccgccaccgcgcccagctaattttttttttttgtatttttagtaaagacggggtttcacggtgttagccaggatggtctcaatctcctgacctcgtgatccgcccgccttggcctcccaaagtgctgggatcacaggcgtgagccaccgcgcccggcctattttttgtatttttagtagagactgggtttcatcatgttggtcgggctggtctccaactcctgacctcaggtgatccacctgccccgccccccaaagtgctagtgttacaggtgcgagccaccgtgtctggccgattctgaacagttttaataccattgctatttttgtgtttttcctgggccttttttttttttttttttttttttgagacagtctcgctctgttgcccaggctagagtgcaatggtgcaatctcagctcactgcaacctccaccccccacccccacaccccgttcaagtaattctcctgcctcagcctcccaaatagctgggattacaggtgtccgccaccacacccagctaatttttgttatttttagtagagatggggtttcactgtgttggtcaggctggtctccaactgttgccctcaggtgagccactgtgccccaccttttcctgggtttcataaggatctgaagtggtggattccttgtttttgctagtgtctcatttagagttgagatggaccttaaaactcatctgttttaactcactttttaatagatgagttaaacttaatttacttaaggatgtacagttagagcctggaacttcaaccattattcactccccatgccctgtttccccccacttcgaaattaaatgcggttagcatcatatagttcattttccccctccatgctgctgtgtgattcttgactttgggtatgagtttttcatccttcatgcagggttctgtcagttcatggtatagtgattcagtgttaaaatggtggtgtctcagctgtgctgtgcacattccaaccttgtcaaattaatagtcctgagcaagcaagaaaaagaggtaataacatacccatttctttatgaatataagcttataatattttttcatgtgctatttttactgagcaaattgtatgtctcacatgttaacacaataaatatcttgacaattttattttctaccataaaaaaaaaaaaaaaa

SEQ ID NO: 82 - protein, 177 aa, homo sapiens
MSSNECFKCGRSGHWARECPTGGGRGRGMRSRGRGGFTSDRGFQFVSSSLPDICYRCGESGHLAKDCDLQEDACYNCGRGGHIAKDCKEPKREREQCCYNCGKPGHLARDCDHADEQKCYSCGEFGHIQKDCTKVKCYRCGETGHVAINCSKTSEVNCYRCGESGHLARECTIEATA

SEQ ID NO: 83 - DNA, 8726 bp, homo sapiens
gacaatgctggggagatgaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggccaccgtcccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcagatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacctcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctccaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccaggcatatgcaatgatgctgtccctttccgaagacactcctctccacaccccctcccagggttctctggacgcttggttgaaggtcactggagccacgtcggagtctggagcatttcaccccatcaaccacctctgacgggctgggcagccgggggccggtggccagagcgagggcaccagccacgaaggacggaggcgggcggggccccggagaaccctgtccctgcgtgtggccactcctcagcatcctccccacccaccatggttcattccgacttttccaatggaaactcagatcccaaaagtccctaaagcagtcgtagagtctcaccatctccaaggattggtcttgagaacactgttcagtgacggccatgcaggtggccgtccaaagacagccaacggagctgcctcgcagaatcagccagtgggcaggtggacgctctgctgagacagaagctggtggccactgccgggtgcccgcgtggggtcgcggaagggaatggatagactggtgtgctcaaaagagagagatcactcaaatgatttttataatgaaatgacaagaataacccttttggtaaccgtattgactgcagagtctatttaagcatgtggttttaaaaatagacagtattttttaaaaatcaaaaaatgacttgcaaattgttttttaaaagtaattttgcattgctttgaaatttgagctcatttgcaaacccgagtctgcctgggaacccgcactgtgcctgggtgtattctttatactgtagataatggagaaattttctatctctgtccctatttgtataagccaaggtgatgctgggtgccccgaggcagaacaagaggcgcggggccacacccgtgaaccatgcagacggccgaagaagtcttaggcagggcgccctgggctgcaggcctgcccgaggctgggatgggaagtgtgcctgccctcgtgtgacatggaattggtgtcaggaccgccacgtggccttcagaggaatccacaggtccccacccaagatccctcaattatatggggaagtcgagggcctgtggcttggatccgccatgcagagatgtggccgggcacccatcttccttccctcctctgtccctgcctcggccaccccacgcgggaacccagcgccgtcctctgaaggcagggccttggccacgtcctgggtctcccacctcccacctgaccccagcggctccggtgtcctccacgtggctgccctggggagcaatcccagcggatcgctccgggccaccaagccgcacctgtgcctgagactccggatggacgacacagtcgtcacgtcgctcttcctgcgggttcttggcgagacacagcttgagaacagaagggcgtcgggggaacctgccgcaaggagcagagacagcacagccccccgggcccagccgcctccctctcttgggacgcaacttcttccccactcggatgggctttaaattattcccataggggccaatttcaaataataatttttttccctgatggaatttaccttaatctgtatataacttgtaattttttctaattcatttcttttcttattttatttcctccttaacagtatttttggcattagacattcttattgtgaagaaataatgttaatataagtatctggtgaaggaccaaaaccgtgtgataaggttgtgtgtcgtgtgggagtggggcgattttttatgtgccaaatacccccgtcccccccatgaatcctgctgtccctgctgccgtttaccagacaatcatatgtttttgttaaatttgcgtttcagttacatttgcatttaagacaagtgttctatttatttcttgtattgtttggaagaaaaaatgatgatagagtcccaaaaagaagagaaaaaaaatgcccaagttgccctttaaaaaaaaagagcgtaaatacaaacaggagtggtgcaagccgccttggtgtgggtttgtgtcacgtgtggacatctcctcaggctttgtgtcacgcgtggacatctcctcaggctgtccccagcggtgacgggaggtgtcctggctgctccaggacaaaagacaatcgtctctgtgggtgccgggtggtccaggcttgcactgaagacgtgccacggggaggctcctgcaggaggctcaacccgacggatcacagtgaaagggattcctcccacgccagatctgcacaacgaggcaagacaggacccacctgtgcgtgcgctggggccatggggtggccccgccggggcagcgggggagctgcctgcagaagagccagctggcgtgtcgggaaggatccaggatctgcaaacacaactgctcaggccttctcacgcgtttccacaacatcccctgggtcagacccaccaggtaccccgtaggaatttccagtttcccttgatctagatgggattcttataaaaattcaacctcagacataaacaccccatttctgtaaacccaaattatatggtttcttctgcgaaagagtaaggtgtgtgcttttttttttttgcaatatgaccccgtctctctgaagtgggacattcggacggatggagccctcagcgtgtcttttcagcaggagcagaaccgatgagagccgcccttaccgttggtctccggatcccccagtcccatcccgccgttttcggctgtcttcctaaccgtcctgtcttctcttggcgctctttccttccacctttcccaagagtcctggttgcacgttttaagtcatatattttcgtccccctgaaaatgatggcaagcccagtttctcctgagcattcagacccccaggccccagcacttggcgttttcaggaggccctgttcttagagcccctgacaaaggcagcacttatttcctgggctggtgcgccccaaaacacggccccgacacttagtgtggccccaggccccagcgagcctcgccctcccagttttgctctgcccagcagtgttggtgcccagagatgacaagggccagggagcctggcccgggtgtgagaattcagagattctggcctccagctgtcaccacaccgtaacggggccatgtaactgtgcagcatggacagggatgcgacggggcagctggctgtgtccatggccaggtggccagggtcagggctgcaagccaggggtccagggcccttccgttcagcccaaatgctgccccaatgctaactccttggattgtcaacccccatcccccaaatggaaattccgaaggaggcctcctcgcacctgccctccgctgctcctcagaccccagcccccagcgagccgacgtccccacccgttcctgctctcatccccaggttgggcacgtggggttcctcctctgtgggcctggcagacccttcatgagtgggacccaagatatcactgacttcaacccagaggatcgagcccctgcaccctgcctggggccctggggtgtggagcagtggctggggtgggcgtggtgtggcctgagagactgcccagctggagaggccttcctttacaaggccacgcgtgcagctgtcccatccagaccccgactggccaagacctccacgtccccagagtccagccctggaaattccaagggccctggcgtcctctgccttccccgcttccccatgagcgtctgcaaaacacttgcctgaatacatatcacgtattttagactcgaagcctcaaagcactggattgtggtcccctgccccctctgtcccgtccccctgcccaagtgactgaaacctactgagctatattcactgtgctgtcctagggggagggagagcagagctcgcccctgcactgcagccttgtgggggagggcaaggctctcctcccagccagggacgccaggacatagctgctcctggtcagtggaggtcagccgggtatcaaaagccatgaaactgtgtctctgtagcaatgagtgatactgtgacaaaaccatccttgcattcttcctagaagagttcctctgctccttccattccatttttgtgtttgttttgttcttttctgtcactgatccgtattaccacttttggaaaaaaataaataaataaataaataaaaggcagcttgagtttccaaacgtgtgattcacttgtgaacaaaagtcattctaacaattgccttcagcgtcacgtgcattgccactgcgctttcggcacgagggatgctgagccctggtgtcagagtcgtaatttaaagcgtgtgtgtatatggactttgtcccttaaggtcgatataaagaatcctcgcagaatcacagacctgtgccgcccgccaccttctgccattgttacattacagatttggtttagttttgttttgttttgttttttcttttagaactgtatagtattgaaaaagaaatcaaatgtaaatgtctggttttcatataatgtttaaaaagaccattgagaaggaggctggcgctcgccccatgtcccccttgattgtaaattgcttctgttctgtttataagtaaactgtgcatgactcctgcttagcggtcattatcgtgtctgttggtgaaatttttattaaaaggaaaattctgtagatgcacttattgaatatgtgattaggatctacgtctgagactaggagtcctgaactgctgacgcgaaagaggcgcagttcccaattaatacggaaatcgctgtgggagaagaatgaaataagacgtgaagtgtaggaaatcatgaaaagaacaattttgcaaattgcattctgatgcttgtgatgaacacaaatgtacttgtgtagagacatttccttaagagaaagcctaggagaagccgatttggaggttaatgctgtagaataggactgtataccaaatgtaatctttccaatgctccaatgaatttatacatgagattgatatgcaataaatctgtgtgcttttctaaaaaaaaaa

SEQ ID NO: 84 - protein, 1275 aa, homo sapiens
MRSKARARKLAKSDGDVVNNMYEPNRDLLASHSAEDEAEDSAMSPIPVGPPSPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNLIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPSRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQAYAMMLSLSEDTPLHTPSQGSLDAWLKVTGATSESGAFHPINHL

SEQ ID NO: 85 - DNA, 8669 bp, homo sapiens
gacaatgctggggagatgaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggccaccgtcccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcagatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacctcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctccaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccagggttctctggacgcttggttgaaggtcactggagccacgtcggagtctggagcatttcaccccatcaaccacctctgacgggctgggcagccgggggccggtggccagagcgagggcaccagccacgaaggacggaggcgggcggggccccggagaaccctgtccctgcgtgtggccactcctcagcatcctccccacccaccatggttcattccgacttttccaatggaaactcagatcccaaaagtccctaaagcagtcgtagagtctcaccatctccaaggattggtcttgagaacactgttcagtgacggccatgcaggtggccgtccaaagacagccaacggagctgcctcgcagaatcagccagtgggcaggtggacgctctgctgagacagaagctggtggccactgccgggtgcccgcgtggggtcgcggaagggaatggatagactggtgtgctcaaaagagagagatcactcaaatgatttttataatgaaatgacaagaataacccttttggtaaccgtattgactgcagagtctatttaagcatgtggttttaaaaatagacagtattttttaaaaatcaaaaaatgacttgcaaattgttttttaaaagtaattttgcattgctttgaaatttgagctcatttgcaaacccgagtctgcctgggaacccgcactgtgcctgggtgtattctttatactgtagataatggagaaattttctatctctgtccctatttgtataagccaaggtgatgctgggtgccccgaggcagaacaagaggcgcggggccacacccgtgaaccatgcagacggccgaagaagtcttaggcagggcgccctgggctgcaggcctgcccgaggctgggatgggaagtgtgcctgccctcgtgtgacatggaattggtgtcaggaccgccacgtggccttcagaggaatccacaggtccccacccaagatccctcaattatatggggaagtcgagggcctgtggcttggatccgccatgcagagatgtggccgggcacccatcttccttccctcctctgtccctgcctcggccaccccacgcgggaacccagcgccgtcctctgaaggcagggccttggccacgtcctgggtctcccacctcccacctgaccccagcggctccggtgtcctccacgtggctgccctggggagcaatcccagcggatcgctccgggccaccaagccgcacctgtgcctgagactccggatggacgacacagtcgtcacgtcgctcttcctgcgggttcttggcgagacacagcttgagaacagaagggcgtcgggggaacctgccgcaaggagcagagacagcacagccccccgggcccagccgcctccctctcttgggacgcaacttcttccccactcggatgggctttaaattattcccataggggccaatttcaaataataatttttttccctgatggaatttaccttaatctgtatataacttgtaattttttctaattcatttcttttcttattttatttcctccttaacagtatttttggcattagacattcttattgtgaagaaataatgttaatataagtatctggtgaaggaccaaaaccgtgtgataaggttgtgtgtcgtgtgggagtggggcgattttttatgtgccaaatacccccgtcccccccatgaatcctgctgtccctgctgccgtttaccagacaatcatatgtttttgttaaatttgcgtttcagttacatttgcatttaagacaagtgttctatttatttcttgtattgtttggaagaaaaaatgatgatagagtcccaaaaagaagagaaaaaaaatgcccaagttgccctttaaaaaaaaagagcgtaaatacaaacaggagtggtgcaagccgccttggtgtgggtttgtgtcacgtgtggacatctcctcaggctttgtgtcacgcgtggacatctcctcaggctgtccccagcggtgacgggaggtgtcctggctgctccaggacaaaagacaatcgtctctgtgggtgccgggtggtccaggcttgcactgaagacgtgccacggggaggctcctgcaggaggctcaacccgacggatcacagtgaaagggattcctcccacgccagatctgcacaacgaggcaagacaggacccacctgtgcgtgcgctggggccatggggtggccccgccggggcagcgggggagctgcctgcagaagagccagctggcgtgtcgggaaggatccaggatctgcaaacacaactgctcaggccttctcacgcgtttccacaacatcccctgggtcagacccaccaggtaccccgtaggaatttccagtttcccttgatctagatgggattcttataaaaattcaacctcagacataaacaccccatttctgtaaacccaaattatatggtttcttctgcgaaagagtaaggtgtgtgcttttttttttttgcaatatgaccccgtctctctgaagtgggacattcggacggatggagccctcagcgtgtcttttcagcaggagcagaaccgatgagagccgcccttaccgttggtctccggatcccccagtcccatcccgccgttttcggctgtcttcctaaccgtcctgtcttctcttggcgctctttccttccacctttcccaagagtcctggttgcacgttttaagtcatatattttcgtccccctgaaaatgatggcaagcccagtttctcctgagcattcagacccccaggccccagcacttggcgttttcaggaggccctgttcttagagcccctgacaaaggcagcacttatttcctgggctggtgcgccccaaaacacggccccgacacttagtgtggccccaggccccagcgagcctcgccctcccagttttgctctgcccagcagtgttggtgcccagagatgacaagggccagggagcctggcccgggtgtgagaattcagagattctggcctccagctgtcaccacaccgtaacggggccatgtaactgtgcagcatggacagggatgcgacggggcagctggctgtgtccatggccaggtggccagggtcagggctgcaagccaggggtccagggcccttccgttcagcccaaatgctgccccaatgctaactccttggattgtcaacccccatcccccaaatggaaattccgaaggaggcctcctcgcacctgccctccgctgctcctcagaccccagcccccagcgagccgacgtccccacccgttcctgctctcatccccaggttgggcacgtggggttcctcctctgtgggcctggcagacccttcatgagtgggacccaagatatcactgacttcaacccagaggatcgagcccctgcaccctgcctggggccctggggtgtggagcagtggctggggtgggcgtggtgtggcctgagagactgcccagctggagaggccttcctttacaaggccacgcgtgcagctgtcccatccagaccccgactggccaagacctccacgtccccagagtccagccctggaaattccaagggccctggcgtcctctgccttccccgcttccccatgagcgtctgcaaaacacttgcctgaatacatatcacgtattttagactcgaagcctcaaagcactggattgtggtcccctgccccctctgtcccgtccccctgcccaagtgactgaaacctactgagctatattcactgtgctgtcctagggggagggagagcagagctcgcccctgcactgcagccttgtgggggagggcaaggctctcctcccagccagggacgccaggacatagctgctcctggtcagtggaggtcagccgggtatcaaaagccatgaaactgtgtctctgtagcaatgagtgatactgtgacaaaaccatccttgcattcttcctagaagagttcctctgctccttccattccatttttgtgtttgttttgttcttttctgtcactgatccgtattaccacttttggaaaaaaataaataaataaataaataaaaggcagcttgagtttccaaacgtgtgattcacttgtgaacaaaagtcattctaacaattgccttcagcgtcacgtgcattgccactgcgctttcggcacgagggatgctgagccctggtgtcagagtcgtaatttaaagcgtgtgtgtatatggactttgtcccttaaggtcgatataaagaatcctcgcagaatcacagacctgtgccgcccgccaccttctgccattgttacattacagatttggtttagttttgttttgttttgttttttcttttagaactgtatagtattgaaaaagaaatcaaatgtaaatgtctggttttcatataatgtttaaaaagaccattgagaaggaggctggcgctcgccccatgtcccccttgattgtaaattgcttctgttctgtttataagtaaactgtgcatgactcctgcttagcggtcattatcgtgtctgttggtgaaatttttattaaaaggaaaattctgtagatgcacttattgaatatgtgattaggatctacgtctgagactaggagtcctgaactgctgacgcgaaagaggcgcagttcccaattaatacggaaatcgctgtgggagaagaatgaaataagacgtgaagtgtaggaaatcatgaaaagaacaattttgcaaattgcattctgatgcttgtgatgaacacaaatgtacttgtgtagagacatttccttaagagaaagcctaggagaagccgatttggaggttaatgctgtagaataggactgtataccaaatgtaatctttccaatgctccaatgaatttatacatgagattgatatgcaataaatctgtgtgcttttctaaaaaaaaaa

SEQ ID NO: 86 - protein, 1256 aa, homo sapiens
MRSKARARKLAKSDGDVVNNMYEPNRDLLASHSAEDEAEDSAMSPIPVGPPSPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNLIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPSRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQGSLDAWLKVTGATSESGAFHPINHL

SEQ ID NO: 87 - DNA, 2091 bp, homo sapiens
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct

SEQ ID NO: 88 - protein, 482 aa, homo sapiens
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 89 - DNA, 5613 bp, homo sapiens
cggggcggcggcgggggccgggggggcccgggcgcgcgggagcgggagcggccgggggagccggagcgcaccatggaggcggcggcaggcggccgcggctgtttccagccgcacccggggctgcagaagacgctggagcagttccacctgagctccatgagctcgctgggcggcccggccgctttctcggcgcgctgggcgcaggaggcctacaagaaggagagcgccaaggaggcgggcgcggccgcggtgccggcgccggtgcccgcagccaccgagccgccgcccgtgctgcacctgcccgccatccagccgccgccgcccgtgctgcccgggcccttcttcatgccgtccgaccgctccaccgagcgctgcgagaccgtactggaaggcgagaccatctcgtgcttcgtggtgggaggcgagaagcgcctgtgtctgccgcagattctcaactcggtgctgcgcgacttctcgctgcagcagatcaacgcggtgtgcgacgagctccacatctactgctcgcgctgcacggccgaccagctggagatcctcaaagtcatgggcatcctgcccttctcggcgccctcgtgcgggctcatcaccaagacggacgccgagcgcctgtgcaacgcgctgctctacggcggcgcctacccgccgccctgcaagaaggagctggccgccagcctggcgctgggcctggagctcagcgagcgcagcgtccgcgtgtaccacgagtgcttcggcaagtgtaaggggctgctggtgcccgagctctacagcagcccgagcgccgcctgcatccagtgcctggactgccgcctcatgtacccgccgcacaagttcgtggtgcactcgcacaaggccctggagaaccggacctgccactggggcttcgactcggccaactggcgggcctacatcctgctgagccaggattacacgggcaaggaggagcaggcgcgcctcggccgctgcctggacgacgtgaaggagaaattcgactatggcaacaagtacaagcggcgggtgccccgggtctcctctgagcctccggcctccataagacccaaaacagatgacacctcttcccagtcccccgcgccttccgaaaaggacaagccgtccagctggctgcggaccttggccggctcttccaataagagcctgggctgtgttcaccctcgccagcgcctctctgctttccgaccctggtcccccgcagtgtcagcgagtgagaaagagctctccccacacctcccggccctcatccgagacagcttctactcctacaagagctttgagacagccgtggcgcccaacgtggccctcgcaccgccggcccagcagaaggttgtgagcagccctccgtgtgccgccgccgtctcccgggcccccgagcctctcgccacttgcacccagcctcggaagcggaagctgactgtggacaccccaggagccccagagacgctggcgcccgtggctgccccagaggaggacaaggactcggaggcggaggtggaagttgaaagcagggaggaattcacctcctccttgtcctcgctctcttccccgtcctttacctcatccagctccgccaaggacctgggctccccgggtgcgcgtgccctgccctcggccgtccctgatgctgcggcccctgccgacgcccccagtgggctggaggcggagctggagcacctgcggcaggcactggagggcggcctggacaccaaggaagccaaagagaagttcctgcatgaggtggtcaagatgcgcgtgaagcaggaggagaagctcagcgcagccctgcaggccaagcgcagcctccaccaggagctggagttcctacgcgtggccaagaaggagaagctgcgggaggccacggaggccaagcgtaacctgcggaaggagatcgagcgtctccgcgccgagaacgagaagaagatgaaagaggccaacgagtcacggctgcgcctgaagcgggagctggagcaggcgcggcaggcccgggtgtgcgacaagggctgcgaggcgggccgcctgcgcgccaagtactcggcccagatcgaagacctgcaggtgaagctgcagcacgcggaggcggaccgggagcagctgcgggccgacctgctgcgggagcgcgaggcccgggagcacctggagaaggtggtgaaggagctgcaggaacagctgtggccgcgggcccgccccgaggctgcgggcagcgagggcgctgcggagctggagccgtagattccgtgcctgccgccgcagcgccgccgacaacgcgggtgcaggggggcgcggctgggcggtgcagctccgcccggctccgcccctgcagcccacacagcacaacgtcttaccgtgcctattaccaagcgagtgtttgtaaccatgtagttttggaacccactgcaaaattttctactggccaagttcaagtgagtaagccgcgtcccccaactacagctggagacggggccagctcggcggcctgctggtcctctgcttgctggaacattctaacatttacacttttgttataagctatttaaaaccagtaaggagacttgaaattcagaaaatcaacacatttttaaatgactaacttctaaaagccccaacacatgacgccatctgaagacccgcaacggagtgggggtggcggccgccccaccctccccacccggggaagccatcacagctcatctgcccgcggctgcgtgaggacagcaggggtttttcttcagagtctattttttcagcgacaaggacccaggtcttcctgctgctgccagggagagcagggacagtgccgcgtgcgagatgagctcgaacactgcccgccttactgccgcctaccccgcccgccacgccgccgtcgatgccagcgctgtccccacgggtaccaggaagtgcagagccgcacaggagctgccccggagctgaggggacggtcttcggctcctctgcaccccgtgattctgcccacgctcctccaccacgaggcactgacctgcgtcgggtggtgaccgtggctggcggtcacgccctcagcccctccgggcacacgtgccgcctgaccgggcgacccttttcagttcggcaaacgtcgctcccttcattttgggactgaggctgcagcattggaacaaaagagcattatttcaatttttctttctttttttttgttcgttcatttaaacgtatatttagaactgcactttgtccacaaccttcccttctctttctattccccagtgaactgaggtttttaccgatttatagagcagtcaaatccgaagtgctcgagtgcttagaaaccccctctggtgcttggttgaacaagggaatcacaagaaaacgaaaatgcaaaaactgaacttcgggggtcgttctgtgccttccagcatcttgtacagcaaatcctgactcgtgtctttttacccccaagatatctgtcttcagtagcgactgaatctgccactctcagaataagttccttgcatttattccaaataatctcgtttactctcacctgtttatgcaaattgtataaggtttcttatgcccaagcttgaaaaatgatttcccagtagacaagaggcggctacctatcctacagttacggtatttatttacataagaagatcttacaggagttctttgcttgaatccgttctaacacccgcggcagctgcacgcgctcacagaaggtggaggttacttgcccaggtacagacgacctcggggcagtgacgagcaaagaccagagactgctgagccctcgcatctgggtggcggaattgcctgcggggttttgcccttggtttactgaggggggtcttggttgctgctgaagccccccaccccttctaaagtgcaatgcaaaagggacatcatgtatatgcagcgtttgtttggaattttctttgcttttgttttctttgcggttgttctgtgtgcatggattccacacctctgccgtaggtagatccgtcagcgggcattattaccgtgtcttgtaaagggtcggttttgttatgcaacatgcagaatgctgtttttagccttgttttaccagagttgttttttttttcagttatttcttcaagggaaactaaatgatttagttggagcaaagctttaagtgtgttggcgtgcttctgtgtggctgtcctgtgtcgccaggtcgaagatcacagtgaggtagaggccctgccccatccccagggccgccaggcttgctccgtttgctttgagtttttagaccccagagggagatgagcttttccaagctgtgtctgggccagagcctctccttgcccttgctccatcccgacggtcaccgttgggtccacgcctccaccgccccatcttgccccaaacggaaagcgctgtatctgcagtggcagcccttccccacttcggctctgggagggtccagccagtgtcacctgggcccaccctttcctgcagctgccaggcccgtgcggtcagtgggacccggacgtgggcaggcgagctcgggaccctcccaggcagttcccacagctcttgcctcggctcaccagggtcacttccactgtcaggggcctgagggggcagctgtggctgcagggctgctctggactgaggggtcccaggccccgaggggtgcacgcctggctccccttggcacaggtgcgagtccgtttcttttcagcagaagggggaagaggtgtccgctgtgtgggctgctgactcctctgtgtgtgaggcccttcatctaagtgattgtgtattcagtttaattctcattatatttctatactgaaagaagatttttaacgaagggaaaaacaacagcaataacattcatatctctggagcagctaactcatacacgtaatgtctgcttttcgtacagaactagccaatgtaaaaacagttcacctgtaaatactttttcctttttcaccgttgtattatacatgtatatgctgggtcctttttcagaaactcttttcttacctgagagttgtcttgttttctgggctgtttttaactgaggaaaaaaaaaatgctttcctgccggggggcaggggagacggagaaacccatgtgcgtttcccatgtgaccccctcctccctgtgggtctgagccccggccccccccacctcctcctccctgtgggtccgaaccccggcccccccaccccctcctccctgtgggtccgaaccccggcccccccaccccctcctccctgtgggtccgatccccggccccccccaccccctcctccctgtgggtccgaaccccggccccccccccaccccctcctccctgtgggtccgaaccccggcccccccacccctccctcagcccaccagggtccagggagatgttcgttctcgctttaagtcaggagtcacaaatgacttttttttttcaattaaggaaaaagctccatctctacctttaacatcacccagacccccgcccctgcccgtgccccacgctgctgctaacgacagtatgatgcttactctgctactcggaaactatttttatgtaattaatgtatgctttcttgtttataaatgcctgatttaaaaagaaaa

SEQ ID NO: 90 - protein, 728 aa, homo sapiens
MEAAAGGRGCFQPHPGLQKTLEQFHLSSMSSLGGPAAFSARWAQEAYKKESAKEAGAAAVPAPVPAATEPPPVLHLPAIQPPPPVLPGPFFMPSDRSTERCETVLEGETISCFVVGGEKRLCLPQILNSVLRDFSLQQINAVCDELHIYCSRCTADQLEILKVMGILPFSAPSCGLITKTDAERLCNALLYGGAYPPPCKKELAASLALGLELSERSVRVYHECFGKCKGLLVPELYSSPSAACIQCLDCRLMYPPHKFVVHSHKALENRTCHWGFDSANWRAYILLSQDYTGKEEQARLGRCLDDVKEKFDYGNKYKRRVPRVSSEPPASIRPKTDDTSSQSPAPSEKDKPSSWLRTLAGSSNKSLGCVHPRQRLSAFRPWSPAVSASEKELSPHLPALIRDSFYSYKSFETAVAPNVALAPPAQQKVVSSPPCAAAVSRAPEPLATCTQPRKRKLTVDTPGAPETLAPVAAPEEDKDSEAEVEVESREEFTSSLSSLSSPSFTSSSSAKDLGSPGARALPSAVPDAAAPADAPSGLEAELEHLRQALEGGLDTKEAKEKFLHEVVKMRVKQEEKLSAALQAKRSLHQELEFLRVAKKEKLREATEAKRNLRKEIERLRAENEKKMKEANESRLRLKRELEQARQARVCDKGCEAGRLRAKYSAQIEDLQVKLQHAEADREQLRADLLREREAREHLEKVVKELQEQLWPRARPEAAGSEGAAELEP

SEQ ID NO: 91 - DNA, 2042 bp, homo sapiens
ggaaaacgagtcaggggtcggaataaattttagtatattttgtgggcaattcccagaaattaatggctatgagttcttttttgatcaactcaaactatgtcgaccccaagttccctccatgcgaggaatattcacagagcgattacctacccagcgaccactcgcccgggtactacgccggcggccagaggcgagagagcagcttccagccggaggcgggcttcgggcggcgcgcggcgtgcaccgtgcagcgctacgcggcctgccgggaccctgggcccccgccgcctccgccaccacccccgccgcccccgccaccgcccggtctgtcccctcgggctcctgcgccgccacccgccggggccctcctcccggagcccggccagcgctgcgaggcggtcagcagcagccccccgccgcctccctgcgcccagaaccccctgcaccccagcccgtcccactccgcgtgcaaagagcccgtcgtctacccctggatgcgcaaagttcacgtgagcacggtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctagtgcccccgcacgcgggagccacgaacctcggggtgggggtgggcagtgagtgcaggggatggggtggggggacaggagggggccctggggcctgggccccggaaaaatctatctgccctcccccacactttatatacgaataaacgcagaagagggggaggggaagctttatttatagaaatgacaatagagggccacggggaggcccccccagaagcaagattcaaatctcttgctttctttcttaaaaaaaagaaaaagaaaaagcaagaagaaggaagaaagaaaaagacagaaagagaaataggaggaggctgcagctcctcgttttcagctttggcgaagatggatccacgtttcatctttaatcacgccaggtccaggcccatctgtcttgtttcctctgccgaggagaagacgggcctcggtggcgaccattacctcgacacccgctaacaaatgaggcccggctcggccgcctccgcctctgctactgccgctgctggaagacagcctggatttcctttctttgtcccccactcccgatacccagcgaaagcaccctctgactgccagatagtgcagtgttttggtcacggtaacacacacacactctccctcatctttcgtgcccattcactgagggccagaatgactgctcacccacttccaccgtggggttgggggtgggcaacagaggaggggagcaagtagggaagggggtggccttgacaactcaggagtgagcaggaaaattgagtccaaggaaaaagagagactcagagacccgggagggccttcctctgaaaggccaagccaagccatgcttggcagggtgaggggccagttgagttctgggagctgggcactactctgccagtccagagttgtacagcagaagcctctctcctagactgaaaatgaatgtgaaactaggaaataaaatgtgcccctcccagtctgggaggaggatgttgcagagccctctcccatagtttattatgttgcatcgtttattattattattgataatattattattactatttttttgtgtcatgtgagtcctctctccttttctctttctgacattccaaaaccaggccccttcctacctctggggctgcttgagtctagaacccttcgtatgtgtgaatatctgtgtgctgtacagagtgacaatagaaataaatgtttggtttcttgtgaccagcaaaaaaaaaa

SEQ ID NO: 92 - protein, 251 aa, homo sapiens
MAMSSFLINSNYVDPKFPPCEEYSQSDYLPSDHSPGYYAGGQRRESSFQPEAGFGRRAACTVQRYAACRDPGPPPPPPPPPPPPPPPGLSPRAPAPPPAGALLPEPGQRCEAVSSSPPPPPCAQNPLHPSPSHSACKEPVVYPWMRKVHVSTVNPNYAGGEPKRSRTAYTRQQVLELEKEFHYNRYLTRRRRVEIAHALCLSERQIKIWFQNRRMKWKKDHKLPNTKIRSGGAAGSAGGPPGRPNGGPRAL

SEQ ID NO: 93 - protein, 286 aa, Homo sapiens
  MISC_FEATURE (1)..(1): x = m, p or absent
XXXXXXXXXXXXXXXXXXXXXXXXXXXXXXXXXXMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 94 - protein, 347 aa, homo sapiens
  misc_feature (61)..(69): Xaa can be any naturally occurring amino acid
MTLDHQIINPTLKWSQPAVPSGGPLVQHAHTTLDSDAGLTENPLTKLLAIGKEDDNAQWHXXXXXXXXXXXXXXXXXXXXXXXXXXXXXXXGSILDVYSGEQGISPINMGLTSASCPSSLPMKREITETDTRALAKERQKKDNHNLIERRRRYNINYRIKELGTLIPKSNDPDMRWNKGTILKASVEYIKWLQKEQQRARELEHRQKKLEQANRRLLLRIQELEIQARTHGLPTLASLGTVDLGAHVTKQQSHPEQNSVDYCQQLTVSQGPSPELCDQAIAFSDPLSYFTDLSFSAALKEEQRLDGMLLDDTISPFGTDPLLSATSPAVSKESSRRSSFSSDDGDEL

SEQ ID NO: 95 - protein, 479 aa, homo sapiens
  MISC_FEATURE (1)..(1): x = m or absent
XXXXXXXXXXXEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 96 - protein, 187 aa, homo sapiens
  MISC_FEATURE (1)..(1): x = a or v
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEXRXXQXXXXXXXXXXXXXXXXX

SEQ ID NO: 97 - protein, 1275 aa, homo sapiens
  misc_feature (1230)..(1249): Xaa can be any naturally occurring amino acid
MRSKARARKLAKSDGDVVNNMYEPNRDLLASHSAEDEAEDSAMSPIPVGPPSPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNLIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPSRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKXXXXXXXXXXXXXXXXXXXXQGSLDAWLKVTGATSESGAFHPINHL

SEQ ID NO: 98 - protein, 72 aa, homo sapiens
MARLLQASCLLSLLLAGFVSQSRGQEKSKMDCHGGISGTIYEYGALTIDGEEYIPFKQYAGKYVLFVNVASY

SEQ ID NO: 99 - DNA, 2076 bp, homo sapiens
agttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgtacccgctgcggtgtaccaccaccatcaccaccacccctacgtgcacccccaggcgcccgtggcggcggcggcgccggacggcaggtacatgcgctcctggctggagcccacgcccggtgcgctctccttcgcgggcttgccctccagccggccttatggcattaaacctgaaccgctgtcggccagaaggggtgactgtcccacgcttgacactcacactttgtccctgactgactatgcttgtggttctcctccagttgatagagaaaaacaacccagcgaaggcgccttctctgaaaacaatgctgagaatgagagcggcggagacaagccccccatcgatcccaataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtgatgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttctaaaaaaaaaaaaaaaaaaaa

SEQ ID NO: 100 - protein, 272 aa, homo sapiens
MATTGALGNYYVDSFLLGADAADELSVGRYAPGTLGQPPRQAATLAEHPDFSPCSFQSKATVFGASWNPVHAAGANAVPAAVYHHHHHHPYVHPQAPVAAAAPDGRYMRSWLEPTPGALSFAGLPSSRPYGIKPEPLSARRGDCPTLDTHTLSLTDYACGSPPVDREKQPSEGAFSENNAENESGGDKPPIDPNNPAANWLHARSTRKKRCPYTKHQTLELEKEFLFNMYLTRDRRYEVARLLNLTERQVKIWFQNRRMKMKKINKDRAKDE

SEQ ID NO: 101 - DNA, 3251 bp, homo sapiens
cagcaactatgaaataatcgtagtatgagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcatgccattacagctttctagatgctaatacatgtgactatcgtccaatttgctttcttttgtagtgacattaaatttggctataaaagatggactacatgtgatactcctatggacgttaattgaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaacaagtgctttttatcttgaaattcaaccaacggaaagaatatgcatagaataatgcattctatgtagccatgtcactgtgaataacgatttcttgcatatttagccattttgattcctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatttataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaaaattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcacccatcagttatttgtgagggtgtttattctatatgaatattgtttcatgtttgtatgggaaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaaaaaaaaaaaaaaaaaaaaaaaaaaaaaaaaaaaaaaaaaaaaaaaaaa

SEQ ID NO: 102 - protein, 326 aa, homo sapiens
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKVNKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNAGELESDSGSDKANSPAGGIPSTSSCLPSPSTPVQSPHPQFPHISSTMNGTSNSPSGNHQSSFANRPRKSSVNGSSATSSG

SEQ ID NO: 103 - DNA, 2834 bp, homo sapiens
tttgctaggcagcggcagtggcggcggcagcggcggctggagcctcctgattgggtttcggagtccggtactggagccaatcagcgcgggcagcgaaccgggggagcgaggcacggagtgtacctcacagccttctaggatctccagagtggacaggaatctcacttggagggaccatggagcagtatacagcaaacagcaatagttcgacagagcagattgttgtccaggcaggacagattcagcagcagcagcagggtggtgtcactgctgtgcagttgcagactgaggcccaggtggcatccgcctcaggccagcaagtccagaccctccaggtagtccaagggcagccattaatggtgcaggtcagtggaggccagctaatcacatcaactggccaacccatcatggtccaggctgtccctggtggacaaggtcaaaccatcatgcaagtacctgtttctggaacacagggtttgcagcaaatacagttggtcccacctggacagatccagatccagggtggacaggctgtgcaggtgcagggccagcagggccagacccagcagatcatcatccagcagccccagacggctgtcactgctggccagactcagacacagcagcagattgctgtccagggacagcaagtggcacagactgctgaagggcagaccatcgtctatcaaccagttaatgcagatggcaccattctccagcaagttacagtccctgtttcaggcatgatcactatcccagcagccagtttggcaggagcacagattgttcaaacaggagccaataccaacacaaccagcagtgggcaagggactgtcactgtgacactaccagtggcaggcaatgtggtcaattcaggagggatggtcatgatggttcctggggctggctctgtgcctgctatccaaagaatccctctacctggagcagagatgcttgaagaagagcctctctacgtgaatgccaaacaataccaccgtattcttaagaggaggcaagcccgagctaaactagaggcagaagggaaaattccaaaggagagaaggaaatacctgcatgagtctcggcaccgtcatgccatggcacggaagcgtggtgaaggtggacgatttttctctccaaaggaaaaggatagtccccatatgcaggatccaaaccaagccgatgaagaagcaatgacacagatcatccgagtgtcctaaccccacgccatgtgatggagctgatcaaggtcatgtttctcactgttccaggaaattgatcaactcttccaatgggacattgatgatcacattctgccctttactacaggacagaaaccacttagtttttaataagtggctcagtattacaattgcagcgatgcctggcaaattgaagttgcaagcctttgtcttactctttcagtcaggacctatttcagactgttgatgacattgacatttcatggattcggatgatgcaaggagacacatgccaccccagcagtacacaaagcacttaccttgactggtgaagtcagccagccatctcagcagggaagaacaaccttctcaaccaaaactgcaatcaggaaagcagcagcccactccttcccatggaatctagatagcatttatccctctggtgggggaatctcacactgactaactgaatggacgctttgtattcagagatggcttccaggcaaggaagttctatcttgtgatacggaaacaataaattcctgagatacattcttccaatctgtggtaataataagaattcctctctgcccagatggtaggttgatatttctggactctagccaagaaaaagctaattctggggatagaatgataaaagactcaagcactaaaaatatacattgccgaatctctttttctagctttgaaaagttgtggcctgcttgtccctctgttgactggtcatctggaccatcgtacttgctgtggctacttctaagacaatgtagagggttattaaaccttgaaactgcctttcctaagtagagaacaagactattcaacaacttctttgctgaagcactgaggagatttgtaatactcctaaaggaagggccaaactagagattttcaatcatagactttgtgacagcatttggggaactaaaagattcatgtgtttcagcctagtgggagagagtgggggagaggaagagagagagagagcatgtatacccgtatgttatcatagagcacgattctccagtggatggatacctggaatggatcattaagatgaagagagtaattcacatttactctagaacctttaacaagcactgaaaggaagaagcctgagatttgatccttgacaatttctggaaagcactggtcagtcacactgtcctggacagagtccaagttacccactaaagcttgaggtggatgttggcttcactcctgcagcacaatgctggccctagaaagcgagcattttcattcttcccatggcagctgattctttggtgccagttttcatgttttctccataggccttgtgcagaaaggttttctcattgcatccatactgtgagaataaactgcagactgaaagaaacatgcctgcagggtcaggaactgggccactgttcccagaacgggttgtgcctcagaaatctatttctcttttagatgttgaaccctgagagaactaagatgatatttcagaagagttggctgccttctctggaaacaaggaaaacccaataattccgaggaacattgggggttaaataccaagaataaaaattggttcttctttgaaaaaaaaaaagaaaaaaaaaaaaaaaa

SEQ ID NO: 104 - protein, 347 aa, homo sapiens
MEQYTANSNSSTEQIVVQAGQIQQQQQGGVTAVQLQTEAQVASASGQQVQTLQVVQGQPLMVQVSGGQLITSTGQPIMVQAVPGGQGQTIMQVPVSGTQGLQQIQLVPPGQIQIQGGQAVQVQGQQGQTQQIIIQQPQTAVTAGQTQTQQQIAVQGQQVAQTAEGQTIVYQPVNADGTILQQVTVPVSGMITIPAASLAGAQIVQTGANTNTTSSGQGTVTVTLPVAGNVVNSGGMVMMVPGAGSVPAIQRIPLPGAEMLEEEPLYVNAKQYHRILKRRQARAKLEAEGKIPKERRKYLHESRHRHAMARKRGEGGRFFSPKEKDSPHMQDPNQADEEAMTQIIRVS

SEQ ID NO: 105 - DNA, 3720 bp, homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 106 - protein, 781 aa, homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 107 - DNA, 4298 bp, homo sapiens
cagacaggatattcactgctgtggcaaggcctgtagagagtttcgaagttaggaggactcaagacggtccctccctggacttttctgaaggggctcaaaagatgacacgcgccagagctggaaggcgtcgccaattggtccaacttttccctcctccctttttgcggatgagaaaaactgaggcccaggtttgggatttccagagcccgggatttcccggcaacgccgacaaccacattcccccggctattctgacccgccccggttccgggacgctccctgggagccgccgccgagggcctgctgggactcccggggaccccgccgtcggggcagcccccacgcccggcgccgcccgccggaacggcgccgctgttgcgcacttgcaggggagccggcgactgagggcgaggcagggagggagcaagcggggctgggagggctgctggcgcgggctcgccggctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggttgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctgggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcat

SEQ ID NO: 108 - protein, 794 aa, homo sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

(19) United States
(12) Patent Application Publication
(10) Pub. No.: US 2009/0215875 A1
(43) Pub. Date: Aug. 27, 2009

(54) METHODS AND KITS FOR EXPANDING HEMATOPOIETIC STEM CELLS

(76) Inventors: Eric DENEAULT, St-Chrysostome (CA); Sonia Cellot, Montreal (CA); Guy Sauvageau, Montreal (CA)

Correspondence Address: GOUDREAU GAGE DUBUC, 2000 MCGILL COLLEGE, SUITE 2200, MONTREAL, QC, H3A 3H3, CA

(21) Appl. No.: 12/192,701

(22) Filed: Aug. 15, 2008

Related U.S. Application Data
(60) Provisional application No. 61/031,106, filed on Feb. 25, 2008.

(51) Int. Cl. A61K 31/7088; C12N 15/86; C12N 5/08; A61P 7/00

(52) U.S. Cl. 514/44 R; 435/456; 435/372

(57) ABSTRACT

A method of increasing the expansion and/or differentiation of a hematopoietic stem cell (HSC) comprising: (a) increasing the level and/or activity of a polypeptide encoded by at least one gene selected from trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant of said polypeptide, in said cell; (b) increasing the level of a nucleic acid encoding the polypeptide or functional variant of (a) in said cell; or (c) any combination of (a) and (b).

### CROSS REFERENCE TO RELATED APPLICATIONS

This application claims priority, under 35 U.S.C. §119(e), of U.S. provisional application Ser. No. 61/031,106 filed on Feb. 25, 2008. All documents above are incorporated herein in their entirety by reference.

### FIELD OF THE INVENTION

The present invention relates to hematopoietic stem cells (HSCs). More specifically, the present invention is concerned with methods and reagents for expanding HSCs.

### BACKGROUND OF THE INVENTION

The mature cell contingent of adult hematopoietic tissue is continuously replenished in the lifespan of an animal, due to periodical supplies from hematopoietic stem cells (HSC) that reside permanently in the niche. To maintain blood homeostasis, these primitive cells rely on two critical properties, namely multipotency and self-renewal (SR). The former enables differentiation into multiple lineages, the latter ensures preservation of fate upon cellular division. By definition, a self-renewal division implies that a HSC is permissive to cell cycle entry, while restrained from engaging in differentiation, apoptosis or senescence pathways. The transcriptional regulatory network of HSC self-renewal still remains largely undefined, an observation that contrasts with that of embryonic stem cells (ESC) for which self-renewal and pluripotency are increasingly dissected molecularly (1, 2). Only few nuclear factors have been documented as inducers of HSC expansion when overexpressed, i.e., Hoxb4 (3) and NF-Ya (4), or activated, i.e., β-catenin (5) and STAT5a (6). Of these factors, Hoxb4 and its derivatives (Hoxa9, NA10HD) are among the most potent and best documented (7, 8).

Hematopoietic stem cells (HSCs) are rare cells that have been identified in fetal bone marrow, umbilical cord blood, adult bone marrow, and peripheral blood, which are capable of differentiating into each of myeloerythroid (red blood cells, granulocytes, monocytes), megakaryocyte (platelets) and lymphoid (T-cells, B-cells, and natural killer cells lineages) cells. In addition these cells are long-lived, and are capable of producing additional stem cells (self-renewal). Stem cells initially undergo commitment to lineage restricted progenitor cells, which can be assayed by their ability to form colonies in semisolid media. Progenitor cells are restricted in their ability to undergo multi-lineage differentiation and have lost their ability to self-renew. Progenitor cells eventually differentiate and mature into each of the functional elements of the blood.

HSCs are used in clinical transplantation protocols to treat a variety of diseases including malignant and non-malignant disorders.

HSCs obtained directly from the patient (autologous HSCs) are used for rescuing the patient from the effects of high doses of chemotherapy or used as a target for gene-therapy vectors. HSCs obtained from another person (allogeneic HSCs) are used to treat haematological malignancies by replacing the malignant haematopoietic system with normal cells. Allogeneic HSCs can be obtained from siblings (matched sibling transplants), parents or unrelated donors (mismatched unrelated donor transplants). About 45,000 patients each year are treated by HSC transplantation. Although most of these cases have involved patients with haematological malignancies, such as lymphoma, myeloma and leukemia, there is growing interest in using HSC transplantation to treat solid tumours and non-malignant diseases. For example, erythrocyte disorders such as β-thalassaemia and sickle-cell anemia have been successfully treated by transplantation of allogeneic HSCs.

Therefore, there is a need for novel methods and reagents for expanding HSCs.

The present description refers to a number of documents, the content of which is herein incorporated by reference in their entirety.

### SUMMARY OF THE INVENTION

Accordingly, the present invention concerns a novel in vitro→in vivo functional screen which identified a series of HSC regulators (nuclear factors and asymmetrical cell division factors) which induce high levels of HSC activity similar to that previously achieved with Hoxb4. In total, 22 new determinants have emerged. Eleven of the 18 nuclear factors-HSC regulators act in a cell autonomous manner, while the remaining 7 provide a non-autonomous influence on HSC activity. Clonal and phenotypic analyses of hematopoietic tissues derived from selected recipients confirmed that the majority of the identified factors induced HSC expansion in vitro without perturbing their differentiation in vivo. Epistatic analyses further revealed that 3 of the most potent candidates, namely Ski, Prdm16 and Klf10 may exploit both mechanisms. The present invention thus presents a novel methodology to screen for determinants of HSC regulators as well and methods of expanding and/or differentiating HSCs.

More specifically, in accordance with an aspect of the present invention, there is provided a method of increasing the expansion and/or differentiation of a hematopoietic stem cell (HSC) comprising: (a) increasing the level and/or activity of at least one HSC regulator polypeptide encoded by at least one HSC regulator gene selected from trim27, xbp1, sox4, smarcc1, sfpi1, fos, hmgb1, hnrpdl, vps72, tcfec, klf10, zfp472, ap2a2, gpsm2, gpx3, erdr1, tmod1, pml, cnbp, prdm16, hdac1 and ski, or a functional variant of said polypeptide, in said cell; (b) increasing the level of a nucleic acid encoding the HSC regulator polypeptide or functional variant of (a) in cell; or (c) any combination of (a) and (b).

In a specific embodiment of the method, said at least one polypeptide comprises the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 or 97.

In a specific embodiment, the method comprises increasing the level of said nucleic acid in said cell. In another specific embodiment, said nucleic acid encodes a HSC regulator polypeptide comprising the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 or 97.

In another specific embodiment, said nucleic acid comprises the coding region of the nucleotide sequence set forth in NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

In another specific embodiment, said differentiation is multilineage differentiation and said at least one HSC regulator gene is selected from trim27 (SEQ ID NO: 1), xbp1 (SEQ ID NOs: 3 and 5), sox4 (SEQ ID NO: 7), hnrpdl (SEQ ID NO: 19), vps72 (SEQ ID NO: 21) and gpx3 (SEQ ID NOs: 75 and 98).

In another specific embodiment, the method further comprises (a) increasing the level and/or activity of at least one further HSC regulator polypeptide; (b) increasing the level of a nucleic acid encoding the at least one further HSC regulator polypeptide or functional variant of (a) in said cell; or (c) any combination of (a) and (b). In a specific embodiment the further HSC regulator polypeptide is selected from Hoxb4, Hoxa9, Bmi1, NF-YA, β-catenin and STAT5A. In a specific embodiment the HSC regulator polypeptide comprises a sequence as set forth in SEQ ID NO: 92 (Hoxb4), SEQ ID NO: 99 (Hoxa9), SEQ ID NO: 101 (Bmi1), SEQ ID NO: 103 (NF-YA), SEQ ID NO: 105 (β-catenin) or SEQ ID NO: 107 (STAT5A). In another specific embodiment, said further HSC regulator polypeptide is Hoxb4 and comprises the amino acid sequence set forth in Genbank accession No: NP_{—}076920 (SEQ ID NO: 92).

In another specific embodiment, said expansion is multiclonal expansion and said at least one HSC regulator gene is selected from trim27, xbp1, sox4, smarcc1, hnrpdl, vps72, klf10, ap2a2, gpsm2 and gpx3.

In another specific embodiment, the method comprises transfecting or transforming said cell with a vector comprising said nucleic acid. In another specific embodiment, said vector is a viral vector. In another specific embodiment, said viral vector is an adenoviral vector.

In accordance with another aspect of the present invention, there is provided a use of an agent capable of: (a) increasing the level and/or activity of at least one HSC regulator polypeptide encoded by at least one HSC regulator gene selected from trim27, xbp1, sox4, smarcc1, sfpi1, fos, hmgb1, hnrpdl vps72, tcfec, klf10, zfp472, ap2a2, gpsm2, gpx3, erdr1, tmod1, cnbp1, prdm16, hdac1, pml and ski, or a functional variant of said polypeptide; (b) increasing the level of a nucleic acid encoding the at least one HSC regulator polypeptide or functional variant of (a); or (c) any combination of (a) and (b), for increasing the expansion and/or differentiation of a hematopoietic stem cell (HSC).

In accordance with another aspect of the present invention, there is provided a use of an agent capable of: increasing the level and/or activity of at least one HSC regulator polypeptide encoded by at least one HSC regulator gene selected from trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1 and ski, or a functional variant of said polypeptide, in a cell; increasing the level of a nucleic acid encoding the at least one polypeptide or functional variant of (a) in a cell; or any combination of (a) and (b), for the preparation of a medicament for increasing the expansion and/or differentiation of a hematopoietic stem cell (HSC).

In a specific embodiment of the use, said polypeptide comprises the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96, 97 or 98.

In another specific embodiment, said agent is capable of increasing the level of said nucleic acid in said cell. In another specific embodiment, said agent is a nucleic acid encoding at least one of trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant thereof. In another specific embodiment, said nucleic acid encodes a polypeptide comprising the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96, 97 or 98.

In another specific embodiment, said nucleic acid comprises the coding region of nucleotide sequence set forth in Genbank accession Nos: NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

In another specific embodiment, said differentiation is multilineage differentiation and said at least one HSC regulator gene is selected from trim27, xbp1, sox4, hnrpdl, vps72 and gpx3.

In another specific embodiment, said expansion is multiclonal expansion and said at least one HSC regulator gene is selected from trim27, xbp1, sox4, smarcc1, hnrpdl, vps72, klf10, ap2a2, gpsm2 and gpx3.

In another specific embodiment, said nucleic acid is comprised within a vector. In another specific embodiment, said vector is a viral vector. In another specific embodiment, said viral vector is an adenoviral vector.

In another specific embodiment, the use further comprises (a) increasing the level and/or activity of a further HSC regulator polypeptide encoded a further HSC regulator gene; (b) increasing the level of a nucleic acid encoding the further HSC regulator polypeptide or functional variant of (a) in said cell; or (c) any combination of (a) and (b). In a particular embodiment the further HSC regulator is selected from Hoxb4, Hoxa9, Bmi1, NF-YA, β-catenin and STAT5A. In a specific embodiment, the HSC regulator nucleic acid comprises a sequence encoding the sequence as set forth in SEQ ID NO: 92 (Hoxb4), SEQ ID NO: 100 (Hoxa9), SEQ ID NO: 102 (Bmi1), SEQ ID NO: 104 (NF-YA), SEQ ID NO: 106 (β-catenin) or SEQ ID NO: 108 (STAT5A). In another specific embodiment, said further HSC regulator polypeptide is Hoxb4 and comprises the amino acid sequence set forth in Genbank accession No: NP_{—}076920 (SEQ ID NO: 92).

In accordance with another aspect of the present invention, there is provided a composition for increasing the expansion and/or differentiation of a hematopoietic stem cell (HSC) comprising: (a) an agent capable of: (i) increasing the level and/or activity of at least one polypeptide encoded by at least one gene selected from trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant of said polypeptide, in a cell; (ii) increasing the level of a nucleic acid encoding the at least one polypeptide or functional variant of (a) in a cell; or (iii) any combination of (i) and (ii); and (b) a pharmaceutically acceptable carrier or excipient.

In a specific embodiment, this use comprises (a) an agent capable of increasing the level of at least one nucleic acid encoding at least one of trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski; and (b) a pharmaceutically acceptable carrier or excipient.

In another specific embodiment, said agent is nucleic acid encoding at least one of trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant thereof.

In another specific embodiment, said nucleic acid encodes a HSC regulator polypeptide comprising the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96, 97 or 98.

In another specific embodiment, said nucleic acid comprises the coding region of the nucleotide sequence set forth in Genbank accession Nos: NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

In another specific embodiment, said differentiation is multilineage differentiation and said at least one gene is selected from trim27, xbp1, sox4, hnrpdl, vps72 and gpx3.

In another specific embodiment, said expansion is multiclonal expansion and said at least one gene is selected from trim27, xbp1, sox4, smarcc1, hnrpdl, vps72, klf10, ap2a2, gpsm2 and gpx3.

In another specific embodiment, said agent is a vector comprising said nucleic acid. In another specific embodiment, said vector is a viral vector. In another specific embodiment, said viral vector is an adenoviral vector.

In another specific embodiment, the composition comprises a further agent capable of: (a) increasing the level and/or activity of at least one further HSC regulator polypeptide; (b) increasing the level of a nucleic acid encoding the HSC regulator polypeptide or functional variant of (a) in a cell; or (c) any combination of (a) and (b). In another specific embodiment said at least one further HSC regulator polypeptide is selected from Hoxb4, Hoxa9, Bmi1, NF-YA, β-catenin and STAT5A. In another specific embodiment, said further agent is a Hoxb4 nucleic acid encoding the amino acid sequence set forth in Genbank accession No: NP_{—}076920 (SEQ ID NO: 92).

In accordance with another aspect of the present invention, there is provided an hematopoietic stem cell transformed or transduced with a vector comprising a nucleic acid encoding at least one HSC regulator selected from trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, and a functional variant thereof.

In a specific embodiment of the cell, said nucleic acid encodes a HSC regulator polypeptide comprising the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 97 or 98.

In another specific embodiment, said nucleic acid comprises the coding region of the nucleotide sequence set forth in Genbank accession Nos: NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

In another specific embodiment, said vector is a viral vector. In another specific embodiment, said viral vector is an adenoviral vector. In another specific embodiment, the vector further comprises a nucleic acid encoding a further HSC regulator selected from Hoxb4, Hoxa9, Bmi1, NF-YA, β-catenin and STAT5A. In another specific embodiment, the further HSC regulator is Hoxb4. In another specific embodiment, said nucleic acid encodes a Hoxb4 polypeptide comprising the amino acid sequence set forth in Genbank accession No: NP_{—}076920 (SEQ ID NO: 92).

In accordance with another aspect of the present invention, there is provided a method for increasing the number of blood cells in a subject comprising administering to said subject the hematopoietic stem cell of the present invention.

In accordance with another aspect of the present invention, there is provided a method for reconstituting the hematopoietic system or tissue of a subject comprising administering to said subject the hematopoietic stem cell of the present invention.

In accordance with another aspect of the present invention, there is provided a use of the hematopoietic stem cell of the present invention for hematopoietic stem cell transplantation.

In accordance with another aspect of the present invention, there is provided a use of the hematopoietic stem cell of the present invention for reconstituting the hematopoietic system or tissue of a subject.

In accordance with another aspect of the present invention, there is provided a use of the hematopoietic stem cell of the present invention for the preparation of a medicament for reconstituting the hematopoietic system or tissue of a subject.

In accordance with another aspect of the present invention, there is provided a use of the hematopoietic stem cell of the present invention for increasing the number of blood cells in a subject.

In accordance with another aspect of the present invention, there is provided a use of the hematopoietic stem cell of the present invention for the preparation of a medicament for increasing the number of blood cells in a subject.

In accordance with another aspect of the present invention, there is provided a method for increasing the number of blood cells in a subject comprising administering to said subject the composition of the present invention.

In accordance with another aspect of the present invention, there is provided a method for reconstituting the hematopoietic system or tissue of a subject comprising administering to said subject the composition of the present invention.

In accordance with another aspect of the present invention, there is provided a use of the composition of the present invention for hematopoietic stem cell transplantation.

In accordance with another aspect of the present invention, there is provided a use of the composition of the present invention for reconstituting the hematopoietic system or tissue of a subject.

In accordance with another aspect of the present invention, there is provided a use of the composition of the present invention for the preparation of a medicament for reconstituting the hematopoietic system or tissue of a subject.

In accordance with another aspect of the present invention, there is provided a use of the composition of the present invention for increasing the number of blood cells in a subject.

In accordance with another aspect of the present invention, there is provided a use of the composition of the present invention for the preparation of a medicament for increasing the number of blood cells in a subject.

In accordance with another aspect of the present invention, there is provided a method of increasing the expansion and/or differentiation of a hematopoietic stem cell (HSC) comprising: (a) increasing the level and/or activity of at least one HSC regulator polypeptide encoded by at least one HSC regulator gene selected from erdr1, tmod1, cnbp1, prdm16, hdac1 and ski, or a functional variant of said polypeptide, in said cell; (b) increasing the level of at least one nucleic acid encoding the at least one polypeptide or functional variant of (a) in said cell; or (c) any combination of (a) and (b).

In accordance with another aspect of the present invention, there is provided an hematopoietic stem cell transformed or transduced with a vector comprising a nucleic acid encoding at least one of erdr1, tmod1, cnbp1, prdm16, hdac1 and ski, or a functional variant thereof.

As used herein, “expansion” and “self-renewal” are used interchangeably and refer to the propagation of a cell or cells without terminal differentiation and “differentiation” refers to the developmental process of lineage commitment. A “lineage” refers to a pathway of cellular development, in which precursor or “progenitor” cells undergo progressive physiological changes to become a specified cell type having a characteristic function (e.g., a T cell, a macrophage). Differentiation occurs in stages, whereby cells gradually become more specified until they reach full maturity.

Accordingly, the methods of the invention can be used to treat a disease or disorder in which it is desirable to increase the number of HSCs or their progenitors. Frequently, subjects in need of the inventive treatment methods will be those undergoing or expecting to undergo a blood cell (e.g., an immune cell) depleting treatment, such as chemotherapy.

Thus, methods of the invention can be used, for example, to treat patients requiring a bone marrow transplant or a hematopoietic stem cell transplant (e.g., to reconstitute the hematopoietic system/tissue), such as cancer patients undergoing chemo and/or radiation therapy. Disorders treated by methods of the invention can be the result of an undesired side effect or complication of another primary treatment, such as radiation therapy, chemotherapy, or treatment with a bone marrow suppressive drug. Methods of the invention can further be used as a means to increase the number of mature cells derived from HSCs (e.g., erythrocytes, lymphocytes). For example, disorders or diseases characterized by a lack of, or low levels of, blood cells, or a defect in blood cells, can be treated by increasing the pool of HSCs. Such conditions include, for example, thrombocytopenia, anemias and lymphopenia. The disorder to be treated may also be the result of an infection causing damage to blood/lymphoid cells and/or stem cells.

Hematopoietic stem cell progenitors include virtually any cell capable of giving rise to a hematopoietic stem cell (e.g., mesenchymal stem cells, embryonic stem cells). The hematopoietic stem cell, which may be isolated from bone marrow, blood, umbilical cord blood, peripheral blood, fetal liver and yolk sac for example, is the progenitor cell that generates blood cells or following transplantation reinitiates multiple hematopoietic lineages and can reinitiate hematopoiesis for the life of a recipient. When transplanted into lethally irradiated subjects (e.g., animals, humans), hematopoietic stem cells can repopulate the erythroid, neutrophil-macrophage, megakaryocyte and/or lymphoid hematopoietic cell pool.

It is well known in the art that hematopoietic cells include pluripotent stem cells, multipotent progenitor cells (e.g., a lymphoid stem cell), and/or progenitor cells committed to specific hematopoietic lineages. The progenitor cells committed to specific hematopoietic lineages maybe of T cell lineage, B cell lineage, dendritic cell lineage, Langerhans cell lineage and/or lymphoid tissue-specific macrophage cell lineage. Where the stem cells to be provided to a subject in need of such treatment are hematopoietic stem cells, they are most commonly obtained from the bone marrow of the subject (autologous) or a compatible donor (heterologous). Bone marrow cells can be easily isolated using methods known in the art.

Hematopoietic stem cells can also be obtained from biological samples (e.g., blood products). A “blood product” as used in the present invention defines a product obtained from the body or an organ of the body containing cells of hematopoietic origin. Such sources include unfractionated bone marrow, umbilical cord, peripheral blood, liver such as fetal liver, thymus, lymph, spleen and yolk sac. It will be apparent to those of ordinary skill in the art that all of the aforementioned crude or unfractionated blood products can be enriched for cells having “hematopoietic stem cell” characteristics in a number of ways. For example, the blood product can be depleted from the more differentiated progeny. The more mature, differentiated cells can be selected against, via cell surface molecules they express (e.g., by FACS). Unfractionated blood products can be obtained directly from a donor or retrieved from cryopreservative storage.

Once obtained from a desired source, contacting of HSCs with a polypeptide and/or nucleic acid molecule and/or agent may, if desired, occur in culture (e.g., ex vivo or in vitro). Employing the polypeptides or nucleic acid molecules of the present invention, it is possible to stimulate the expansion and/or differentiation of hematopoietic stem cells. The media used is that which is conventional for culturing cells. Appropriate culture media can be a chemically defined serum-free media, such as the chemically defined media RPMI, DMEM, Iscove's, etc or so-called “complete media”. Typically, serum-free media are supplemented with human or animal plasma or serum. Such plasma or serum can contain small amounts of hematopoietic growth factors. If desired, a hematopoietic or other stem cell may be treated with additional agents that promote stem cell maintenance and expansion. It is well within the level of ordinary skill in the art for practitioners to vary the parameters accordingly. The growth agents of particular interest in connection with the present invention are hematopoietic growth factors. By hematopoietic growth factors, it is meant factors that influence the survival or proliferation of hematopoietic stem cells. Growth agents that affect only survival and proliferation, but are not believed to promote differentiation, include the interleukins 3, 6 and 11, stem cell factor and FLT-3 ligand. The foregoing factors are well known to those of ordinary skill in the art and most are commercially available. They can be obtained by purification, by recombinant methodologies or can be derived or synthesized synthetically.

By the term “HSC regulator polypeptide” is meant to include any polypeptide of the present invention which increases directly or indirectly (e.g., cell-autonomous vs non-cell autonomous) HSC expansion and/or differentiation. These include trim27, xbp1, sox4, smarcc1, sfpi1, fos, hmgb1, hnrpdl, vps72, tcfec, klf10, zfp472, ap2a2, gpsm2, gpx3, erdr1, tmod1, pml, cnbp, prdm16, hdac1 and ski, or a functional variant of thereof. In a specific embodiment, the HSC regulator polypeptide of the present invention comprise a sequence comprise a sequence as set forth in SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 84, 86, 88, 90, and 98) Similarly, the term “HSC regulator gene” or “HSC regulator nucleic acid” includes any gene or nucleic acid which when expressed in cells increases directly or indirectly (e.g., cell-autonomous vs non-cell autonomous) HSC expansion and/or differentiation. These include nucleic acids encoding trim27, xbp1, sox4, smarcc1, sfpi1, fos, hmgb1, hnrpdl, vps72, tcfec, klf10, zfp472, ap2a2, gpsm2, gpx3, erdr1, tmod1, pml, cnbp, prdm16, hdac1 and ski, or a functional variant of thereof. In a specific embodiment, HSC regulator nucleic acids of the present invention comprise a sequence as se forth in SEQ ID NOs: 1, 3, 5, 7, 9, 11, 13, 15, 17, 19, 21, 23, 25, 27, 29, 31, 33, 35, 57, 39, 41, 43, 45, 47, 49, 51, 53, 55, 57, 59, 61, 63, 65, 67, 69, 71, 73, 75, 77, 79, 81, 83, 85, 87 or 89).

Thus, the present invention includes HSC regulator polypeptides having altered amino acid sequences (e.g., functional variants) as compared to those of the “natural” or “wild-type” polypeptides due to the artificial or natural substitution, deletion, addition, and/or insertion of amino acids as long as they have the activity of the natural polypeptides (i.e., can promote the expansion and/or differentiation of a HSC). Preferably, an amino acid can be substituted with the one having similar property to that of the amino acid to be substituted. It has been shown that recombinant TAT-HOXB4 protein, when added to the HSC culture, could penetrate the cell membrane and provides significant HSC expansion stimuli ((24); US 2004/0082003) and similar effect of stroma cell derived HOXB4 on human HSC has also been reported (10). Human HSCs, assessed with NOD/SCID SRC assay, can be efficiently and significantly expanded ex vivo using TAT-HOXB4 protein (11). The present invention thus encompasses recombinant polypeptides comprising a protein encoded by the genes of Table II below or functional variants thereof and a motif enhancing penetration of the protein into the HSC cell membranes, and their use for administration to HSC culture.

The present invention also includes polypeptides variants comprising an amino acid sequence having at least 50% identity, preferably at least 60%, preferably at least 75% identity, more preferably at least 90%; at least 95% and at least 98% identity to the polypeptides of the present invention (e.g., polypeptides comprising the sequence set forth in NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 97 or 98.

The term functional variants also includes fragment of the polypeptides of the invention. Such fragments may be truncated at the N-terminus or C-terminus, or may lack internal residues, for example, when compared with a full length native polypeptide. Certain fragments lack amino acid residues that are not essential for a desired biological activity of the polypeptides. For examples, when several functional variants of a polypeptide exists, one skilled in the art can readily identify residues which are not essential for a given biological activity by aligning the variants and identifying the residues which are different (see for example FIG. 28). Alternatively, residues that can be modified without affecting the biological activity of a gene can be identified by comparing the polypeptide sequences of several species (e.g., mouse, rats, human, pigs, primates, cats dogs, cows etc) and determining the residues which are different. Residues which are not conserved between the species are those that are likely not to affect the biological activity of the gene if modified. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like as well known by the skilled artisan.

Preferred variants of the present invention are those which retain their biological activity (e.g., promoting expansion/self-renewal and/or differentiation into blood cells) and whose nucleic acid sequence can specifically hybridize under high stringency conditions to HSC regulator nucleic acid sequences of the present invention (e.g., SEQ ID NOs: 1, 3, 5, 7, 9, 11, 13, 15, 17, 19, 21, 23, 25, 27, 29, 31, 33, 35, 57, 39, 41, 43, 45, 47, 49, 51, 53, 55, 57, 59, 61, 63, 65, 67, 69, 71, 73, 75, 77, 79, 81, 83, 85, 87 and, 89). Hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. (see Ausubel, et al. (eds), 1989). Hybridization conditions may be modified in accordance with known methods depending on the sequence of interest (28). Generally, stringent conditions are selected to be about 5° C. lower than the thermal melting point for the specific sequence at a defined ionic strength and pH.

The present invention also relates to a nucleic acid molecule encoding the above-mentioned polypeptides or functional variants thereof. The type of the nucleic acid molecule encoding the polypeptides of this invention is not limited as long as they are capable of encoding the polypeptides, and includes cDNA, genomic DNA, RNA (e.g., mRNA), synthetic or recombinantly produced nucleic acid, and nucleic acids comprising nucleotide sequences resulted from the degeneracy of genetic codes, all of which can be prepared by methods that are well-known in the art. The nucleic acid molecules of the present invention also encompass those having nucleotide sequences altered from those of the natural nucleic acids due to the insertions, deletions, or substitutions of nucleotide, as long as the polypeptides encoded by these altered nucleic acids encode polypeptides having the activity of the natural polypeptides (e.g., promoting expansion or differentiation of HSCS).

In an embodiment, the above-mentioned nucleic acid encodes a polypeptide comprising the sequence set forth in NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 97 or 98.

In a further embodiment, the above-mentioned nucleic acid comprises the coding region of nucleotide sequence set forth in Genbank accession Nos: NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

The nucleic acid molecules encoding the above-mentioned polypeptides may also be applied to the gene therapy of disorders caused by lack of expression of the polypeptides (e.g., a disease or condition associated with altered expansion and/or differentiation of HSCs), or in gene therapy applications where expansion and/or differentiation of HSCs is desirable (e.g., bone marrow/stem cell transplantion). Examples of vectors used for the gene therapy are viral vectors such as retroviral vector, adenoviral vector, adeno-associated viral vector, vaccinia viral vector, lentiviral vector, herpes viral vector, alphaviral vector, EB viral vector, papillomaviral vector, and foamyviral vector, and non-viral vector such as cationic liposome, ligand DNA complex, and gene gun. Gene transduction may be carried out in vivo and ex vivo, and also co-transduction with one or more gene of interest may be carried out. In an embodiment, the above-mentioned gene transduction is performed ex vivo and the transduced cells (i.e., expressing one or more of the polypeptide(s)) are administered to a subject.

Hematopoietic stem cells, progenitor cells, or a mixture comprising such cell types may be administered to a subject according to methods known in the art. Such compositions may be administered by any conventional route, including injection or by gradual infusion over time. The administration may, depending on the composition being administered, for example, be, pulmonary, intravenous, intraperitoneal, intramuscular, intracavity, subcutaneous, or transdermal. The stem cells are administered in “effective amounts”, or the amounts that either alone or together with further doses produce the desired therapeutic response. Administered cells of the invention can be autologous (“self”) or heterologous/non-autologous (“non-self,” e.g., allogeneic, syngeneic or xenogeneic). Generally, administration of the cells can occur within a short period of time following the induction of an increase in polypeptide activity/expression (or of increase in expression of a nucleic acid encoding the polypeptide (e.g., 1, 2, 5, 10, 24, 48 hours, 1 week or 2 weeks after the induction/increase)) and according to the requirements of each desired treatment regimen. For example, where radiation or chemotherapy is conducted prior to administration, treatment, and transplantation of stem cells of the invention should optimally be provided within about one month of the cessation of therapy. However, transplantation at later points after treatment has ceased can be done with derivable clinical outcomes.

Following harvest and treatment with a suitable agent, polypeptide or nucleic acid, hematopoietic stem cells or their progenitors, or a mixture of cells that include these cells may be combined with pharmaceutical carriers/excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene, glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells (e.g., the HSCs) into a subject in need thereof is to incorporate the cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein. Those skilled in the art will recognize that the components of the compositions should be selected to be chemically inert and will not affect the viability or efficacy of the stem cells or their progenitors as described in the present invention.

The quantity of cells to be administered will vary for the subject being treated. The precise determination of what would be considered an effective dose may be based on factors individual to each patient, including their size, age, sex, weight, and condition of the particular patient. As few as 100-1000 cells can be administered for certain desired applications among selected patients. Therefore, dosages can be readily ascertained by those skilled in the art from this disclosure and the knowledge in the art. The skilled artisan can readily determine the amount of cells and optional additives, vehicles, and/or carrier in compositions and to be administered in methods of the invention.

The pharmaceutical composition of the present invention (e.g., comprising an agent capable of increasing the expression and/or activity of at least one polypeptide encoded by at least one gene selected from trim27, xbp1, pml, sox4, smarcc1, sfpi1, fos, hmgb1, hnrpdl, vps72, tcfec, klf10, zfp472, ap2a2, pml, gpsm2, gpx3, erdr1, tmod1, cnbp1, prdm16, hdac1 and ski) is administered in a manner compatible with the dosage formulation, and in a therapeutically effective amount, for example intravenously, intraperitoneally, intramuscularly, subcutaneously, and intradermally. It may also be administered by any of the other numerous techniques known to those of skill in the art, see for example the latest edition of Remington's Pharmaceutical Science, the entire teachings of which are incorporated herein by reference. For example, for injections, the pharmaceutical composition of the present invention may be formulated in adequate solutions including but not limited to physiologically compatible buffers such as Hank's solution, Ringer's solution, or a physiological saline buffer. The solutions may contain formulatory agents such as suspending, stabilizing, and/or dispersing agents. Alternatively, the pharmaceutical composition of the present invention may be in powder form for combination with a suitable vehicle, e.g., sterile pyrogen-free water, before use. Further, the composition of the present invention may be administered per se or may be applied as an appropriate formulation together with pharmaceutically acceptable carriers, diluents, or excipients that are well-known in the art. In addition, other pharmaceutical delivery systems such as liposomes and emulsions that are well-known in the art, and a sustained-release system, such as semi-permeable matrices of solid polymers containing the therapeutic agent, may be employed. Various sustained-release materials have been established and are well-known to one skilled in the art. Further, the composition of the present invention can be administered alone or together with another therapy conventionally used for the treatment of a disease/condition associated with poor expansion and/or differentiation of HSCs, or in which expansion and/or differentiation of HSCs is desirable.

The quantity to be administered and timing may vary within a range depending on the formulation, the route of administration, and the tissue or subject to be treated, e.g., the patient's age, body weight, overall health, and other factors. The dosage of protein or nucleic acid of the present invention preferably will be in the range of about 0.01 ug/kg to about 10 g/kg of patient weight, preferably 0.01 mg/kg to 100 mg/kg. When using the pharmaceutical composition of the invention as a gene therapeutic agent, the pharmaceutical composition may be administered directly by injection or by administering a vector integrated with the nucleic acid. For the nucleic acid molecule, the amount administered depends on the properties of the expression vector, the tissue to be treated, and the like. For viral vectors, the dose of the recombinant virus containing such viral vectors will typically be in the range of between about 0.1 to about 100 pfu/kg per kg of body weight, in an embodiment between about 1 to about 50 pfu/kg per kg of body weight (e.g., about 10 pfu/kg per kg of body weight).

The agent useful for the method of the present invention includes, but is not limited to, that which directly or indirectly modifies the activity of the protein and that which modulates the production (i.e., expression) and/or stability of the protein (e.g., at the level of transcription, translation, maturation, post-translational modification, phosphorylation and degradation). In general, compounds/agents capable of modulating (e.g., increasing) the expression or activity of one or more polypeptide and/or nucleic acid of the present invention may be identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the experimental design of the nuclear factors screening strategy of the present invention. (A) A list of candidate genes was generated combining data from available stem cell databases, literature searches, and expression profiling results of a HoxA9-Meisl induced fetal liver leukemia (FLA2) highly enriched in Leukemia Repopulating Cells (LRC, frequency of −1:1.5) available from the inventors laboratory. The putative nuclear factors were subsequently ranked based on an algorithm that would stratify them according to self-renewal properties. Highest scoring candidates (n=139) were further selected for functional assessment using a retroviral over-expression approach. 103 of the 139 genes selected were tested. (B) The coding sequence of each candidate was PCR-amplified, FLAG-tagged and subcloned into 1 out of 3 modified MSCV vectors containing a different reading frame (pKOF-1, -2 and -3). Respective retroviral producers were plated in a single well of a 96-well dish and co-cultured for 5 days with freshly sorted (CD150⁺CD48⁻Lin) bone marrow cells. Immediately upon infection (Day 0), a fraction of each well was transplanted into sublethally irradiated congenic recipient mice along with competitor cells (Ly5.2⁺ helpers). A similar assay was performed following an additional week of ex vivo culture (Day 7). (C) Expression of candidate proteins in retroviral producing cells (GP+E86) was confirmed by western immunobloting and revealed using an anti-FLAG antibody. Corresponding molecular weights are shown on FIG. 4. (D) Range of retroviral gene transfer efficiencies of sampled gene candidates based on GFP epifluorescence assessment of (Day 0) cultured BM cells;

FIG. 2 shows the scoring system used in candidate selection. Candidates were selected using microarray gene expression profiling from a pure stem cell leukemia (FLA2, submitted), the Stem Cell Database (SCDb) of Princeton University available online (http://stemcell.princeton.edu/) and expression profiles performed on enriched stem cells populations (9, 13-23). The 103 genes in grey have been tested in the screen;

FIG. 3 shows the subcloning strategy and protein expression of candidates. The accession number corresponding to each cDNA used as a template for PCR amplification are shown in addition to the sequences of forward and reverse primers used, with restriction sites used for subcloning underlined;

FIG. 4 shows white blood cell chimerism data obtained from competitive repopulation assays with 103 genes. The second column shows gene transfer efficiencies for each nuclear gene candidate during the primary screen based on the level of GFP⁺ HSC derivatives at day 4. The other values represent the reconstitution levels of Ly5.1+ cells in each independent experiment presented as the mean of 2 (day 0) or 3 (day 7) mice per experiment. Some mice were already eliminated from the screen at 8 or 12 weeks because they did not meet our selection criteria for positive outcome, mainly based on peripheral blood reconstitution by Ly5.1⁺ cells above 10% at 8 weeks and 30% at ≧12 weeks (see FIG. 3A for competitive repopulation assays). Exp=experiment; w=weeks; inf. lev.=infection level; blank in exp1=mice died or were sacrificed (low level of Ly5.1⁺ cells); blank in exp2-5=genes eliminated after the primary screen; not all data are shown for vector (which has n=8);

FIG. 5 shows competitive repopulation assays as a measurement of HSC activity. (A) Graft-derived hematopoiesis was evaluated at 4-week intervals in primary recipient mice of cultured BM cells by determining the percentage of Ly5.1 positive cells (donor-derived) in peripheral blood (PB) using FACS analysis. As a set of reference values, left panel indicates PB reconstitution levels from cultures initiated with a positive regulator of self-renewal (Hoxb4) after a 7-day expansion, in relation to values observed with an empty vector (mean of pKOF-1, -2 and -3) at initiation (day 0) and termination of cultures (day 7). Day 7 values for the screened 103 candidates are compiled and presented in the middle panel, with the established cut-off level for a gain-of-function readout. Values from one experiment, presented as mean±SD for left panel: n=2 mice for Hoxb4, n=6 mice for day 0 empty vector, n=9 mice for day 7 empty vector; and as mean for middle panel: n=3 mice for each candidate cDNA. (B) Values are reported as peripheral blood reconstitution of Ly5.1⁺ cells following a 7 day of ex vivo culture (solid line) compared to empty vector (dashed line=day 7). Number of independent experiment per candidate gene equals 4 except for pKOF (vector control) n=8; Hoxb4, Cnbp and Prdm16: n=3, Ski: n=1. Each experiments: mean 3 mice per gene. Values=mean±SEM except for Ski: mean±SD. WBC=white blood cells;

FIG. 6 shows nuclear candidates providing net increase in HSC activity in vitro. (A) When using vector control at day 0, a peripheral blood reconstitution at 14.4±2.2% in recipients transplanted 16 weeks earlier was observed, which provides a reliable estimation of the level of HSC activity present at the initiation of the 7-day culture. Based on this, genes that provide a significantly net increase in HSC activity (black solid lines) above that measured at the initiation of the culture (black dotted line) from those which do not (grey solid lines) were identified. (B) Table with p values for day 0 and day 7 data from vector in comparison with day 7 data from each validated hit. Framed values correspond to genes that provide a significantly net increase in HSC activity (black solid lines in (A)). WBC=white blood cells;

FIG. 7 shows that enhanced HSC activity is supported by intrinsic and extrinsic groups of effectors. (A) Southern blot analysis showing the presence of the expected proviral DNA in the BM of selected recipients that were highly reconstituted (between 10 to 85% of Ly5.1+ cells) at 20 weeks post-transplantation. For 11 of the 18 genes identified in the screen proviral DNA was observed in 58 of the 65 recipients that were analyzed at this late time point (46 are shown in the 2 upper panels, presented as the cell autonomous group). The analysis of proviral DNA integration patterns in selected hematopoietic tissues from these mice revealed that several different clones with long-term reconstitution ability contributed to hematopoiesis for each of these 11 genes. This was true for different recipients within the same experiments and, obviously from different experiments, thus supporting that insertional mutagenesis is not responsible for these results. In several instance, the same proviral integrations in the DNA from 2 different mice reconstituted by cells derived from the same culture could be identified, demonstrating that LT-HSC self-renewal has occurred in the cultures (a-i). Bottom panel shows the other 7 of the 18 validated genes, namely Fos, Hmgb1, Tcfec, Sfpi1, Zfp472, Hdac1 and Pml, and that only a minority of the highly reconstituted recipients (between 10 to 85% of Ly5.1⁺ cells) at 20 weeks post-transplantation contained integrated proviral DNA in their BM raising the possibility of non-cell autonomous activity in the cultures in which these HSCs were kept prior to transplantation (non-cell autonomous group). Each blot was systematically exposed for the same period of time (3 days). To ensure the absence of bands in bottom panel, the brightness and contrast of the images was enhanced. Below each blot is presented the level of peripheral blood Ly5.1⁺ or GFP⁺ cell reconstitution of recipient mice 20 weeks post-transplantation. (B) Compiled features of newly identified HSC self-renewal determinants. From left to right: individual gene candidates were evaluated for gene transfer efficiencies (mean±SD of % GFP⁺ HSC in culture at day 4) in experiments containing selected mice mentioned in A (3rd column), followed by peripheral blood cell reconstitution of the same mice (mean±SD of % Ly5.1⁺ cells, 4th column). Proportion of mice containing proviral DNA in their BM on the total of selected mice analysed is indicated in the 5th column, and the number of independent clones identified per gene is shown in the 6th column. In the 7th and 8th column, the peripheral blood cell reconstitution of every mice transplanted for each gene at day 0 and day 7 (mean±SEM of % Ly5.1+ cells) is shown. Finally, the last column indicates the conclusion about the cell autonomous or non-autonomous effect of each gene on enhanced HSC activity.X=GFP expression not reliable for these constructs/clones; PBR=peripheral blood reconstitution; n/a=not applicable; CA=cell autonomous; NCA=non-cell autonomous;

FIG. 8 shows the morphological analysis by Wright staining of derivatives of HSC populations overexpressing self-renewal candidates (upper-left inserts) at day 7 of ex vivo culture. Proportions of immature blasts vs terminally differentiated cells (neutrophils, monocytes and masts cells: black arrows in upper-left inserts) for respective cultures are depicted in right panel. Values are presented as mean±SD and a field comprising 100 cells were examined per independent experiment (n); n=3, except for vector: n=6; Ski, Hoxb4, Tcfec, Sfpi1 and Hmgb1: n=1; *p≦0.05 in right panel (relative to vector). (B) In vivo differentiation potential along the lympho-myeloid lineages was assessed in long-term recipients (20 weeks post-transplantation) of HSC transduced with Trim27 used as an example: immnophenotypic analysis by flow cytometry was performed using specific antibodies against B, T and myeloid cell surface markers (B220, CD3 and CD11b, respectively) on Ly5.1⁺ cells derived from the peripheral blood, bone marrow and thymus of these mice (and on Ly5.1⁺/GFP⁺ cells in FIG. 28A). (C) Summary of results obtained in B for most of the validated candidates. Values are presented as mean±SD of different selected mice (n) for each gene; n=2, except for vector: n=6; NA10HD, Trim27, Prdm16, Erdr1, Zfp472, Cnbp, Xbp1 and Hdac1: n=3. Only Pml is absent. Dashed lines are presented to compare values of each gene with those of vector in different hematopoietic tissues. (D) Southern blots showing the proviral DNA integrations in the BM (left panel) and in the thymus (right panel) of mice transplanted with Trim27-overexpressing HSCs indicating that the same clones have contributed to repopulation of these two different hematopoietic tissues. Note that these mice are the same mice presented in FIG. 3A. The same analysis for other validated hits is available in FIG. 9B;

FIG. 9 shows the in vivo differentiation of HSCs transduced with newly identified cell autonomous genes. (A) Differentiation potential along the lympho-myeloid lineages in long-term recipients (20 weeks post-transplantation) of HSC transduced with cell autonomous hits. Immnophenotypic analysis by flow cytometry was performed using specific antibodies against B, T and myeloid cell surface markers (B220, CD3 and CD11b, respectively) and gated on Ly5.1⁺/GFP⁺ populations derived from the peripheral blood, bone marrow and thymus of these mice. These data are not available for few genes (Smarcc1 and Prdm16 in all tissues analysed; Ski, Klf10, and Erdr1 in the thymus) due to absence of EGFP expression in the transduced cells. Values represent mean±SD and the number of mice analyzed (n) per candidate gene was n=2 except for vector: n=5; NA10HD and Cnbp: n=3; Trim27 and Klf10: n=1. (B) Southern blot analysis showing the proviral DNA in the BM (upper panel) and in the thymus (bottom panel) of selected recipients that were highly reconstituted at 20 weeks post-transplantation, corresponding to the cell autonomous group. Transduced HSCs remain competent in T cell differentiation although they displayed enhanced reconstitution activity for each gene except for Ski, Prdm16 and Erdr1. n/a=not available;

FIG. 10 shows a schematic representation of the network of HSC activity (A) Quantitative analysis of gene-expression levels in HSC enriched population singly overexpressing all 18 newly identified nuclear HSC activity regulators determined by Q-RT-PCR. RNA was extracted from CD150⁺CD48⁻Lin⁻Kit⁺Sca⁺ bone marrow cells co-cultured with retroviral producers for 5 days, and sorted for the GFP positive fraction. Average ΔCt values were determined with β-actin serving as endogenous control to normalize levels of target gene expression. Relative fold differences (RQ) were determined and corresponding empty vector (mean of pKOF-1, -2, and -3) was used as reference calibrator to assess relative fold differences in expression levels of each candidate in HSC. Reactions were done in triplicate, and average values were calculated for each independent experiment (n); n=3, except for Ski and Sfpi1: n=1. Relative fold differences were determined using the ΔΔCt method. ND (not determined) values are shown in white. The legend colouring is based on the scaled values of each row for ΔCt heatmap, and on the log₂ of all values in the plot with a maximum value of 13.5 for RQ heatmap. (B) An integrative diagram is presented, correlating mRNA transcript upregulation by overexpression of a hit (black solid arrows) and cell fate determination (grey dotted arrows). Numbers indicate relative fold differences (≧3-fold) observed in (A);

FIG. 11 shows two different forms of Trim27 with different potential. (A) Two different forms of Trim27 have been tested, i.e., one containing a frame-shift error (truncated; accession number BC085503; upper panel) preserving intact only the RING, B-box and first Coiled-coil domains, and another full-length form (accession number BC003219; bottom panel) containing moreover the second Coiled-coil and the SPRY domains. (B) Competitive repopulation assays reporting the differential reconstitution level of recipient mice by HSCs transduced with the different forms of Trim27. Note that the SPRY domain within the full-length form of Trim27 seems to limit the potential of this gene in HSC expansion. WBC=white blood cells;

FIG. 12 shows HSCs depletion following transformation with empty vectors (A) and vectors expressing control genes (B);

FIG. 13 shows HSC expansion following transformation with an empty vector and a vector expressing different genes (A) sfb1, xbp, fos, trim27, ap2a2, sox4; and B) klf1;

FIG. 14 shows the differentiation of HSCs transformed with a vector expressing different genes (xbp, trim27, sox4 and hnrpdl) into various cell types/lineages in different tissues (blood, bone marrow and thymus). B220 is a B-cell lineage marker, CD11b is a myeloid lineage marker and CD4/CD8 are T-cell lineage markers;

FIG. 15 shows the differentiation of HSCs transformed with a vector expressing different genes (xbp1, trim27, sox4, pbx2, meis, klf10, spns1 and cbfb) into various cell types/lineages in the blood. PKOF=empty vector;

FIG. 16 shows the differentiation of HSCs transformed with a vector expressing different genes (xbp1, trim27, sox4, pbx2, meis, klf10, spns1 and cbfb) into various cell types/lineages in the bone marrow. PKOF=empty vector;

FIG. 17 shows the differentiation of HSCs transformed with a vector expressing different genes (xbp1, trim27, sox4, pbx2, meis, klf10, spns1 and cbfb) into various cell types/lineages in the thymus. PKOF=empty vector;

FIG. 18 shows the clonality of the differentiated HSCs transformed with a vector expressing different genes (xbp1, sox4, hnrpdl, gpsm2 and ap2a2) in the bone marrow (BM) and the thymus;

FIG. 19 shows the differentiation (20 weeks post-transplantation) of HSCs transformed with a vector expressing xbp1 into various cell types/lineages;

FIG. 20 shows the differentiation (20 weeks post-transplantation) of HSCs transformed with a vector expressing trim27 into various cell types/lineages;

FIG. 21 shows the differentiation (20 weeks post-transplantation) of HSCs transformed with a vector expressing sox4 into various cell types/lineages;

FIG. 22 shows the differentiation (20 weeks post-transplantation) of HSCs transformed with a vector expressing cbfb into various cell types/lineages;

FIG. 23 shows the differentiation (20 weeks post-transplantation) of HSCs transformed with a vector expressing pbx2 into various cell types/lineages;

FIG. 24 shows the differentiation (20 weeks post-transplantation) of HSCs transformed with a vector expressing klf10 into various cell types/lineages;

FIG. 25 shows the expansion of HSCs transduced with a vector expressing ap2a2 as compared to HSCs transduced with an empty vector;

FIG. 26 shows that screening strategy improves the signal to noise ratio of the results after ex vivo culture (A) WBC chimerism showing that HSCs overexpressing Hoxb4 transplanted without ex vivo culture gives reconstitution levels similar to that observed with vector alone, making signal and noise difficult to separate. (B) WBC chimerism showing that a 7-day ex vivo culture prior to transplantation enhances considerably the signal to noise ratio due to a better reconstitution ability of HSCs overexpressing Hoxb4 coupled with a depletion of HSC activity with control vector. These results, from which the screen has been planned, have been previously obtained using whole (not sorted) BM cells from 5-fluorouracil-pre-treated donor mice, which differ from FIG. 5A; WBC=white blood cells;

FIG. 27 shows the expression of ap2a2 protein in the virus producers. (A) HSCs transformed with a vector expressing ap2a2; (B) the clonality; and (C) the differentiation into various cell types/lineages at twenty weeks are shown; and

FIG. 28 shows nucleic acids and proteins sequence alignments performed using Clustal W™ between variants of HSC regulators of the present invention. (A) Xbp1; (B) tgif; (C) Pml; (D) tcfec; (E) Klf10 (F) cbfb; and (G) Prdm16.

### DESCRIPTION OF ILLUSTRATIVE EMBODIMENTS

The present invention is illustrated in further details by the following non-limiting examples.

### EXAMPLE 1

### Experimental Model

Sélection and Ranking of Candidates

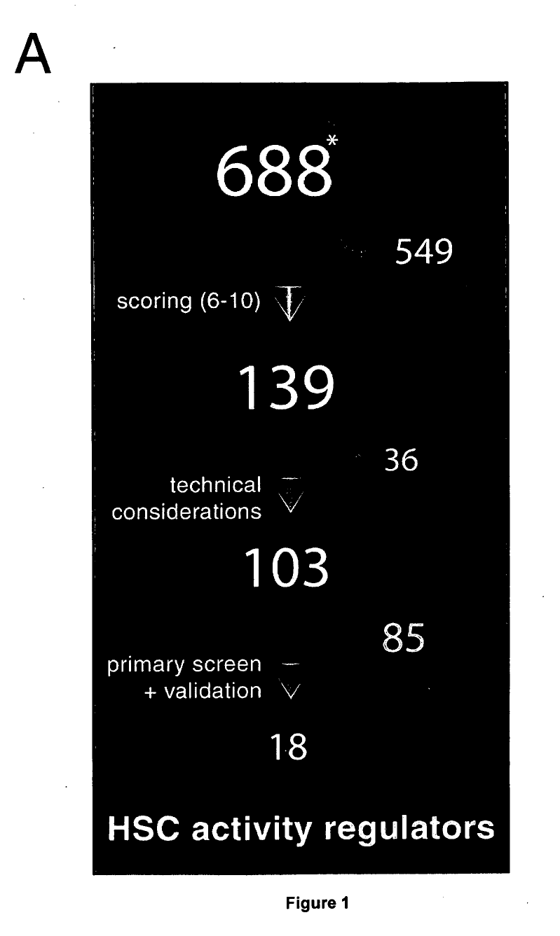

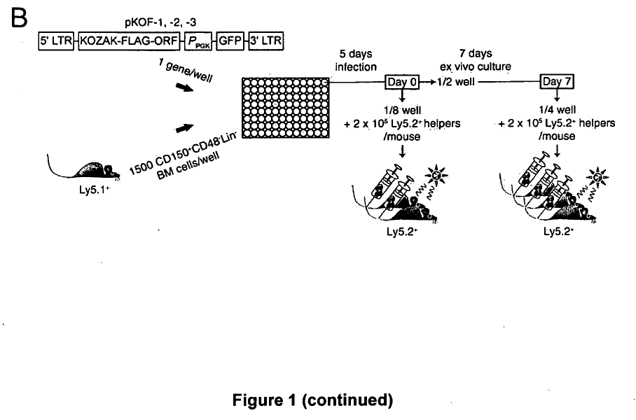

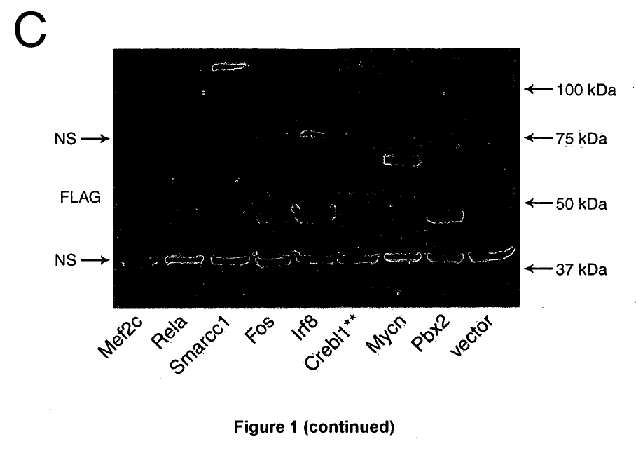

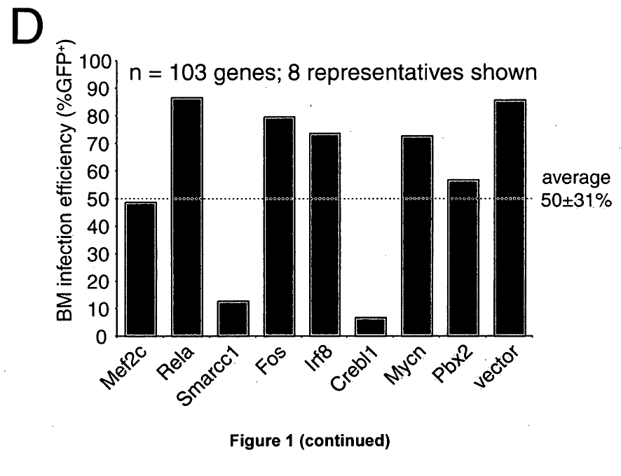

As a corollary to ESC studies, it can be stipulated that HSC fate is controlled by a series of master regulators, analogous to October 4, and several subordinate effectors, providing sound basis to the generation of a stem cell nuclear factors database. Towards this end, we created a database consisting of 688 nuclear factors (see www.132.204.81.89:8088; FIG. 1A), considered candidate regulators of HSC activity. This list was mostly derived from microarray gene expression profiling of normal and leukemia stem cells including our recently generated FLA2 leukemia (1 in 1.5 cells are leukemia stem cells). This database was also enriched by genes obtained following a review of the literature on HSC self-renewal (15-21). A similar approach was used to identify candidate genes which are asymmetrical cell division regulators.

Candidate genes were next ranked from 1 (lowest priority) to 10 (highest priority) based on 3 factors: 1) differential expression between primitive and more mature cellular fractions (e.g., LT-HSC-enriched); 2) expression levels (high, highest priority); and 3) consistency of findings between datasets.

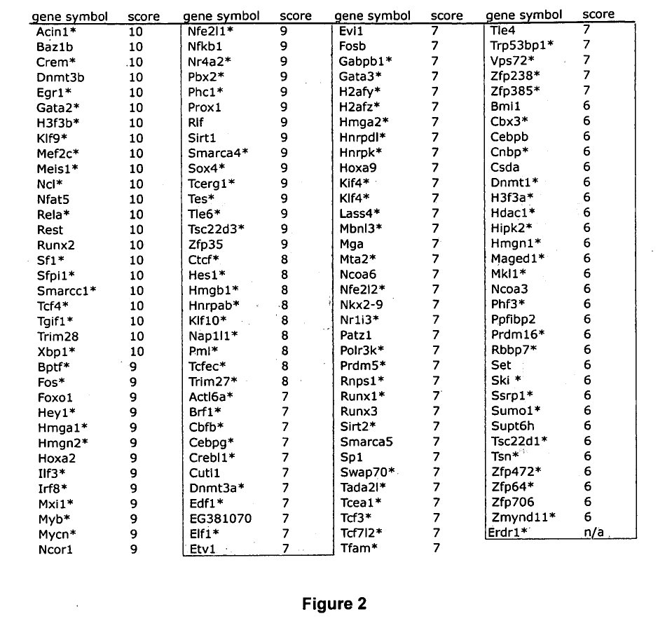

Rank 1=Factors expressed only in one database/report and at relatively low level; Rank 2=Factors expressed in two different contexts (e.g., 2 probesets or 2 libraries); Rank 3=Factors expressed in three different contexts; Rank 4=Factors selected for their function (e.g., stem cell regulator); Rank 5=Factors highly expressed in a given database/report (i.e., top 10%); Rank 6=(Rank 4 or Rank 5)+(Rank 2 or Rank 3); Rank 7=Factors expressed in 2 independent databases/reports or [Rank 3+(2×(Rank 4 or Rank 5))]; Rank 8=[Factors expressed in 4 different contexts+(3×(Rank 4 or Rank 5))] or (Rank 7+Rank 2); Rank 9=Rank 7+[(Rank 4 or Rank 5) or (Rank 2+(Rank 4 or Rank 5)) 3]; Rank 10=Factors expressed in 3 independent databases/reports or [Rank 7+((Rank 2+(2×(Rank 4 or Rank 5))) or (Rank 3+(Rank 4 or Rank 5)))]. Genes with a score of 6 and above (n=139) were selected for functional studies, of which 103 were tested. See FIG. 2.

### EXAMPLE 2

### Primary Screen

As a primary screen, a competitive repopulation assay was used for measurement of HSC activity to validate candidates previously identified.

The ability of the 139 highest scored candidates to affect hematopoietic stem cell (HSC) self-renewal and/or proliferation in vitro and in vivo was evaluated.

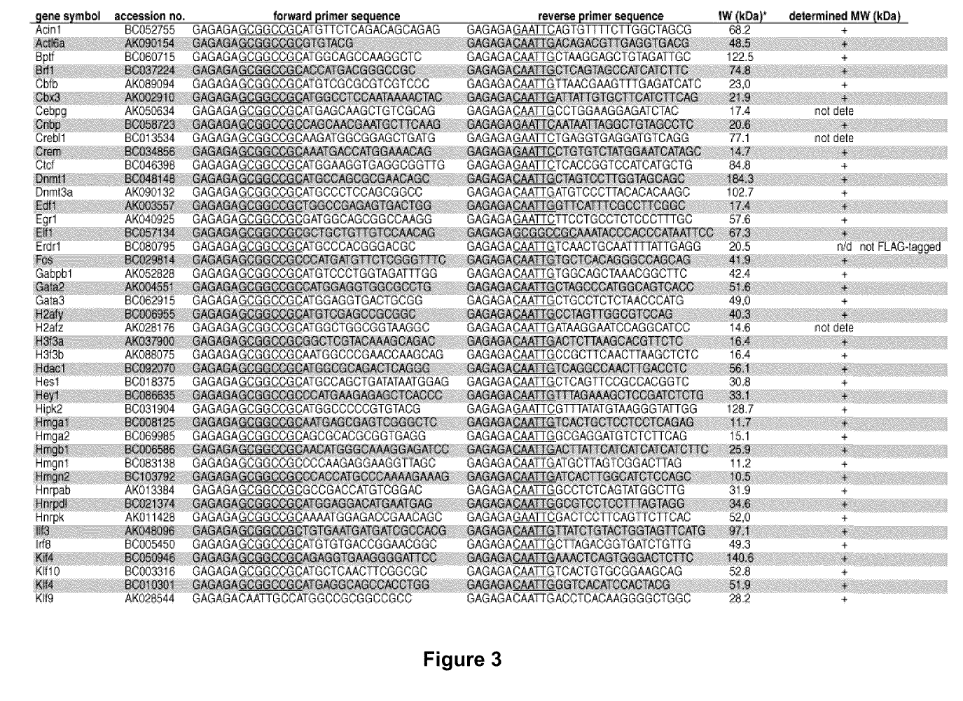

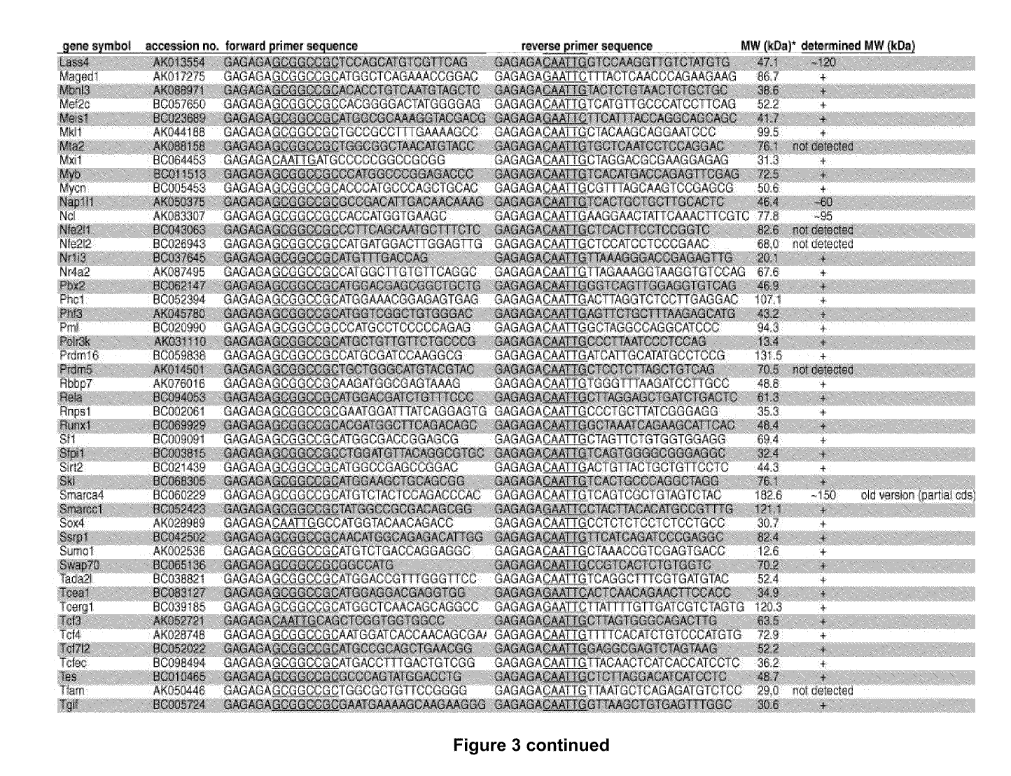

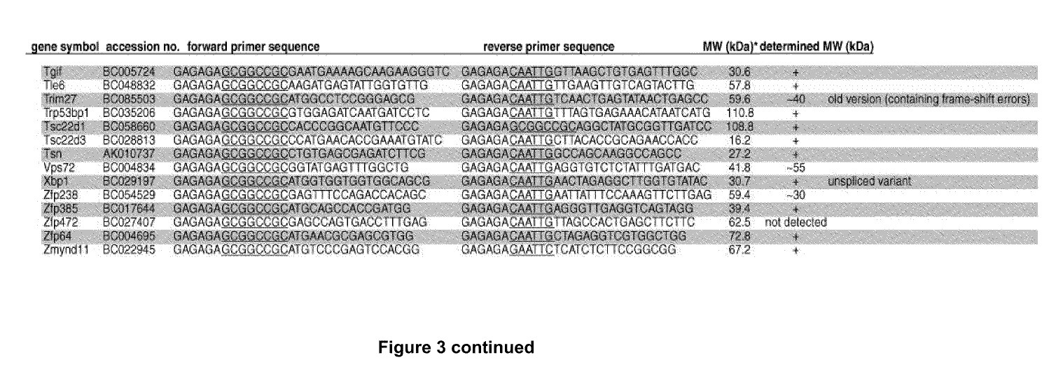

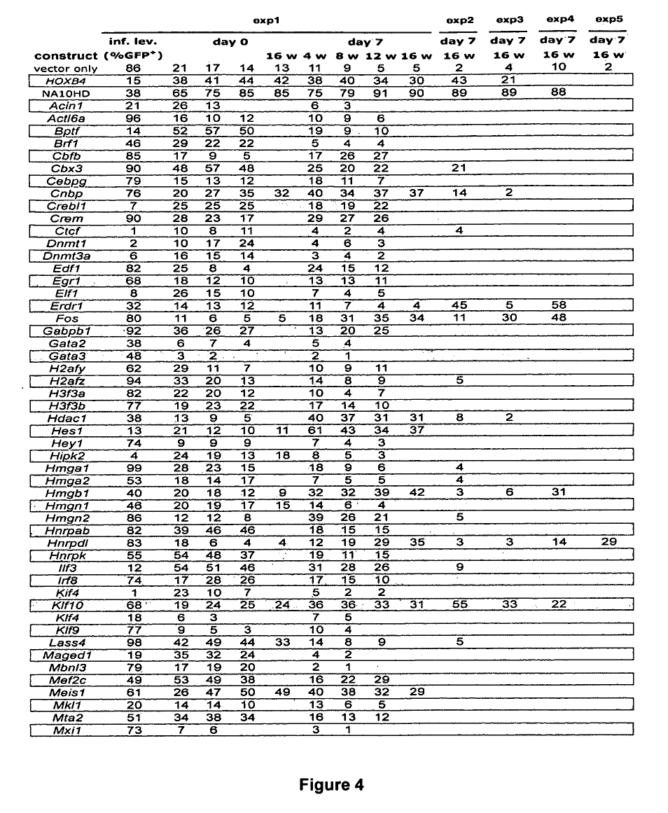

The screening protocol is outlined in FIG. 1B. In brief, the cDNA corresponding to the open reading frames for each of these genes was amplified by PCR, FLAG-tagged and subcloned into 1 out of 3 modified MSCV vectors containing a different reading frame (pKOF-1, pKOF-2 and pKOF-3) that includes a GFP reporter cassette (FIG. 1B). High-titer retroviruses were produced in 96 well plates seeded with viral producer cells using a procedure optimized locally. Protein extracts derived from producer cells in each of the 103 wells were analyzed by western blotting which confirmed the presence of a FLAG-protein in 88% of the cases (FIGS. 1C and 3), with 92% of these proteins showing the expected molecular size (FIG. 3). Respective retroviral producers were plated in a single well of a 96-well dish and co-cultured for 5 days with freshly sorted (CD150⁺CD48⁻Lin⁻) bone marrow cells. Immediately upon infection (Day 0), half of each well was transplanted into sublethally irradiated congenic recipient mice along with competitor cells (Ly5.2+ helpers). A similar assay was performed following an additional week of ex vivo culture (Day 7). FIGS. 1D and 4 show the retroviral gene transfer efficiencies of sampled gene candidates based on GFP epifluorescence assessment of (Day 0) cultured BM cells. A List of predicted and observed molecular weights for most proteins tested in the present invention is shown in FIG. 3. Retroviral gene transfer to freshly isolated mouse bone marrow cells enriched for HSC activity (Lin⁻CD150⁺CD48⁻) varied significantly with an average of 50.0% ±31% (FIGS. 1D and 4). For each gene analyzed, a proportion of the transduced cells was transplanted into lethally irradiated recipients along with competitor cells immediately at the end of retroviral gene transfer (day 0) and after an additional 7 days of ex vivo culture (day 7) (FIG. 1B). Peripheral blood cell reconstitution was then assessed after short (4 and 8 weeks) and long periods of time (12 and 16 weeks) post-transplantation to evaluate the impact of each candidate to affect in vivo (day 0) and ex vivo (day 7) expansion of short and long-term repopulating cells. MSCV-Hoxb4-GFP was used as a positive control in these experiments and 3 different MSCV-GFP viruses were used as negative controls.

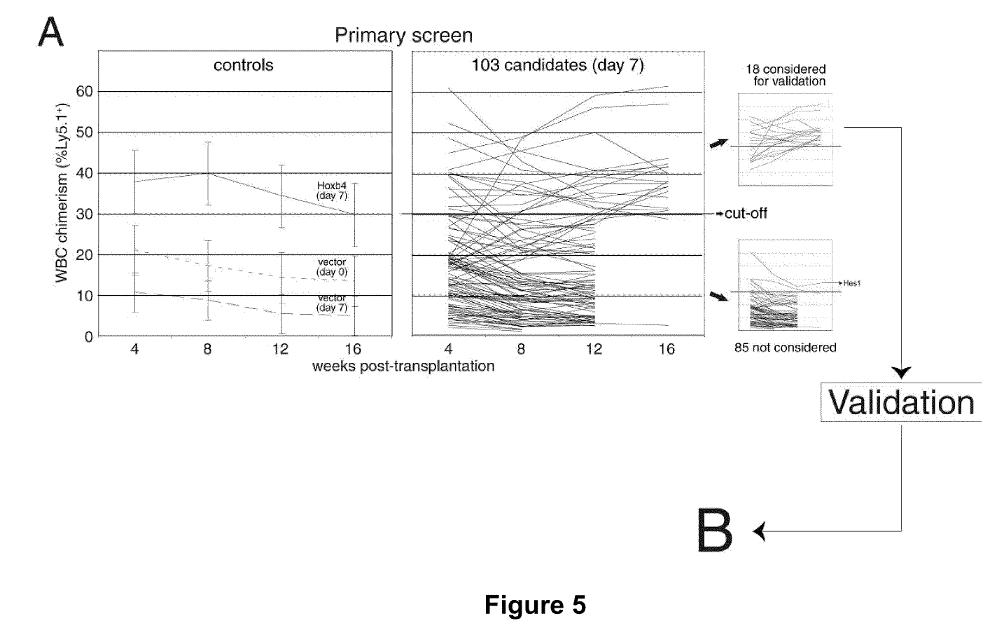

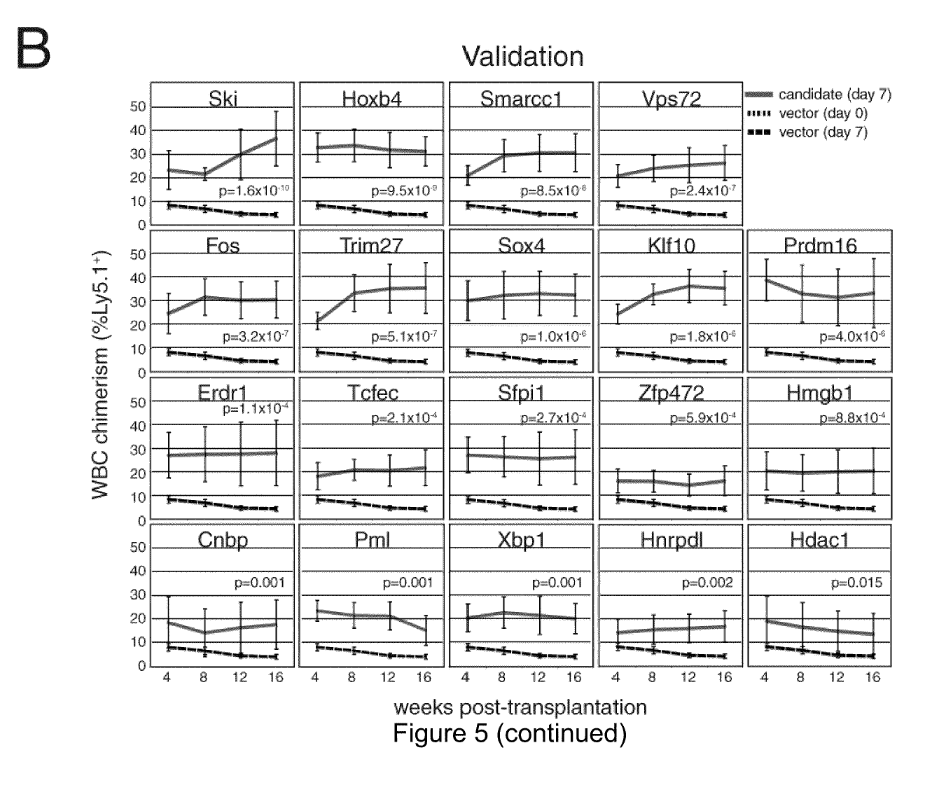

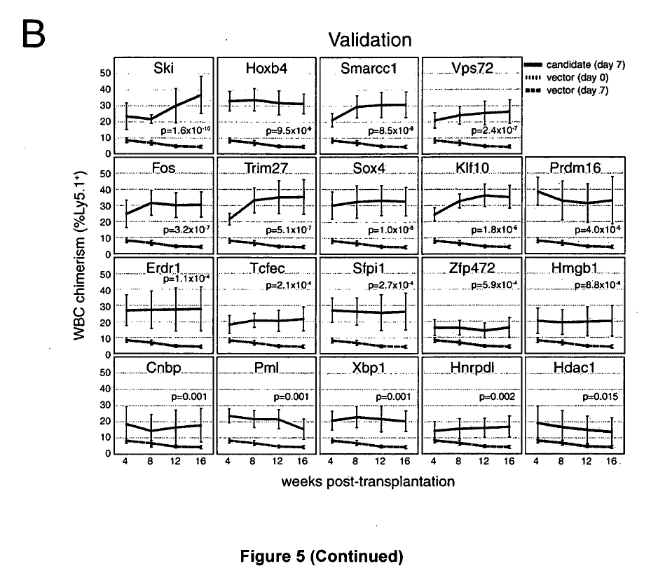

As indicated above, graft-derived hematopoiesis was evaluated at 4-week intervals in primary recipient mice of cultured BM cells by determining the percentage of Ly5.1 positive cells (donor-derived) in peripheral blood (PB) using FACS analysis (FIG. 5). Day 7 values for the screened 103 candidates are compiled and presented in FIG. 5A, with the established cut-off level for a gain-of-function readout. Criteria used for hit selection were: peripheral blood reconstitution by Ly5.1⁺ cells above 10% at 8 weeks and 30% at 12 weeks. Candidates clustering above this level were selected for confirmatory experiments, while those below were disregarded (see right panels). One hit (Hesl) was eliminated based on the marked reduction in repopulation noted between early and late time points (upper line in FIG. 5A, right lower panel).

Recipients of HSCs transduced with Hoxb4 (positive control) or with the backbone vectors in all 3 frames (pKOF1, 2, 3: negative controls) were thus used to set the cut off for selecting the candidates needing further validation. As expected from previous results (13), depletion of HSC activity was verified during 7 day cultures since peripheral blood reconstitution of recipients transplanted 16 weeks earlier with pKOF-transduced cells decreased from 13.3±6.2% (day 0 cells, dotted line in FIG. 5A) to 4.9±4.8% (day 7 cells, dashed line in FIG. 5A). This led to estimate that approximately 3 Ly5.1⁺ HSCs were competing with 20 Ly5.2⁺ HSCs to repopulate each mouse transplanted with <<day 0>>cells and approximately 1 HSC after 7 days of ex vivo culture. These data also suggests that reconstitution from non-infected Ly5.1⁺ cells, even at 0% infection rate, would be consistently below 10% (see dashed line in FIG. 5A) indicating that the 30% cut off used in the primary screen (FIG. 5A, middle panel) was stringent enough to identify genes that confer enhanced in vitro/in vivo activity to transduced HSCs. Notably, the 30% cut off value represents the average reconstitution observed in recipients of Hoxb4-transduced cells in these conditions (see FIG. 5A, left panel). Based on these criteria, we expect that the newly identified candidates should be equivalent to—or more potent than—Hoxb4 in inducing enhanced HSC activity.

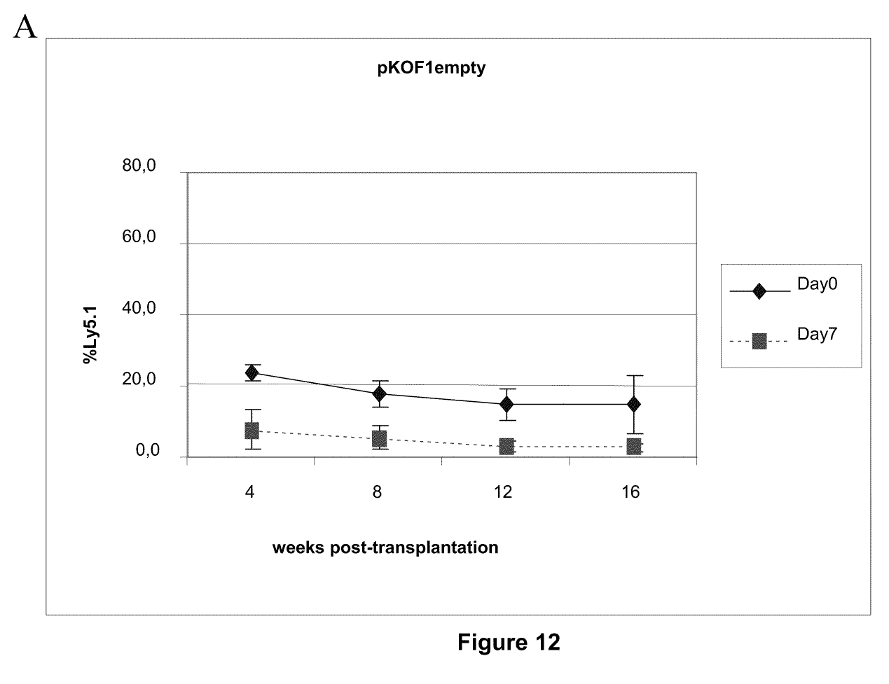

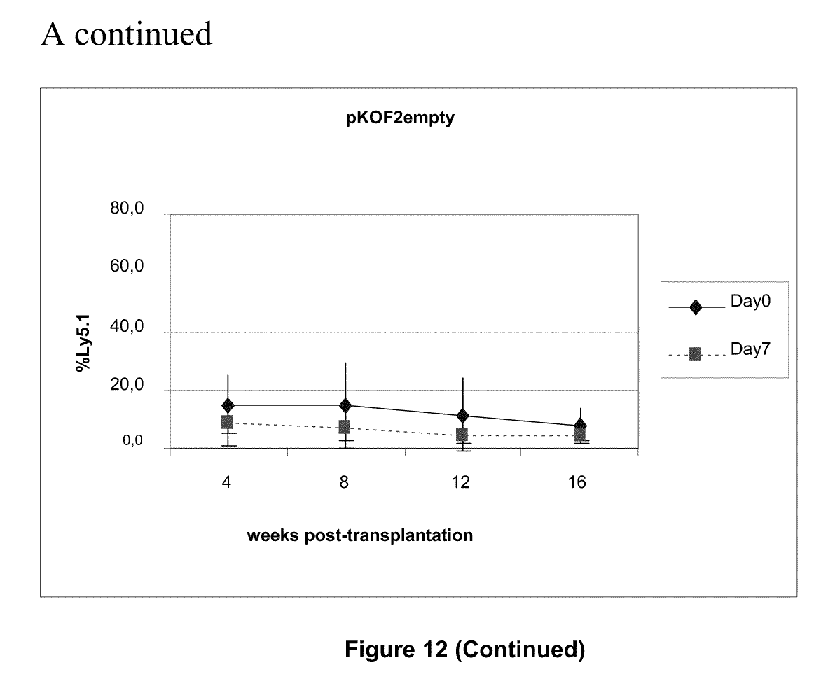

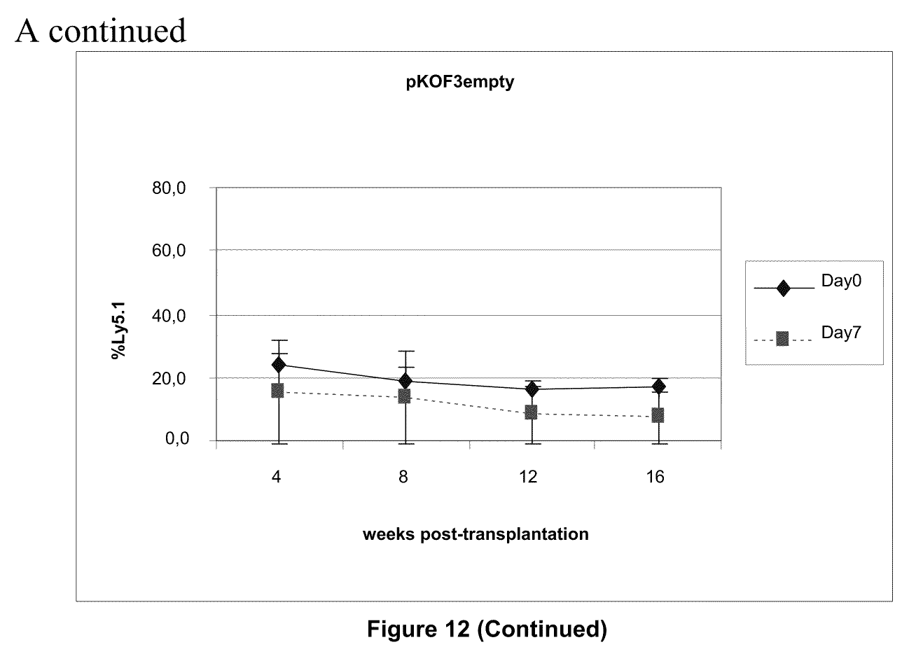

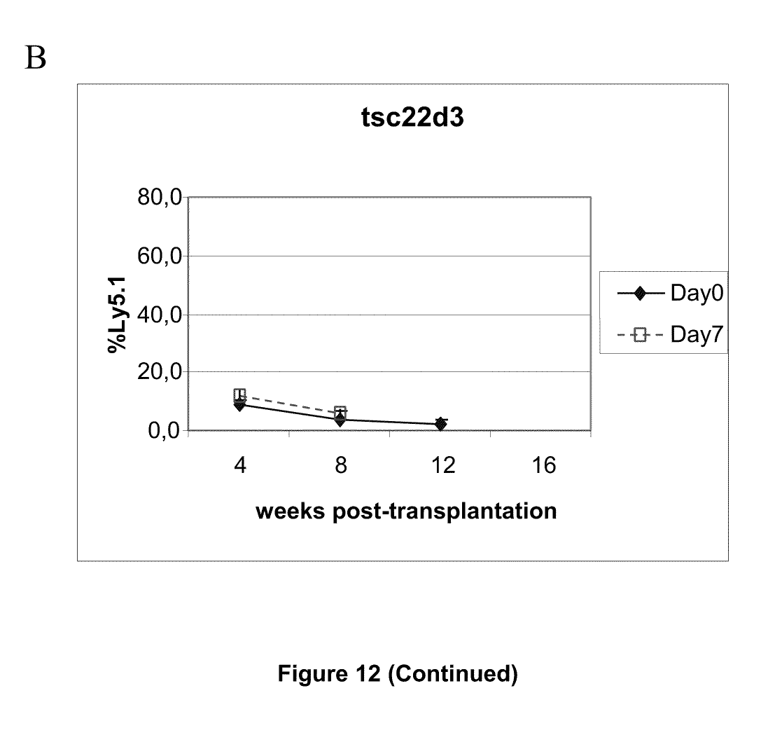

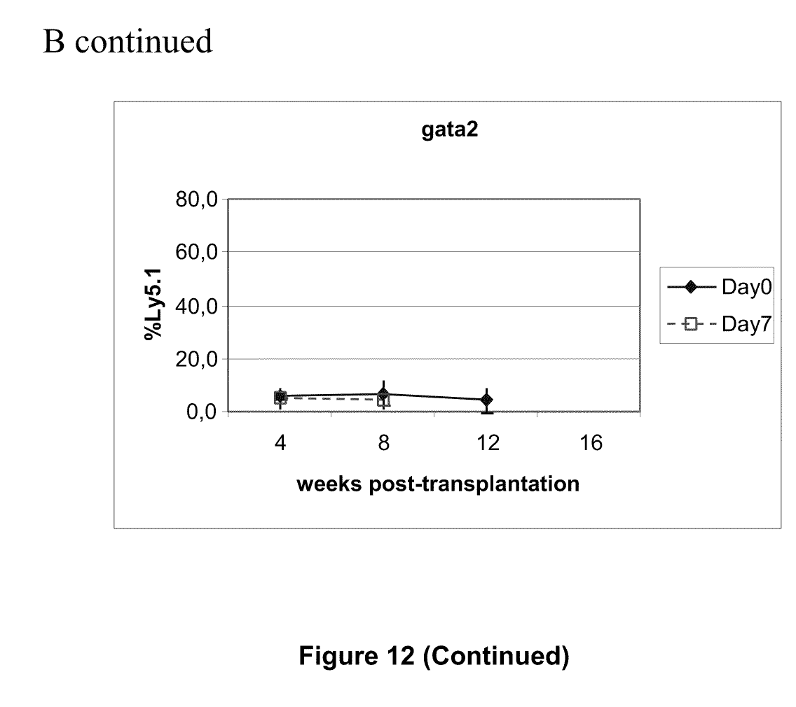

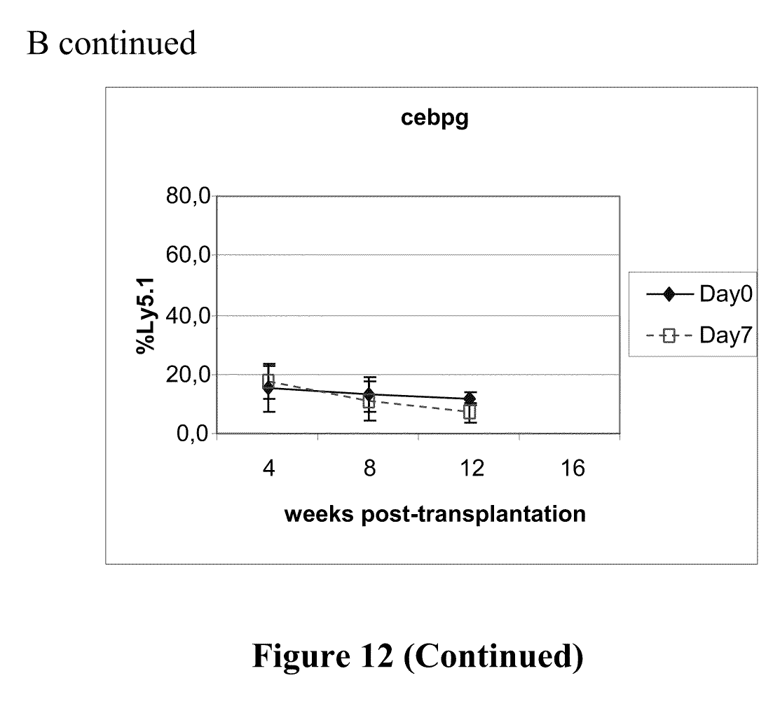

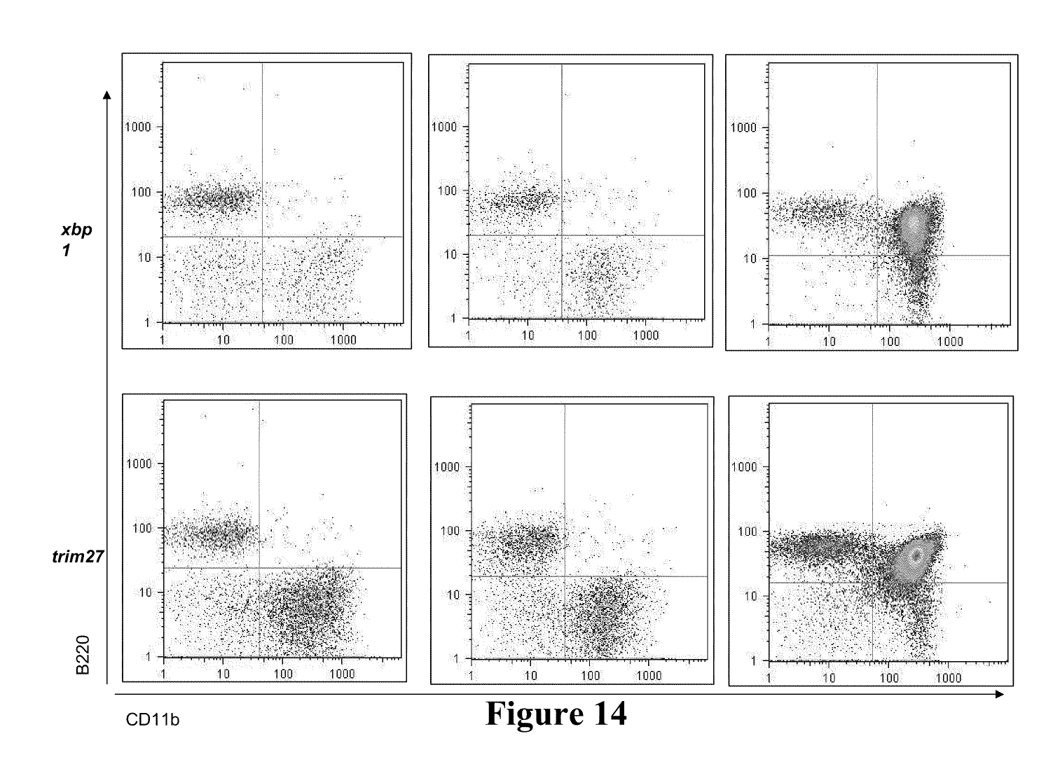

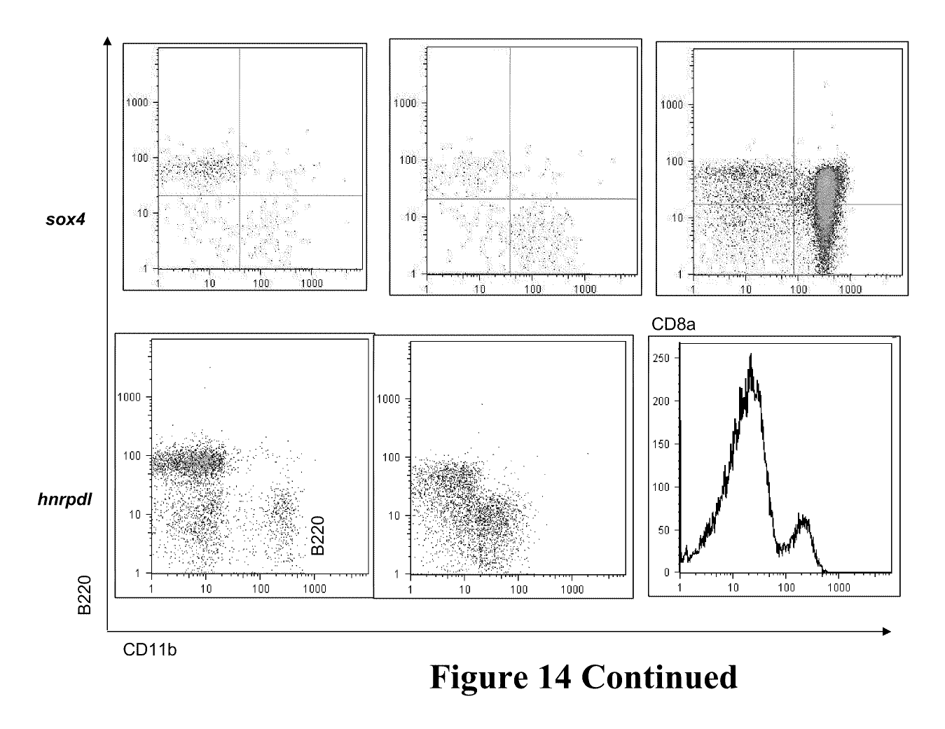

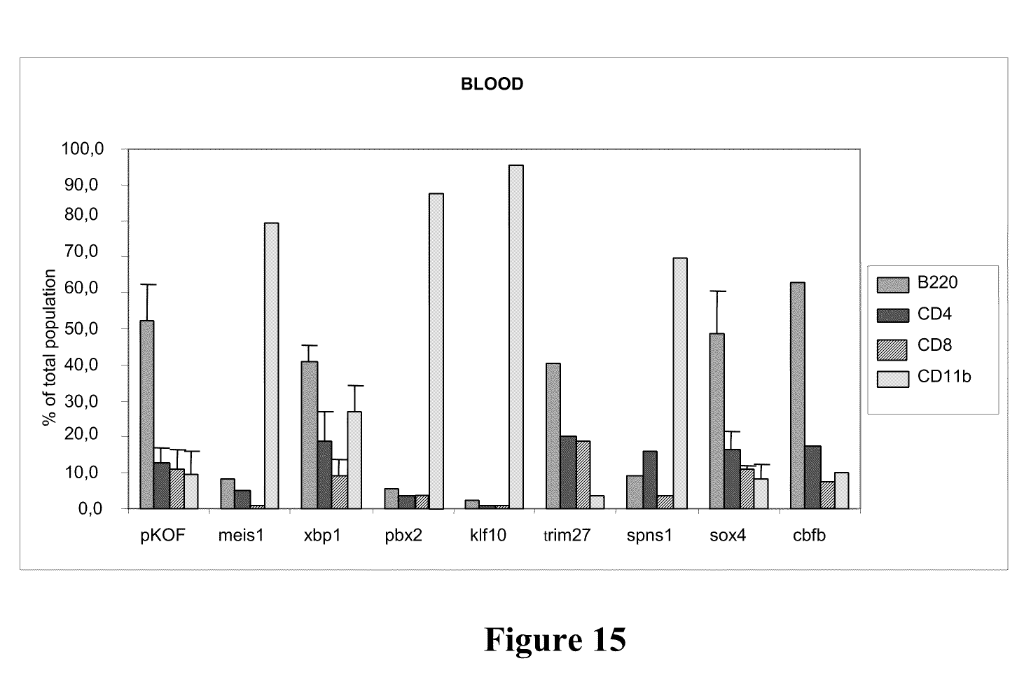

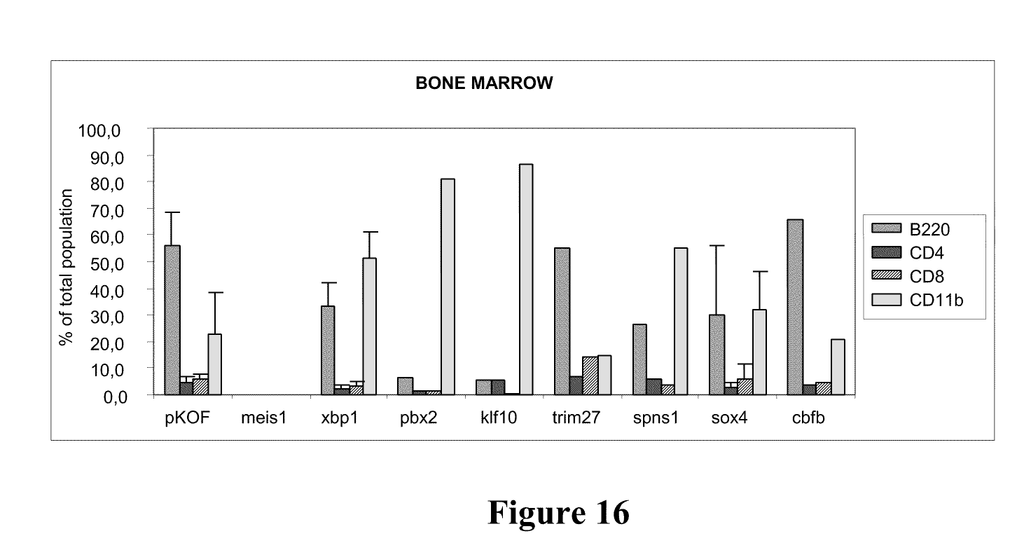

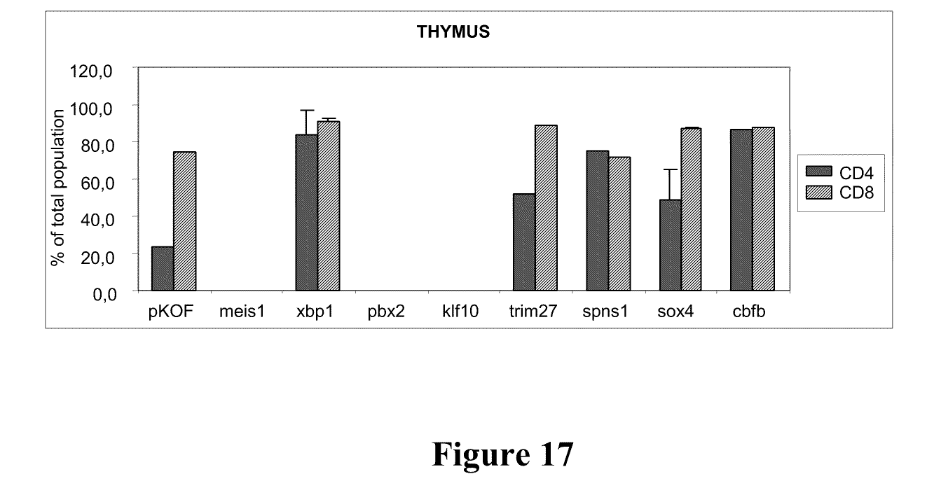

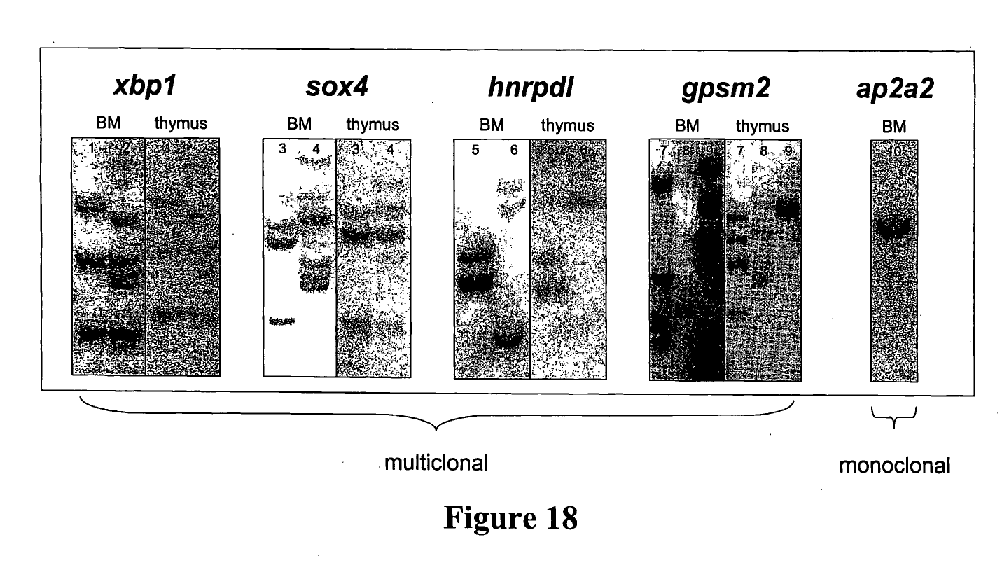

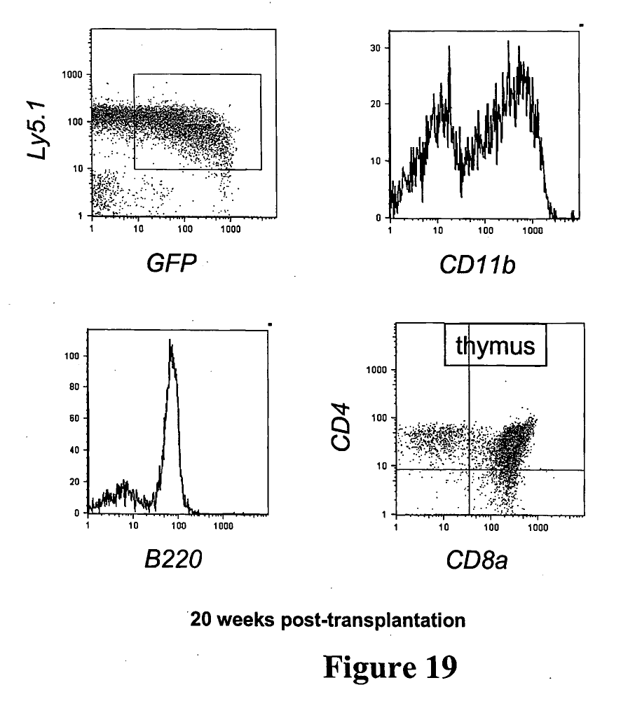

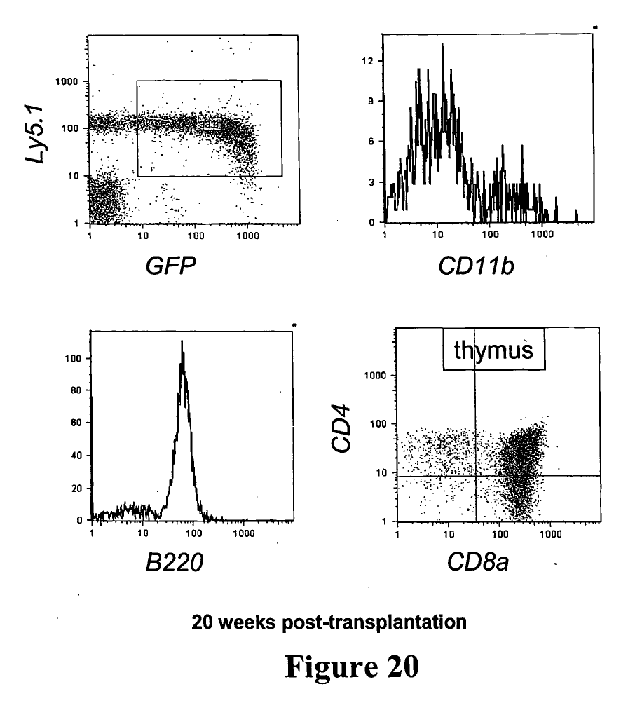

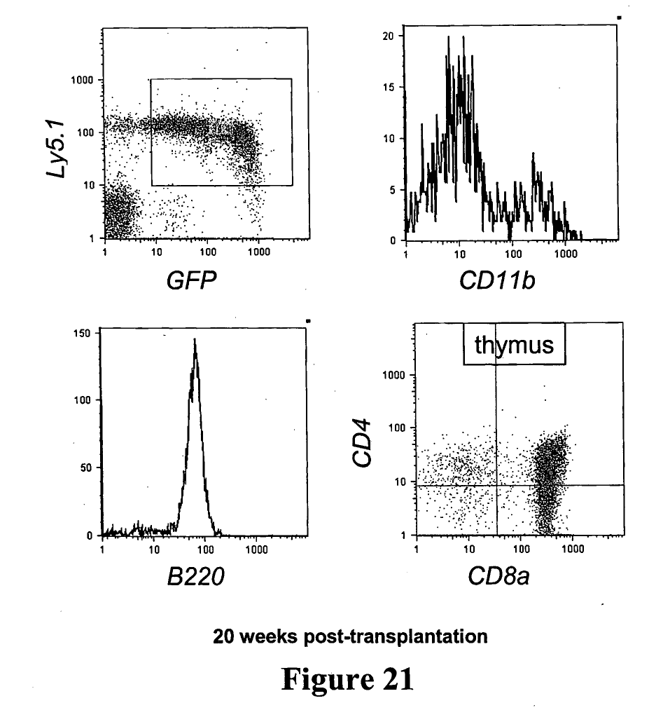

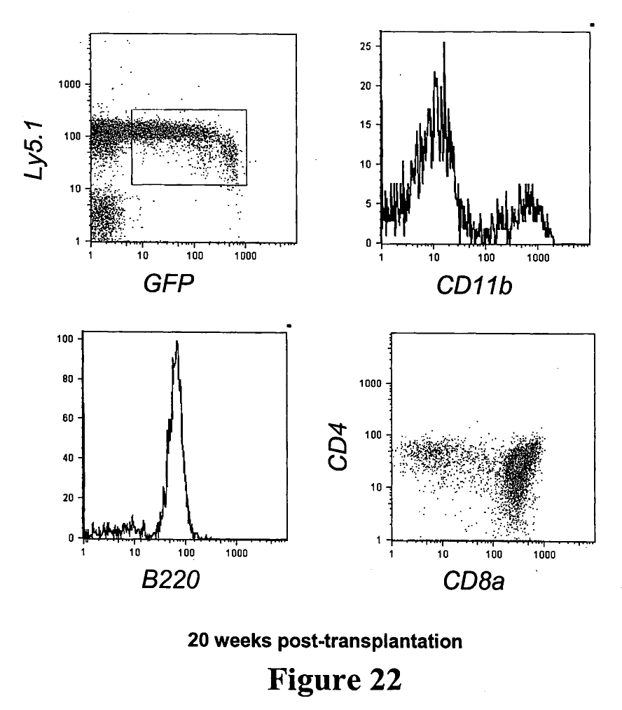

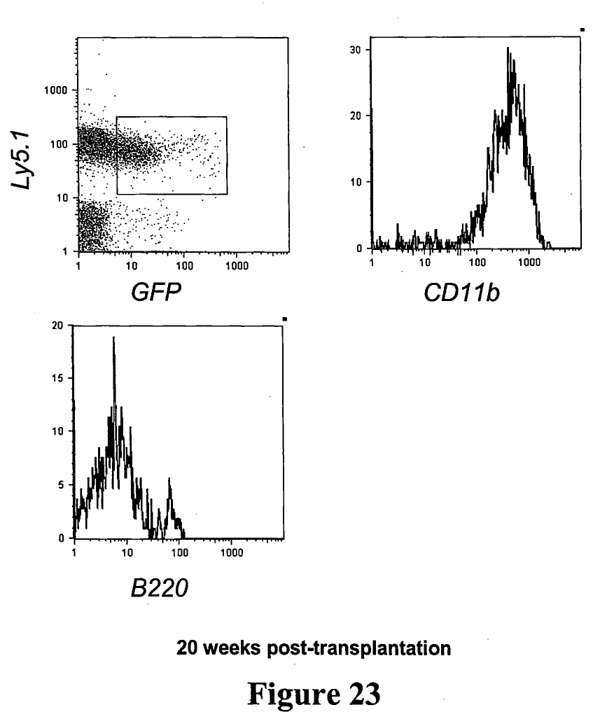

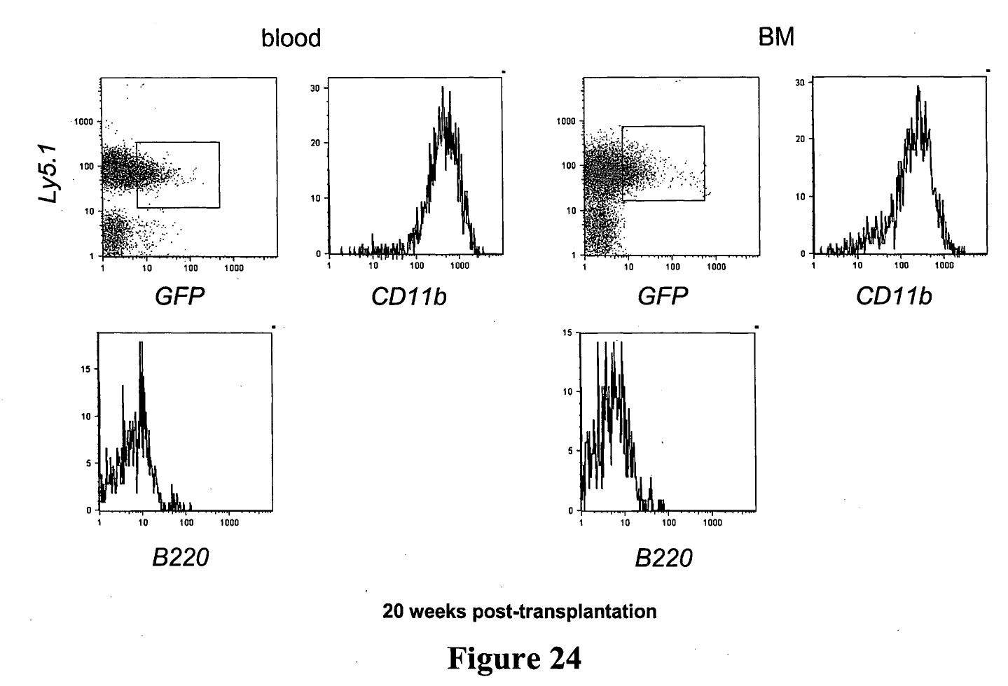

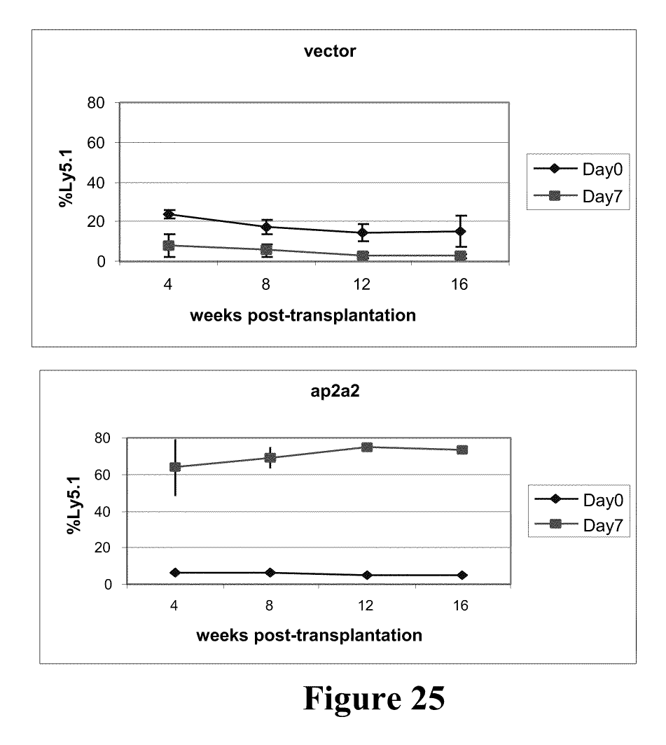

In total, 18 nuclear factor genes hits were identified in this primary screen for a frequency of 17% ( 18/103) (FIG. 5A, upper right panel; and FIG. 4, FIGS. 12-26 as well as Tables 1 and 2). These included Cnbp, Erdrl, Fos, Hdacl, Hmgbl, Hnrpdl, K1flO, Pml, Prdm16, Sfpil (PU.1), Ski, Smarccl (Baf155), Sox4, Tcfec, Trim27, Vps72, Xpbl, and Zfp472.

Using the same approach as described above, 4 additional genes encoding factors controlling assymetrical cell division (ap2a2, tmdo1, gpsm2 and gpx3) were also identified. Together, these 22 selected candidate genes provided competitive advantage (i.e., promoting expansion) to transduced HSC to levels similar to those observed with Hoxb4-transduced HSCs.

Table I presents expansion results for genes providing competitive advantage to transduced HSCs.

| | | Day 0 | Day 0 | Day 0 | Day 0 | Day 7 | Day 7 | Day 7 | Day 7 |
|---|---|---|---|---|---|---|---|---|---|
| | inf. rate | 4 w | 8 w | 12 w | 16 w | 4 w | 8 w | 12 w | 16 w |
| vector | | | | | | | | | |
| exp4pKOF1 | 73 | 23.8 | 17.5 | 14.7 | 14.9 | 7.7 | 5.5 | 2.7 | 2.7 |
| exp4pKOF2 | 85 | 14.8 | 14.4 | 11.6 | 8.1 | 8.4 | 6.6 | 4.2 | 4.4 |
| exp4pKOF3 | 99 | 24.2 | 19.3 | 16.4 | 16.8 | 15.7 | 13.8 | 9.0 | 7.5 |
| exp8pKOF1 | 48.2 | 3.9 | 9.7 | 21.9 | 15.5 | 4.1 | 2.1 | 3.7 | 2.0 |
| exp10pKOF1 | 30.9 | 12.6 | 16.6 | 20.5 | 22.6 | 6.4 | 5.5 | 3.4 | 4.0 |
| exp11apKOF1 | 30.1 | | | | | 13.3 | 13.2 | 9.1 | 9.5 |
| exp11bpKOF1 | 30.1 | | | | | 2.7 | 4.0 | 2.4 | 2.0 |
| mean | 56.6 | 15.9 | 15.5 | 17.0 | 15.6 | 8.3 | 7.2 | 4.9 | 4.6 |
| SEM | | 3.79 | 1.65 | 1.89 | 2.31 | 1.77 | 1.71 | 1.08 | 1.09 |
| hoxb4 | | | | | | | | | |
| exp6 | 15 | 38.4 | 41.2 | 43.8 | 42.4 | 37.7 | 39.7 | 34.2 | 29.6 |
| exp7 | 18.6 | 20.0 | 30.3 | 34.9 | 28.5 | 40.0 | 41.3 | 43.3 | 42.6 |
| exp8 | 5.3 | 17.5 | 21.3 | 24.5 | 24.0 | 20.6 | 19.9 | 17.8 | 21.3 |
| mean | 13.0 | 25.3 | 30.9 | 34.4 | 31.6 | 32.8 | 33.6 | 31.8 | 31.2 |
| SEM | | 6.60 | 5.75 | 5.58 | 5.55 | 6.10 | 6.87 | 7.48 | 6.18 |
| NA10hd | | | | | | | | | |
| exp10 | 37.6 | 64.6 | 74.7 | 84.8 | 84.8 | 75.1 | 79.0 | 91.0 | 90.4 |
| exp11a | 82.2 | | | | | 69.9 | 89.1 | 90.7 | 89.1 |
| exp11b | 82.2 | | | | | 75.1 | 88.5 | 90.6 | 89.0 |
| mean | 67.3 | 64.6 | 74.7 | 84.8 | 84.8 | 73.4 | 85.5 | 90.7 | 89.5 |
| SEM | | 0.00 | 0.00 | 0.00 | 0.00 | 1.73 | 3.28 | 0.12 | 0.46 |
| smarcc1 | | | | | | | | | |
| exp5 | 13.2 | 46.2 | 61.4 | 56.8 | 49.7 | 26.8 | 37.2 | 40.7 | 39.9 |
| exp10 | 6.2 | 13.8 | 13.9 | 14.1 | 14.1 | 17.3 | 33.7 | 35.6 | 34.3 |
| exp11a | 3.3 | | | | | 11.1 | 8.9 | 7.5 | 7.0 |
| exp11b | 3.3 | | | | | 29.0 | 37.6 | 38.2 | 41.1 |
| mean | 6.5 | 30.0 | 37.7 | 35.5 | 31.9 | 21.0 | 29.3 | 30.5 | 30.6 |
| SEM | | 16.20 | 23.74 | 21.36 | 17.80 | 4.17 | 6.86 | 7.74 | 8.00 |
| xbp1 | | | | | | | | | |
| exp4 | 80 | 13.1 | 9.3 | 7.4 | 7.2 | 40.7 | 45.4 | 50.0 | 39.8 |
| exp10 | 75.7 | 18.3 | 12.7 | 7.2 | 7.2 | 16.7 | 11.8 | 6.8 | 6.8 |
| exp11a | 80.4 | | | | | 8.1 | 8.3 | 5.5 | 6.0 |
| exp11b | 80.4 | | | | | 25.8 | 26.3 | 23.7 | 22.9 |
| mean | 79.1 | 15.7 | 11.0 | 7.3 | 7.2 | 22.8 | 23.0 | 21.5 | 18.9 |
| SEM | | 2.57 | 1.73 | 0.11 | 0.00 | 6.98 | 8.44 | 10.36 | 7.99 |
| fos | | | | | | | | | |
| exp4 | 80 | 11.4 | 6.0 | 4.7 | 4.6 | 18.3 | 30.6 | 35.1 | 33.9 |
| exp10 | 63.2 | 7.5 | 6.7 | 5.9 | 5.9 | 8.1 | 13.0 | 10.4 | 10.6 |
| exp11a | 63.3 | | | | | 24.2 | 32.2 | 28.2 | 29.5 |
| exp11b | 63.3 | | | | | 48.4 | 50.8 | 47.5 | 48.4 |
| mean | 67.5 | 9.5 | 6.4 | 5.3 | 5.2 | 24.8 | 31.7 | 30.3 | 30.6 |
| SEM | | 1.93 | 0.38 | 0.63 | 0.67 | 8.54 | 7.73 | 7.75 | 7.79 |
| hmgb1 | | | | | | | | | |
| exp4 | 40 | 19.7 | 18.4 | 12.0 | 9.4 | 31.7 | 32.1 | 38.7 | 42.2 |
| exp10 | 7 | 12.0 | 10.5 | 9.1 | 9.1 | 5.4 | 4.2 | 2.6 | 2.6 |
| exp11a | 17.3 | | | | | 8.0 | 8.4 | 6.0 | 6.0 |
| exp11b | 17.3 | | | | | 36.5 | 33.5 | 32.9 | 30.9 |
| mean | 20.4 | 15.9 | 14.5 | 10.5 | 9.2 | 20.4 | 19.6 | 20.0 | 20.4 |
| SEM | | 3.85 | 3.91 | 1.47 | 0.19 | 7.98 | 7.72 | 9.20 | 9.63 |
| tcfec | | | | | | | | | |
| exp4 | 76 | 13.9 | 17.3 | 17.0 | 17.0 | 29.2 | 30.4 | 36.4 | 41.5 |
| exp10 | 53.4 | 1.1 | 3.8 | 6.5 | 6.5 | 7.6 | 13.4 | 7.2 | 7.6 |
| exp11a | 27.7 | | | | | 26.6 | 26.5 | 26.1 | 25.2 |
| exp11b | 27.7 | | | | | 9.2 | 13.0 | 12.5 | 12.5 |
| mean | 46.2 | 7.5 | 10.6 | 11.7 | 11.7 | 18.2 | 20.8 | 20.6 | 21.7 |
| SEM | | 6.38 | 6.78 | 5.26 | 5.26 | 5.67 | 4.49 | 6.60 | 7.57 |
| klf10 | | | | | | | | | |
| exp4 | 68 | 19.0 | 23.7 | 24.5 | 23.8 | 35.9 | 35.6 | 32.8 | 31.2 |
| exp10 | 47.4 | 7.7 | 7.5 | 7.4 | 7.4 | 25.1 | 43.4 | 56.4 | 55.3 |
| exp11a | 47 | | | | | 20.0 | 29.4 | 32.0 | 33.4 |
| exp11b | 47 | | | | | 17.1 | 23.0 | 23.9 | 21.6 |
| mean | 52.4 | 13.4 | 15.6 | 16.0 | 15.6 | 24.5 | 32.8 | 36.3 | 35.4 |
| SEM | | 5.69 | 8.09 | 8.54 | 8.16 | 4.15 | 4.35 | 7.01 | 7.10 |
| trim27 | | | | | | | | | |
| exp4 | 97 | 12.3 | 5.6 | 3.5 | 4.3 | 18.5 | 48.4 | 59.1 | 61.3 |
| exp10 | 73 | 14.1 | 13.4 | 12.7 | 13.4 | 22.2 | 34.7 | 35.5 | 32.4 |
| exp11a | 44.1 | | | | | 31.5 | 38.5 | 37.3 | 39.0 |
| exp11b | 44.1 | | | | | 14.1 | 11.8 | 9.2 | 9.2 |
| mean | 64.6 | 13.2 | 9.5 | 8.1 | 8.9 | 21.6 | 33.4 | 35.3 | 35.5 |
| SEM | | 0.87 | 3.88 | 4.59 | 4.55 | 3.70 | 7.74 | 10.20 | 10.72 |
| ap2a2 | | | | | | | | | |
| exp4 | 62 | 20.6 | 25.4 | 27.1 | 27.9 | 64.1 | 69.4 | 75.0 | 73.6 |
| exp10 | 48.1 | 20.0 | 18.8 | 17.6 | 17.6 | 27.3 | 33.8 | 37.4 | 40.8 |
| exp11a | 52.1 | | | | | 23.8 | 41.9 | 44.7 | 46.8 |
| exp11b | 52.1 | | | | | 56.9 | 70.3 | 71.8 | 75.4 |
| mean | 53.6 | 20.3 | 22.1 | 22.4 | 22.8 | 43.0 | 53.8 | 57.2 | 59.2 |
| SEM | | 0.31 | 3.30 | 4.76 | 5.14 | 10.22 | 9.40 | 9.48 | 8.97 |
| gpsm2 | | | | | | | | | |
| exp5 | 33.6 | 32.9 | 35.5 | 23.6 | 19.3 | 25.2 | 35.1 | 45.8 | 43.6 |
| exp10 | 18.6 | 10.2 | 9.6 | 8.9 | 8.9 | 5.7 | 4.2 | 2.4 | 2.4 |
| exp11a | 4.9 | | | | | 42.8 | 56.5 | 59.1 | 60.9 |
| exp11b | 4.9 | | | | | 37.3 | 42.8 | 39.3 | 40.0 |
| mean | 15.5 | 21.6 | 22.5 | 16.3 | 14.1 | 27.8 | 34.7 | 36.7 | 36.7 |
| SEM | | 11.36 | 12.96 | 7.35 | 5.17 | 8.22 | 11.07 | 12.14 | 12.32 |
| sox4 | | | | | | | | | |
| exp4 | 97 | 12.2 | 13.1 | 11.6 | 10.4 | 18.0 | 23.4 | 28.5 | 30.7 |
| exp10 | 50.6 | 20.9 | 25.3 | 29.7 | 31.4 | 38.7 | 36.7 | 40.8 | 42.3 |
| exp11a | 72.2 | | | | | 52.6 | 63.8 | 60.7 | 57.0 |
| exp11b | 72.2 | | | | | 36.3 | 35.9 | 32.4 | 29.2 |
| mean | 73.0 | 16.5 | 19.2 | 20.6 | 20.9 | 36.4 | 39.9 | 40.6 | 39.8 |
| SEM | | 4.33 | 6.09 | 9.03 | 10.49 | 7.11 | 8.52 | 7.17 | 6.44 |
| hnrpdl | | | | | | | | | |
| exp6 | 83 | 18.3 | 6.0 | 4.2 | 4.2 | 12.0 | 18.6 | 29.2 | 35.4 |
| exp10 | 94.4 | 5.5 | 4.3 | 3.2 | 3.2 | 6.5 | 4.8 | 2.5 | 2.5 |
| exp11a | 74.5 | | | | | 13.3 | 17.5 | 14.5 | 14.4 |
| exp11b | 74.5 | | | | | 36.3 | 36.0 | 31.0 | 28.9 |
| mean | 81.6 | 11.9 | 5.2 | 3.7 | 3.7 | 17.0 | 19.2 | 19.3 | 20.3 |
| SEM | | 6.37 | 0.82 | 0.50 | 0.53 | 6.60 | 6.40 | 6.71 | 7.38 |
| vps72 | | | | | | | | | |
| exp6 | 53 | 24.7 | 15.4 | 12.1 | 8.8 | 20.0 | 30.4 | 40.3 | 41.4 |
| exp10 | 64.8 | 27.2 | 21.0 | 14.9 | 14.9 | 15.1 | 17.5 | 17.7 | 20.2 |
| exp11a | 49.6 | | | | | 34.5 | 35.8 | 34.9 | 34.9 |
| exp11b | 49.6 | | | | | 13.5 | 12.2 | 8.5 | 8.5 |
| mean | 54.3 | 25.9 | 18.2 | 13.5 | 11.8 | 20.8 | 24.0 | 25.3 | 26.2 |
| SEM | | 1.23 | 2.78 | 1.39 | 3.05 | 4.79 | 5.49 | 7.39 | 7.38 |
| gpx3 | | | | | | | | | |
| exp6 | 96 | 21.3 | 28.5 | 36.6 | 41.0 | 17.3 | 27.9 | 33.3 | 30.1 |
| exp10 | 76.8 | 15.4 | 10.5 | 5.6 | 5.5 | 12.6 | 7.4 | 5.5 | 5.5 |
| exp11a | 71.8 | | | | | 57.7 | 72.4 | 76.3 | 77.3 |
| exp11b | 71.8 | | | | | 17.6 | 13.8 | 10.1 | 10.1 |
| mean | 79.1 | 18.3 | 19.5 | 21.1 | 23.2 | 26.3 | 30.4 | 31.3 | 30.8 |
| SEM | | 2.96 | 9.00 | 15.52 | 17.79 | 10.54 | 14.64 | 16.18 | 16.40 |
| sfpi1 | | | | | | | | | |
| exp4 | 57 | 16.2 | 9.4 | 7.9 | 8.3 | 44.9 | 48.8 | 56.0 | 57.3 |
| exp10 | 48.9 | 10.1 | 7.1 | 4.2 | 4.2 | 16.8 | 15.4 | 9.6 | 9.6 |
| exp11a | 17.8 | | | | | 12.7 | 11.4 | 8.3 | 8.3 |
| exp11b | 17.8 | | | | | 34.2 | 30.0 | 28.6 | 29.8 |
| mean | 35.4 | 13.1 | 8.3 | 6.1 | 6.2 | 27.1 | 26.4 | 25.6 | 26.2 |
| SEM | | 3.09 | 1.15 | 1.88 | 2.06 | 7.52 | 8.46 | 11.15 | 11.47 |
| erdr1 | | | | | | | | | |
| exp10 | 31.8 | 14.3 | 13.1 | 11.9 | 11.9 | 11.1 | 7.0 | 3.6 | 3.6 |
| exp11a | 31.6 | | | | | 44.6 | 44.8 | 45.1 | 45.1 |
| exp11b | 31.6 | | | | | 9.5 | 7.6 | 5.4 | 5.4 |
| mean | 31.7 | 14.3 | 13.1 | 11.9 | 11.9 | 21.8 | 19.8 | 18.0 | 18.0 |
| SEM | | 0 | 0 | 0 | 0 | 11.45 | 12.50 | 13.52 | 13.54 |
| zfp472 | | | | | | | | | |
| exp8 | 2.8 | 7.3 | 5.5 | 11.4 | 11.4 | 17.0 | 26.0 | 27.4 | 37.9 |
| exp10 | 3.1 | 23.7 | 23.7 | 23.7 | 23.2 | 3.2 | 2.7 | 1.5 | 1.5 |
| exp11a | 2.7 | | | | | 30.4 | 24.8 | 20.5 | 19.2 |
| exp11b | 2.7 | | | | | 23.4 | 19.0 | 15.9 | 15.9 |
| mean | 2.8 | 15.5 | 14.6 | 17.6 | 17.3 | 18.5 | 18.1 | 16.3 | 18.6 |
| SEM | | 8.23 | 9.10 | 6.17 | 5.91 | 5.78 | 5.36 | 5.47 | 7.48 |
| tmod1 | | | | | | | | | |
| exp10 | 50.1 | 5.2 | 5.5 | 5.9 | 5.9 | 31.1 | 39.7 | 43.5 | 43.2 |
| cnbp1 | | | | | | | | | |
| exp10 | 75.9 | 20.1 | 27.3 | 34.5 | 31.6 | 40.1 | 34.3 | 37.3 | 37.4 |
| prdm16 | | | | | | | | | |
| exp10 | 3.2 | 33.5 | 34.2 | 34.9 | 36.1 | 52.2 | 45.2 | 40.5 | 43.5 |
| hdac1 | | | | | | | | | |
| exp10 | 38.1 | 12.8 | 9.1 | 5.5 | 5.5 | 39.7 | 36.7 | 31.1 | 30.8 |
| ski | | | | | | | | | |
| exp6 | 5 | 36.0 | 47.1 | 52.0 | 39.4 | 23.2 | 21.5 | 29.9 | 36.6 |
| ctrl neg (rela) | | 20.96 | 10.625 | 6.54 | 5 | 9.1 | 5.1 | 2.9 | 2.4 |

Table II present the Genbank accession numbers for the genes providing competitive advantage to transduced HSC.

| | Genbank accession | Genbank accession | SEQ ID NO: |
|---|---|---|---|
| Gene | number (nucleic | number | Nucleic |
| name | acid) | (polypeptide) | acid/polypeptide |
| trim27 | NM_006510 | NP_006501 | 1/2 |
| Xbp1 | NM_005080 | NP_005071 | 3/4 |
| | NM_001079539 | NP_001073007 | 5/6 |
| Sox4 | NM_003107 | NP_003098 | 7/8 |
| Smarcc1 | NM_003074 | NP_003065 | 9/10 |
| sfpi1 | NM_001080547 | NP_001074016 | 11/12 |
| | NM_003120 | NP_003111 | 13/14 |
| fos | NM_005252 | NP_005243 | 15/16 |
| hmgb1 | NM_002128 | NP_002119 | 17/18 |
| hnrpdl | NM_031372 | NP_112740 | 19/20 |
| vps72 | NM_005997 | NP_005988 | 21/22 |
| tgif | NM_174886 | NP_777480 | 23/24 |
| | NM_173211 | NP_775303 | 25/26 |
| | NM_173209 | NP_775301 | 27/28 |
| | NM_173208 | NP_775300 | 29/30 |
| | NM_170695 | NP_733796 | 31/32 |
| | NM_003244 | NP_003235 | 33/34 |
| | NM_173210 | NP_775302 | 35/36 |
| | NM_173207 | NP_775299 | 37/38 |
| | | | Consensus 93 |
| pml | NM_033250 | NP_150253 | 39/40 |
| | NM_033240 | NP_150243 | 41/42 |
| | NM_033239 | NP_150242 | 43/44 |
| | NM_002675 | NP_002666 | 45/46 |
| | NM_033249 | NP_150252 | 47/48 |
| | NM_033238 | NP_150241 | 49/50 |
| | NM_033244 | NP_150247 | 51/52 |
| | NM_033247 | NP_150250 | 53/54 |
| | NM_033246 | NP_150249 | 55/56 |
| tcfec | NM_012252 | NP_036384 | 57/58 |
| | NM_001018058 | NP_001018068 | 59/60 |
| | | | Consensus: 94 |
| klf10 | NM_001032282 | NP_001027453 | 61/62 |
| | NM_005655 | NP_005646 | 63/64 |
| | | | Consensus 95 |
| cbfb | NM_022845 | NP_074036 | 65/66 |
| | NM_001755 | NP_001746 | 67/68 |
| | | | Consensus: 96 |
| zfp472 | NM_153063 | NP_694703 | 69/70 |
| ap2a2 | NM_012305 | NP_036437 | 71/72 |
| gpsm2 | NM_013296 | NP_037428 | 73/74 |
| Gpx3 | NM_002084 | NP_002075 | 75/76, 98 |
| erdr1 | NM_133362 | NP_579940 | 77/78 |
| tmod1 | NM_003275 | NP_003266 | 79/80 |
| cnbp1 | NM_003418 | NP_003409 | 81/82 |
| Prdm16 | NM_022114 | NP_071397 | 83/84 |
| | NM_199454 | NP_955533 | 85/86 |
| | | | Consensus: 97 |
| hdac1 | NM_004964 | NP_004955 | 87/88 |
| ski | NM_003036 | NP_003027 | 89/90 |
| Hoxb4 | NM_024015 | NP_076920 | 91/92# |

### EXAMPLE 3

### Validation

To validate the candidate genes identified in the above primary screen, additional independent experiments (n=4, unless indicated) were performed using the same 96 well plate protocol described in FIG. 1B. A summary of these results is provided in FIG. 5B. From left to right and top to bottom, genes are presented based on the level of statistical significance at 16 weeks (from highest to lowest) reached in these experiments: Hoxb4 (p=9.5×10⁻⁹) (control); Ski (p=1.6×10⁻¹⁰); Hoxb4 (p=9.5×10⁻⁹); Smarcc1 (p=8.5×10⁻⁸); Vps72 (p=2.4×10⁻⁷); Fos (p=3.2×10⁻⁷); Trim27 (p=5.1×10⁻⁷); Sox4 (p=1.0×10⁻⁶); Klf10 (p=1.8×10⁻⁶); Prdm16 (p=4.0×¹⁰⁻⁶); Erdr1, Tcfec, Sfpi1, Zfp472 and Hmgb1 (all between p=1.1 to 8.8×10⁻⁴); Cnbp, Pml and Xbp1 (p=0.001); Hnrpdl (p=0.002) and Hdac1 (p=0.015). Thus, all of the 18 candidates were confirmed (p≦0.05), for a positive predictive value (PPV) of 100%.

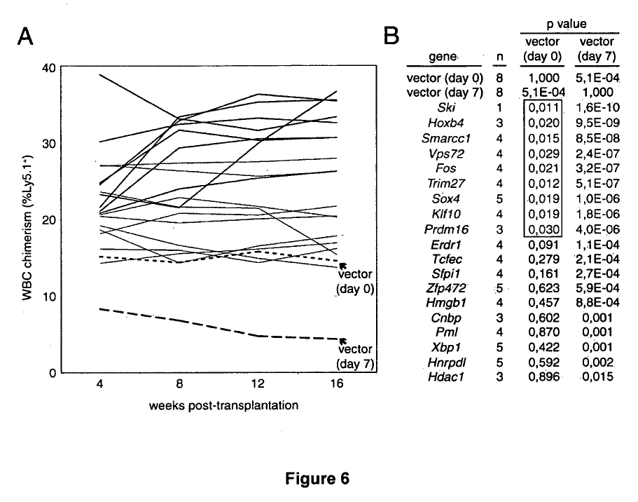

The design of the screen and validation protocol included an assessment of the reconstitution activity of HSCs isolated at the end of the infection—prior to the initiation of the 7 day culture-the so-called “day 0” time point (FIG. 1B). In the case of the negative control experiments, performed with the pKOF vectors alone, peripheral blood reconstitution was observed at 14.4±2.2% in recipients transplanted 16 weeks earlier. This value provides a reliable estimation of the level of HSC activity present at the initiation of the 7 day culture. Based on this, it is possible to identify genes that provide a net increase in HSC activity above that measured at the initiation of the culture from those which do not. In that respect, Hoxb4 is a prototype since transduced HSCs show a net expansion of 1 to 2 logs in short term cultures. The following genes were significantly higher than vector at day 0: Ski, Sox4, Smarcc1, Vps72, Fos, Trim27, Klf10 and Prdm16 (FIG. 6, dotted lines on panel A and boxed values in panel B), indicating a possible ex vivo expansion of HSCs to levels above input numbers, as does Hoxb4.

### EXAMPLE 4

### Evidence that Some Candidates Operates in a Non-Cell Autonomous Manner

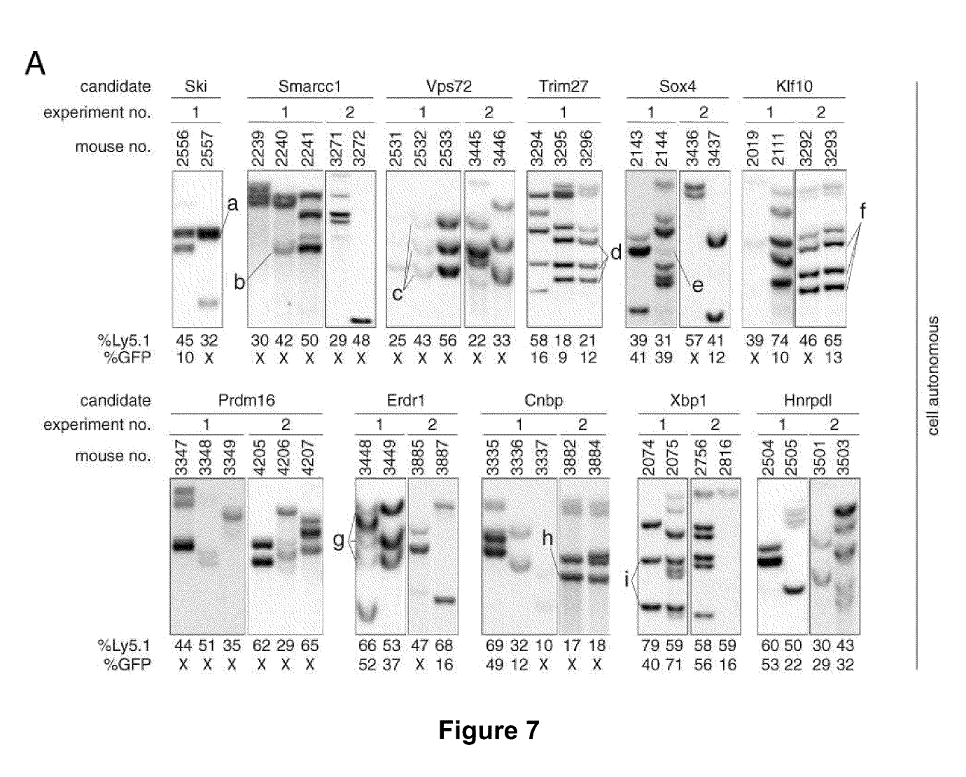

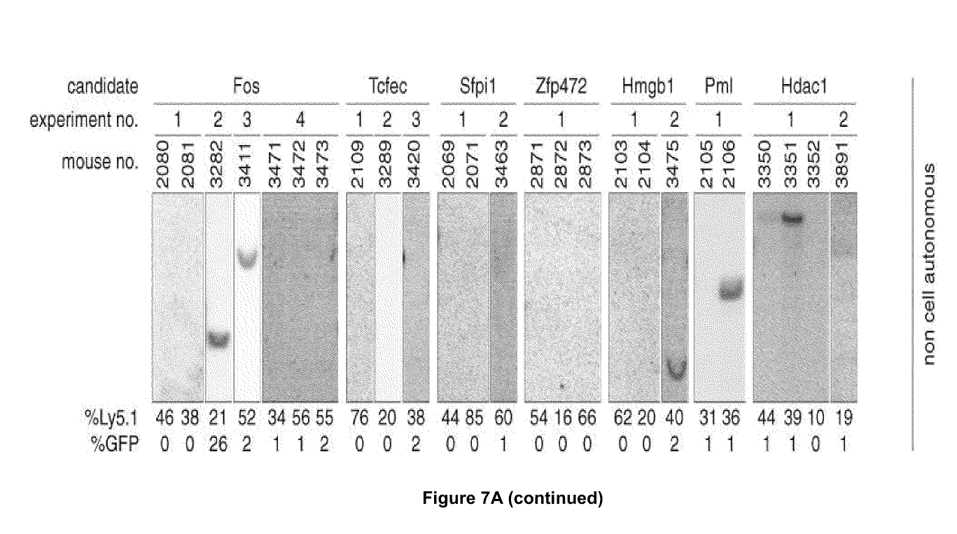

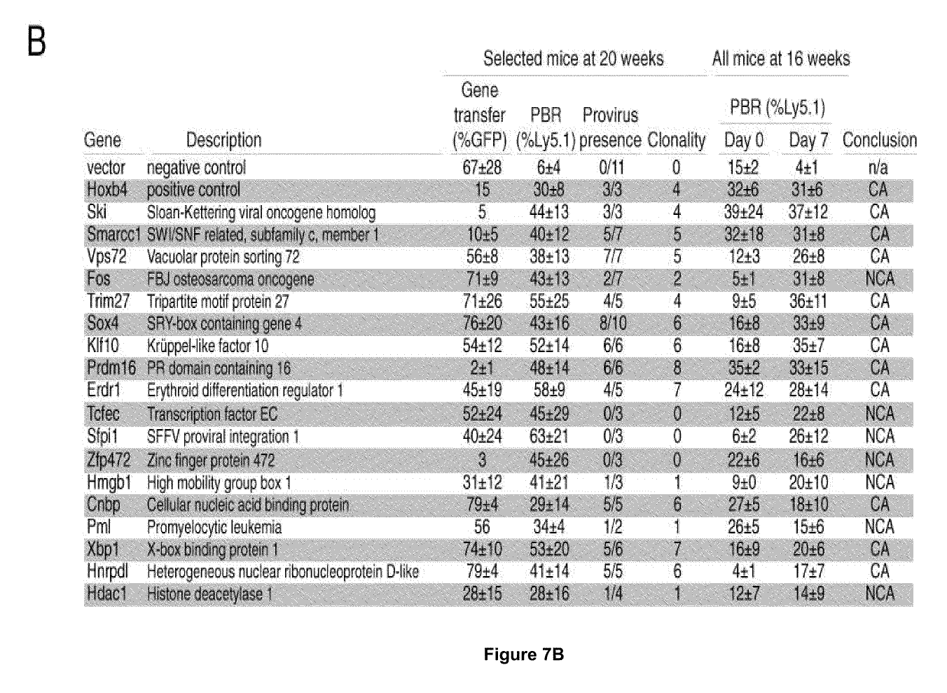

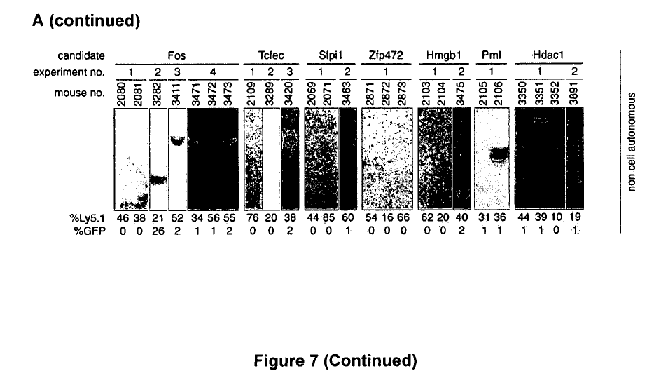

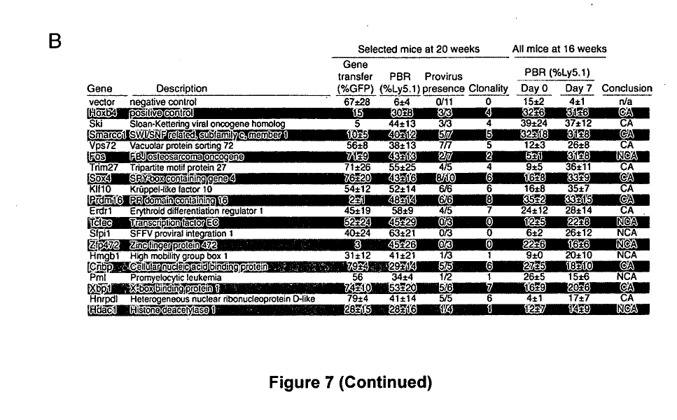

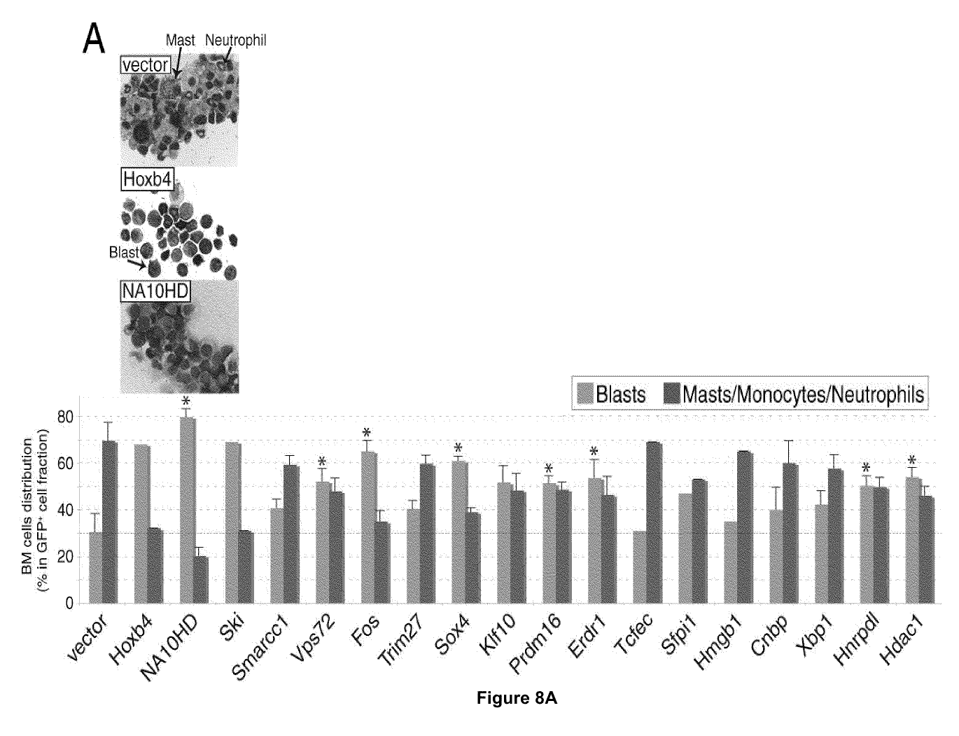

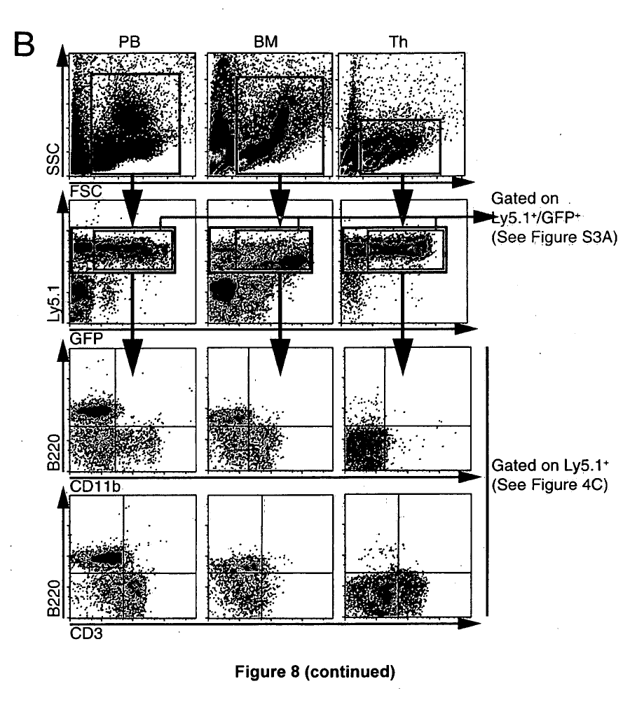

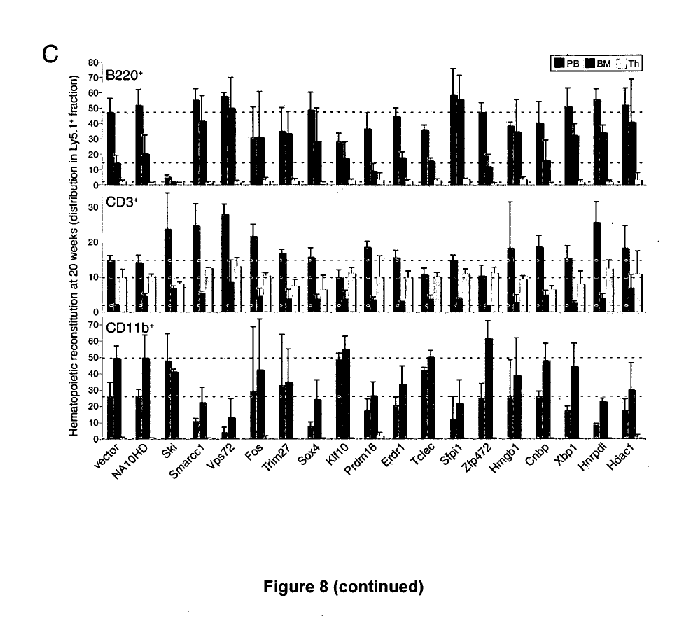

The 7-day ex vivo culture inherent to the screening strategy (FIG. 1B) should, provide sufficient time for extrinsic factors to impact on HSC expansion (13). Based on this, it is possible that non-transduced HSCs would respond favorably to a series of factors secreted by—or present on—adjacent cells (e.g., viral producers or other progenitors) thereby conferring a competitive advantage to all (transduced and untransduced) HSCs in these cultures (Ly5.1⁺). To address this possibility, we analyzed the hematopoietic system of selected recipients that were highly reconstituted (between 10 to 85% of Ly5.1⁺ cells) at 20 weeks post-transplantation, a time point deemed sufficient such that reconstitution is strictly derived from so-called long-term HSCs (LT-HSCs) (27). The presence of the expected proviral DNA in the appropriate reconstituted tissues was first verified. This constitutes a necessary attribute for cell autonomous effects. For 11 of the 18 nuclear genes identified in the present screen, namely Ski, Smarcc1, Vps72, Trim27, Sox4, Klf10, Prdm16, Erdr1, Cnbp, Xbp1 and Hnrpdl, proviral DNA was observed in 58 of the 65 recipients (89%) that were analyzed at this late timepoint (FIG. 7A, 2 upper panels; FIG. 7B, 5th column). Considering that gene transfer efficiency was on average at 50% for the entire gene set and 55% for these 11 genes (FIG. 4, 2nd column) and that a limiting number of transduced HSCs were transferred to each recipient, this observation on its own is compatible with these genes intrinsically enhancing HSC activity. Furthermore, the analysis of proviral DNA integration patterns in selected hematopoietic tissues from these mice revealed that several different clones with long-term reconstitution ability contributed to hematopoiesis for each of these 11 nuclear factor genes (FIG. 7A). This was true for different recipients within the same experiments and, obviously from different experiments, thus supporting that insertional mutagenesis is not responsible for these results. In several instances, the same proviral integrations in the DNA from 2 different mice reconstituted by cells derived from the same culture could be identified, demonstrating that LT-HSC self-renewal has occurred in these cultures (see a-i in FIG. 7A).

Interestingly for 7 of the 18 validated nuclear genes, namely Fos, Hmgb1, Tcfec, Sfpi1, Zfp472, Hdac1 and Pml, it was found that only a minority of the highly reconstituted recipients (between 10 to 85% of Ly5.1⁺ cells at 20 weeks post transplantation; FIG. 7A third panel) contained integrated proviral DNA in their hematopoietic tissues. This observation raises the possibility of a non-cell autonomous activity in cultures in which these HSCs were kept prior to transplantation. A detailed evaluation of these recipients is provided in FIG. 7B to stand comparison with mice that were reconstituted with cells transduced with each of the 11 genes described in the previous paragraph (also presented in this Figure as the “cell autonomous” group). First and foremost, gene transfer efficiencies were similar between both groups or around 40-50% (mean values). Second, the repopulation activity for 4 of the 7 genes with presumed non-cell autonomous activity was enhanced by the 7-day culture prior to transplantation described in FIG. 1B [FIG. 7B, compare % Ly5.1 day 0 (7th column) vs day 7 (8th column) for Fos, Tcfec, Sfpi1 and Hmgb1]. Fos represents a notable example for this: with an initial gene transfer above 70%, it was found that recipients reconstituted with HSCs prior to the 7-day culture were repopulated by Ly5.1⁺ cells at 5±1% whereas, following the 7-day culture, this number increased to 31±8% in 4 independent experiments with 2 mice per experiment at day 0, and 3 at day 7 (FIGS. 4 and 7B). As presented in FIG. 5B, Tcfec, Sfpi1 and Hmgb1 show a similar trend.

Thus, the combination of results from proviral integrations and hematopoietic reconstitution analyses support the existence of 2 broad groups of effectors for the nuclear gene candidates, one which includes 7 genes that appear to extrinsically support enhanced HSC activity and another of 11 genes which seem to provide intrinsic contribution.

### EXAMPLE 5

### Impact of Validated Candidates on HSC Differentiation

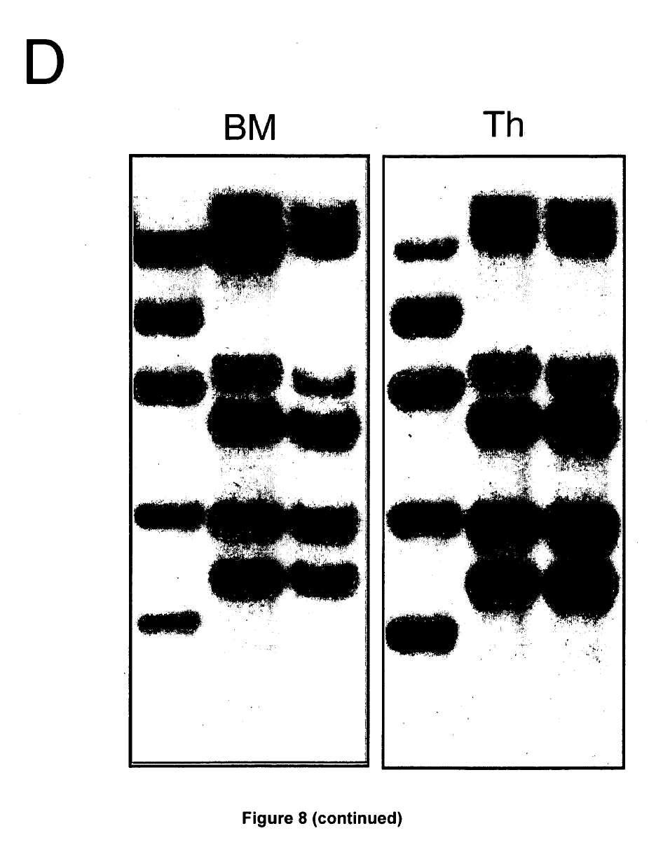

There is growing evidence to suggest that HSC self-renewal involves the active repression of a differentiation program that is coupled to cell division (14). In support of this, the present inventors recently found that Hoxb4 or NA10HD-transduced HSCs, which actively undergo in vitro self-renewal divisions, show evidence of differentiation arrest [FIG. 8A; (14)]. The newly validated candidates were investigated to determine if they behaved similarly. To achieve this, the cytological characteristics of transduced and sorted HSCs was analyzed after a 7-day in vitro culture period (prior to their transplantation). In this context, cultures initiated with control vector-infected HSCs contained differentiated cells in a proportion of 70±8%. These included neutrophils, monocytes and mast cells (FIG. 8A, arrows in upper left panel with summary of results in histogram: grey bars=undifferentiated cells or blasts, and dark grey bars=differentiated cells). Conversely, cellular differentiation was reduced in cultures initiated with HSCs transduced with most of the newly validated candidates (FIG. 8A). The increase in the proportion of undiffentiated to differentiated cells was most important for Ski, Vps72, Fos, Sox4, Klf10, Prdm16, Erdr1, Hnrpdl and Hdac1 when compared to cultures initiated with HSCs infected with the control virus.

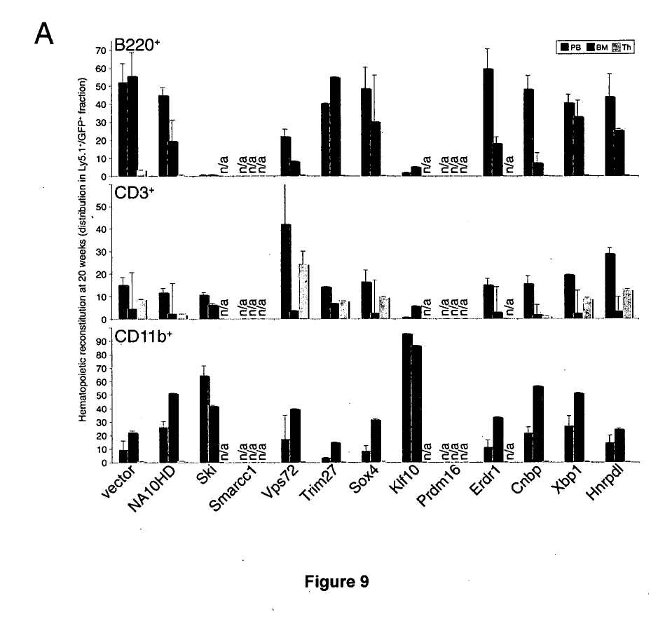

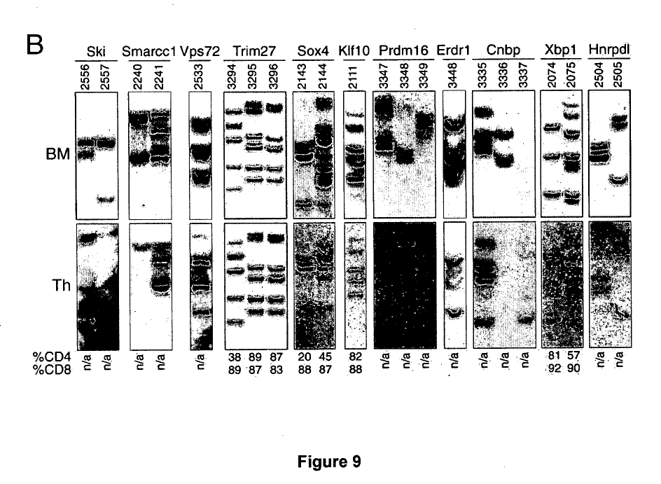

The in vitro differentiation arrest displayed by Hoxb4 or NA10HD-transduced HSCs is eventually reverted following their transplantation in vivo. Thus, depending on the environment, these 2 genes can either interfere (e.g., in vitro in the presence of growth factors) or not (e.g., in vivo under steady state conditions) with HSC differentiation. To determine if the newly identified regulators of HSC activity are similarly permissive to HSC differentiation in vivo, 4 different approaches were used. First, the general health, spleen size and bone phenotype (white vs red) of each recipient was evaluated. Except for recipient of Prdm16-transduced cells, which eventually developed splenomegaly, white femurs and myeloproliferation at 20 weeks (data not shown), none of the mice transplanted with cells expressing the 17 other nuclear genes ever presented this, or any other, hematological phenotype. Second, microscopic evaluation of bone marrow and spleen cytological preparations derived from representative mice for each gene was performed. Results from these analyses were normal for all groups, except for the Prdm16 cohort, which showed an excess of poorly differentiated myeloid cells in their bone marrow and for the Ski cohort in which the number of lymphocytes in the bone marrow was reduced. Besides recipients of Prdm16-transduced cells, spleens were never infiltrated with myeloid cells nor did they include enhanced numbers of erythroblasts. To confirm this, a third approach consisting in performing FACS analysis on donor-derived (Ly5.1⁺) cells from selected recipients in which reconstitution was well above background values (see FIG. 7A for values) was devised. The results, presented in FIG. 8B for the peripheral blood, bone marrow and thymus of a representative mouse (Trim27) and summarized in FIG. 8C for all groups, largely confirmed microscopic evaluation. Indeed, except for recipients of Ski transduced cells which showed a marked reduction in B lymphocytes in their peripheral blood and marrow, with a compensatory increase in other cell types, most groups of mice showed either normal FACS profiles or presented some minor variations (detailed in FIG. 8C). This analysis was further extended by gating only on Ly5.1⁺/GFP⁺ cells with genes for which this was possible and ended with the same conclusion, except that Klf10 tended to act like Ski (FIG. 9). Finally, clonal analyses of recipients that were reconstituted with retrovirally marked cells (mostly from the 11 “cell autonomous genes”) were performed on bone marrow (mostly myeloid, erythoid and B cells) and thymus (less than 5% non-T cells). A representative result is presented in FIG. 8D for Trim27 which shows that identical clones contributed to the reconstitution of these 2 tissues, thus reinforcing the finding that these transduced HSCs remain competent in T cell differentiation although they displayed enhanced reconstitution activity. This finding with Trim27 can be extended to all other genes but Ski, Prdm16 and Erdr1 where it cannot be certain that the same clone contributed to thymic and bone marrow reconstitution (see FIG. 9B).

Together, these results confirm that the majority of the genes identified in the screen conferred enhanced HSC activity without causing hematological disease nor profoundly altering cell differentiation at least until 20 weeks post-transplantation. Prdm16 was a notable exception.

### EXAMPLE 6

### Building a Network of HSC Activity

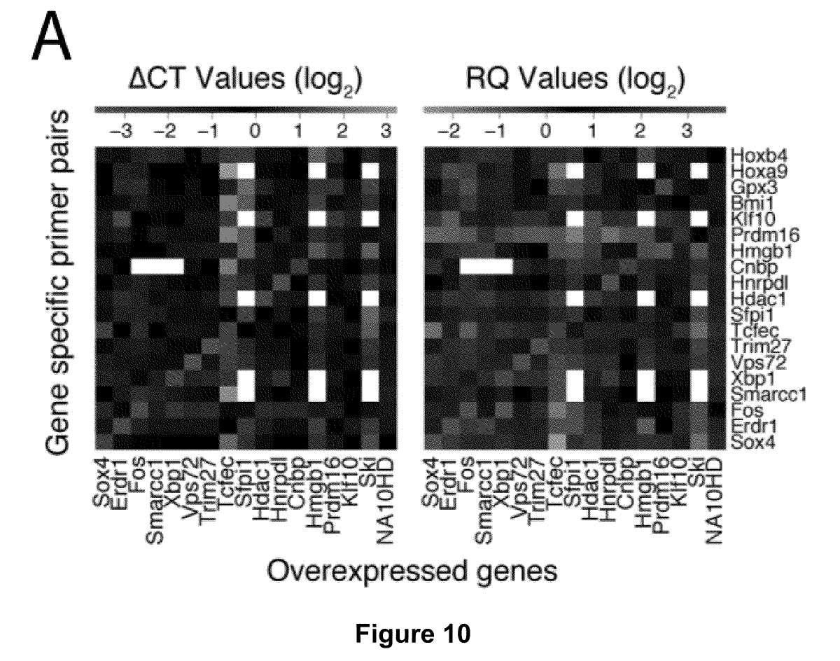

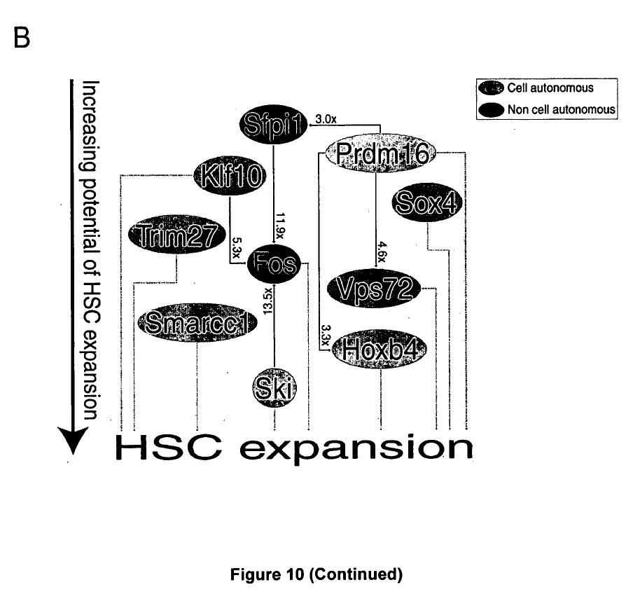

Epistatic studies were performed by analyzing transcription levels of all 18 nuclear genes identified in addition to known regulators of HSC SR, i.e., Hoxb4, Hoxa9, Bmi1 while overexpressing each of them individually, in a matrix-like manner to find any cross-regulation between these genes. Surprisingly, few genes significantly affected transcript levels of tested genes (≧3-fold; black solid arrows in FIG. 10B). Among them, Prdm16 was the most influent as it upregulated the expression of Hoxb4, known SR inducer, and Vps72, a newly identified HSC activity regulator with cell autonomous effect.

Moreover, some of these interactions occurred in the 2 groups of autonomy effectors mentioned above, e.g., Ski, Prdm16 and Klf10 have cell autonomous effect on HSC activity but also regulate factors that have a non-cell autonomous effect, i.e., Fos and Sfpi1.

### EXAMPLE 7

### Two Forms of Trim27

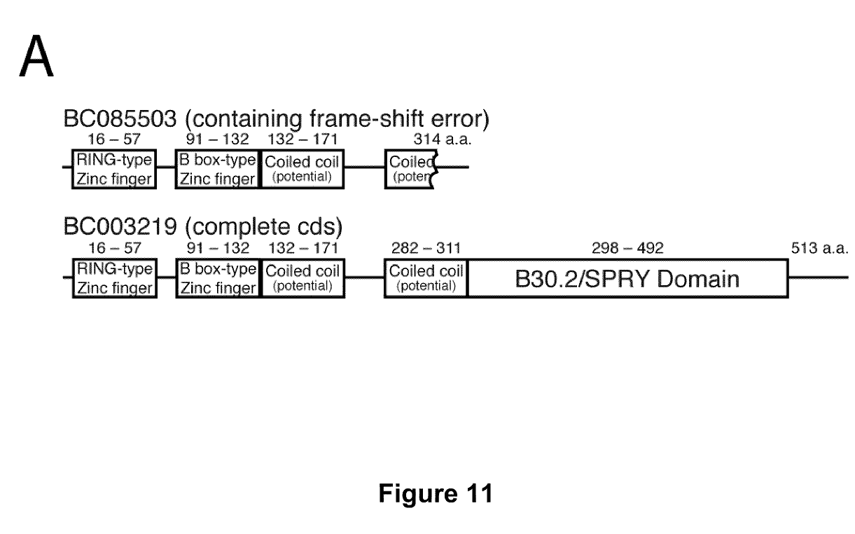

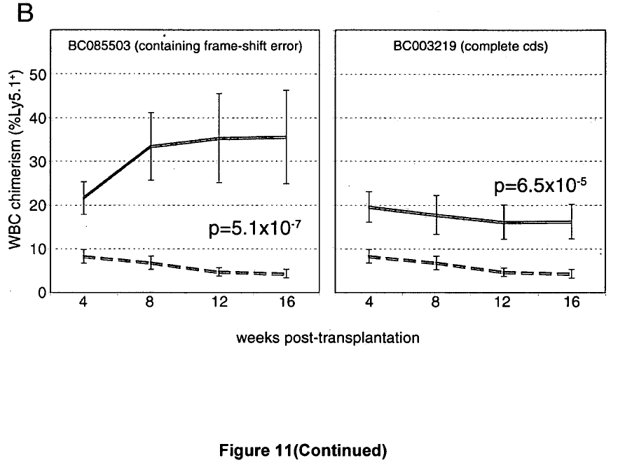

Two different forms of Trim27 have been tested in the competitive repopulation assay of this study. The first one, used in the primary screen, contains a frame-shift error (truncated form; accession number BC085503; FIG. 11A, upper panel) preserving intact only the RING, B-box and first Coiled-coil domains of the entire protein. The other form, latter recognized as the full-length form (accession number BC003219; FIG. 11A bottom panel) also contains the second Coiled-coil and the SPRY domains. The 2 FLAG-Trim27 polypeptides were detected at the expected size and the competitive repopulation assays revealed a different reconstitution potential by the different forms, the highest potential being held by the truncated form (FIG. 11B). Based on this, the second part of the second Coiled-coil domain in combination with the SPRY domain, seem to limit the potential of this gene in HSC expansion.

### EXAMPLE 8

### Clonal Analysis

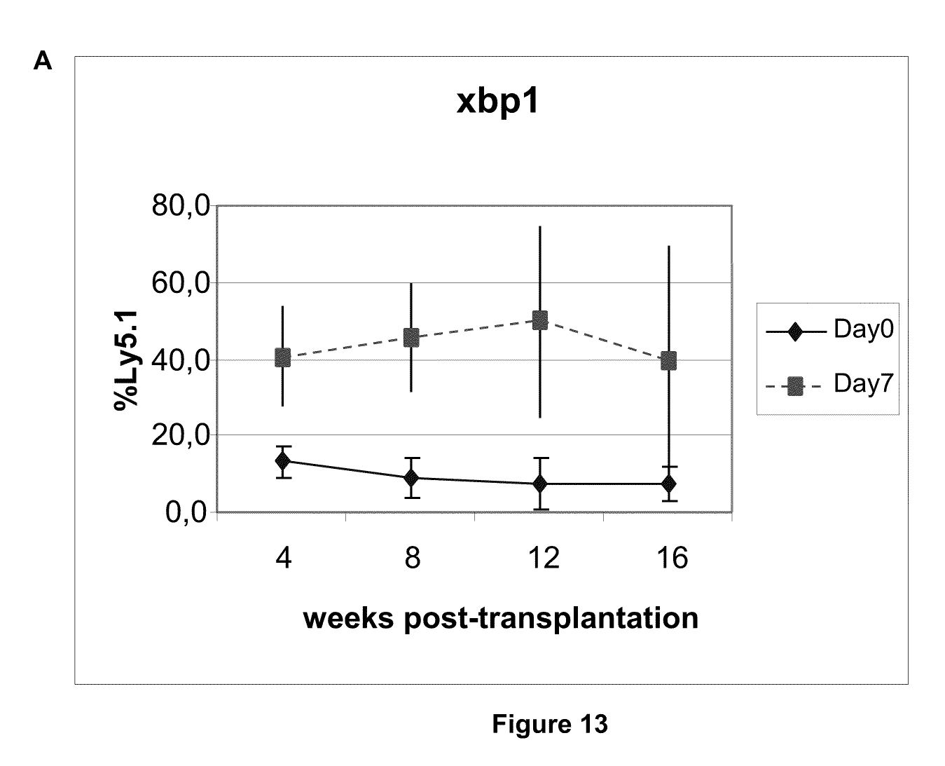

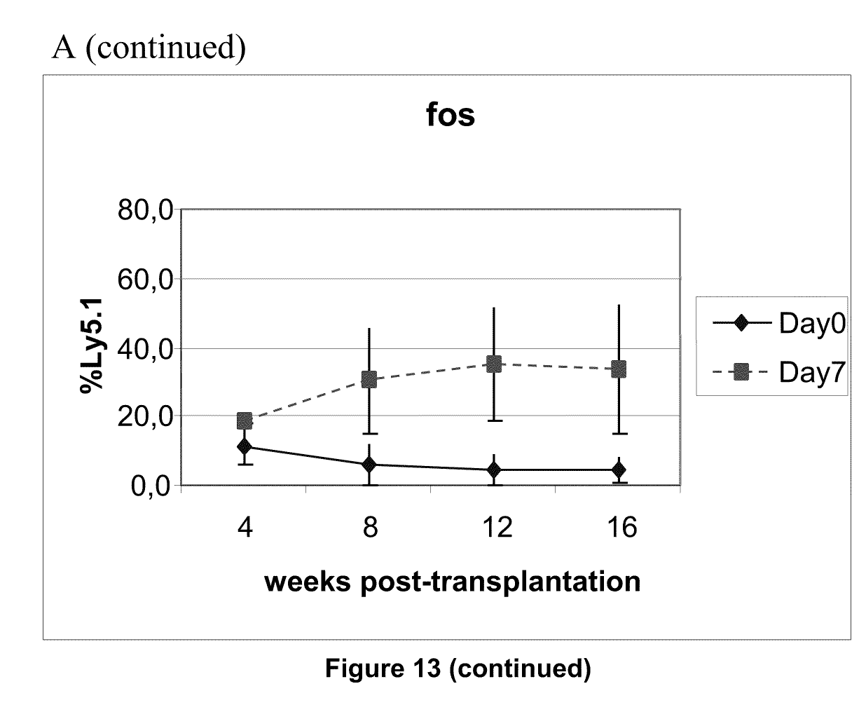

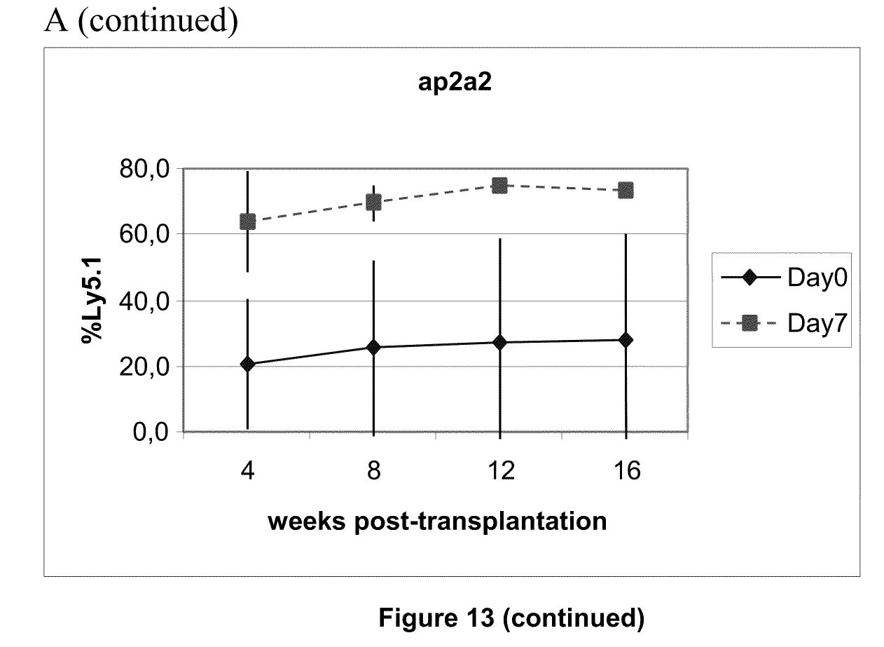

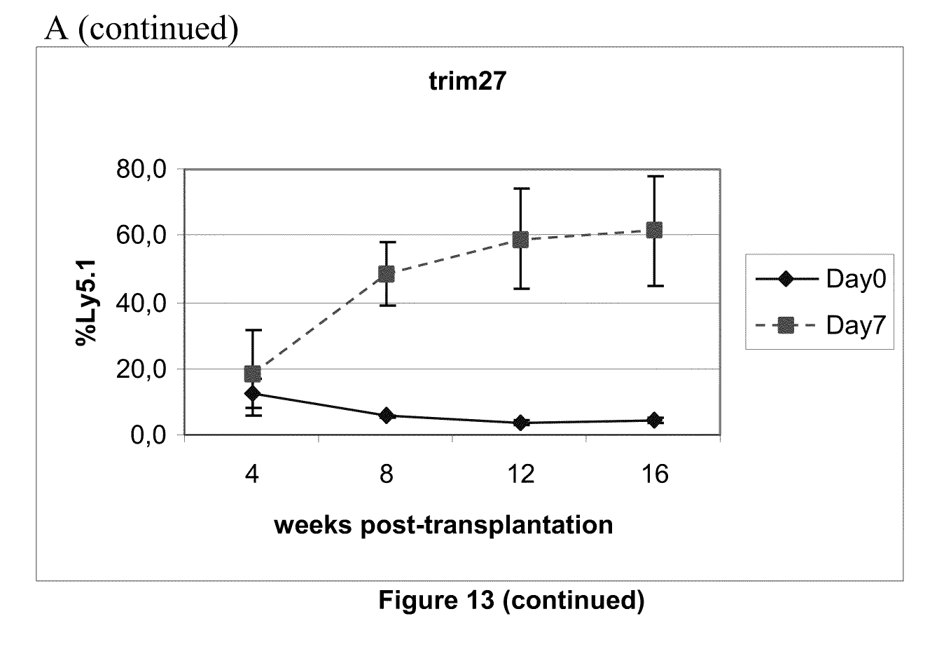

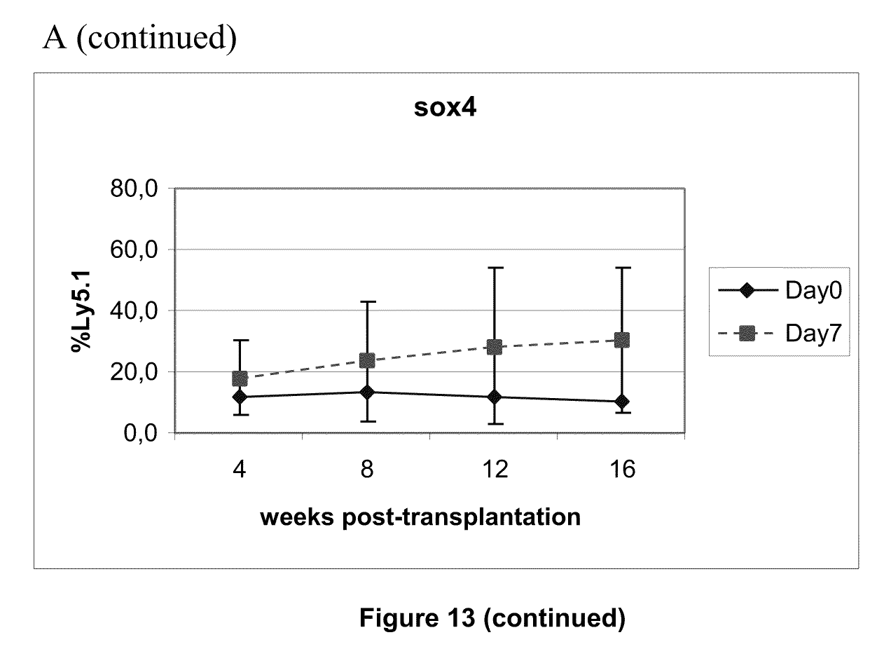

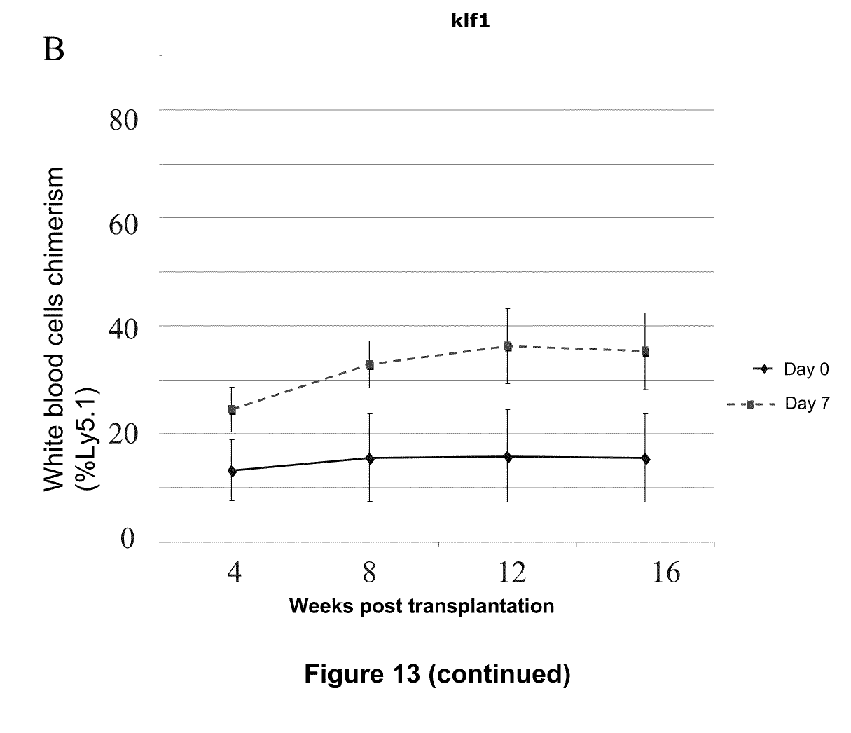

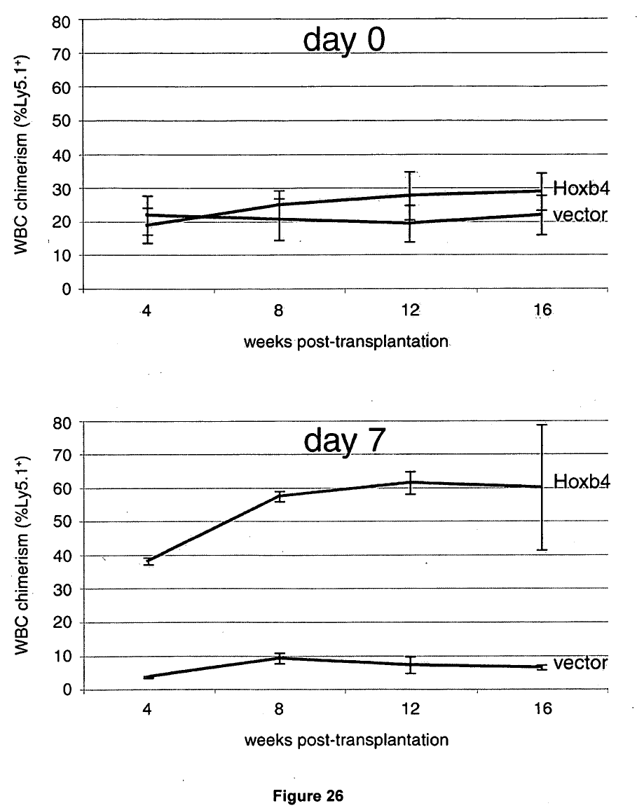

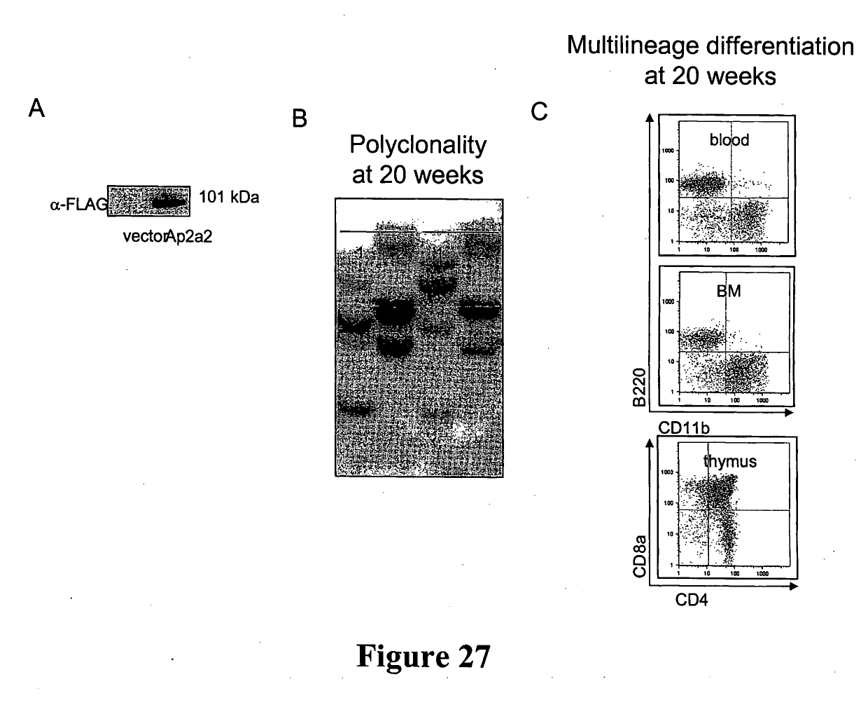

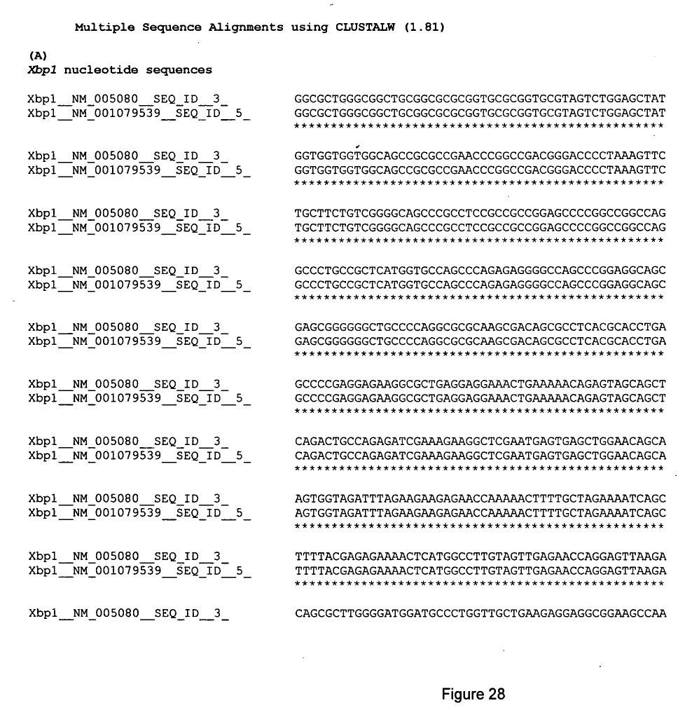

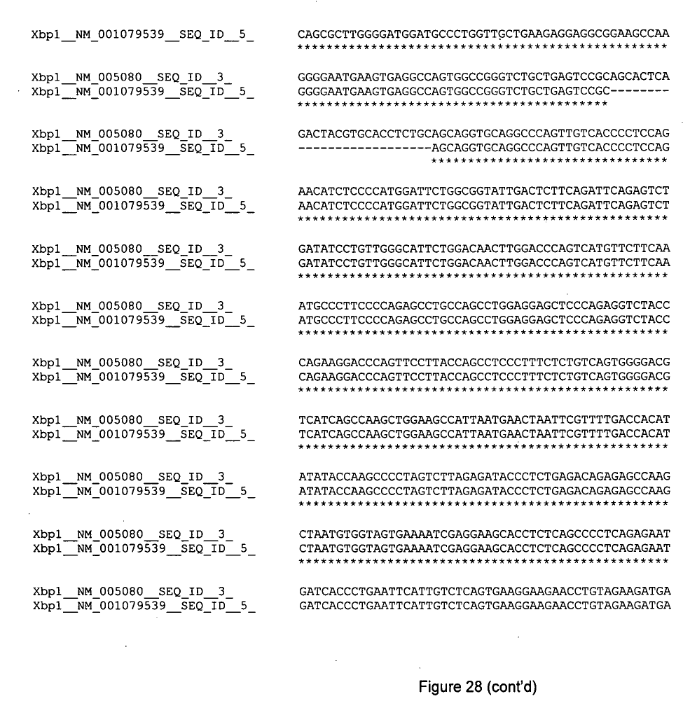

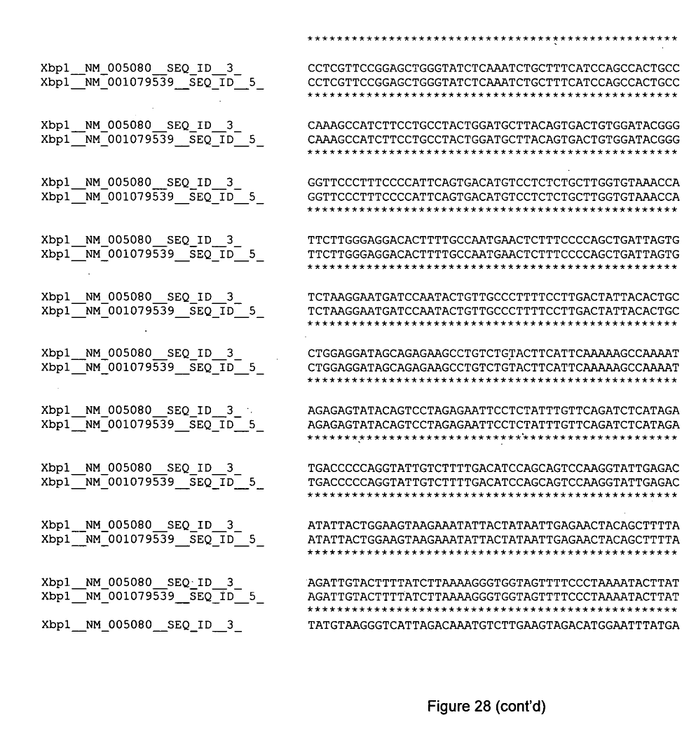

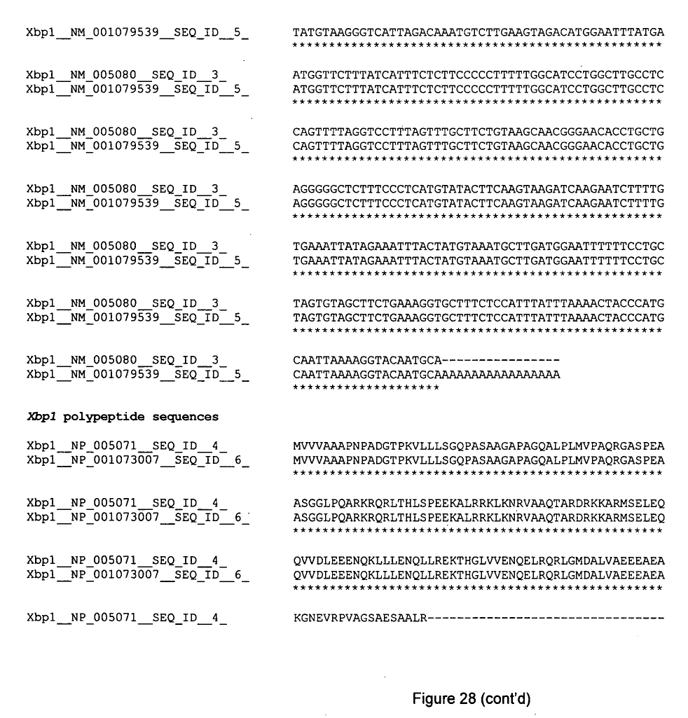

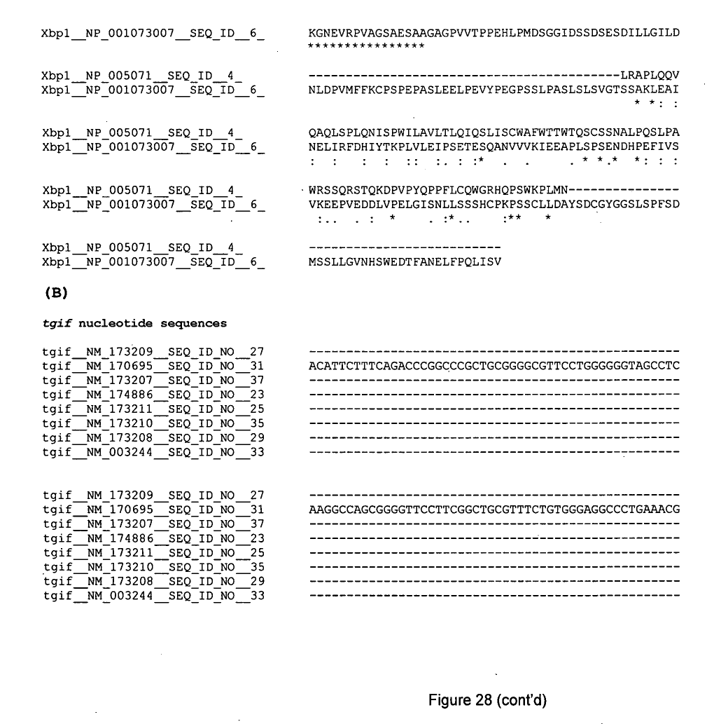

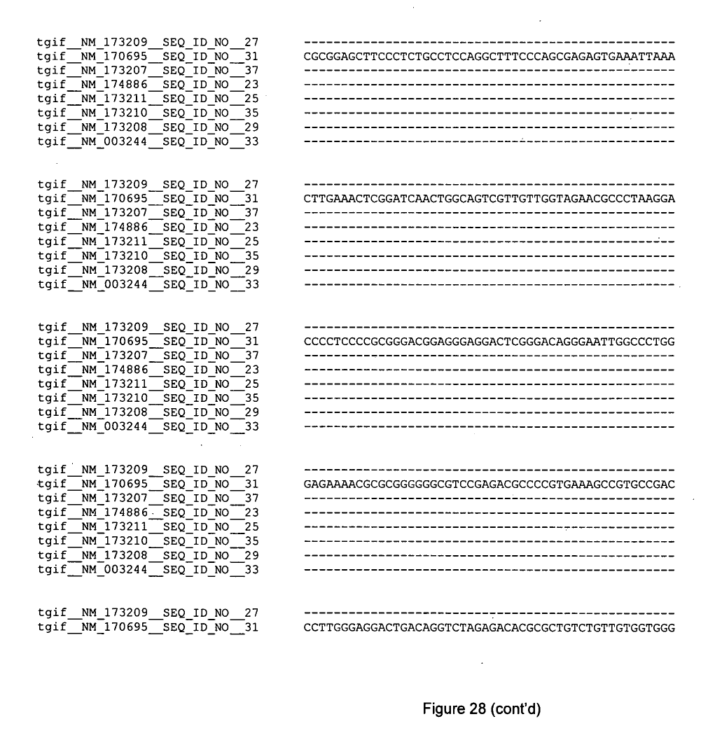

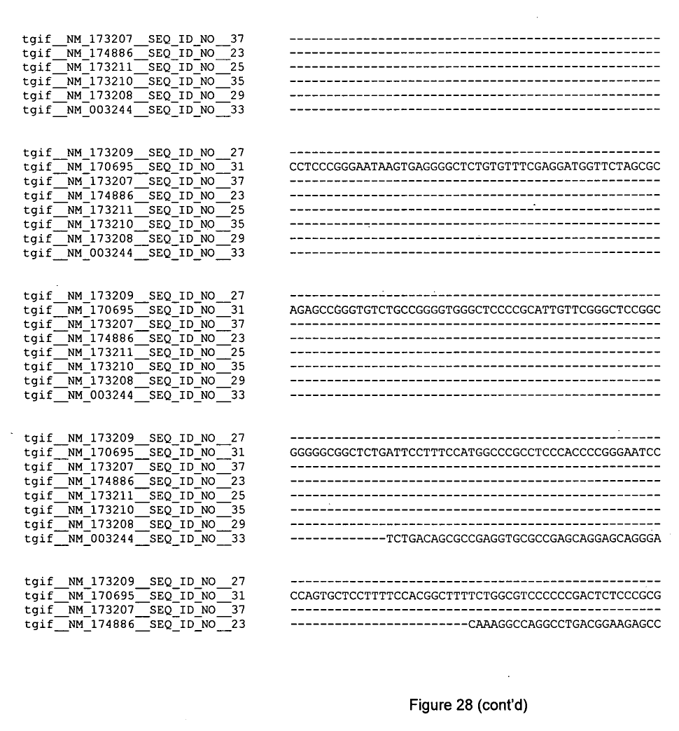

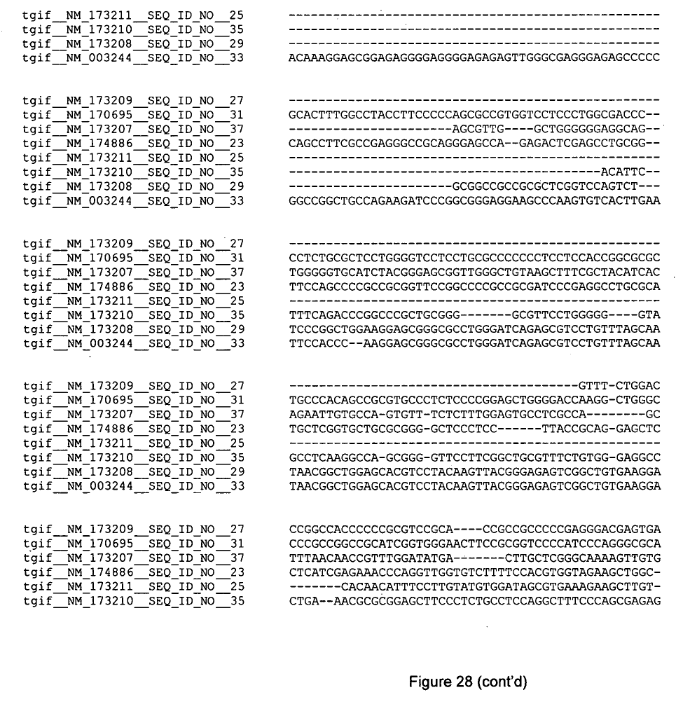

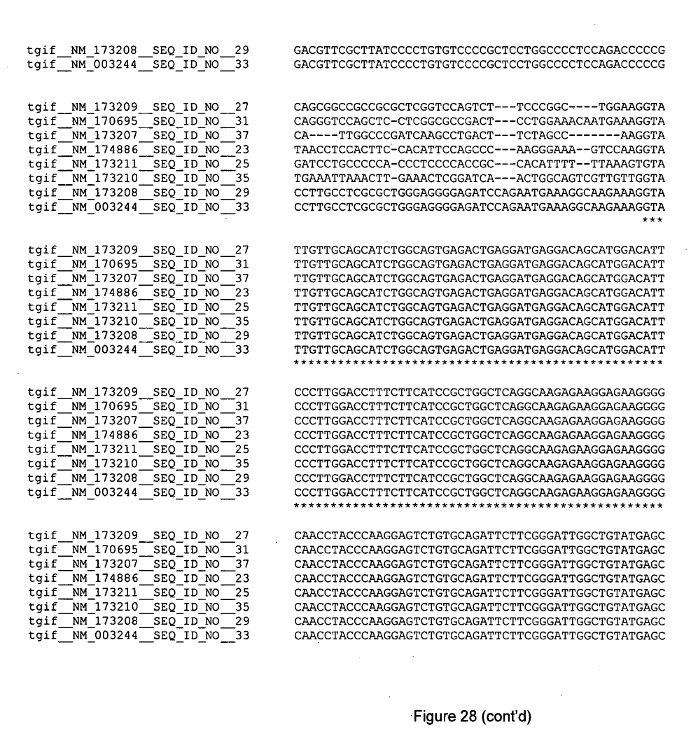

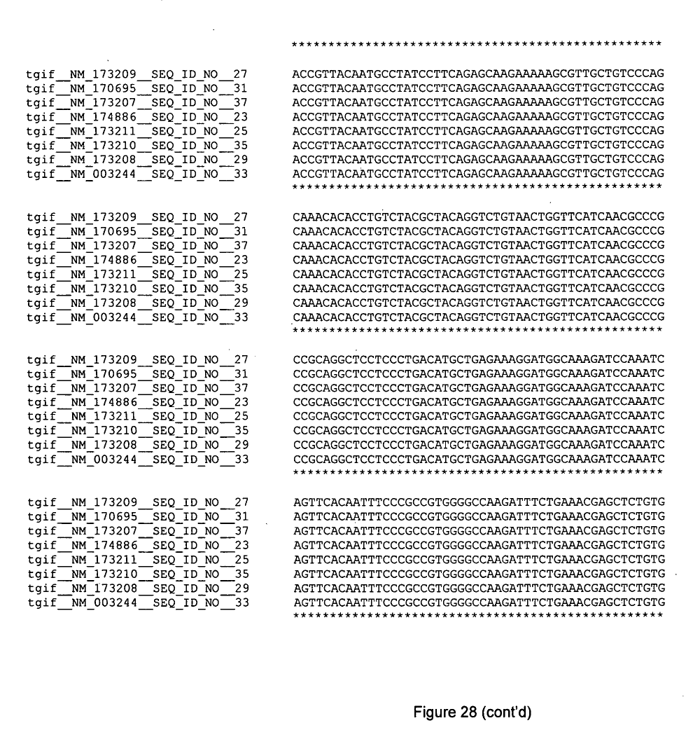

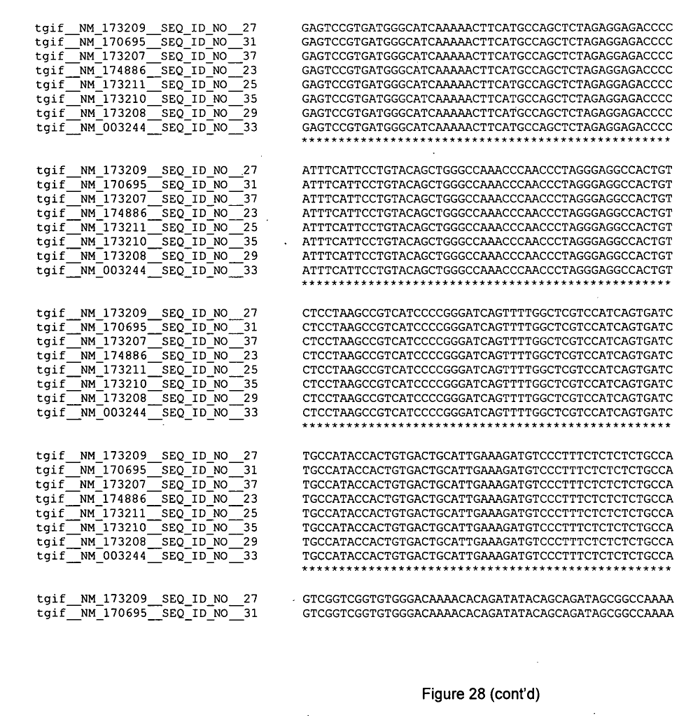

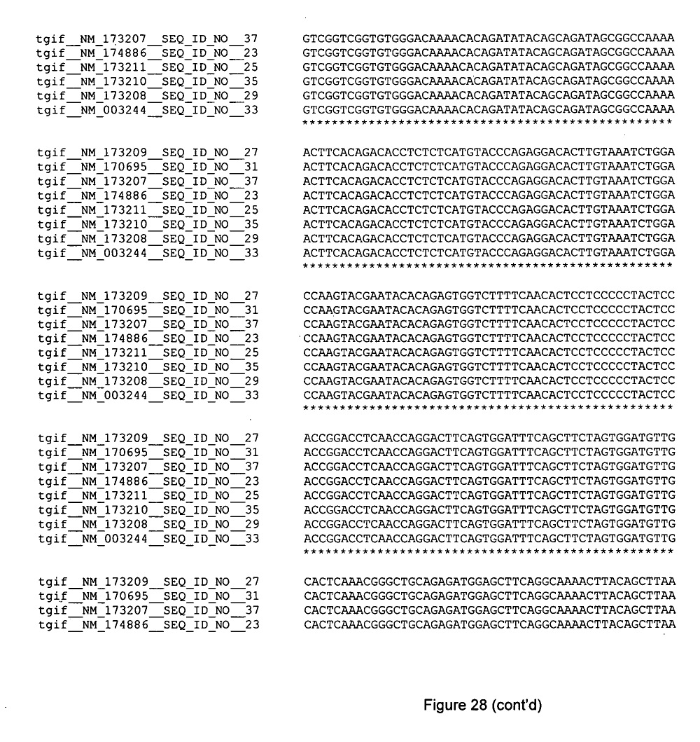

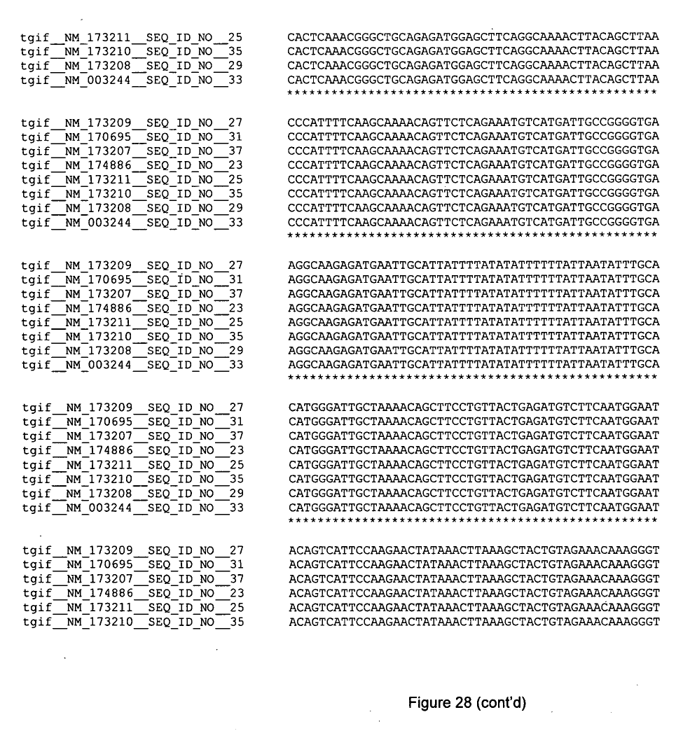

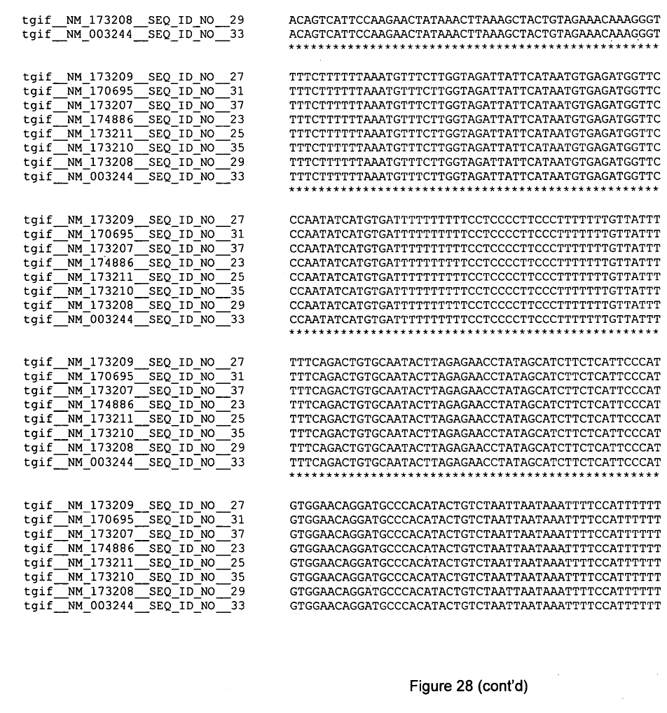

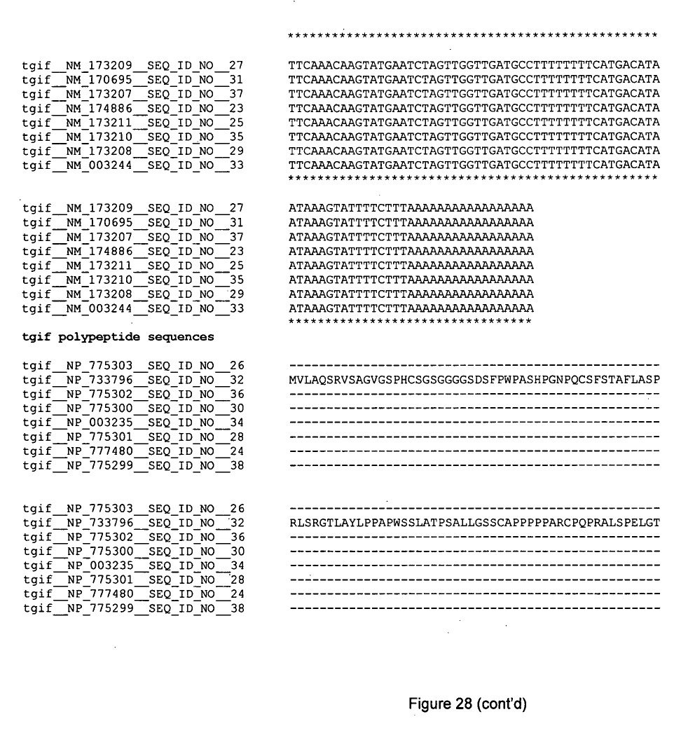

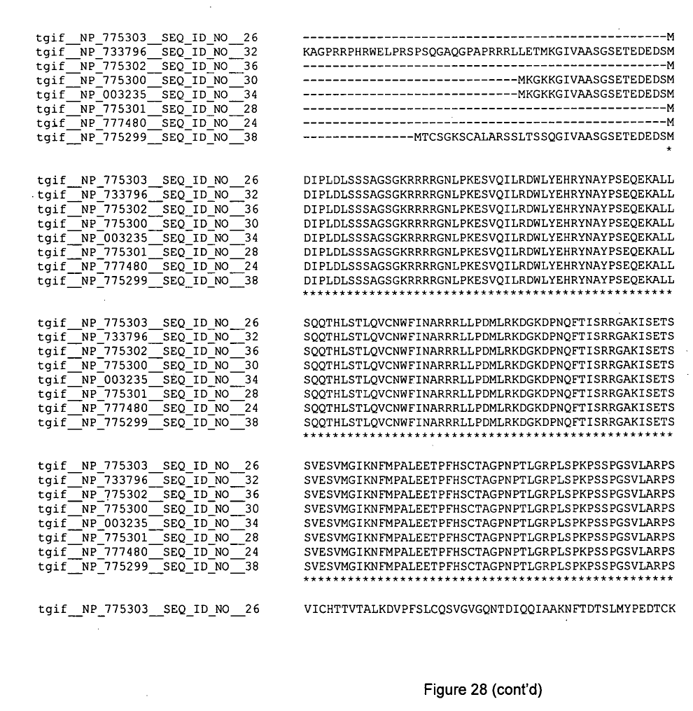

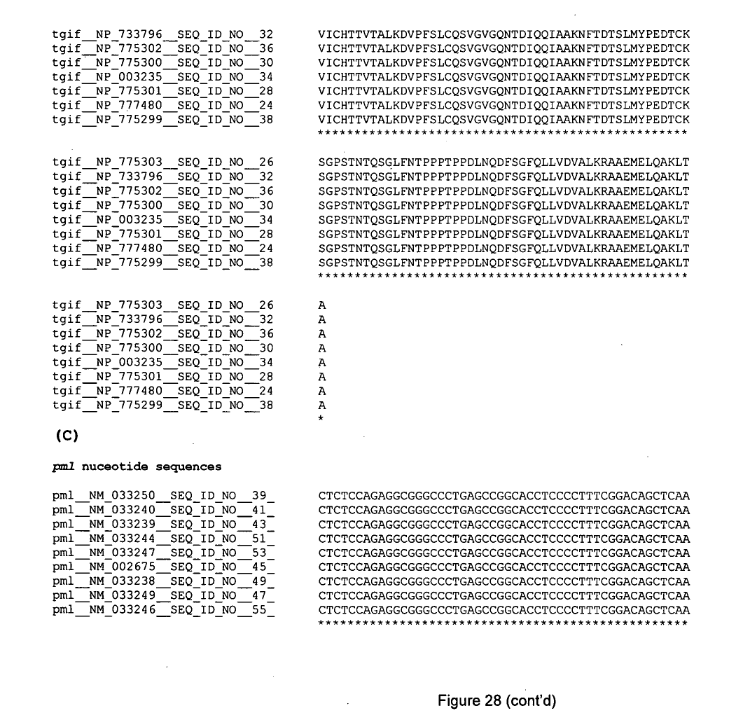

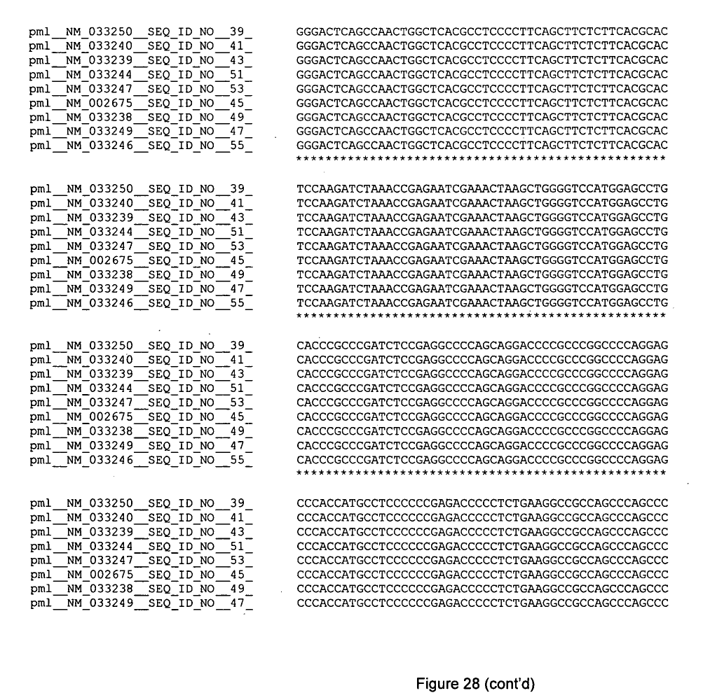

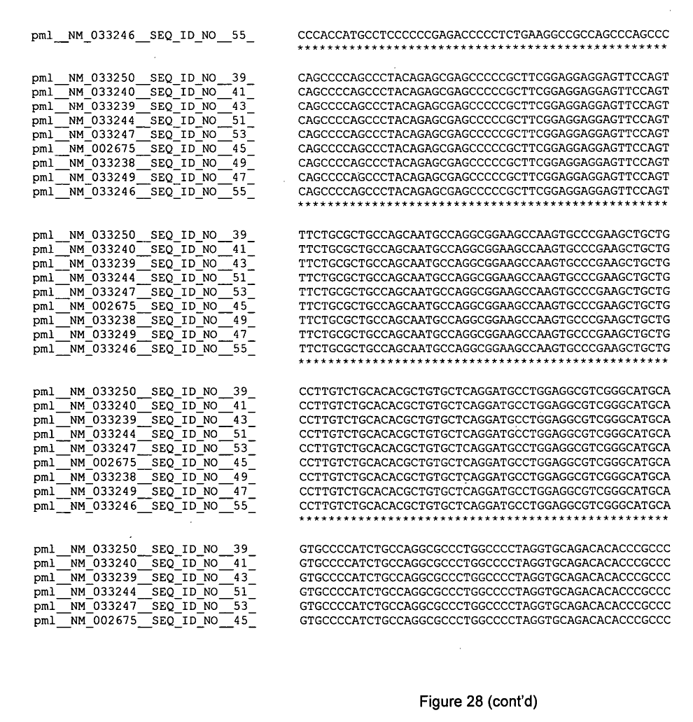

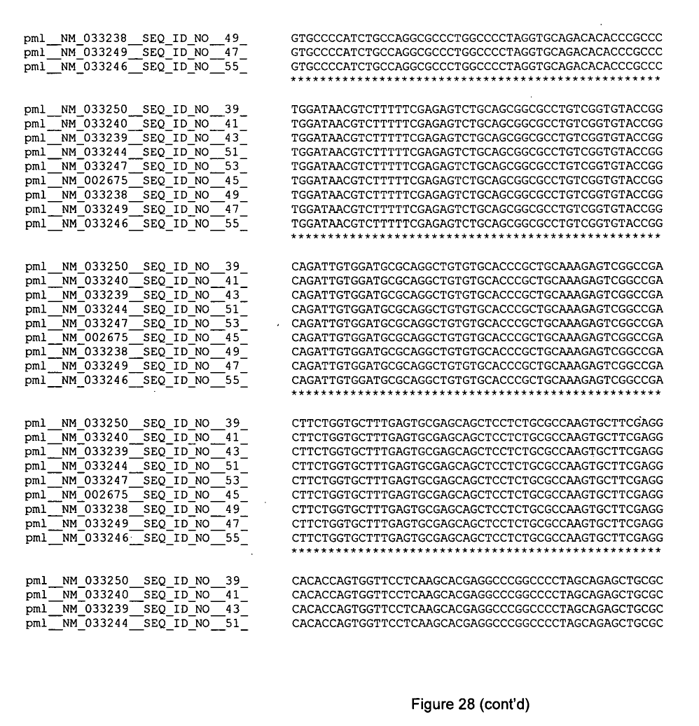

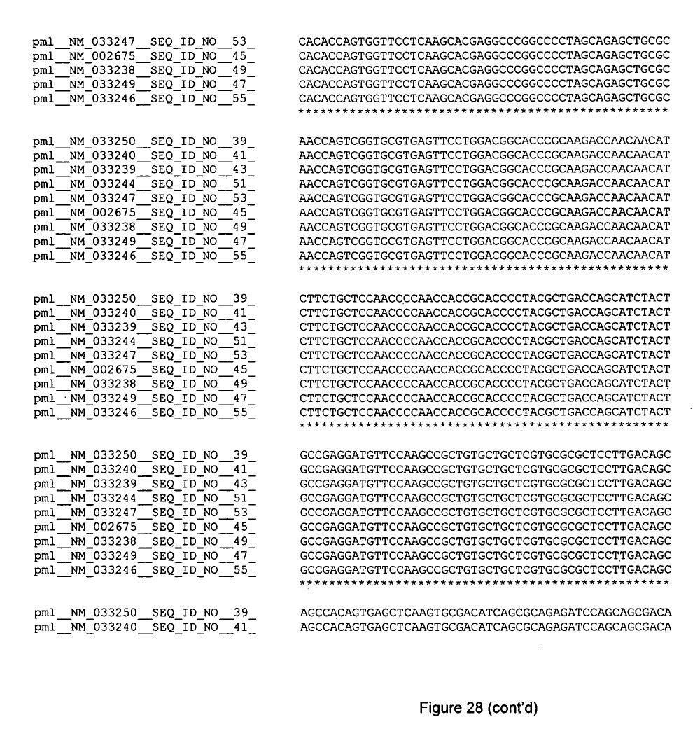

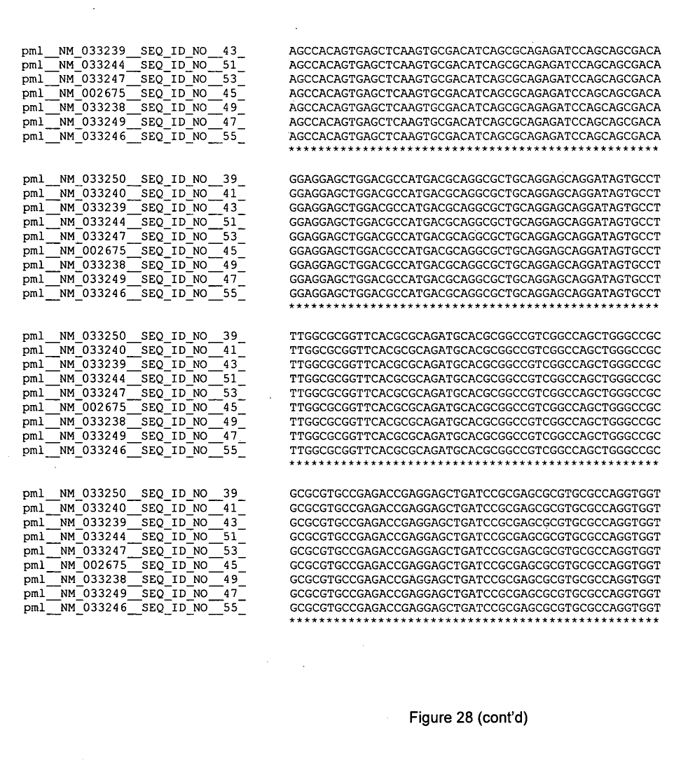

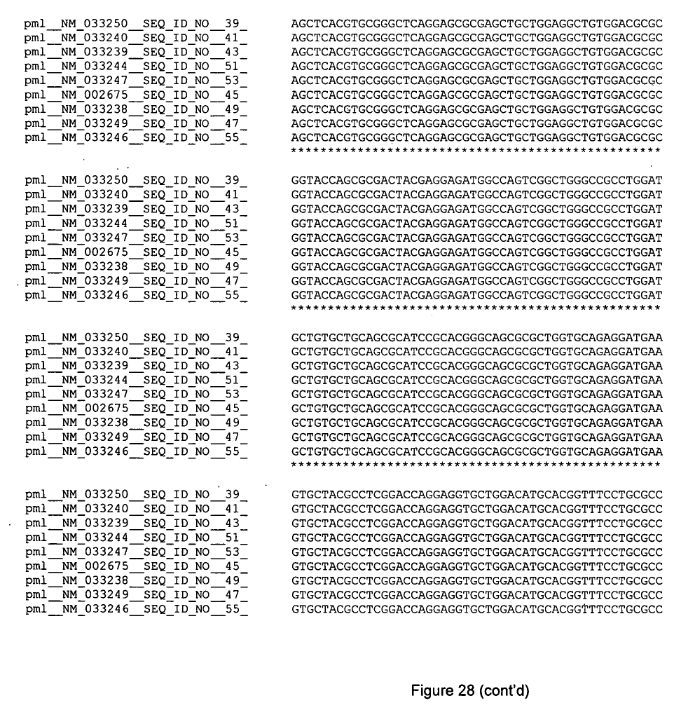

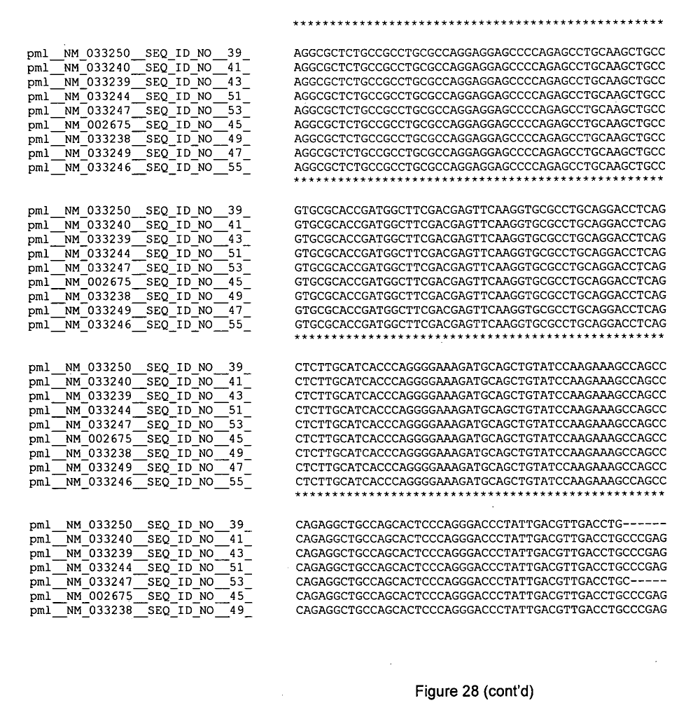

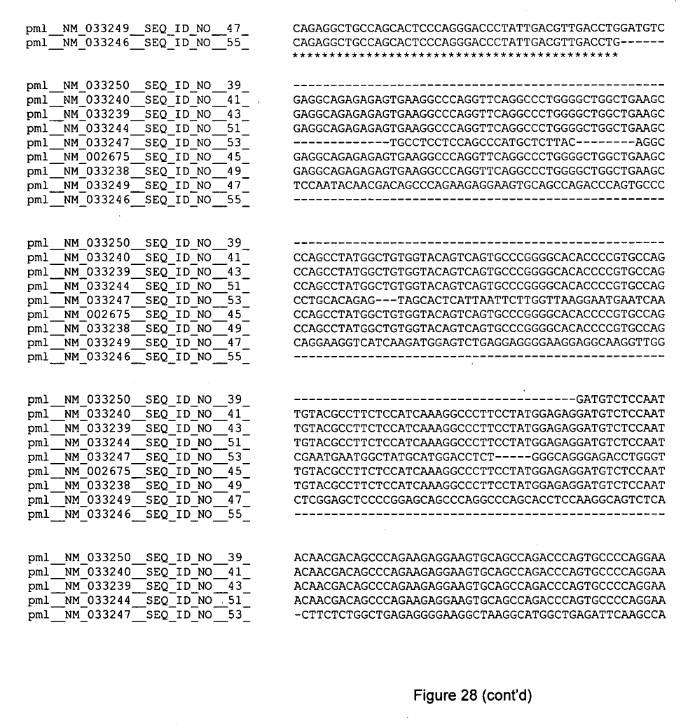

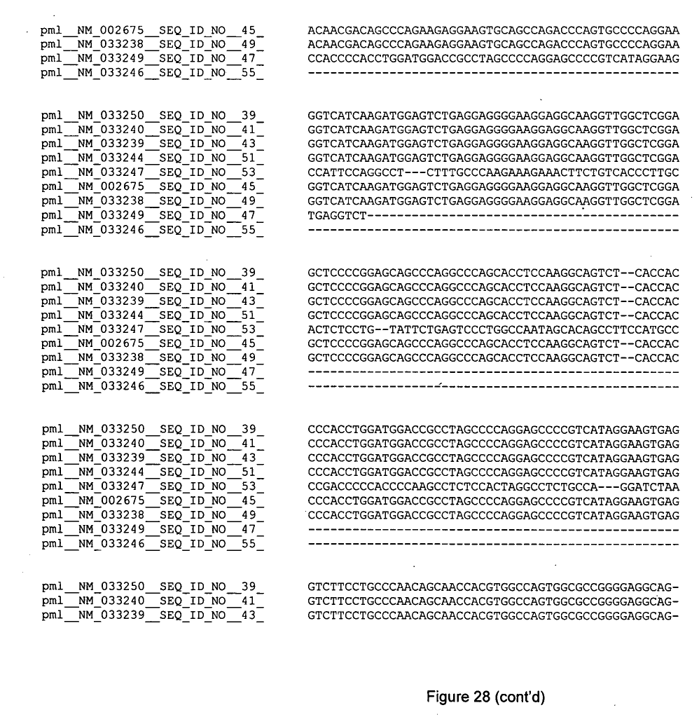

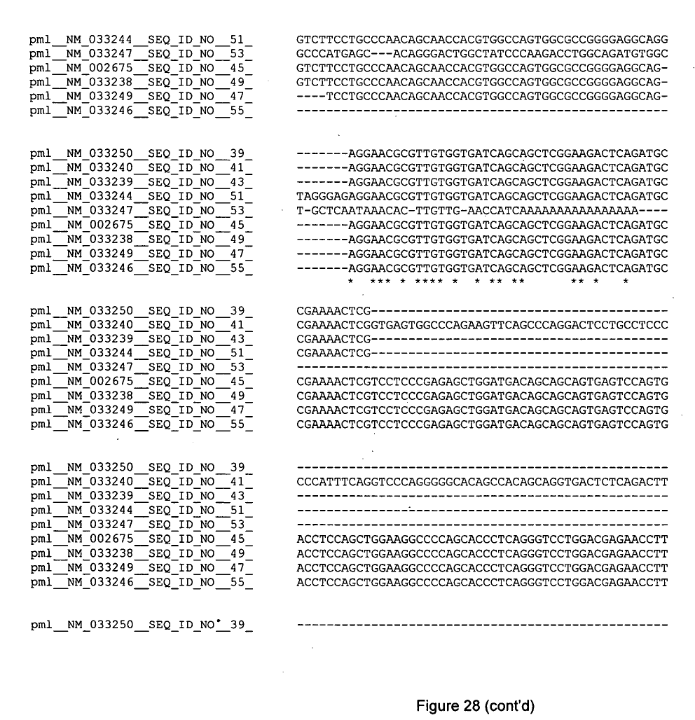

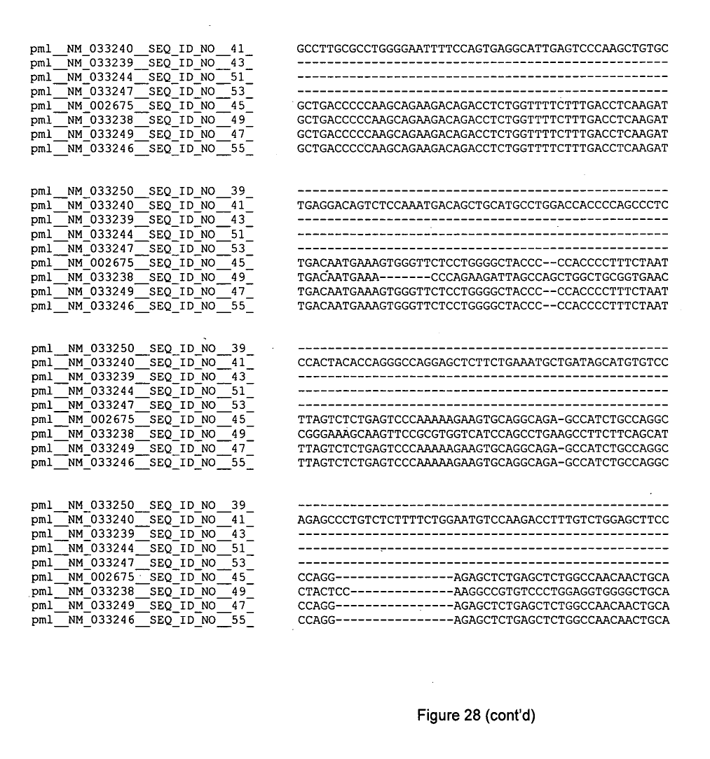

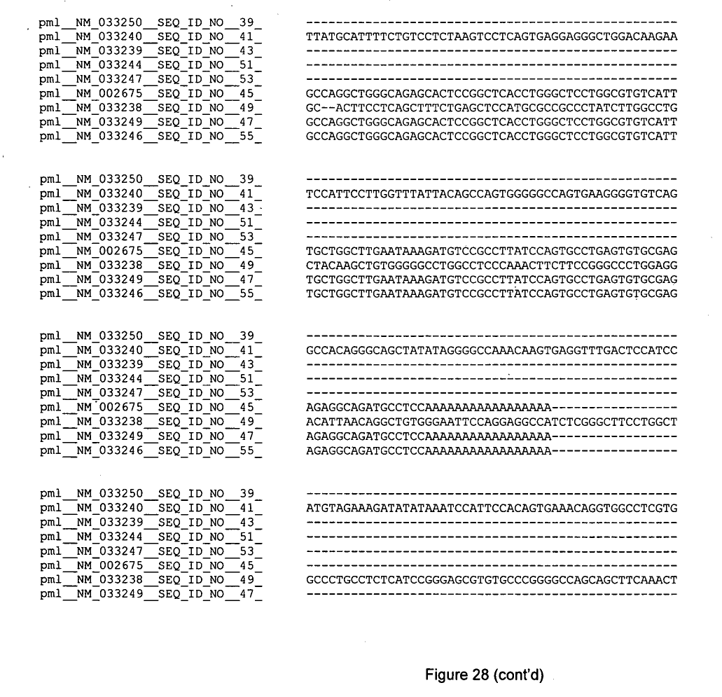

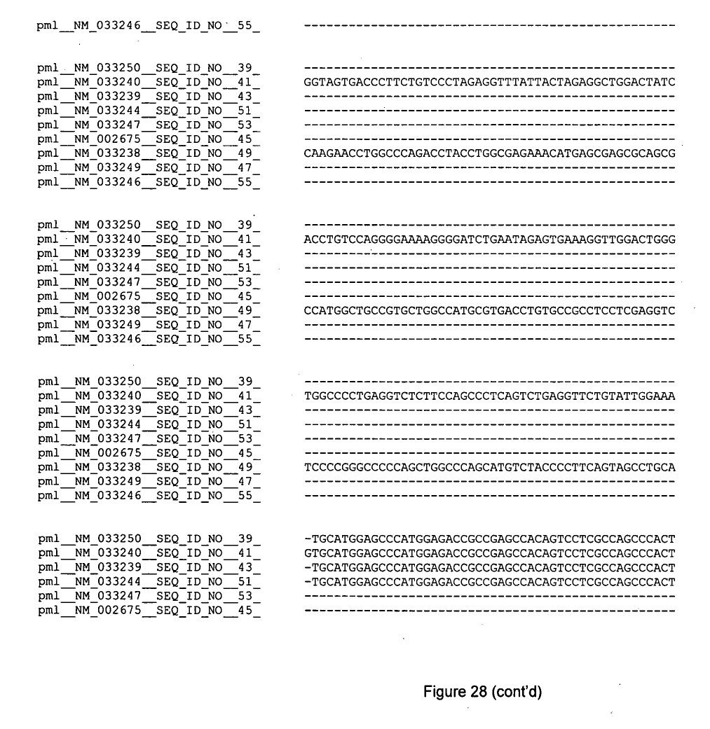

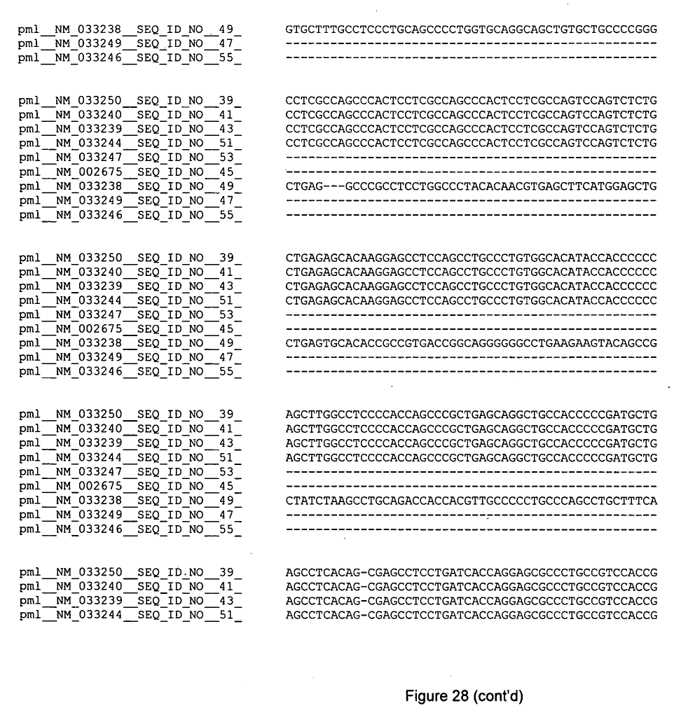

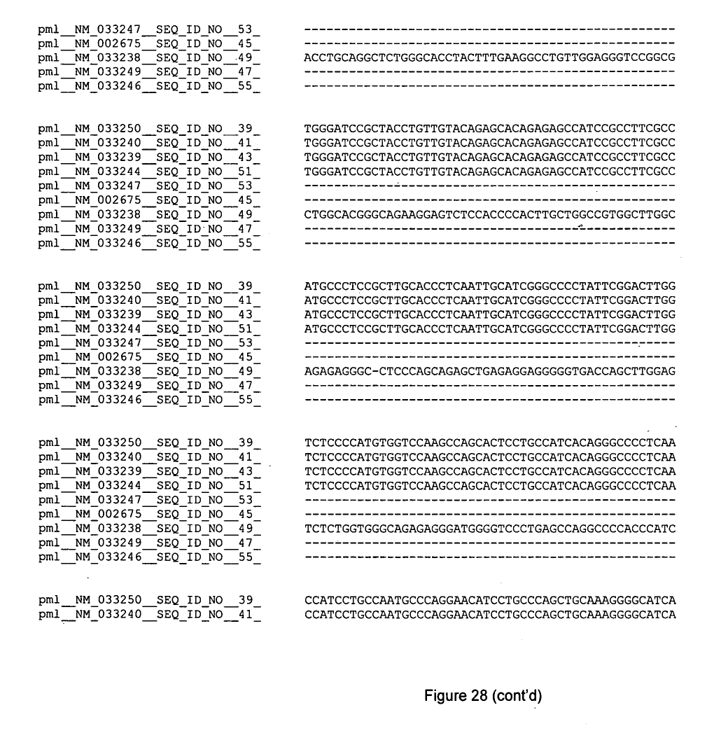

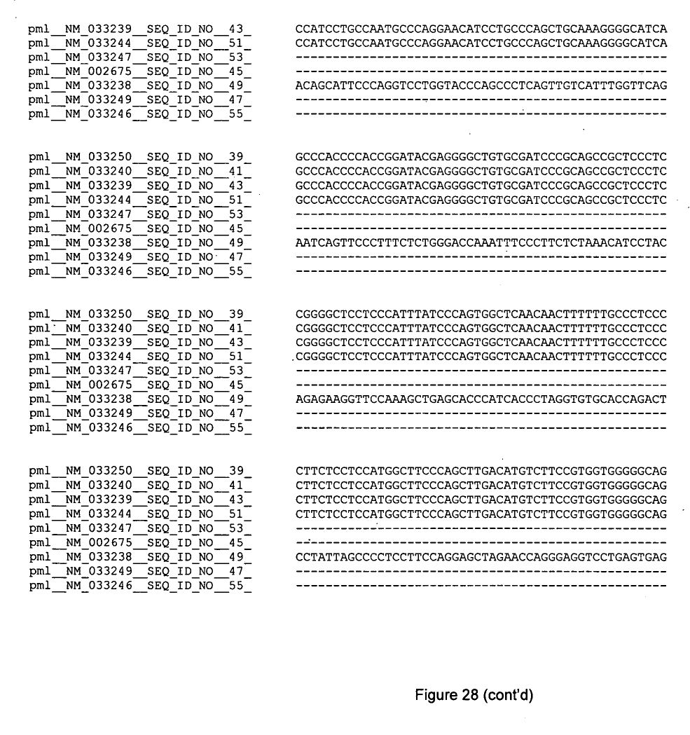

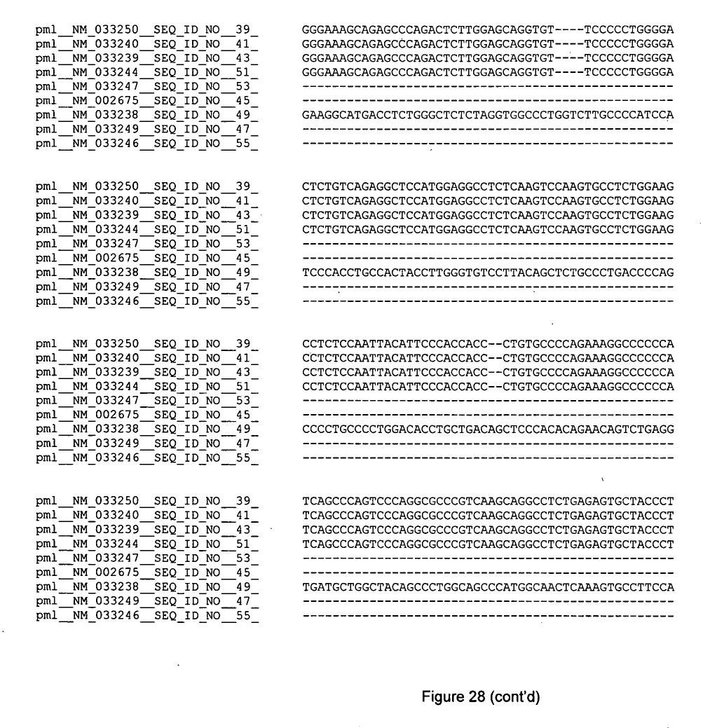

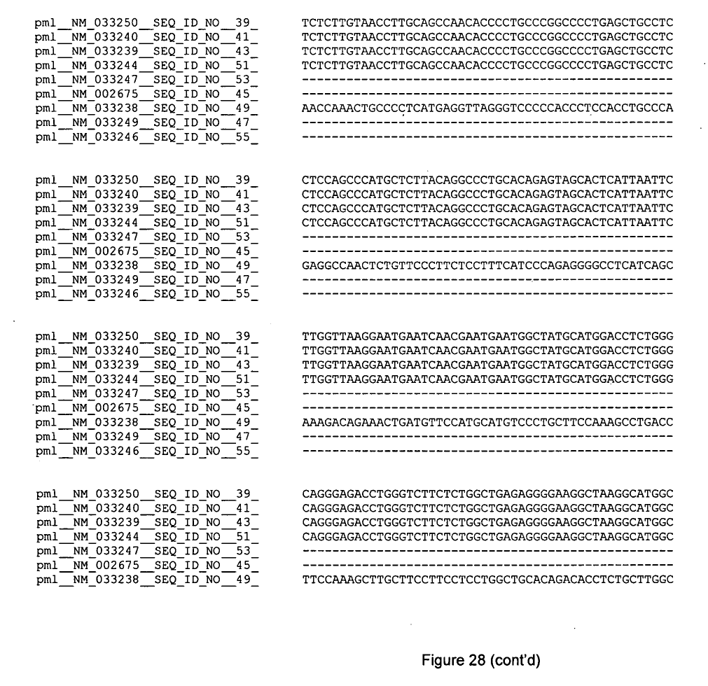

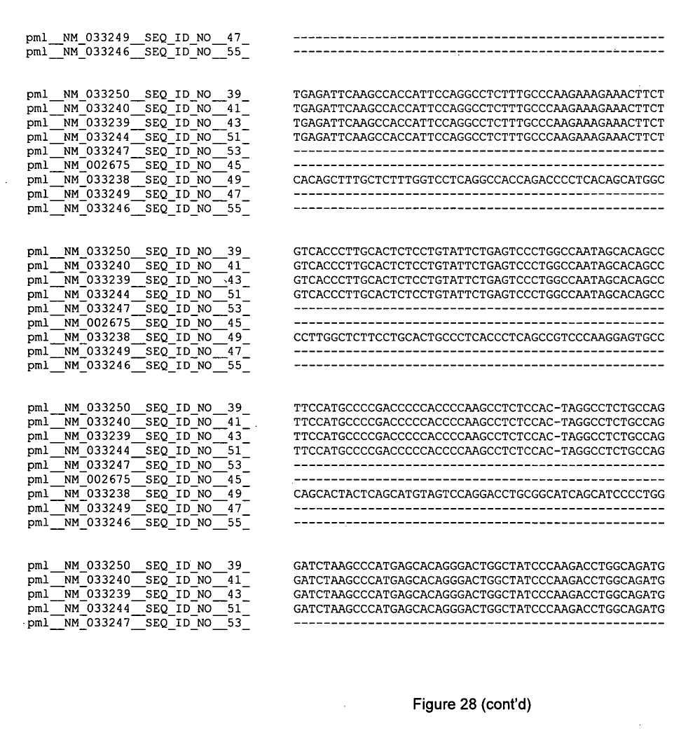

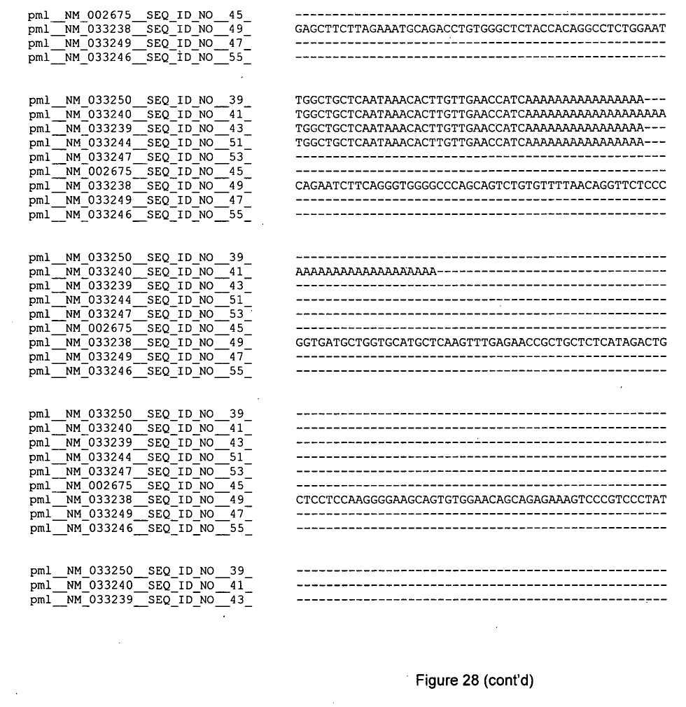

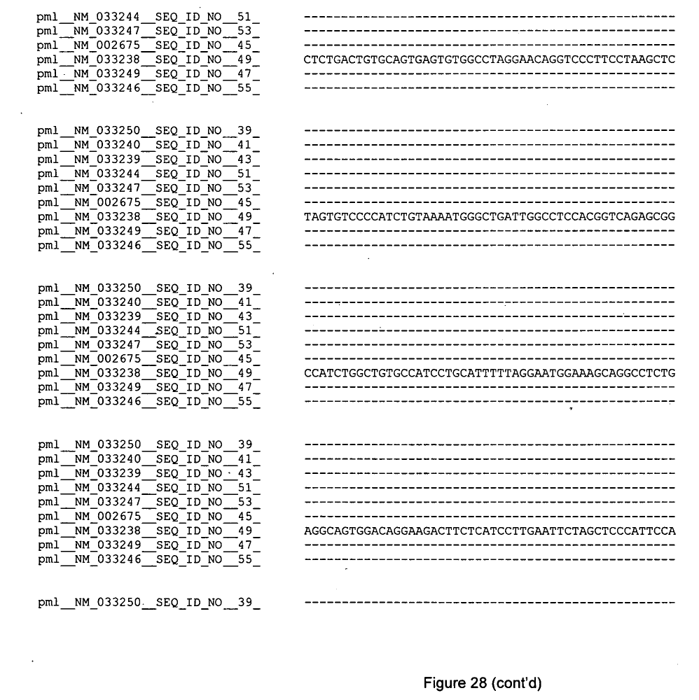

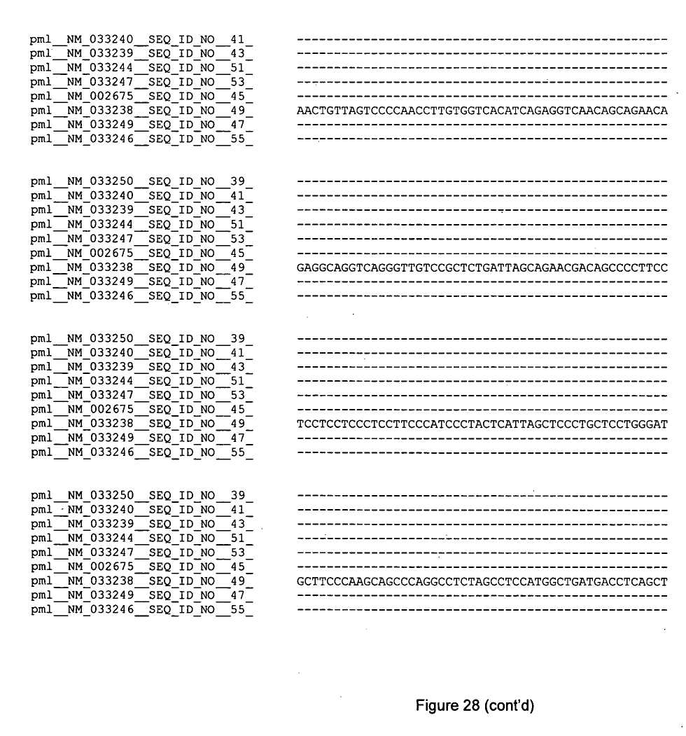

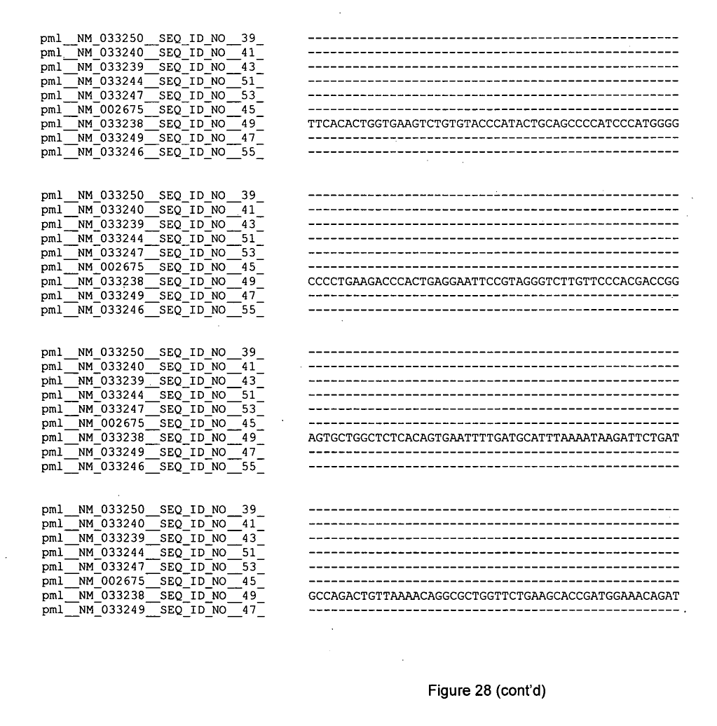

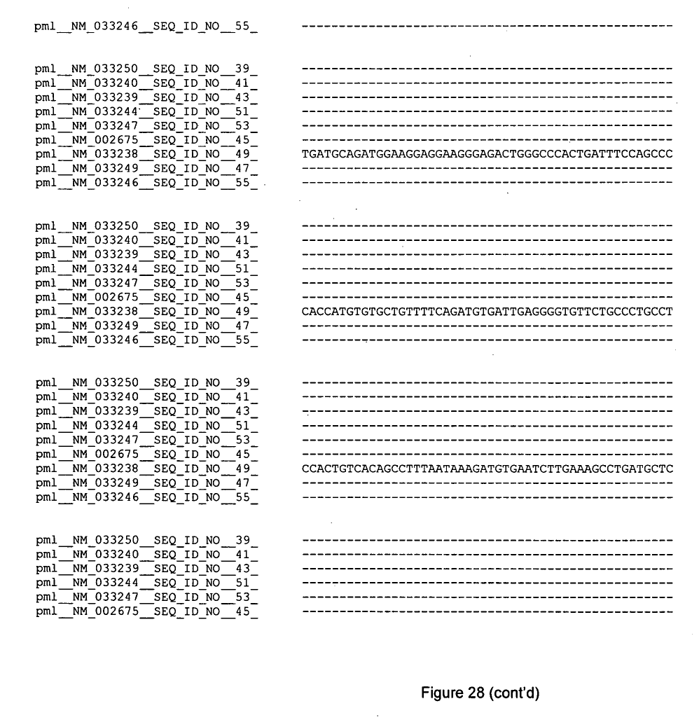

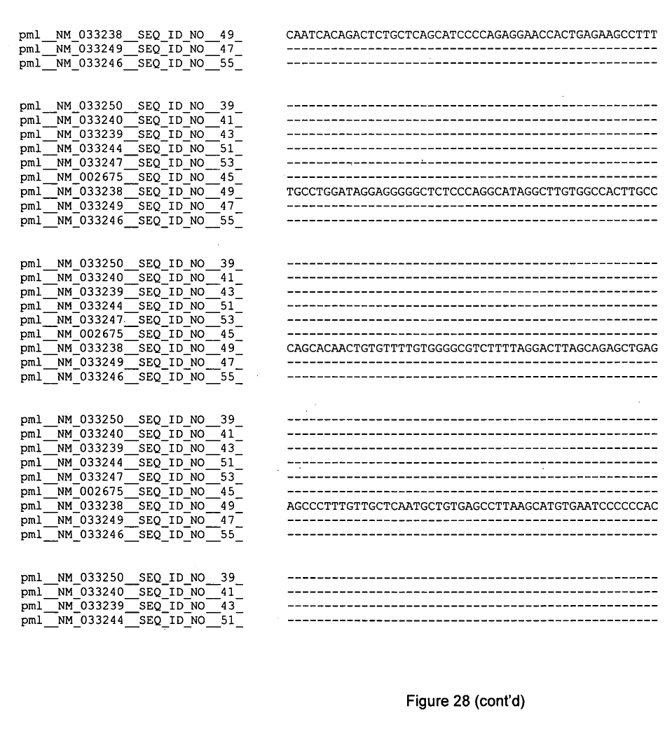

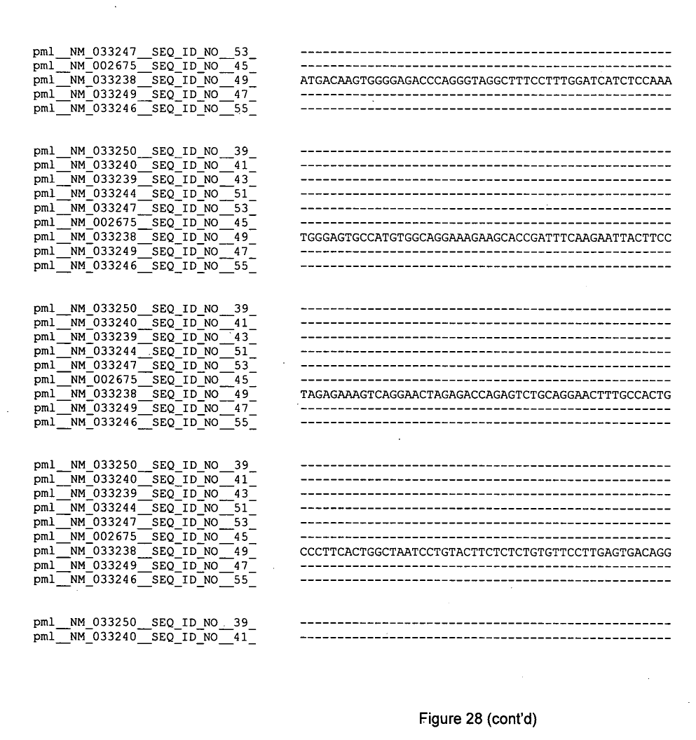

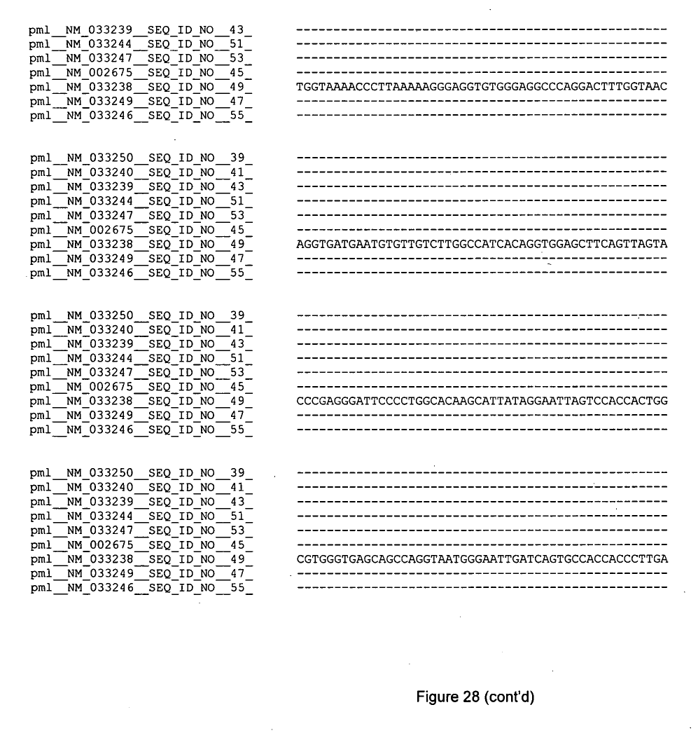

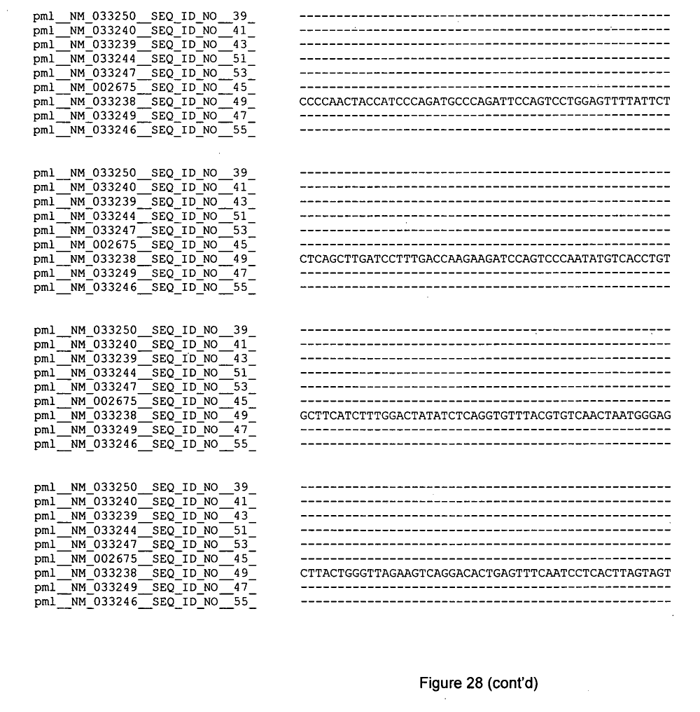

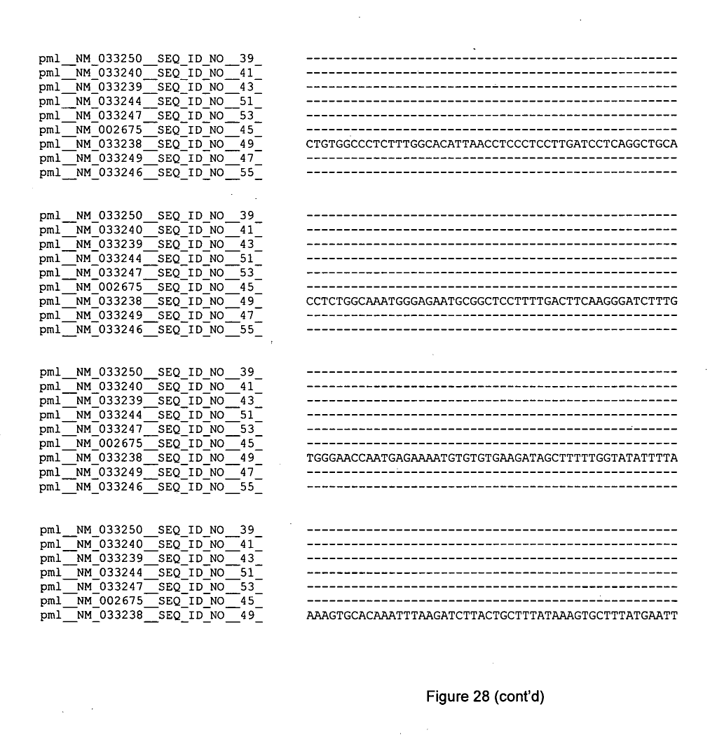

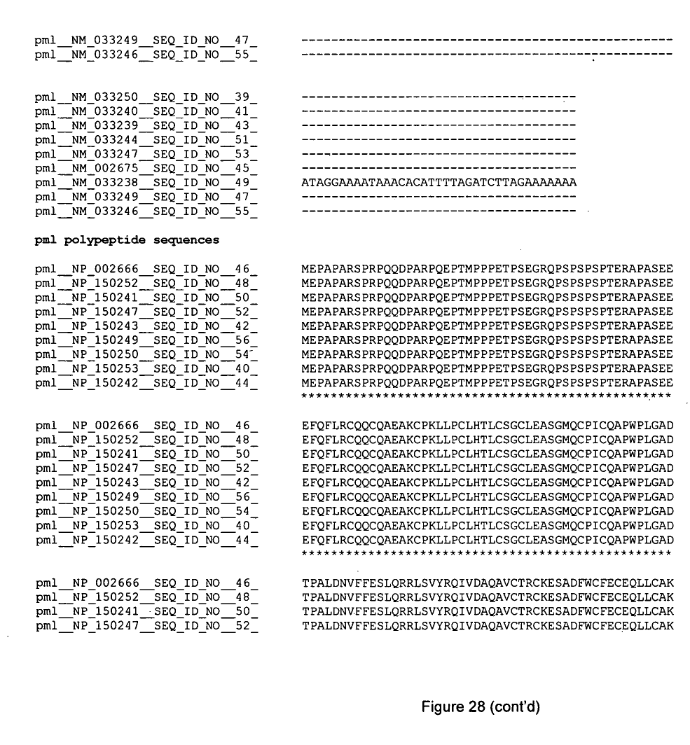

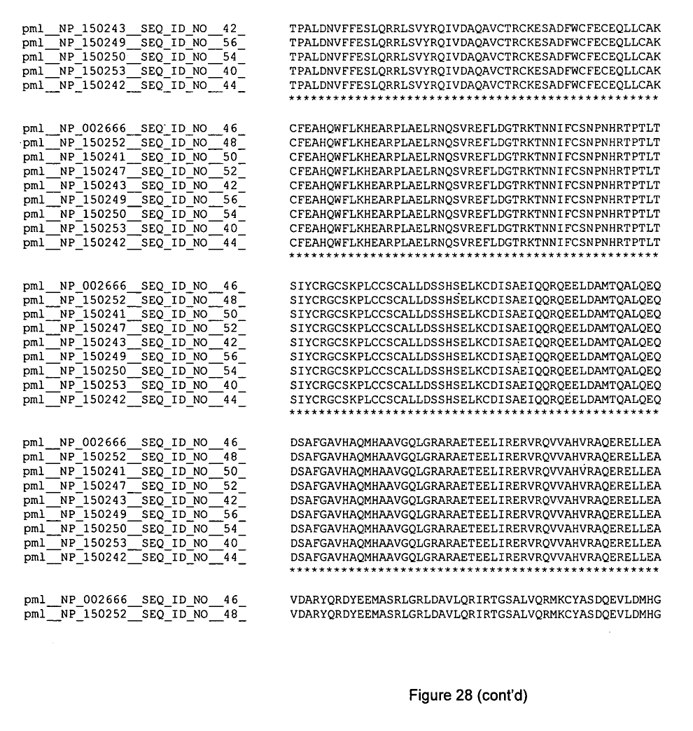

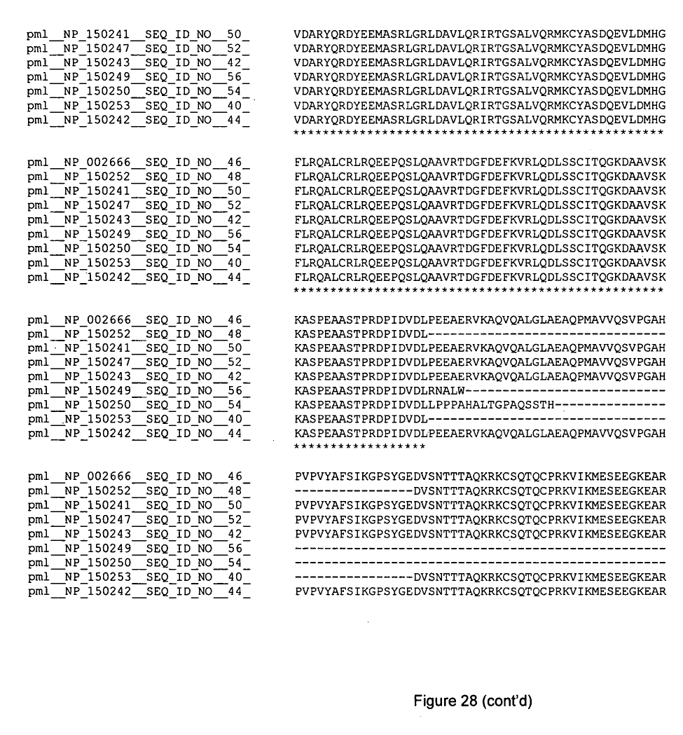

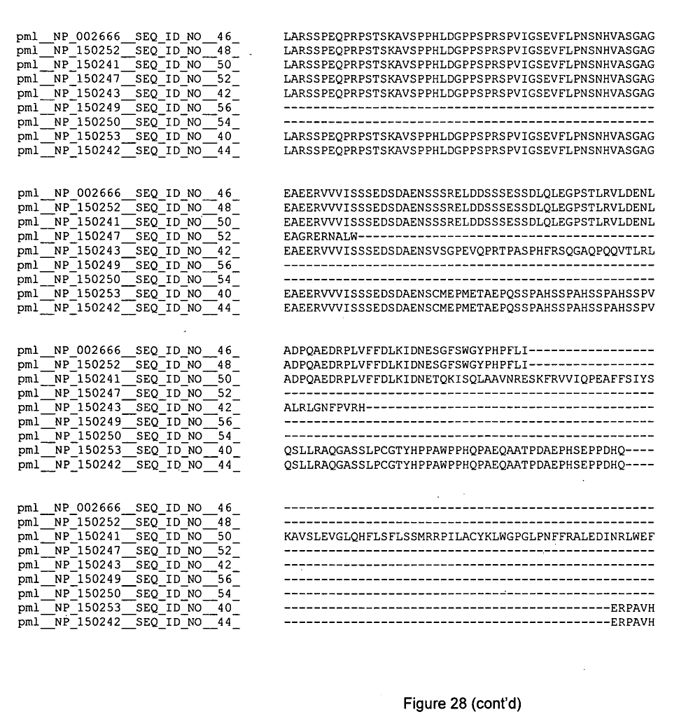

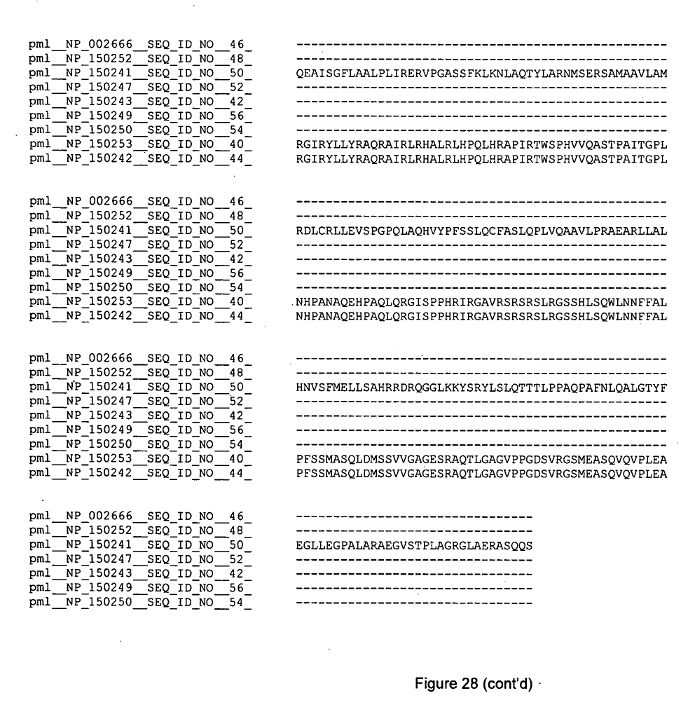

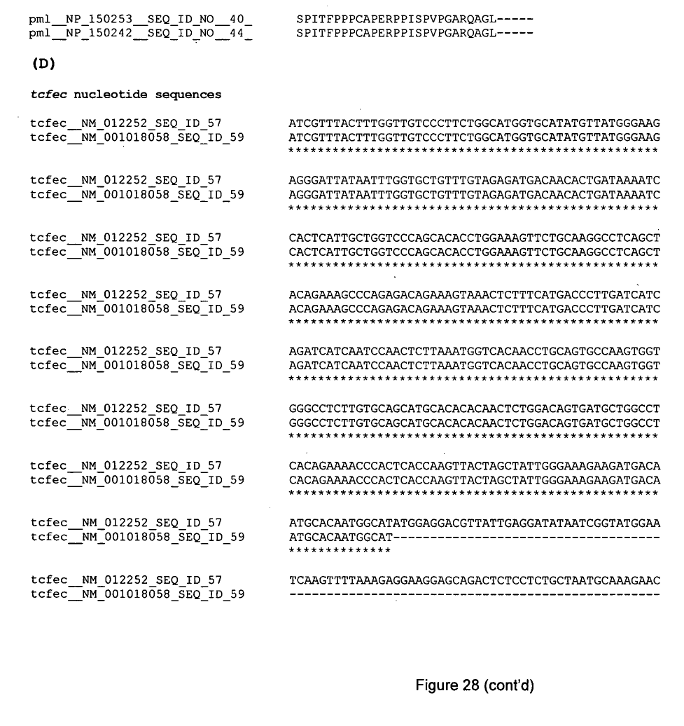

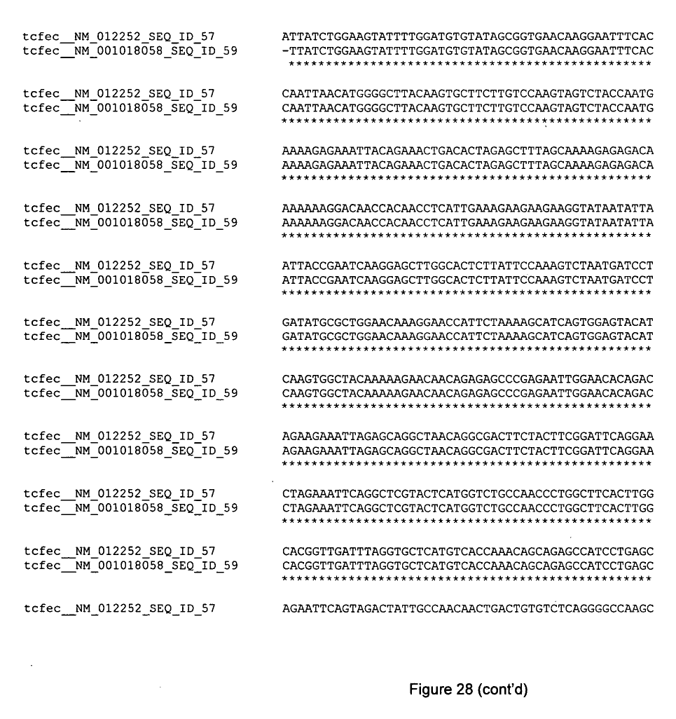

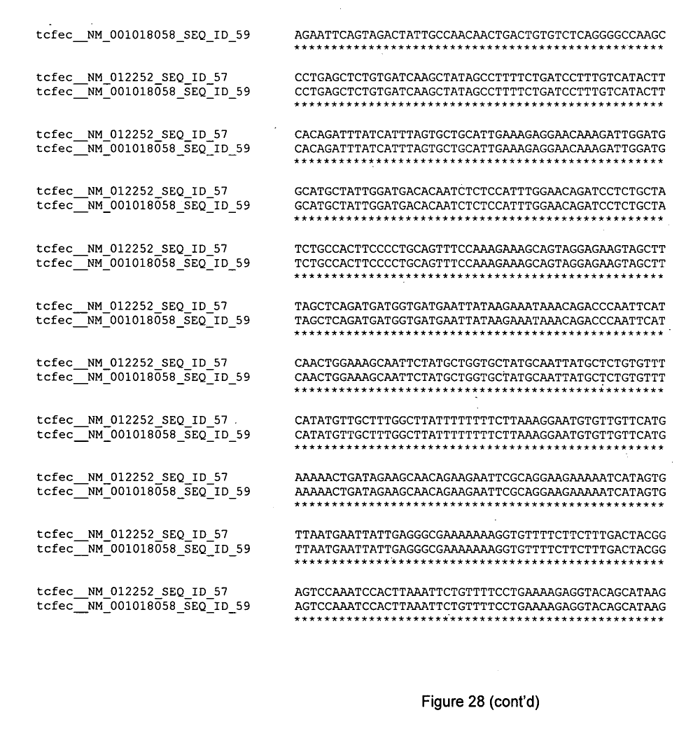

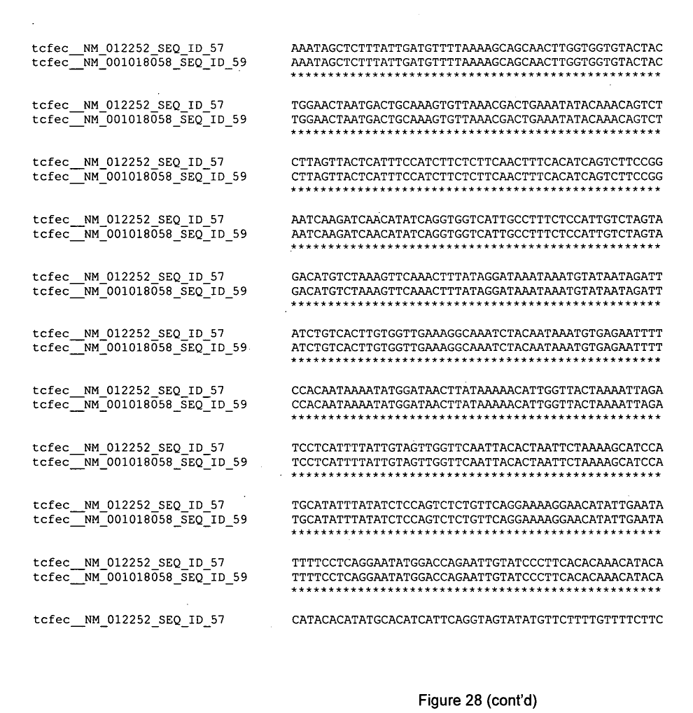

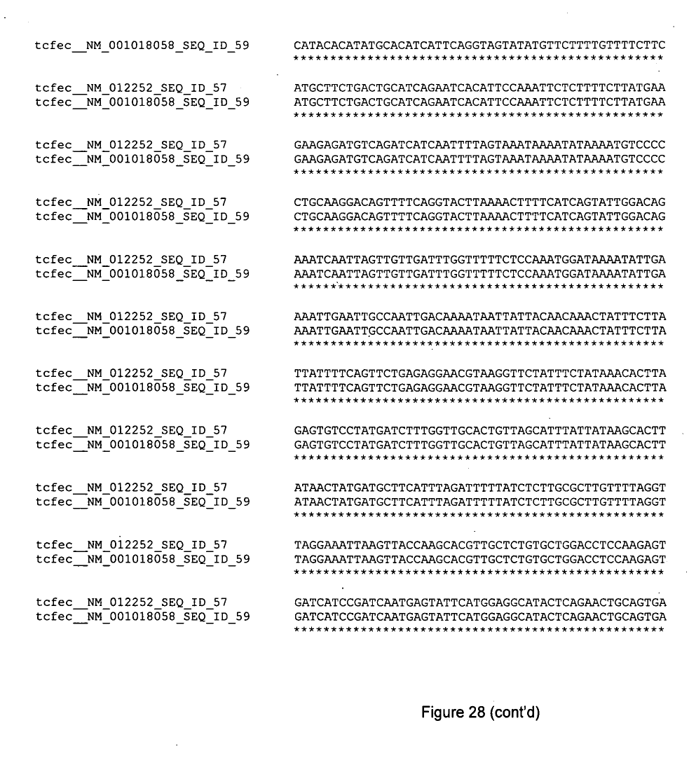

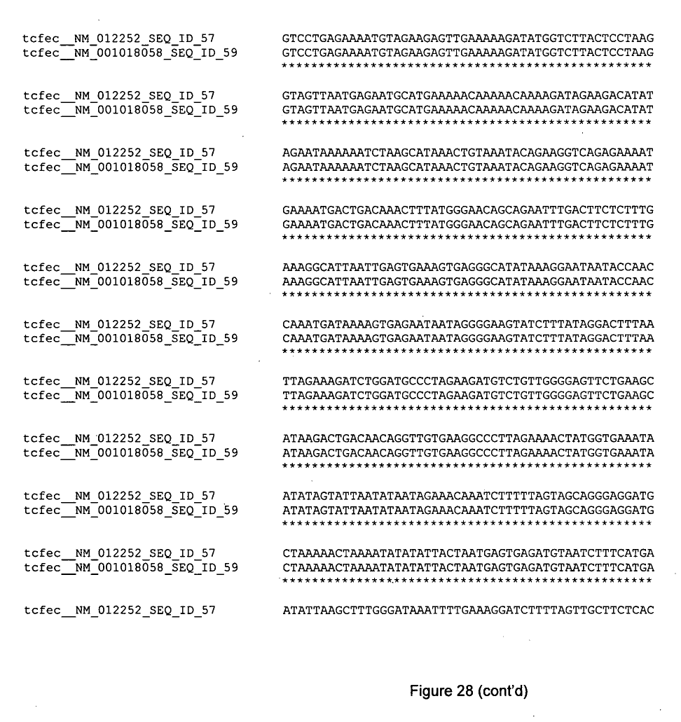

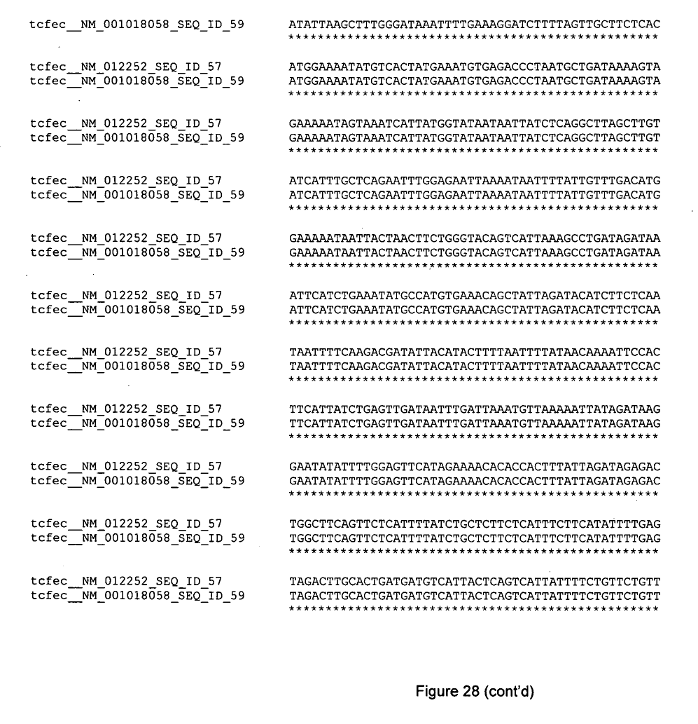

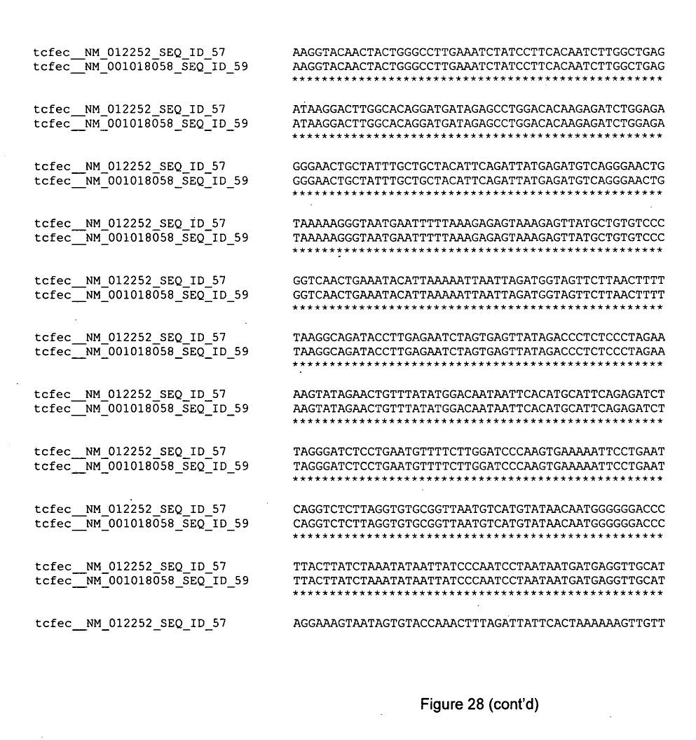

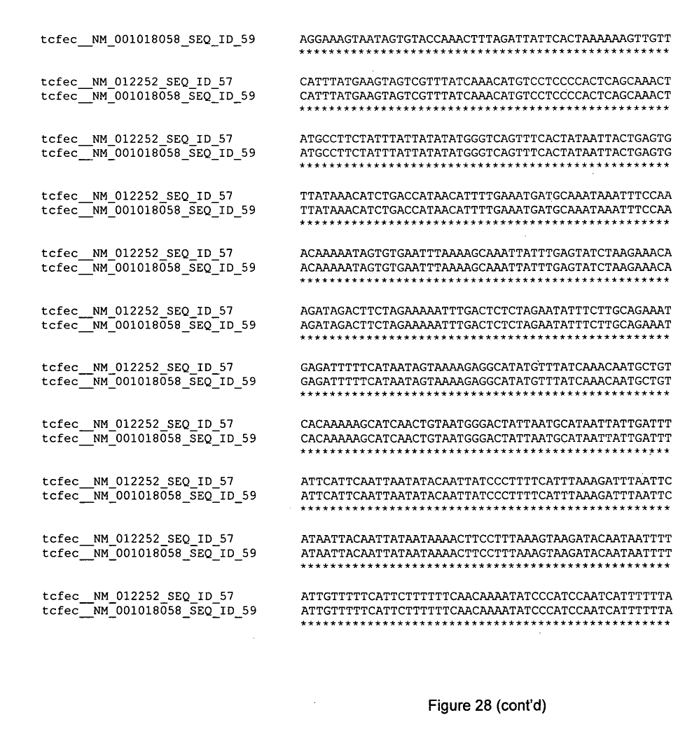

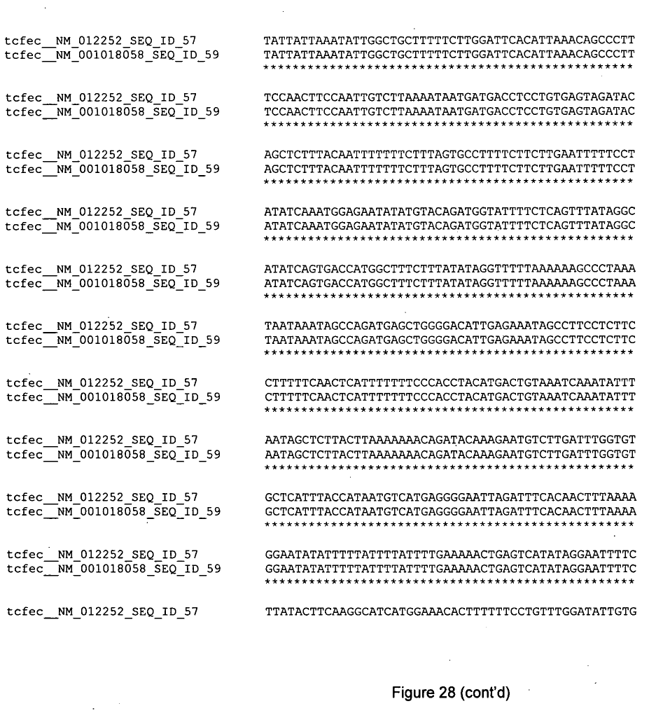

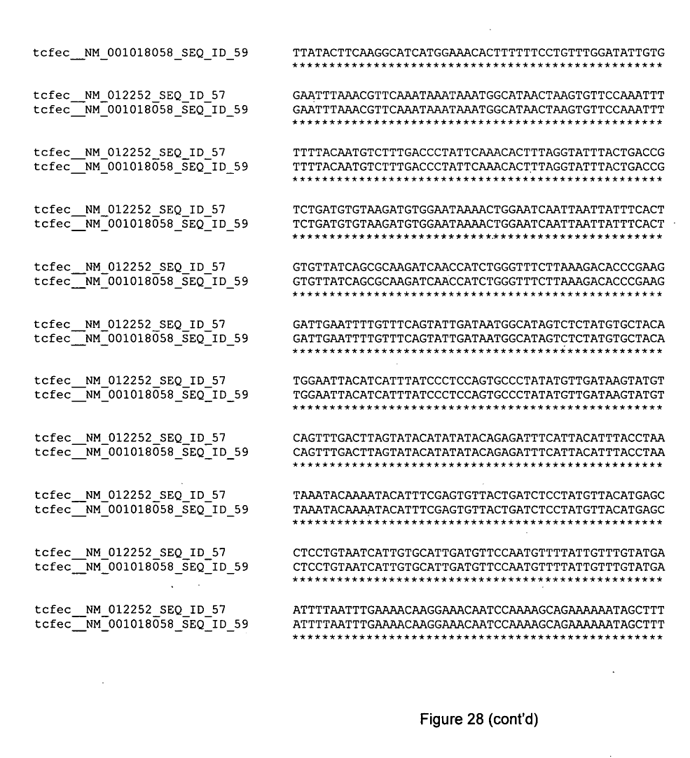

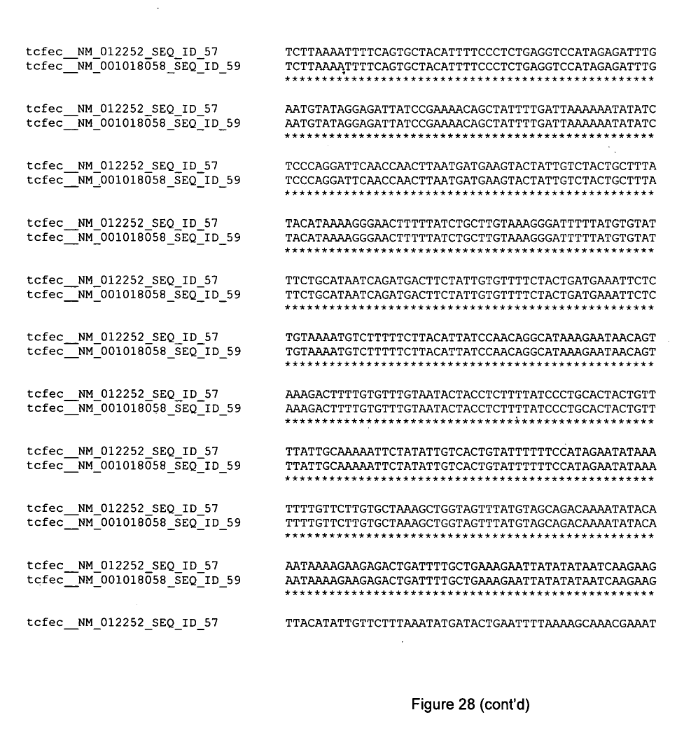

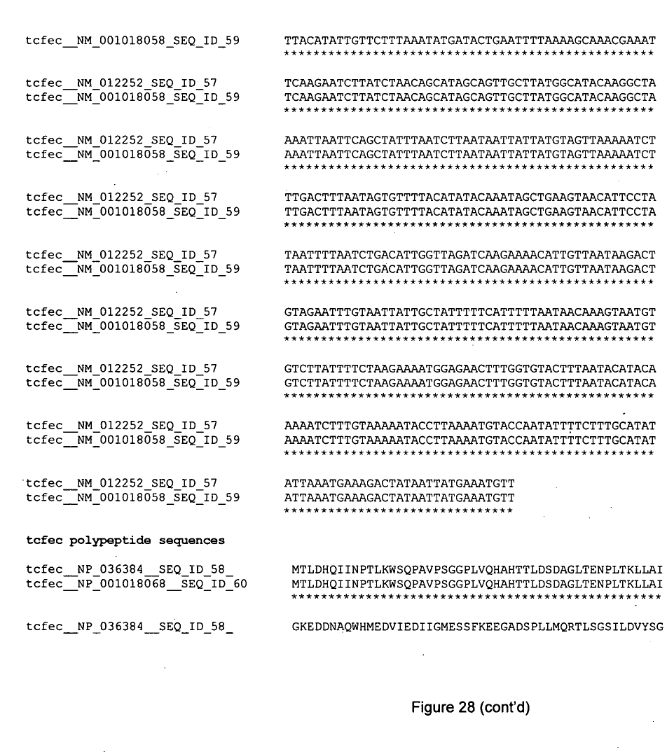

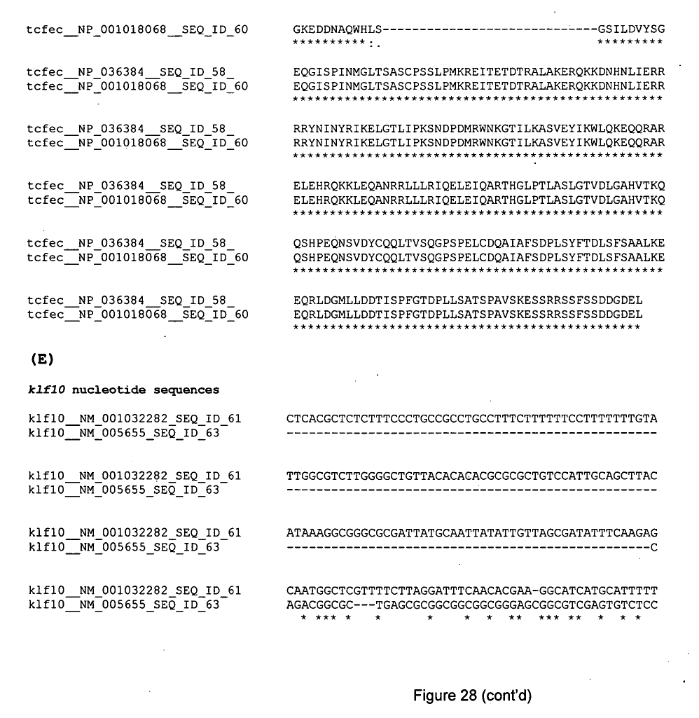

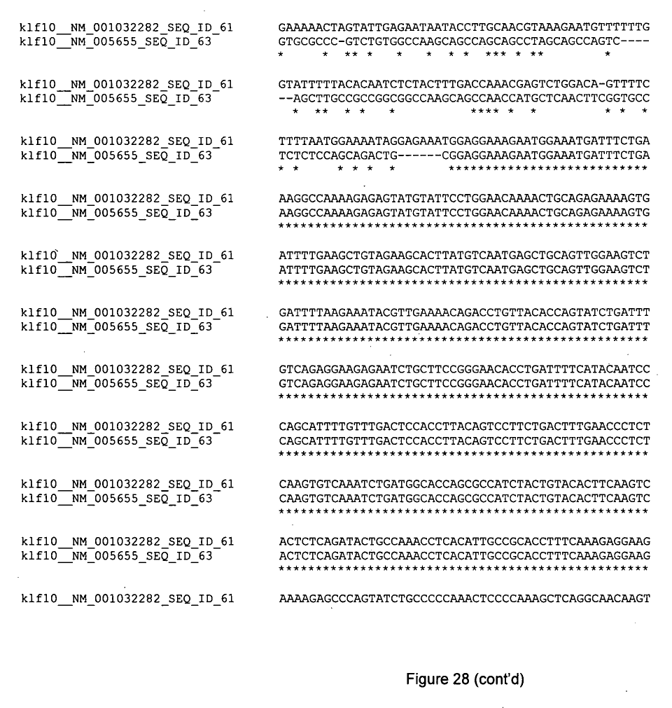

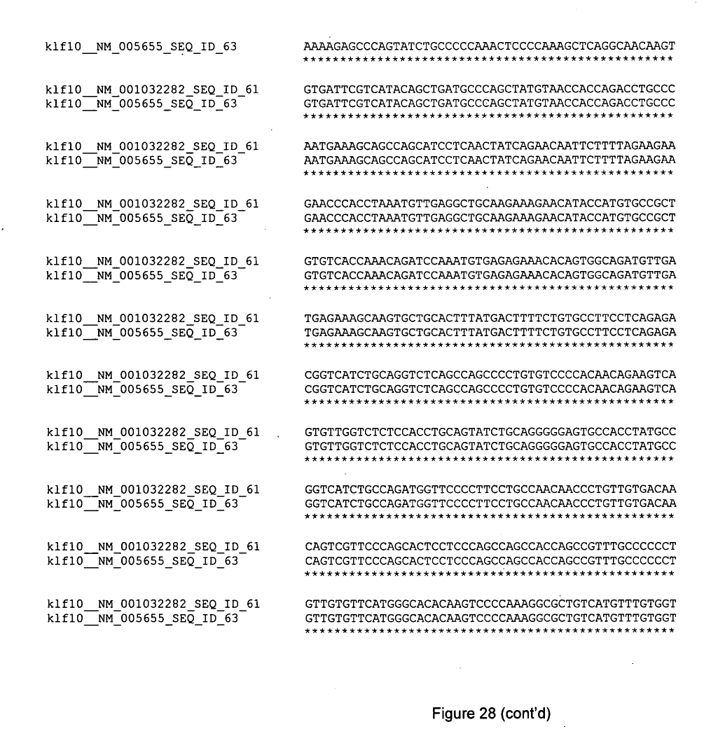

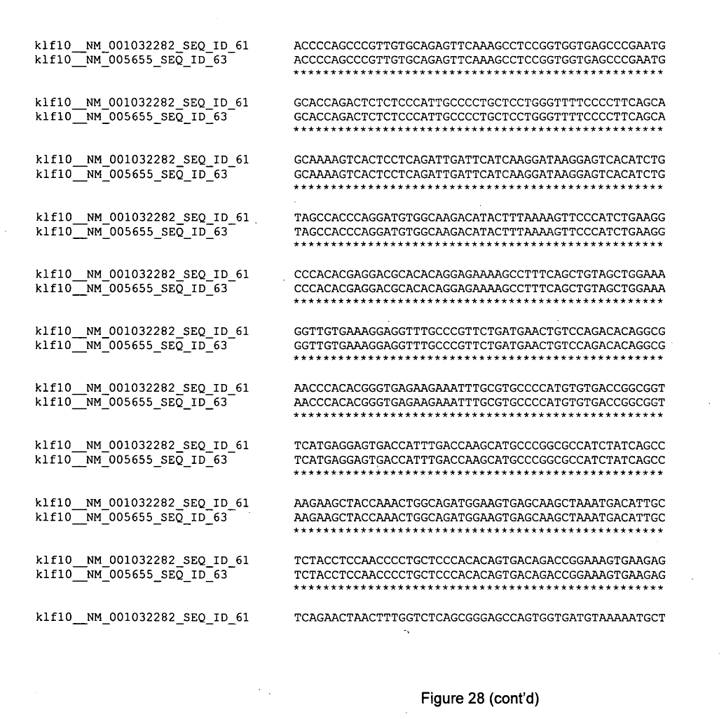

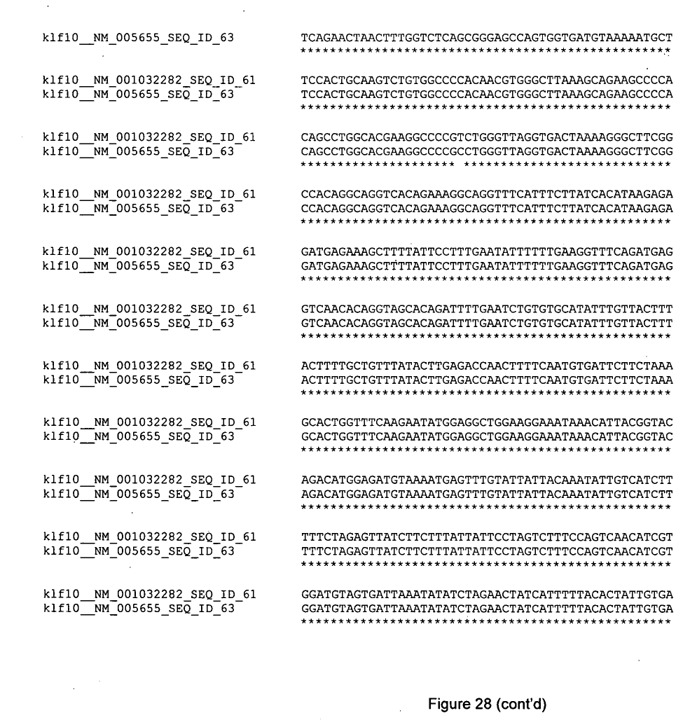

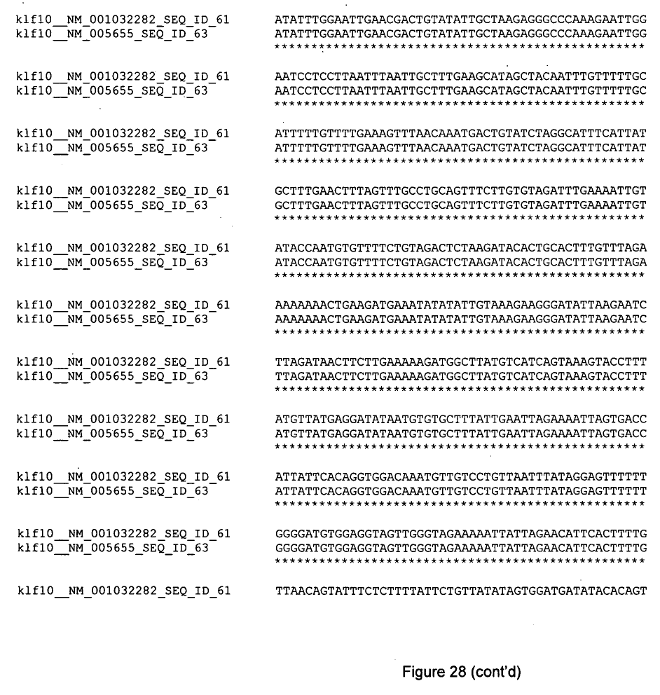

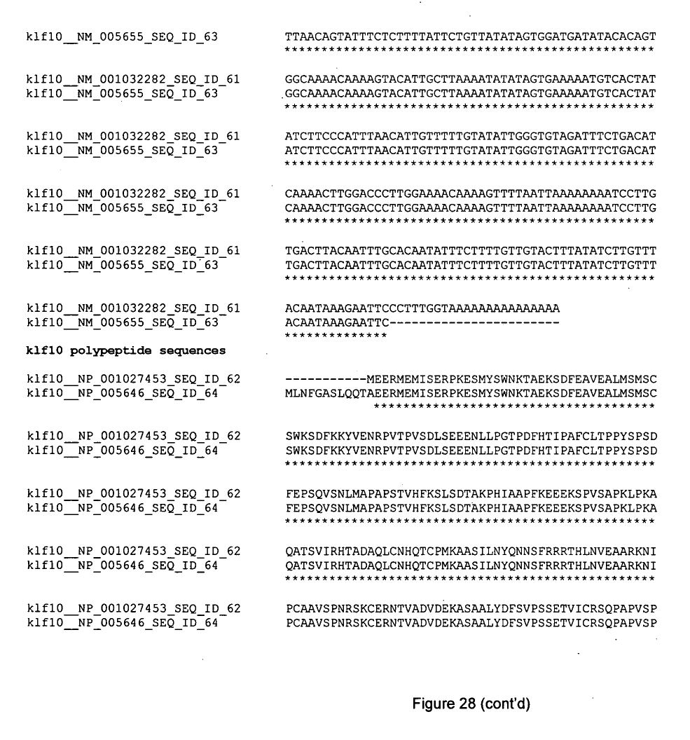

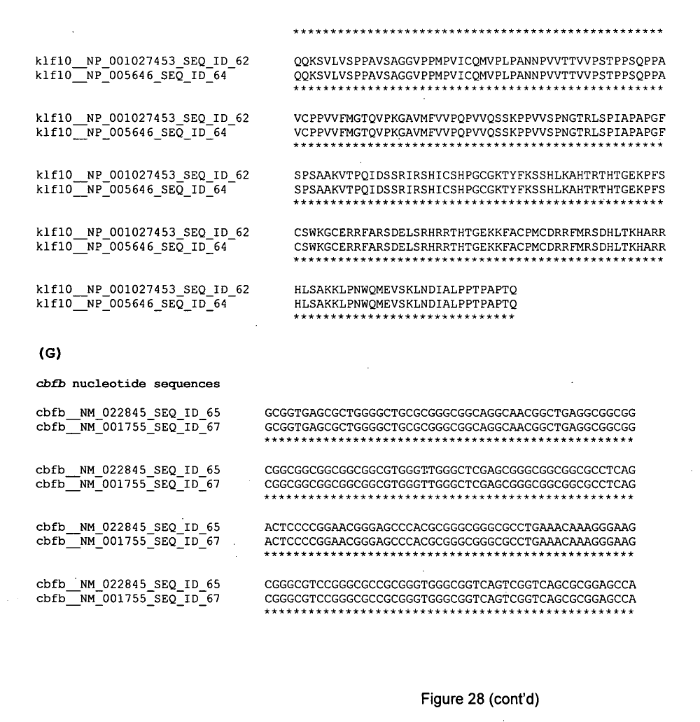

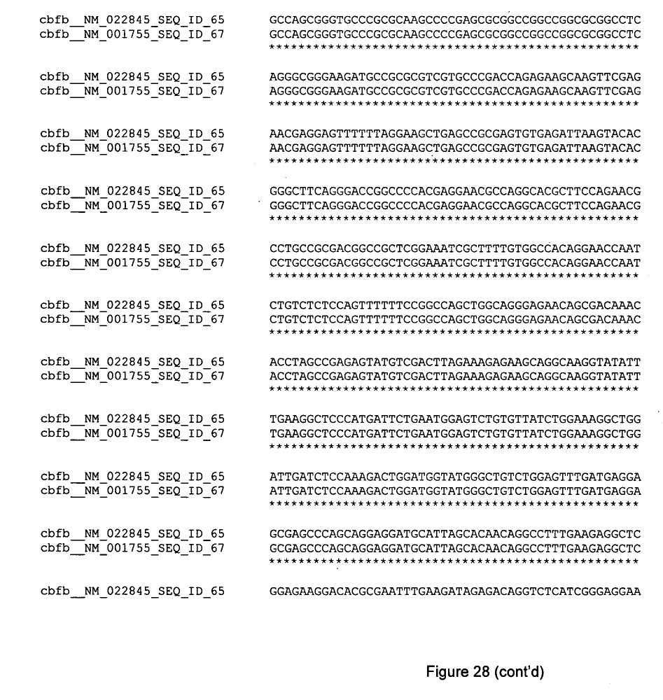

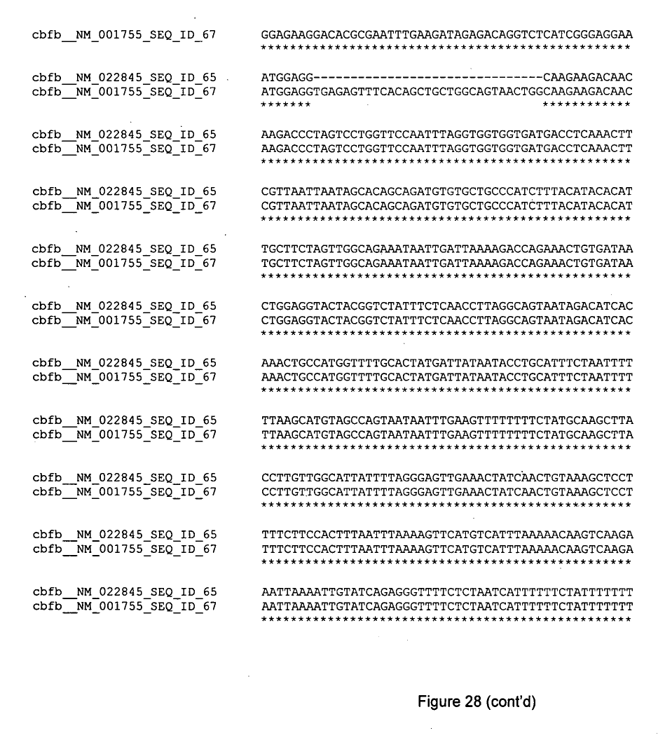

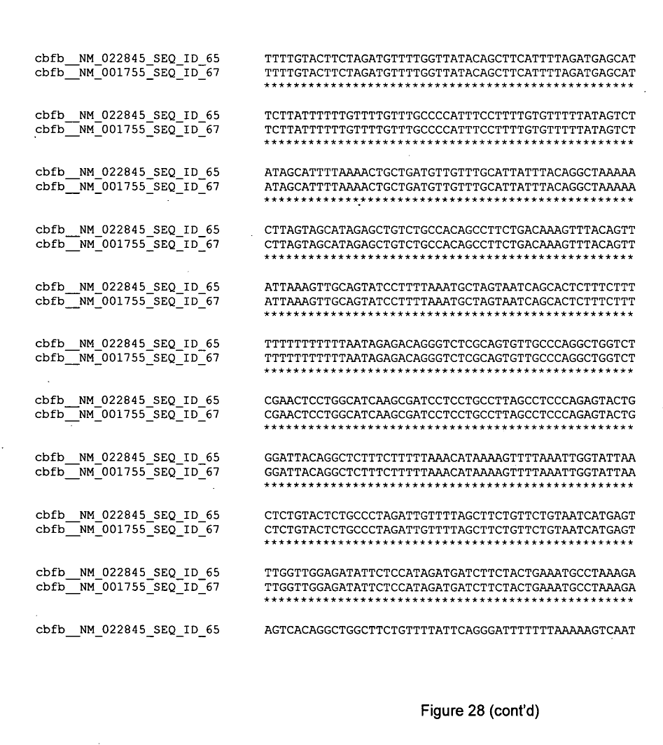

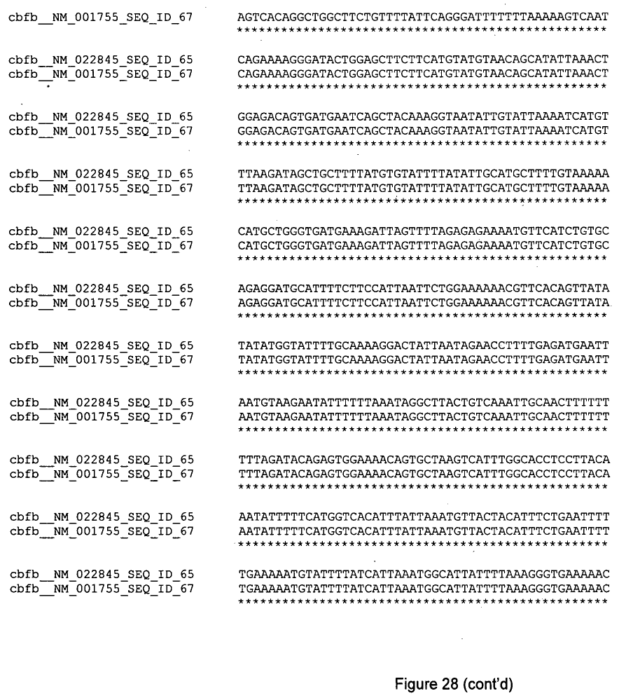

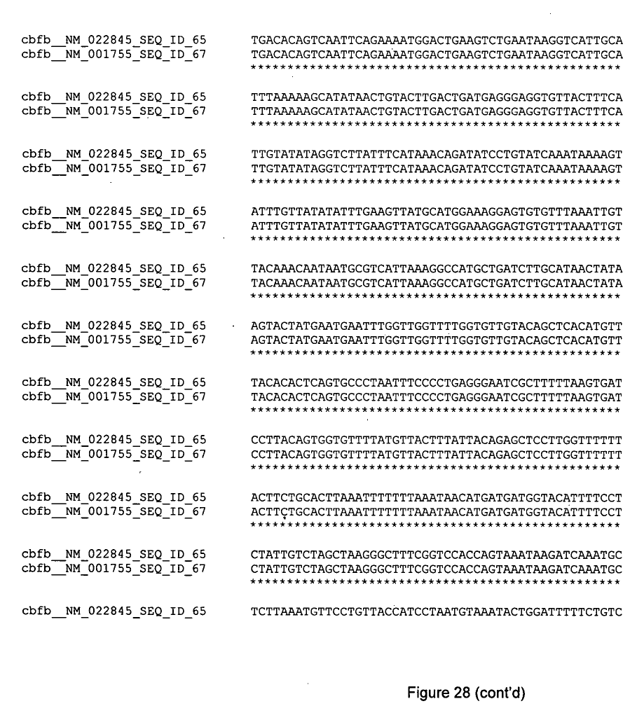

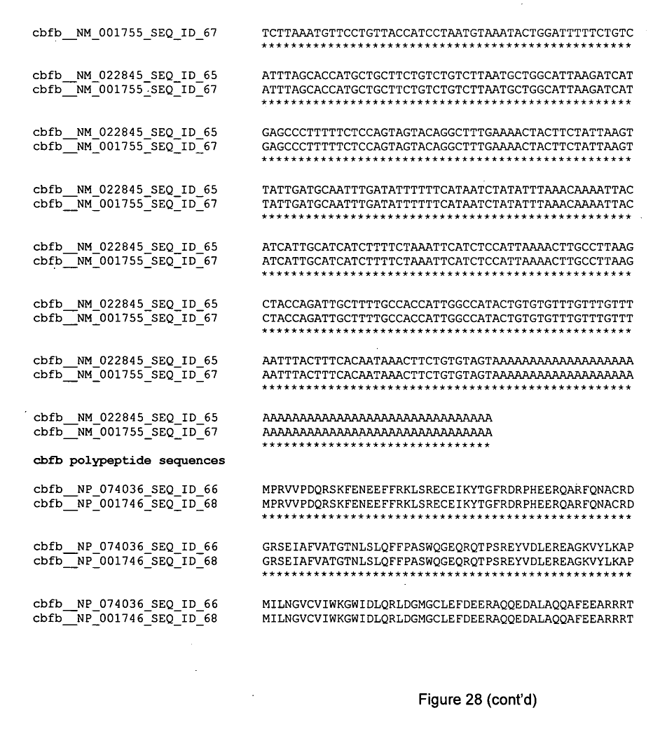

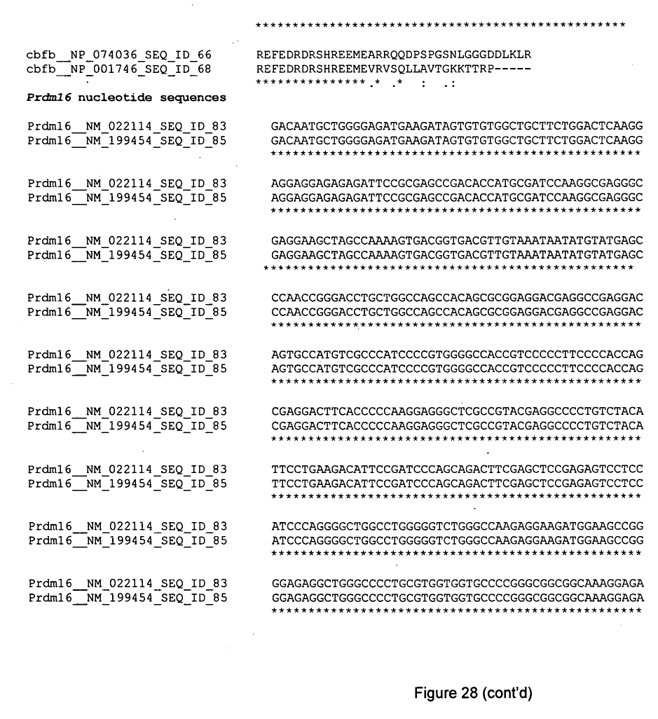

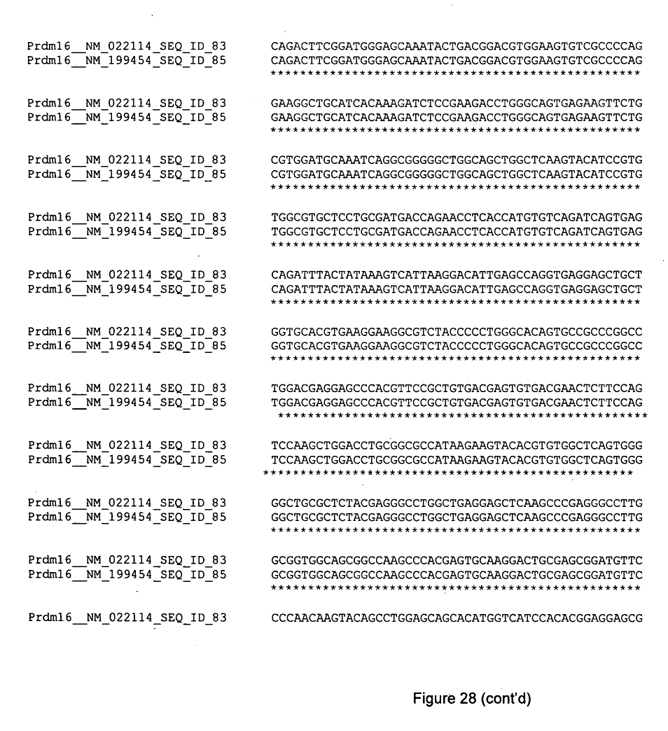

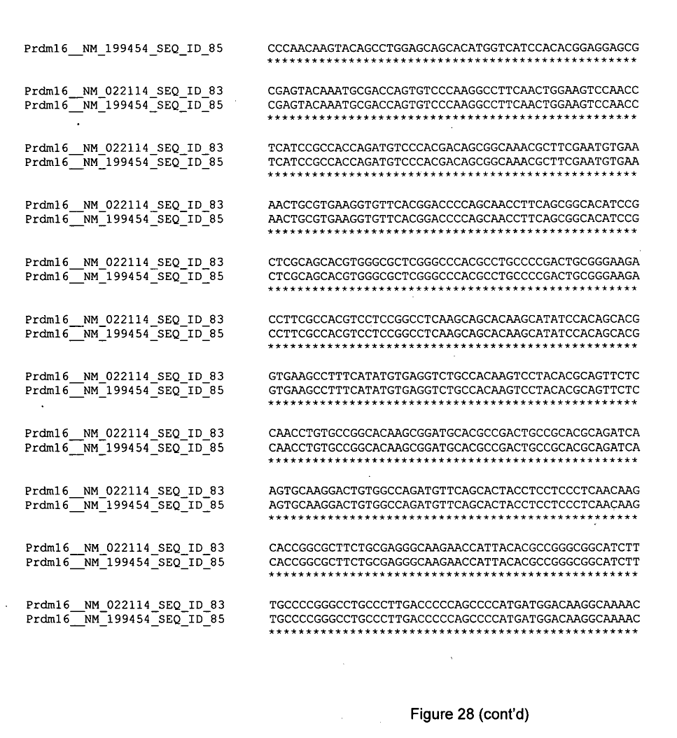

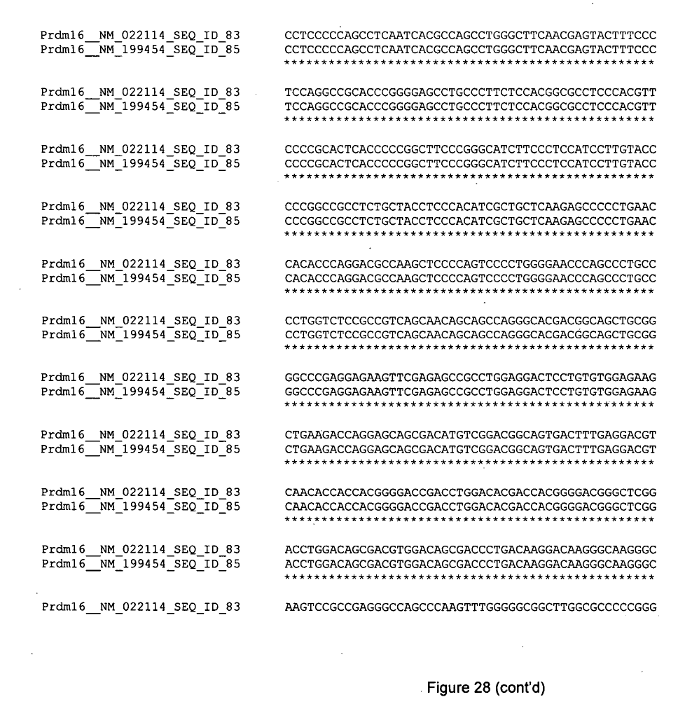

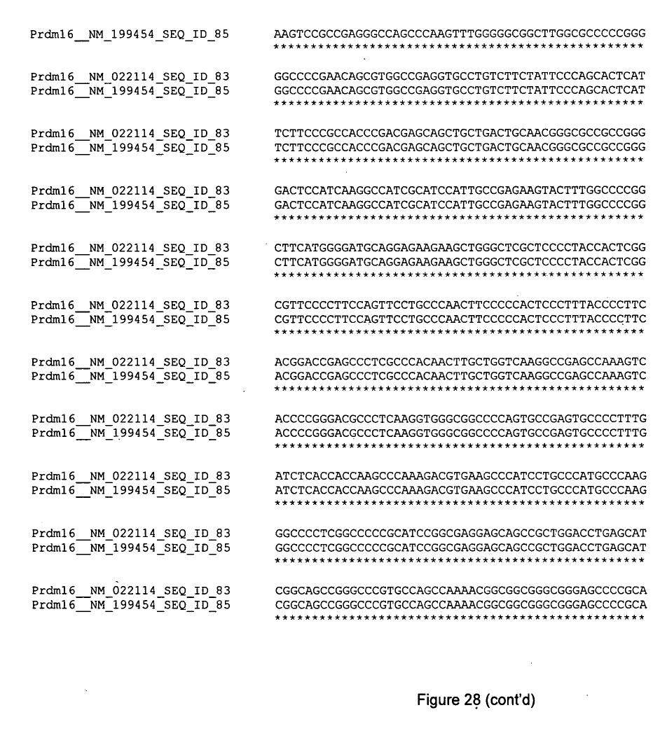

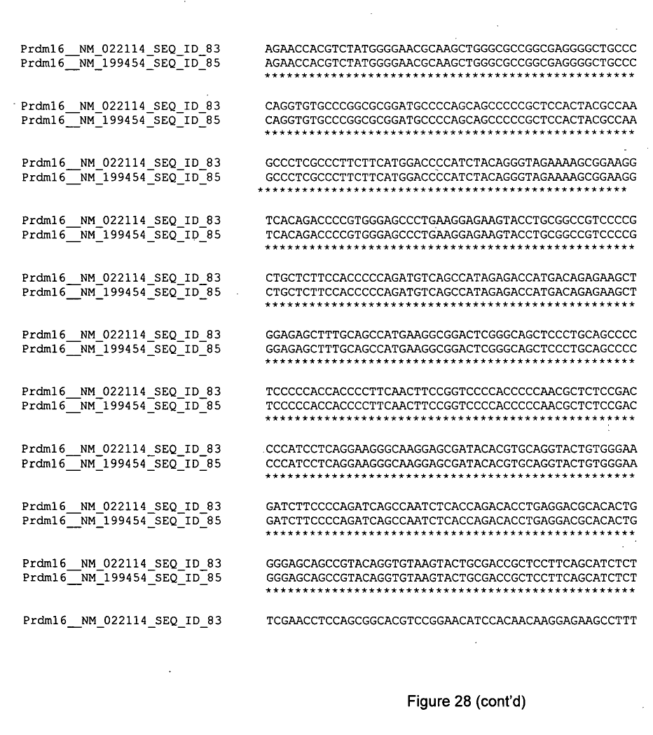

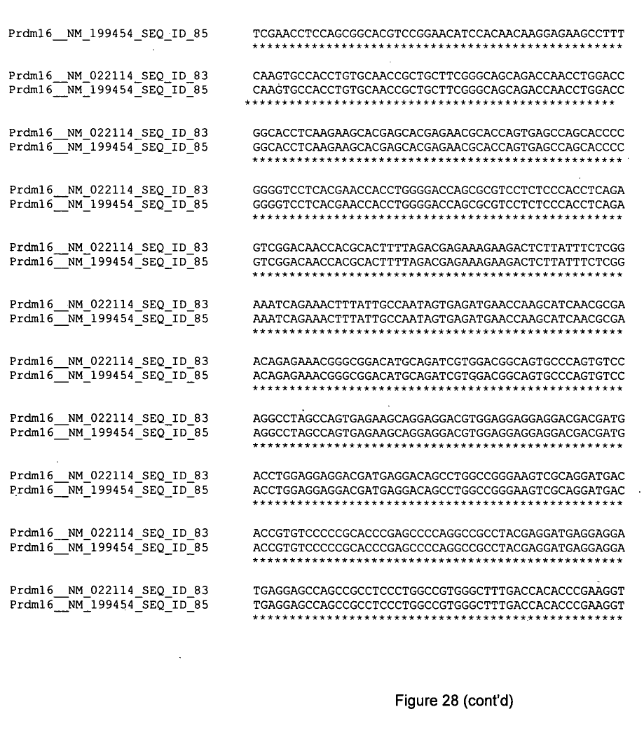

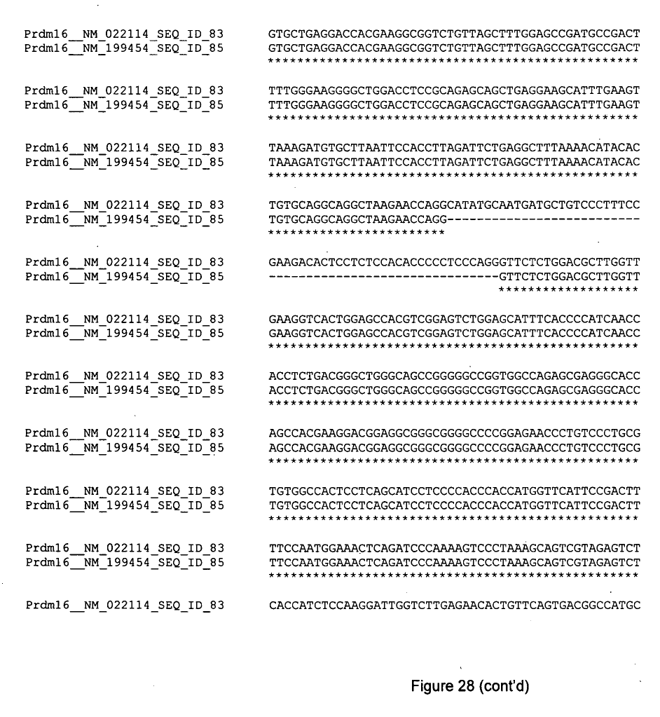

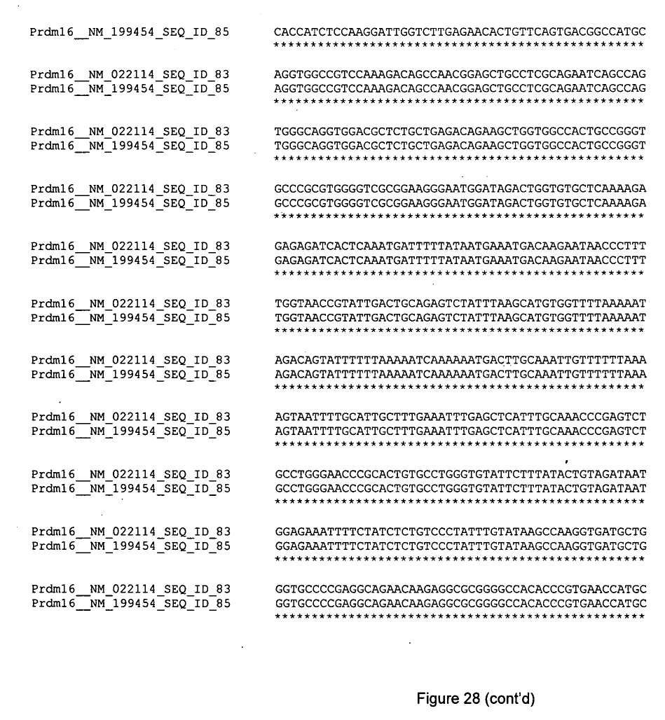

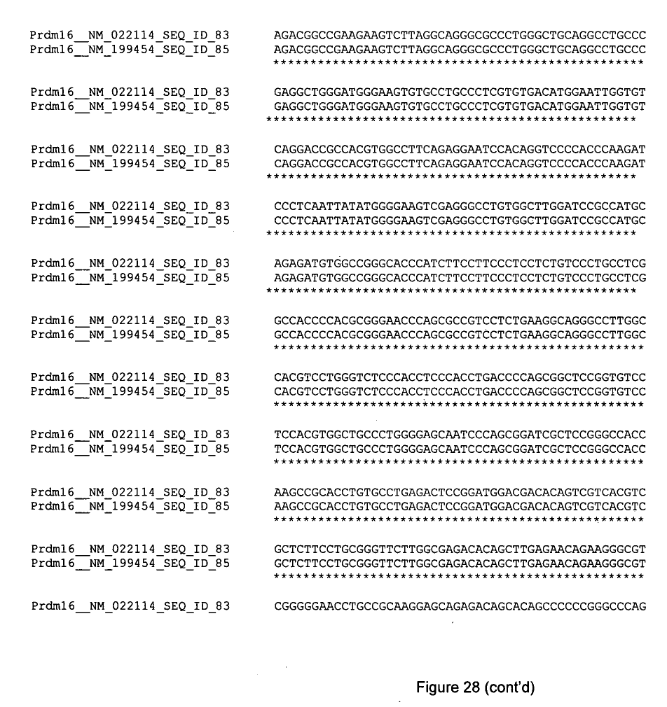

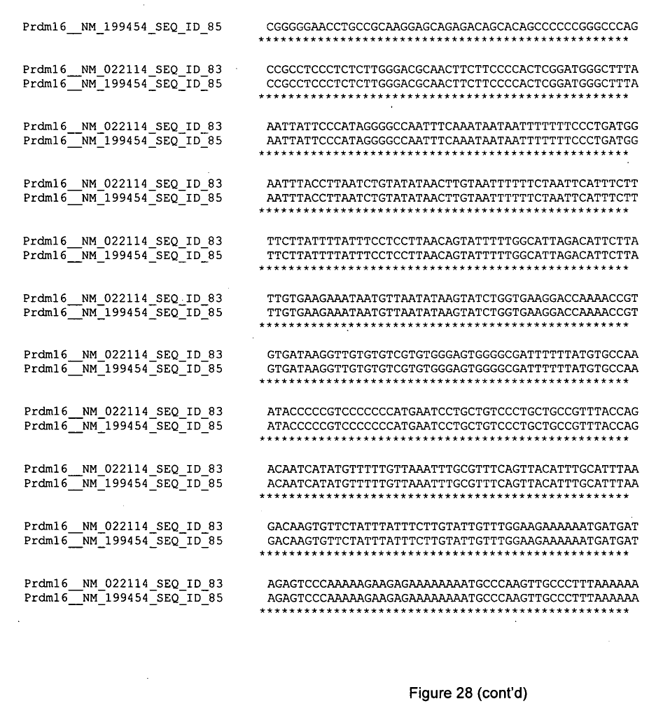

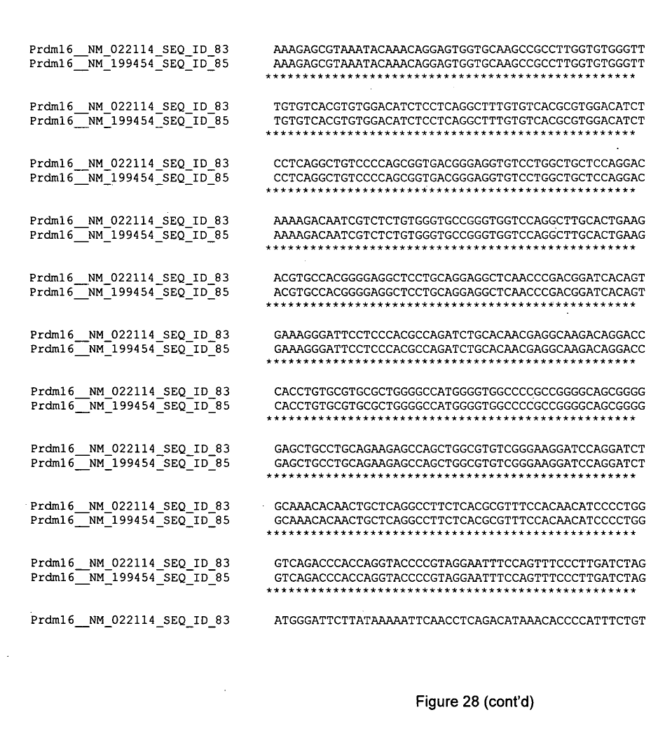

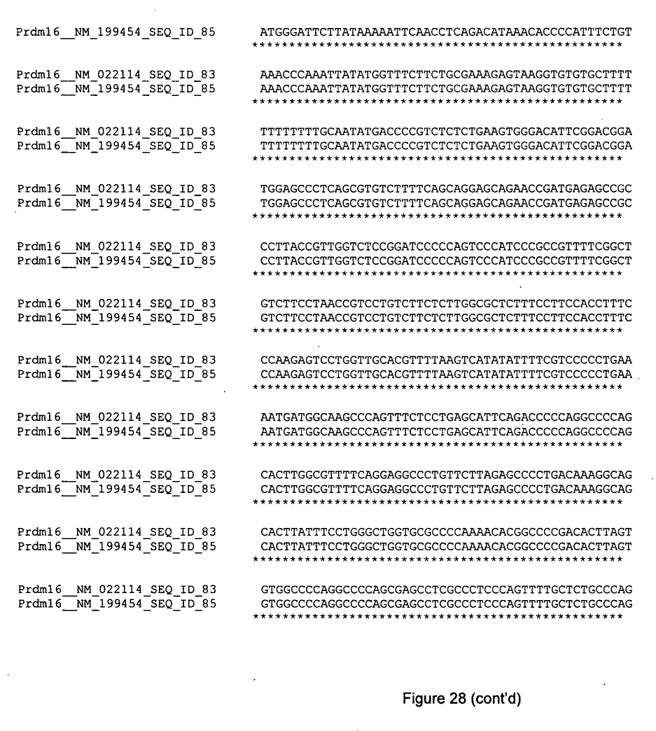

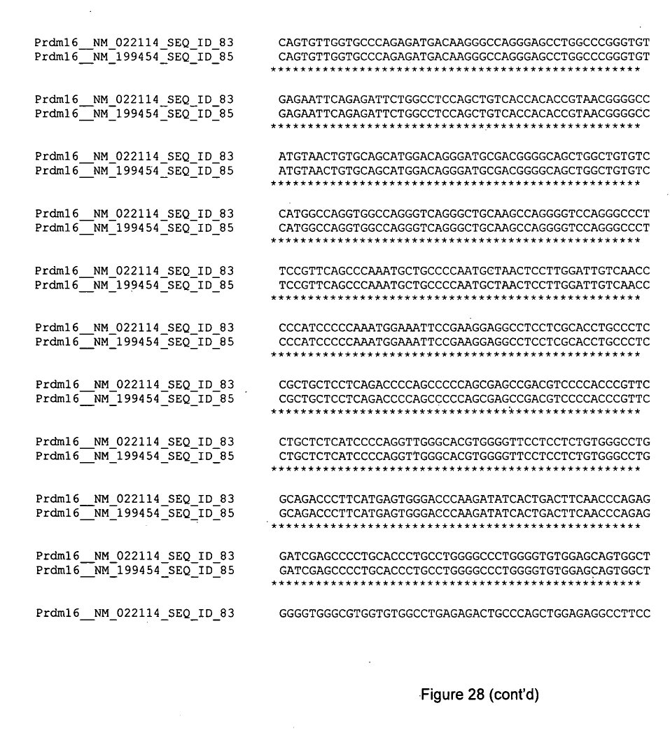

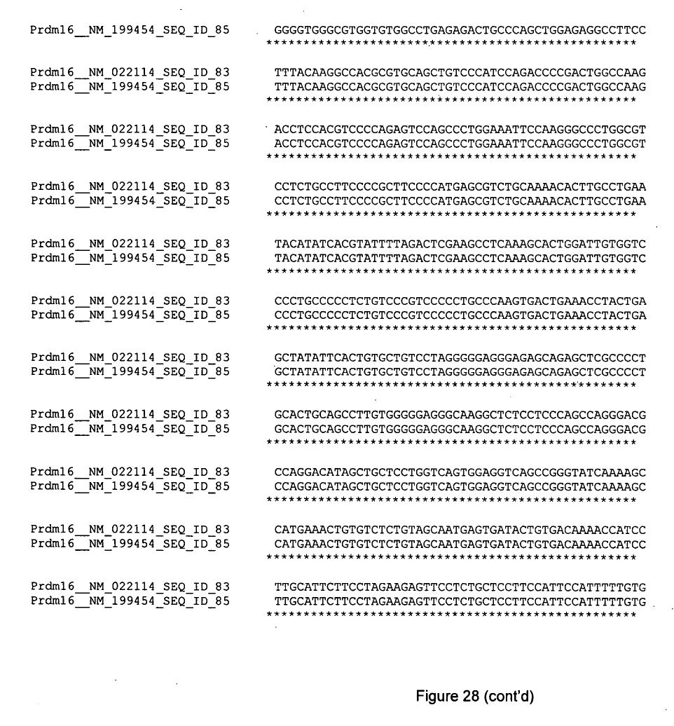

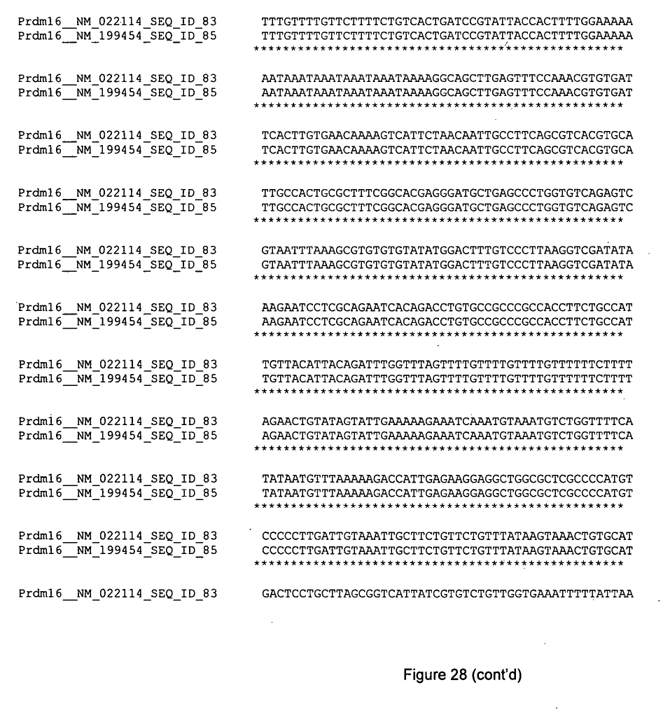

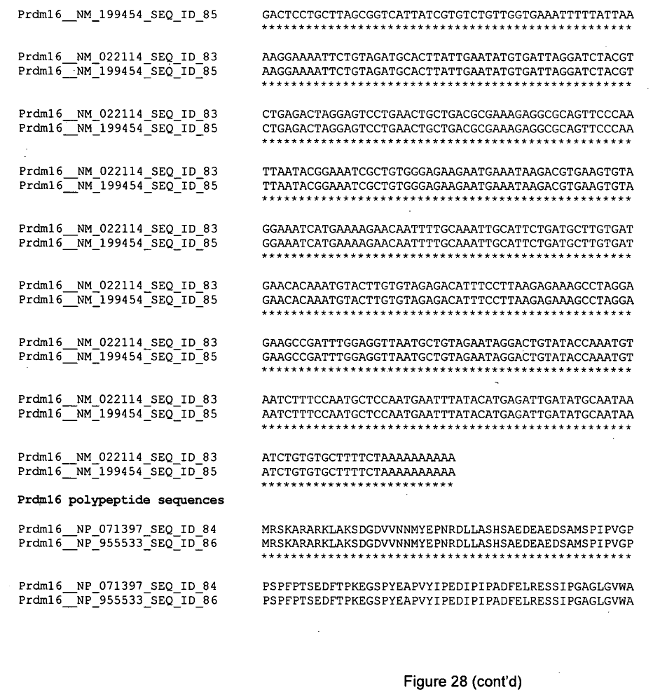

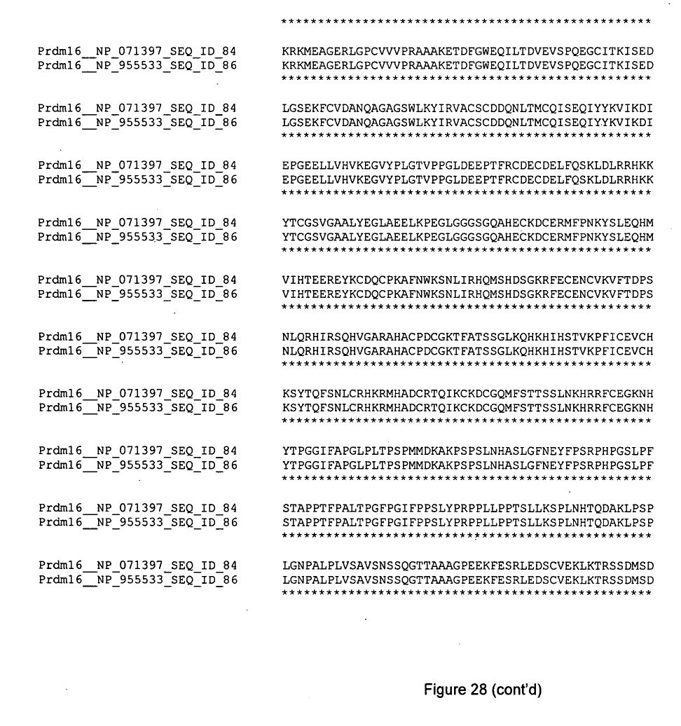

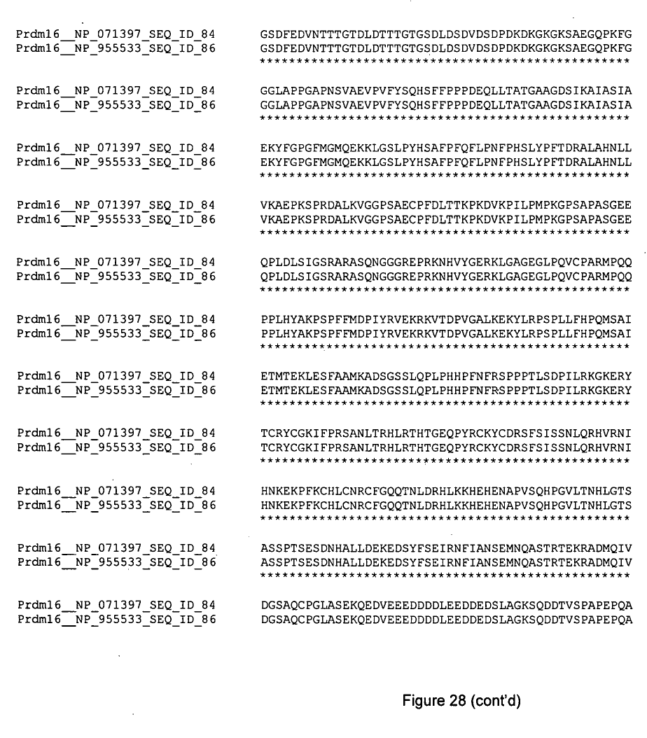

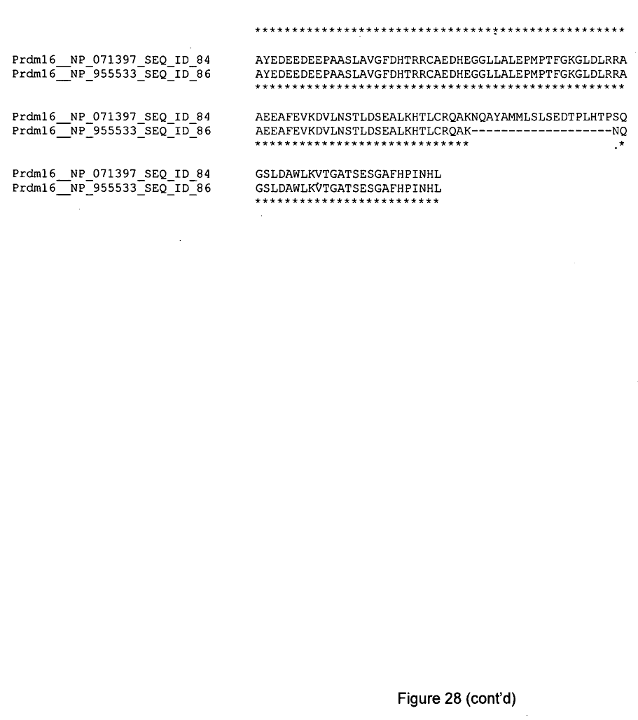

Additional clonal analyses of hematopoietic tissues (bone marrow, blood and thymus) derived from selected recipients sacrificed at 20 weeks post-transplantation confirmed the multi-potentiality and clonality of repopulation, thus indicating that the newly identified genes (nuclear or asymmetrical cell division factors) affect HSC self-renewal or proliferation. Data showing the expansion and/or differentiation of cells transduced with nuclear factors as well as asymmetrical cell division regulators (xbp1, trim27, sox4, fos, pbx2, klf10, hes1, hnrpdl, gpsm2, ap2a2 and cbfb) are presented in FIGS. 13 to 26. Similar experiments were performed using HSCs transformed with smarcc1, sfpi1, hmgb1, vps72, tcfec, zfp472, gpx3, erdr1, tmod1, cnbp1, prdm16, hdac1, erdr1, tmod1, cnbp1, prdm16, hdac1 and ski (FIG. 26).

Thus, the following genes for instance were shown to provide competitive advantage to transduced HSC (e.g., increasing their expansion and/or differentiation) (Table II): trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, pml and hdac1 and ski. Among these genes, trim27, xbp1, sox4, hnrpdl, vps72, gpx3, tmod1, cnbp1 and hdac1 promoted multilineage differentiation. Among these genes, trim27, xbp1, sox4, smarcc1, hnrpdl, vps72, klf10, ap2a2, gpsm2 and gpx3 promoted multiclonal expansion.

### EXAMPLE 9

### Materials and Methods

### Retroviral Vectors

Generation of MSCV-Hoxb4-IRES-GFP was described before (25) and MSCV-NUP98-HOXA10HD-IRES-GFP (NA10HD) was a gift from Dr. Keith Humphries (26) ORF from each candidate gene was amplified by PCR using primers containing restriction sites (underlined in FIG. 3) and template cDNA (FIG. 3; BC accession numbers come from ATCC, Manassas, Va., USA and AK accession numbers come from Riken DNABook, Japan). Digested amplicons were then subcloned into 1 of 3 modified MSCV-PGK-GFP (pKOF-1, -2 or -3, containing different reading frames) according to the reading frame needed for each candidate and sequenced for correct integrity and reading frame.

### Animals

Recipients were C57BL/6 J (B6) mice that express Ly5.2 and transplant donors were C57B1/6Ly-Pep3b (Pep3b) congenic mice that express Ly5.1. All animals were housed in ventilated cages and provided with sterilized food and acidified water at a specific pathogen-free (SPF) animal facility at the Institute for Research in Immunology and Cancer in Montreal.

### Purification of CD150⁺CD48⁻Lin⁻ and CDI50⁺CD48⁻Lin⁻Kit⁺Sca⁺ Cells

Bone marrow cells were stained with allophycocyanin (APC)-labeled anti-Gr-1, -B220, -Ter 19, and depleted using anti-APC magnetic beads and AUTO-MACS system (Becton-Dickinson, San Jose, Calif., USA). Depleted cells were then stained with fluorescein isothiocyanate (FITC)-labeled anti-CD48, and phycoerythrin (PE)-labeled anti-CD150 for CRAs, or in addition to PE-Cy7-labeled c-Kit and PE-Cy5-labeled Sca for q-RT-PCR (BioLegend, San Diego, Calif.). Sorting was performed on a FACSAria system® (Becton-Dickinson, San Jose, Calif., USA).

### Retroviral Infection, Cell Culture and Transplantation

Generation of retrovirus-producing GP+E-86 cells were performed as previously described (9), in 96-well plate, producing one different candidate gene/well. 1500 CD150⁺CD48⁻Lin⁻ sorted Ly5.1⁺ cells/well were cocultured with irradiated (1500 cGy of ¹³⁷Cs gamma radiation) GP+E-86 virus producer cells during 5 days in Dulbecco's modified Eagle's medium (DMEM) supplemented with 15% fetal bovine serum (FBS), 10 ng/mL human interleukin-6 (IL-6), 6 ng/mL murine interleukin-3 (IL-3), 100 ng/mL murine stem cell factor (SF), and 6 μg/ml polybrene, 10 μg/ml ciprofloxacin and 10⁻⁴M β-mercaptoethanol. After trypsinization, ⅜ of each well was prepared for transplantation of 2 sublethally irradiated (800 cGy of ¹³⁷Cs gamma radiation) B6 mice (⅛ per mouse) along with 2×10⁵ whole bone marrow Ly5.2⁺ competitor/helper cells per mouse (Day 0). Also, ½ of each well was kept in culture for an additional 7 days before being prepared for transplantation of 3 sublethally irradiated (800 cGy of ¹³⁷Cs gamma radiation) B6 mice (¼ per mouse) along with 2×10⁵ whole bone marrow Ly5.2⁺ competitor/helper cells per mouse (Day 7). The remaining ⅛ of each well at Day 0 was kept in culture for an additional 4 days before being analyzed by FACS to assess the infection efficiency based on the proportion of GFP+ bone marrow cells.

### Competitive Repopulation Assay and Flow Cytometry

To determine the contributions of the transplanted donor-derived HSCs to hematopoietic reconstitution at various intervals posttransplantation, 50 μL of blood obtained from the tail vein were incubated with excess ammonium chloride (StemCell Technologies, Vancouver, BC, Canada) to lyse erythrocytes, and the proportions of Ly5.1⁺ white blood cells were determined by flow cytometry using a PE-labeled anti-Ly5.1 antibody, and differentiation analysis were determined on whole bone marrow cells 20 weeks post-transplantation using APC-Cy7-labeled anti-B220, PE-Cy5-labeled anti-CD11b and PE-Cy5.5-labeled anti-CD3ε antibodies. Data were acquired using BD LSR II flow cytometer (BD Biosciences, San Jose, Calif., USA) and analyzed using FlowJo® software (Tree Star Inc., Ashland, Oreg., USA).

### Southern Blot Analysis of Genomic DNA

Genomic DNA from 20 week old mice was isolated with DNAzol® reagent (Invitrogen, Carlsbad, Calif., USA), as recommended by the manufacturer. Southern blot analysis was performed as previously described (9). Unique proviral integrations were identified by digestion of DNA with EcoRI, which cleaves once within the provirus and at various distances within the genome. 15 μg of digested whole genomic DNA was then separated in 1% agarose gel by electrophoresis and transferred to zeta-probe membranes (Bio-Rad, Mississauga, ON, Canada) and a and a 710 bp [32P]dCTP EGFP probe, digested from pEYFP-N1 (Clontech Laboratories Inc., Palo Alto, Calif., USA) with EcoRI/HindIII (Invitrogen, Burlington, ON, Canada), was used to reveal the integration pattern.

### Western Blot Analysis

Protein expression of cloned cDNAs was assessed in retroviral producing cell lines. Protein extracts were obtained from transfected GP+E-86 or BOSC cells grown in 96-well plates by incubation with a 30 uL volume of 133 Laemli ( 1/60 β-mercaptoethanol) solution per well, followed by a 10 min boiling step. Western blots analyses were performed as described (9). A mouse anti-FLAG primary antibody used to reveal the presence of the candidate protein, followed by a goat horseradish peroxidase-conjugated anti-mouse secondary antibody (Biolegend San Diego, Calif.).

### Q-RT-PCR Expression Studies

For gene expression profiles analyses of retrovirally transduced BM cells, co-cultures were initiated as described above, but the number of sorted CD150⁺Sca1⁺cKit⁺CD48⁻Lin⁻ cells plated per well increased to 5000. After 5 days of infection, cells were again harvested using trypsinization and individual well contents resubmitted to cell sorting (FACSAria cell sorter, Becton-Dickinson, San Jose, Calif., USA). Gates were set to positively select for GFP+cells, excluding GP+E-86 retroviral producers by forward- and side-scatter criteria. Cells were directly collected in Trizol™ solution to isolate total RNA, according to the manufacturer's protocol (Invitrogen). Reverse transcription of total RNA was performed using the MMLV-reverse transcriptase (RT) and random hexamers according to manufacturer's guidelines (Invitrogen). Resulting cDNA was pre-amplified using a TaqMan® PreAmp (Applied Biosystems, Foster City, Calif.) algorithm in which candidate genes specific oligos were added to the PreAmp Master mix (final concentration of 50 nM). PCR conditions for the pre-amplification reactions were as follows: 95° C. for 10 minutes, followed by 12 cycles of 95° C./15 sec and 60° C./4 min. The ABI Gene Expression Assay was performed to measure gene expression levels using primer and probe sets from Applied Biosystems (primer and probe sequences are available on request). Q-RT-PCR reactions were done on a high-throughput ABI 7900HT™.

### Fast Real-Time PCR System (Applied Biosystems)

Briefly, the Ct (threshold cycle) values of each gene were normalized to the endogenous control gene β-actin (Applied Biosystems; ρCt=Ct_{target}−Ct_{endogenous}) and compared with the mean of our 3 corresponding empty vectors transduced tissue (calibrator sample) using the <<ΔΔCt>> method (ΔΔCt=ΔCtₛₐₘₚₗₑ−ΔCt_{calibrator}). Relative fold difference (RQ) and ΔCt values are provided in FIG. 10. Q-RT-PCR cycling conditions were 2 minutes at 50° C. and 10 minutes at 95° C., followed by 40 cycles of 15 seconds at 95° C. and 1 minute at 59° C. All reactions were done in triplicate. Average values were used for quantification.

Although the present invention has been described hereinabove by way of specific embodiments thereof, it can be modified, without departing from the spirit and nature of the subject invention as defined in the appended claims.

### REFERENCES

1. L. A. Boyer et al., Cell 122, 947 (Sep. 23, 2005).
2. J. Kim, J. Chu, X. Shen, J. Wang, S. H. Orkin, Cell 132, 1049 (Mar. 21, 2008).
3. J. Antonchuk, G. Sauvageau, R. K. Humphries, Cell 109, 39 (Apr. 5, 2002).
4. J. Zhu, Y. Zhang, G. J. Joe, R. Pompetti, S. G. Emerson, Proc Natl Acad Sci USA 102, 11728 (Aug. 16, 2005).
5. T. Reya et al., Nature 423, 409 (May 22, 2003).
6. J. J. Schuringa, K. Y. Chung, G. Morrone, M. A. Moore, J Exp Med 200, 623 (Sep. 6, 2004).
7. U. Thorsteinsdottir et al., Blood 99, 121 (Jan. 1, 2002).
8. H. Ohta et al., Exp Hematol 35, 817 (May, 2007).
9. J. Krosl, N. Beslu, N. Mayotte, R. K. Humphries, G. Sauvageau, Immunity 18, 561 (April, 2003).
10. S. Amsellem, F. Pflumio, D. Bardinet, B. Izac, P. Charneau, P H. Romeo, A. Dubart-Kupperschmitt and S. Fichelson Nat Med 9(11):1423-7 (November 2003).
11. J. Krosl Hematol J.; 5 Suppl 3:S118-21 (2004).
12. Krosl 2005b.
13. Sauvageau, G., Iscove, N. N., and Humphries, R. K. (2004). In vitro and in vivo expansion of hematopoietic stem cells. Oncogene 23, 7223-7232.
14. S. Cellot et al., Exp Hematol 35, 802 (May, 2007).
15. B. Bhattacharya et al., Blood 103, 2956 (Apr. 15, 2004).
16. R. W. Georgantas, 3rd et al., Cancer Res 64, 4434 (Jul. 1, 2004).
17. N. B. Ivanova et al., Science 298, 601 (Oct. 18, 2002).
18. R. L. Phillips et al., Science 288, 1635 (Jun. 2, 2000).
19. M. Ramalho-Santos, S. Yoon, Y. Matsuzaki, R. C. Mulligan, D. A. Melton, Science 298, 597 (Oct. 18, 2002).
20. M. H. Shim, A. Hoover, N. Blake, J. G. Drachman, J. A. Reems, Exp Hematol 32, 638 (July, 2004).
21. F. Shojaei, L. Gallacher, M. Bhatia, Blood 103, 2530 (Apr. 1, 2004).
22. A. V. Terskikh et al., Proc Natl Acad Sci USA 98, 7934 (Jul. 3, 2001).
23. O. P. Pinto do, E. Wandzioch, A. Kolterud, L. Carlsson, Exp Hematol 29, 1019 (August, 2001).
24. J. Krosl, P. Austin, N. Beslu, E. Kroon, R K. Humphries and G. Sauvageau, Nat Med 9 (11), 1428-32 (November, 2003).
25. Cell. Apr. 5, 2002; 109(1):39-45.
26. Exp Hematol. May 2007; 35(5):817-30.
27. Cheshier, S. H., et al. (1999). Proc Natl Acad Sci USA 96, 3120-3125.
28. Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York.
29. Ausubel, et al. (eds), 1989, Current Protocols in Molecular Biology, Vol. 1, Green Publishing Associates, Inc., and John Wiley & Sons, Inc., New York, at p. 2.10.3

1. A method of increasing the expansion and/or differentiation of a hematopoietic stem cell (HSC) comprising:
(a) increasing the level and/or activity of a polypeptide encoded by at least one gene selected from trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant of said polypeptide, in said cell;
(b) increasing the level of a nucleic acid encoding the polypeptide or functional variant of (a) in said cell; or
(c) any combination of (a) and (b).

2. The method of claim 1, wherein said polypeptide comprises the amino acid sequence set forth in Genbank accession Nos: NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 or 97.

3. The method of claim 1, comprising increasing the level of said nucleic acid in said cell.

4. The method of claim 3, wherein said nucleic acid encodes a polypeptide comprising the amino acid sequence set forth in NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 or 97.

5. The method of claim 3, wherein said nucleic acid comprises the coding region of the nucleotide sequence set forth in NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

6. The method of claim 1, wherein said differentiation is multilineage differentiation and wherein said at least one gene is selected from trim27, xbp1, sox4, hnrpdl, vps72 and gpx3.

7. The method of claim 1, further comprising (a) increasing the level and/or activity of at least one further HSC regulator polypeptide selected from Hoxb4, Hoxa9, Bmi1, NF-YA, β-catenin and STAT5A; (b) increasing the level of a nucleic acid encoding the at least one further HSC regulator polypeptide or functional variant of (a) in said cell; or (c) any combination of (a) and (b).

8. The method of claim 7, wherein said further HSC regulator polypeptide is Hoxb4 and comprises the amino acid sequence set forth in Genbank accession No: NP_{—}076920.

9. The method of claim 1, wherein said expansion is multiclonal expansion and wherein said at least one gene is selected from trim27, xbp1, sox4, smarcc1, hnrpdl, vps72, klf10, ap2a2, gpsm2 and gpx3.

10. The method of claim 3, comprising transfecting or transforming said cell with a vector comprising said nucleic acid.

11. The method of claim 10, wherein said vector is a viral vector.

12. The method of claim 11, wherein said viral vector is an adenoviral vector.

13. A composition for increasing the expansion and/or differentiation of a hematopoietic stem cell (HSC) comprising:
(a) an agent capable of:
(i) increasing the level and/or activity of a polypeptide encoded by at least one gene selected from trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant of said polypeptide, in a cell;
(ii) increasing the level of a nucleic acid encoding the polypeptide or functional variant of (a) in a cell; or
(iii) any combination of (i) and (ii); and
(b) a pharmaceutically acceptable carrier or excipient,
further comprising a further agent capable of:(a) increasing the level and/or activity of a polypeptide encoded by Hoxb4; (b) increasing the level of a nucleic acid encoding the polypeptide or functional variant of (a) in a cell; or (c) any combination of (a) and (b).

14. The composition of claim 13, wherein said further agent is a nucleic acid encoding the amino acid sequence set forth in Genbank accession No: NP_{—}076920.

15. The composition of claim 13, comprising (a) an agent capable of increasing the level of a nucleic acid encoding at least one of trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski; and (b) a pharmaceutically acceptable carrier or excipient.

16. The composition of claim 13, wherein said agent is a nucleic acid encoding at least one of trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant thereof.

17. The composition of claim 13, wherein said nucleic acid encodes a polypeptide comprising the amino acid sequence set forth in NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 or 97.

18. The composition of claim 16, wherein said nucleic acid comprises the coding region of the nucleotide sequence set forth in NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

19. The composition of claim 13, wherein said differentiation is multilineage differentiation and wherein said at least one gene is selected from trim27, xbp1, sox4, hnrpdl, vps72 and gpx3.

20. The composition of claim 13, wherein said expansion is multiclonal expansion and wherein said at least one gene is selected from trim27, xbp1, sox4, smarcc1, hnrpdl, vps72, klf10, ap2a2, gpsm2 and gpx3.

21. The composition of claim 13, wherein said agent is a vector comprising said nucleic acid.

22. The composition of claim 21, wherein said vector is a viral vector.

23. The composition of claim 22, wherein said viral vector is an adenoviral vector.

24. An hematopoietic stem cell transformed or transduced with a vector comprising a nucleic acid encoding at least one of trim27, xbp1, sox4, smarcc1, sfpi1, fos, hmgb1, hnrpdl, vps72, tcfec, klf10, zfp472, ap2a2, gpsm2, gpx3, erdr1, tmod1, cnbp1, prdm16, hdac1, pml and ski, or a functional variant thereof.

25. The hematopoietic stem cell of claim 24, wherein said nucleic acid encodes a polypeptide comprising the amino acid sequence set forth in NP_{—}006501 (SEQ ID NO: 2), NP_{—}005071 (SEQ ID NO: 4), NP_{—}001073007 (SEQ ID NO: 6), NP_{—}003098 (SEQ ID NO: 8), NP_{—}003065 (SEQ ID NO: 10), NP_{—}001074016 (SEQ ID NO: 12), NP_{—}003111 (SEQ ID NO: 14), NP_{—}005243 (SEQ ID NO: 16), NP_{—}002119 (SEQ ID NO: 18), NP_{—}112740 (SEQ ID NO: 20), NP_{—}005988 (SEQ ID NO: 22), NP_{—}036384 (SEQ ID NO: 58), NP_{—}001018068 (SEQ ID NO: 60), NP_{—}001027453 (SEQ ID NO: 62), NP_{—}005646 (SEQ ID NO: 64), NP_{—}694703 (SEQ ID NO: 70), NP_{—}036437 (SEQ ID NO: 72), NP_{—}037428 (SEQ ID NO: 74), NP_{—}002075 (SEQ ID NO: 76), NP_{—}579940 (SEQ ID NO: 78), NP_{—}003266 (SEQ ID NO: 80), NP_{—}003409 (SEQ ID NO: 82), NP_{—}071397 (SEQ ID NO: 84), NP_{—}955533 (SEQ ID NO: 86), NP_{—}004955 (SEQ ID NO: 88), NP_{—}003027 (SEQ ID NO: 90), NP_{—}777480 (SEQ ID NO: 24), NP_{—}775303 (SEQ ID NO: 26), NP_{—}775301 (SEQ ID NO: 28), NP_{—}775300 (SEQ ID NO: 30), NP_{—}733796 (SEQ ID NO: 32), NP_{—}003235 (SEQ ID NO: 34), NP_{—}775302 (SEQ ID NO: 36), NP_{—}775299 (SEQ ID NO: 38) NP_{—}150253 (SEQ ID NO: 40), NP_{—}150243 (SEQ ID NO: 42), NP_{—}150242 (SEQ ID NO: 44), NP_{—}002666 (SEQ ID NO: 46), NP_{—}150252 (SEQ ID NO: 48), NP_{—}150241 (SEQ ID NO: 50), NP_{—}150247 (SEQ ID NO: 52), NP_{—}150250 (SEQ ID NO: 54), NP_{—}150249 (SEQ ID NO: 56), SEQ ID NOs: 93, 94, 95, 96 or 97.

26. The hematopoietic stem cell of claim 24, wherein said nucleic acid comprises the coding region of the nucleotide sequence set forth in NM_{—}006510 (SEQ ID NOs: 1), NM_{—}005080 (SEQ ID NOs: 3), NM_{—}001079539 (SEQ ID NOs: 5), NM_{—}003107 (SEQ ID NOs: 7), NM_{—}003074 (SEQ ID NOs: 9), NM_{—}001080547 (SEQ ID NOs: 11), NM_{—}003120 (SEQ ID NOs: 13), NM_{—}005252 (SEQ ID NOs: 15), NM_{—}002128 (SEQ ID NOs: 17), NM_{—}031372 (SEQ ID NOs: 19), NM_{—}005997 (SEQ ID NOs: 21), NM_{—}012252 (SEQ ID NOs: 57), NM_{—}001018058 (SEQ ID NOs: 59), NM_{—}001032282 (SEQ ID NOs: 61), NM_{—}005655 (SEQ ID NOs: 63), NM_{—}153063 (SEQ ID NOs: 69), NM_{—}012305 (SEQ ID NOs: 71), NM_{—}013296 (SEQ ID NOs: 73), NM_{—}002084 (SEQ ID NOs: 75), NM_{—}133362 (SEQ ID NOs: 77), NM_{—}003275 (SEQ ID NOs: 79), NM_{—}003418 (SEQ ID NOs: 81), NM_{—}022114 (SEQ ID NOs: 83), NM_{—}199454 (SEQ ID NOs: 85), NM_{—}004964 (SEQ ID NOs: 87), NM_{—}003036 (SEQ ID NOs: 89), NM_{—}174886 (SEQ ID NO: 23), NM_{—}173211 (SEQ ID NO: 25), NM_{—}173209 (SEQ ID NO: 27), NM_{—}173208 (SEQ ID NO: 29), NM_{—}170695 (SEQ ID NO: 31), NM_{—}003244 (SEQ ID NO: 33), NM_{—}173210 (SEQ ID NO: 35), NM_{—}173207(SEQ ID NO: 37), NM_{—}033250 (SEQ ID NO: 39), NM_{—}033240 (SEQ ID NO: 41), NM_{—}033239 (SEQ ID NO: 43), NM_{—}002675 (SEQ ID NO: 45), NM_{—}033249 (SEQ ID NO: 47), NM_{—}033238 (SEQ ID NO: 49), NM_{—}033244 (SEQ ID NO: 51), NM_{—}033247 (SEQ ID NO: 53) or NM_{—}033246 (SEQ ID NO: 55).

27. The hematopoietic stem cell of claim 23, wherein said vector is a viral vector.

28. The hematopoietic stem cell of claim 27, wherein said viral vector is an adenoviral vector.

29. The hematopoietic stem cell of claim 24, wherein the vector further comprises a nucleic acid encoding Hoxb4.

30. The hematopoietic stem cell of claim 29, wherein said nucleic acid encodes a polypeptide comprising the amino acid sequence set forth in Genbank accession No: NP_{—}076920.

31. A method for increasing the number of blood cells in a subject comprising administering to said subject the hematopoietic stem cell of claim 24.

32. A method for reconstituting the hematopoietic system or tissue of a subject comprising administering to said subject the hematopoietic stem cell of claim 24.

33. A method for increasing the number of blood cells in a subject comprising administering to said subject the composition of claim 13.

34. A method for reconstituting the hematopoietic system or tissue of a subject comprising administering to said subject the composition of claim 13.

35. A method of increasing the expansion and/or differentiation of a hematopoietic stem cell (HSC) comprising:
(a) increasing the level and/or activity of a polypeptide encoded by at least one gene selected from erdr1, tmod1, cnbp1, prdm16, hdac1 and ski, or a functional variant of said polypeptide, in said cell;
(b) increasing the level of a nucleic acid encoding the polypeptide or functional variant of (a) in said cell; or
(c) any combination of (a) and (b).

36. An hematopoietic stem cell transformed or transduced with a vector comprising a nucleic acid encoding at least one of erdr1, tmod1, cnbp1, prdm16, hdac1 and ski, or a functional variant thereof.
